(19) United States
(12) Patent Application Publication
(10) Pub. No.: US 2025/0339402 A1
(43) Pub. Date: Nov. 6, 2025

(54) EFFECTIVE MEANS TO MODULATE NMDA RECEPTOR-MEDIATED TOXICITY

(71) Applicant: FUNDAMENTAL PHARMA GMBH, Heidelberg (DE)

(72) Inventors: Alexander STRAUB, Wuppertal (DE); Hilmar BADING, Heidelberg (DE); Jing YAN, Heidelberg (DE)

(73) Assignee: FUNDAMENTAL PHARMA GMBH, Heidelberg (DE)

(21) Appl. No.: 18/858,532

(22) PCT Filed: Apr. 24, 2023
(86) PCT No.: PCT/EP2023/060683

§ 371 Date: Oct. 21, 2024

(30) Foreign Application Priority Data
Apr. 22, 2022 (EP) 22169433.4

(51) Int. Cl. A61K 31/381; A61K 31/135; A61K 31/275; A61P 25/28; C07C 211/27; C07C 211/29; C07C 211/35; C07C 255/58; C07C 323/25; C07D 333/28

(52) U.S. Cl. CPC A61K 31/381; A61K 31/135; A61K 31/275; A61P 25/28; C07C 211/27; C07C 211/29; C07C 211/35; C07C 255/58; C07C 323/25; C07D 333/28

(57) ABSTRACT

The present invention relates to compounds inhibiting the toxic activity of extrasynaptic NMDA receptors, in particular by inhibiting the formation of NMDA receptor/TRPM4 complexes. In particular, the present invention relates to diamine-based compounds according to general formula I and their use in medicine, in particular for treating neurological diseases such as neurodegenerative diseases.

The present invention relates to the field of neurodegenerative processes and means to provide protection against the same. In particular, the present invention relates to compounds inhibiting the toxic activity of extrasynaptic NMDA receptors, in particular by inhibiting the formation of NMDA receptor/TRPM4 complexes. More specifically, the present invention relates to diamine based compounds according to general formula I and their use in medicine, in particular for treating neurological diseases such as neurodegenerative diseases.

Neurodegenerative diseases are devastating diseases involving the progressive loss of structure or function of neurons and eventual death of neurons. Neurodegeneration may be acute or slowly progressive, but both types of neurodegeneration often involve increased death signalling by extrasynaptic NMDA receptors caused by elevated extracellular glutamate concentrations or relocalization of NMDA receptors to extrasynaptic sites. NMDA receptors are glutamate- and voltage-gated ion channels that are permeable for calcium. They can be categorized according to their subcellular location as synaptic and extrasynaptic NMDA receptors. The subunit composition of the receptors within and outside synaptic contacts is similar, although, in addition to carrying the common Glutamate Ionotropic Receptor NMDA Type Subunit 1 (GRIN1) subunit, extrasynaptic NMDA receptors contain preferentially the GRIN2B subunit, whereas GRIN2A is the predominant subunit in synaptic NMDA receptors. The cellular consequences of synaptic versus extrasynaptic NMDA receptor stimulation are dramatically different. Synaptic NMDA receptors initiate physiological changes in the efficacy of synaptic transmission. They also trigger calcium signalling pathways to the cell nucleus that activate gene expression responses critical for the long-term implementation of virtually all behavioural adaptations. Most importantly, synaptic NMDA receptors, acting via nuclear calcium, are strong activators of neuronal structure-protective and survival-promoting genes. In striking contrast, extrasynaptic NMDA receptors trigger cell death pathways. Within minutes after extrasynaptic NMDA receptor activation, the mitochondrial membrane potential breaks down, followed by mitochondrial permeability transition. Extrasynaptic NMDA receptors also strongly antagonize excitation-transcription coupling and disrupt nuclear calcium-driven adaptogenomics because they trigger a cyclic adenosine monophosphate (cAMP)-responsive element-binding protein (CREB) shutoff pathway, inactivate extracellular signal-regulated kinase (ERK)-MAPK signalling, and lead to nuclear import of class IIa histone deacetylases (HDACs) and the pro-apoptotic transcription factor Foxo3A. This affects activity regulation of many genes, including brain-derived neurotrophic factor (bdnf) and vascular endothelial growth factor D (vegfd), that are vital for the maintenance of complex dendritic architecture and synaptic connectivity as well as the buildup of a neuroprotective shield. In addition, given the short reach of activated ERK1/2, their shut-off by extrasynaptic NMDA receptors disrupts important local signalling events including dendritic mRNA translation and AMPA (α-amino-3-hydroxy-5-methyl-4-isoxazolepropionic acid) receptor trafficking that controls the efficacy of synaptic transmission. Thus, extrasynaptic NMDA receptor signalling is characterized by the initiation of a pathological triad with mitochondrial dysfunction, deregulation of transcription, and loss of integrity of neuronal structures and connectivity.

Several attempts have been made to use blockers of NMDA receptors for treatments of neurological conditions. In general, the results of clinical studies were disappointing largely because of serious side effects caused by interference of the blockers with the physiological function of synaptically localized NMDA receptors (Ogden and Traynelis, 2011). One notable exception is the NMDA receptor antagonist memantine (Bormann, 1989). Beneficial effects of low-dose treatments with memantine have been observed in several animal models of neurodegeneration, which include Alzheimer's disease (AD), Huntington's disease (HD), amyotrophic lateral sclerosis (ALS), and the experimental autoimmune encephalomyelitis (EAE) model of MS. Moreover, memantine is approved since 2002 by the European Medicines Agency and the US Food and Drug Administration (FDA) for the treatment of AD. The discovery that memantine in a certain concentration range blocks preferentially the toxic extrasynaptic NMDA receptors explains why it is effective in a wide range of neurodegenerative conditions that share toxic extrasynaptic NMDA receptor signalling as a pathomechanism (Bading, J Exp Med. 2017 Mar. 6; 214(3):569-578).

It was only recently discovered, that excitotoxicity requires physical coupling of NMDA receptors and TRPM4, a transient receptor potential channel (Yan et al., Science, 2020 Oct. 9; 370 (6513):eaay3302; see also see WO 2020/079244). The NMDA receptor/TRPM4 interaction is mediated by a 57-amino acid intracellular domain of TRPM4, that is positioned just beneath the plasma membrane. Yan et al. also discovered that said interaction can be inhibited by various means and that these provide protection against excitotoxic cell death in cultured neurons and in vivo in mouse models of neurodegeneration. The means suggested by Yan et al. included peptide derived inhibitors of NMDA receptor/TRPM4 interaction as well as small molecule compounds.

However, while the compounds identified by Yan et al. exhibit inhibitory activity, there is still a need in the art for additional means of selectively inhibiting the NMDA receptor/TRPM4 interaction, thereby attenuating specifically the toxic activity of extrasynaptic NMDA receptors. The problem to be solved by the present invention was thus to provide new, preferably improved means to attenuate extrasynaptic toxic NMDA receptor activity.

This problem is solved by the subject-matter as set forth in the appended claims and in the description below.

As will be shown in the following, the inventors of the present invention have identified new compounds, which surprisingly inhibit NMDA receptor mediated toxicity very effectively and are thus particularly useful candidates for treatment and prevention of diseases involving NMDA receptor mediated cytotoxicity.

Therefore, the present invention relates in a first aspect to a compound according to the following general formula I:
wherein:

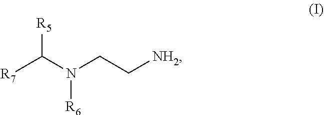

R₇ is selected from

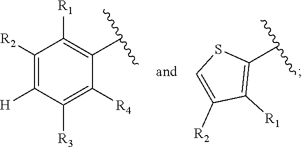

and wherein
R₁, R₂, R₃ and R₄ are each independently selected from H, F, Cl, Br, I, —CN and ethynyl, and wherein at least one of R₁, R₂, R₃ and R₄ is selected from: F, Cl, Br, I, —CN and ethynyl;
R₅ is selected from H, unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl;
R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the proviso that if R₅ is methyl, R₇ is

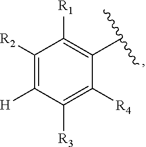

one of R₂ and R₃ is H and the other is Cl and R₁ and R₄ are H, then R₆ is selected from unsubstituted linear C₃-C₆ alkyl, unsubstituted branched C₄-C₆ alkyl, substituted branched or linear C₂-C₆ alkyl, unsubstituted C₄-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl; even preferably with the proviso that if R₅ is methyl, R₇ is

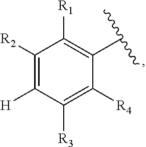

one of R₂ and R₃ is H and the other is F, Cl or —CN and Ri and R₄ are H, then R₆ is selected from unsubstituted linear C₃-C₆ alkyl, unsubstituted branched C₄-C₆ alkyl, substituted branched or linear C₂-C₆ alkyl, unsubstituted C₄-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if Rₛ is methyl, R₇ is

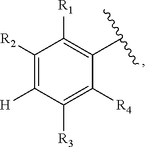

two of R₁, R₂, R₃ and R₄ are Cl, while the other two are H, wherein either R₁ and R₂, R₃ and R₄, R₁ and R₃ or R₂ and R₄ are Cl, then R₆ is selected from unsubstituted branched or linear C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the optional proviso that if R₅ is methyl, R₇ is

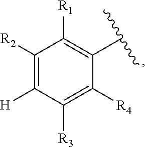

one of R₁ and R₄ is H and the other is F or Br and R₂ and R₃ are H, then R₆ is selected from unsubstituted linear C₃-C₆ alkyl, unsubstituted branched C₄-C₆ alkyl, substituted branched or linear C₂-C₆ alkyl, unsubstituted C₄-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if R₅ is ethyl, R₇ is

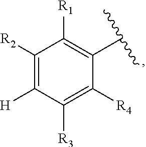

one of R₂ and R₃ is H and the other is Cl and R₁ and R₄ are H, then R₆ is selected from unsubstituted linear C₃-C₆ alkyl, unsubstituted branched C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the further proviso that if R₅ is H and R₇ is

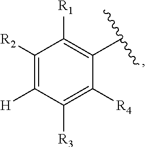

then the compound has one of the following formulas:

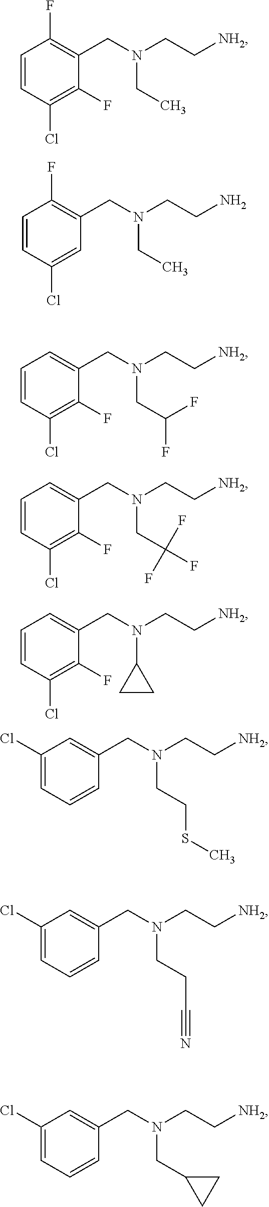

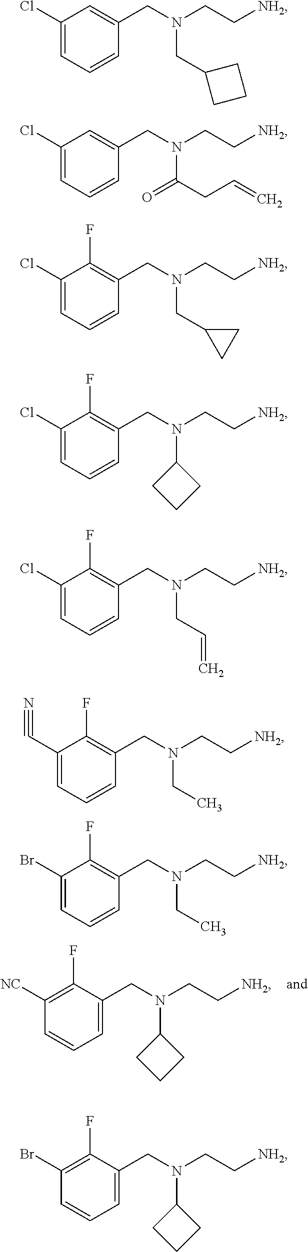

and a pharmaceutically acceptable salt, racemate, (R)- or (S)-enantiomer, hydrate, and/or isotope of any of these compounds. Most preferred is a pharmaceutically acceptable salt of any of the above-mentioned compounds.

The term “unsubstituted alkyl” or “alkyl”, when used without the “substituted” modifier, refers to a monovalent saturated aliphatic group with a carbon atom as the point of attachment, a linear or branched acyclic structure, and no atoms other than carbon and hydrogen. The groups —CH₃ (Me), —CH₂CH₃(Et), —CH₂CH₂CH₃ (n Pr or propyl), —CH(CH₃)₂ (i Pr, iPr or isopropyl), —CH₂CH₂CH₂CH₃ (n Bu), —CH(CH₃)CH₂CH₃ (sec-butyl), —CH₂CH(CH₃)₂ (isobutyl), —C(CH₃)₃ (tert-butyl, t butyl, t Bu or tBu), and —CH₂C(CH₃)₃ (neo-pentyl) are non-limiting examples of alkyl groups. When “alkyl” is used with the “substituted” modifier, and unless specified otherwise, one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂. Preferably, only one hydrogen atom has been replaced. Most preferably, only one hydrogen atom at a terminal carbon atom has been replaced. “Fluoro-substituted” alkyl refers to an alkyl group where one or more hydrogen atoms have been independently replaced by —F. In the case of fluoro-substituted alkyl it is preferred if more than one hydrogen atom has been replaced by —F. Even more preferably, more than two hydrogen atoms have been replaced by —F. Particularly preferred embodiments of fluoro-substituted alkyl are —CF₃, —CHF₂, —CH₂CF₃, —CF₂CH₃, and —CF₂CF₃.

The term “unsubstituted alkenyl” or “alkenyl”, when used without the “substituted” modifier, refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. Preferably, the structure contains only one nonaromatic carbon-carbon double bond, preferably at the terminal end of the structure as in allyl. When “alkenyl” is used with the “substituted” modifier, and unless specified otherwise, one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂. Preferably, only one hydrogen atom has been replaced. Most preferably, only one hydrogen atom at a terminal carbon atom has been replaced. In the case of fluoro-substituted alkenyl it is preferred if more than one hydrogen atom has been replaced by —F. Even more preferably, more than two hydrogen atoms (e.g. 3) have been replaced by —F.

As used herein, the term “unsubstituted cycloalkyl” or “cycloalkyl”, when used without the “substituted” modifier, refers to a monovalent saturated aliphatic group with a carbon atom as the point of attachment, said carbon atom forming part of a single non-aromatic ring structure, no carbon-carbon double or triple bonds, and no atoms other than carbon and hydrogen. Non-limiting examples include: —CH(CH₂)₂ (cyclopropyl), cyclobutyl, cyclopentyl, or cyclohexyl. When “cycloalkyl” is used with the “substituted” modifier, and unless specified otherwise, one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂. Preferably, only one hydrogen atom has been replaced. “Fluoro-substituted” cycloalkyl refers to a cycloalkyl group where one or more hydrogen atoms have been independently replaced by —F. In the case of fluoro-substituted cycloalkyl it is preferred if more than one hydrogen atom has been replaced by —F. Even more preferably, more than two hydrogen atoms (e.g. 3) have been replaced by —F.

As used herein, the term “unsubstituted bicycloalkyl” or “bicycloalkyl”, when used without the “substituted” modifier, refers to a monovalent saturated aliphatic group with a carbon atom as the point of attachment, said carbon atom forming part of two non-aromatic ring structures, no carbon-carbon double or triple bonds, and no atoms other than carbon and hydrogen. A non-limiting example is bicyclo[1.1.1]pentanyl. When “bicycloalkyl” is used with the “substituted” modifier, and unless specified otherwise, one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂. Preferably, only one hydrogen atom has been replaced. In case cycloalkyl is substituted with —F, it is preferred if more than one hydrogen atom has been replaced by —F. Even more preferably, more than two hydrogen atoms (e.g. 3) have been replaced by —F.

As used herein, the term “unsubstituted alkylcycloalkyl”, or “alkylcycloalkyl”, when used without the “substituted” modifier, refers to an alkyl group as defined above with at least two carbon atoms and with a first carbon atom as the point of attachment, wherein a further, terminal carbon atom of the alkyl group forms part of one non-aromatic ring structure. Non-limiting examples include: —CH₂—CH(CH₂)₂ (cyclopropylmethyl), cyclobutylmethyl, cyclopentylethyl, or cyclohexylmethyl. When “alkylcycloalkyl” is used with the “substituted” modifier, and unless specified otherwise, one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂.

Preferably, only one hydrogen atom has been replaced. Most preferably, only one hydrogen atom at a carbon atom of the non-aromatic ring structure has been replaced. In case alkylcycloalkyl is substituted with —F, it is preferred if one or more than one hydrogen atom have been replaced by —F. Even more preferably, more than two hydrogen atoms (e.g. 3) have been replaced by —F.

Examples of compounds according to formula I are compounds according to formulas Ia or Ib:

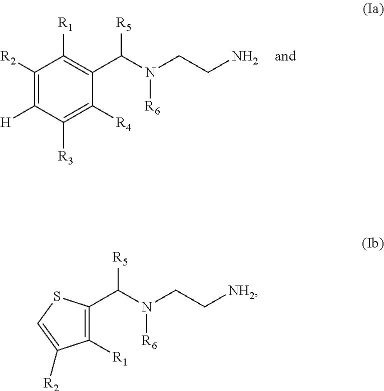

wherein R₁, R₂, R₃, R₄, R₅ and R₆ are defined above for formula I or as more specifically defined below for formula I, Ia and/or Ib, and a pharmaceutically acceptable salt, racemate, (R)- or (S)-enantiomer, hydrate, and/or isotope of any of these compounds.

In preferred embodiments of the invention, R₁, R₂, R₃ and R₄ of the inventive compounds according to formula I, Ia or Ib are each independently selected from H, F, Cl, Br, I and —CN. It will be understood by the skilled person that wherever herein reference is made to “R₁, R₂, R₃ and R₄” this is to be interpreted as reference to “R₁ and R₂” in the context of formula Ib, as there is no R₃ or R₄ in formula Ib. In the context of the aforementioned embodiment, this implies that R₁ and R₂ of formula Ib are each independently selected from H, F, Cl, Br, I and —CN. In some embodiments of the inventive compounds according to formula I, Ia or Ib, at least one of R₁, R₂, R₃ and R₄ is ethynyl, preferably wherein R₂ is ethynyl. In other embodiments of formula I, Ia and Ib, respectively, two of R₁, R₂, R₃ and R₄ are each independently selected from H, F, Cl, Br, I, —CN and ethynyl. In some embodiments of formula Ia, one of R₂ and R₃ is selected from H, F, Cl, Br, I, —CN and ethynyl, while the other is H. In further embodiments of formula Ia, at least two of R₁, R₂, R₃ and R₄ are H and one of R₂ and R₃ is Cl. In some embodiments, R₁ is H or F, preferably F, and R₂ is selected from F, Cl, Br, I, CN and ethynyl, preferably from Cl, Br, CN and ethynyl. In some embodiments of formula Ia, R₄ is H or F, preferably F, and R₃ is selected from F, Cl, Br, I, CN and ethynyl, preferably from Cl, Br, CN and ethynyl. In some embodiments of formula Ia, R₁ is F, R₂ is Cl and R₃ and R₄ are H, or R₁ and R₂ are H, R₃ is Cl and R₄ is F.

R₅ of the inventive compounds according to formula I, Ia or Ib may be selected from H, unsubstituted branched or linear C₁-C₄ alkyl, fluoro-substituted branched or linear C₁-C₄ alkyl, unsubstituted C₃-C₆ cycloalkyl, and fluoro-substituted C₃-C₆ cycloalkyl. In preferred embodiments of the invention, R₅ of the inventive compounds according to formula I, Ia or Ib is selected from unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl. In some embodiments of the inventive compounds according to formula I, Ia or Ib, R₅ is H. In some embodiments of the inventive compounds according to formula I, Ia or Ib, R₅ is methyl, In some embodiments of the inventive compounds according to formula I, Ia or Ib, R₅ is selected from ethyl, isopropyl, —CH₂CF₃, —CF₂CF₃, —CF₂CH₃, —CHF₂, —CF₃, cyclopropyl, fluoro-substituted isopropyl, propenyl, cyclopropyl, cyclobutyl, fluoro-substituted cyclobutyl, and cyclopentyl. In particular in scenarios where R₅ is not H, R₁, R₂, R₃ and R₄ are preferably each independently selected from H, F, Cl, Br and —CN and optionally ethynyl. Similarly, in particular where R₅ is not H, it is also envisioned that at least two of R₁, R₂, R₃ and R₄ are H and one or two, preferably one of R₂ and R₃ is Cl or Br. For example, R₁ may be F, R₂ may be Cl and R₃ and R₄ are H. Another example is where R₁ and R₂ are H, R₃ is Cl and R₄ is F. In particularly preferred embodiments, R₅ is selected from unsubstituted branched or linear C₁-C₄ alkyl, fluoro-substituted branched or linear C₁-C₄ alkyl and unsubstituted propenyl.

Preferably, R₆ of the compounds of the present invention according to formula I, Ia or Ib is not unsubstituted ethyl, i.e. is selected from unsubstituted linear C₃-C₆ alkyl, unsubstituted branched C₄-C₆ alkyl, substituted branched or linear C₂-C₆ alkyl, unsubstituted C₄-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl. In cases where R₆ of the compounds of the present invention according to formula I, Ta or Tb is selected from substituted branched or linear C₂-C₆ alkyl, substituted C₃-C₆ cycloalkyl, substituted C₄-C₈ bicycloalkyl, substituted C₄-C₇ alkylcycloalkyl, and substituted C₃-C₆ alkenyl, the substituents of substituted branched or linear C₂-C₆ alkyl, substituted C₃-C₆ cycloalkyl, substituted bicycloalkyl, substituted C₄-C₇ alkylcycloalkyl, and substituted C₃-C₆ alkenyl are each independently selected from halogen, CN, OH, alkylthio, and alkoxy. Preferably, the substituents of substituted branched or linear C₂-C₆ alkyl, substituted C₃-C₆ cycloalkyl, substituted bicycloalkyl, substituted C₄-C₇ alkylcycloalkyl, and substituted C₃-C₆ alkenyl are each independently selected from F, Cl, CN, —SCH₃ and OH.

Particularly preferred embodiments of the compounds according to the present invention are characterised by R₆ being selected from cyclopropylmethyl, cyclobutylmethyl, cyclopropyl, cyclobutyl, cyclopentyl, bicyclo[1.1.1]pentan-1-yl-, allyl, —CH₂CH₂—S—CH₃, —CH₂CF₂H, —CH₂CF₃, and —CH₂CH₂CN. Most preferably, R₆ is selected from cyclopropyl, cyclobutyl, cyclopentyl, bicyclo[1.1.1]pentan-1-yl-, and allyl. Preferably, the substituent is not present on the carbon atom forming the point of attachment of R₆ to the nitrogen of formula I (or Ia or Ib, respectively).

Particularly preferred combinations of R₅ and R₆ are those where R₅ is not H (e.g. substituted C₁-C₄ alkyl or propenyl), and R₆ is selected from cyclopropyl, cyclobutyl, cyclopentyl, bicyclo[1.1.1]pentan-1-yl-, and allyl.

Examples for compounds of the invention, where R₅ is H, are compounds having one of the following formulas:

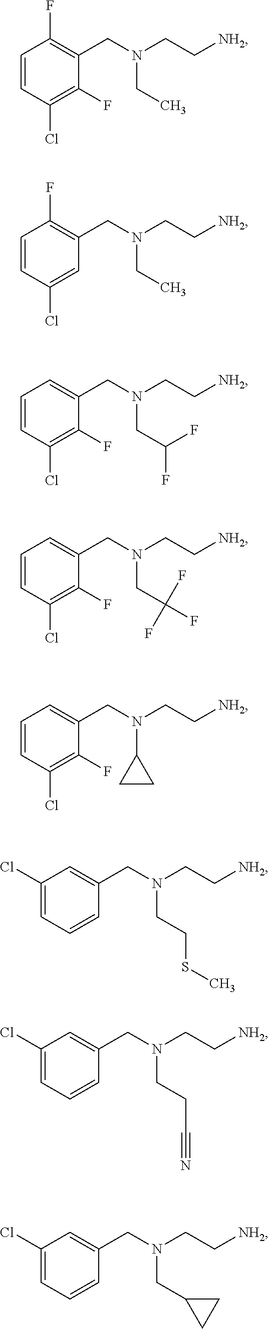

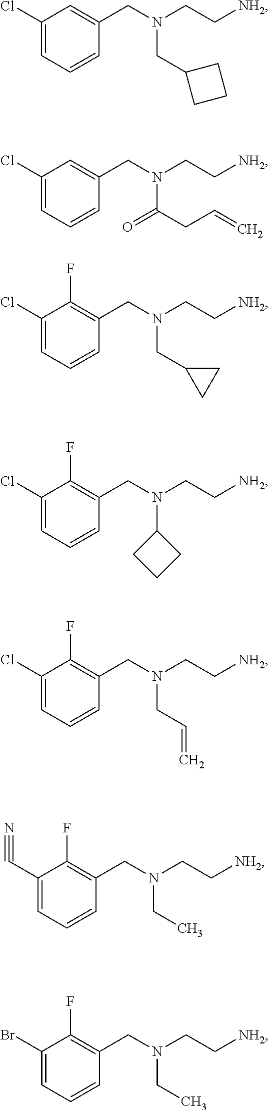

and a pharmaceutically acceptable salt, racemate, (R)- or (S)-enantiomer, hydrate, and/or isotope of any of these compounds. Most preferred is a pharmaceutically acceptable salt of any of the above-mentioned compounds.

Examples for compounds of the invention, where R₅ is not H, are compounds having one of the following formulas:

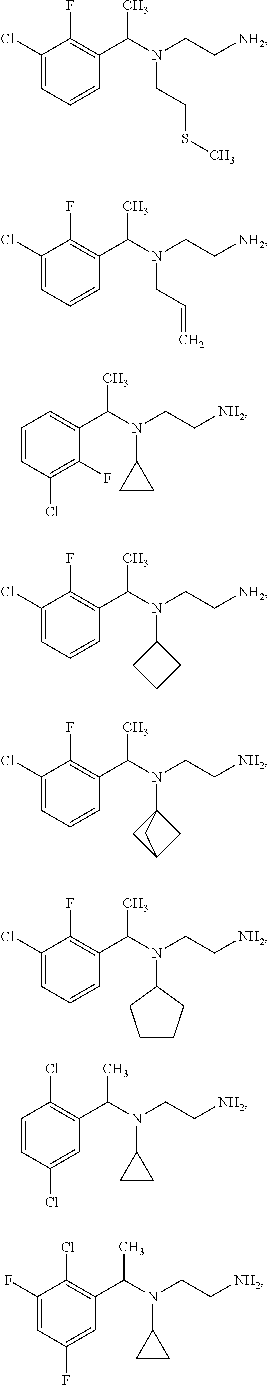

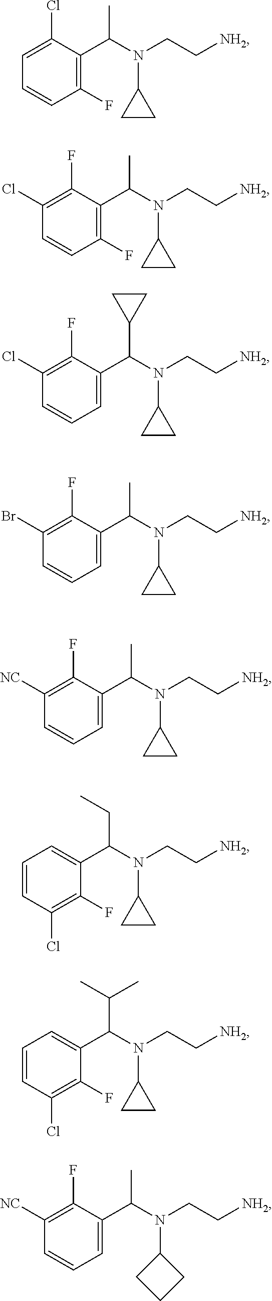

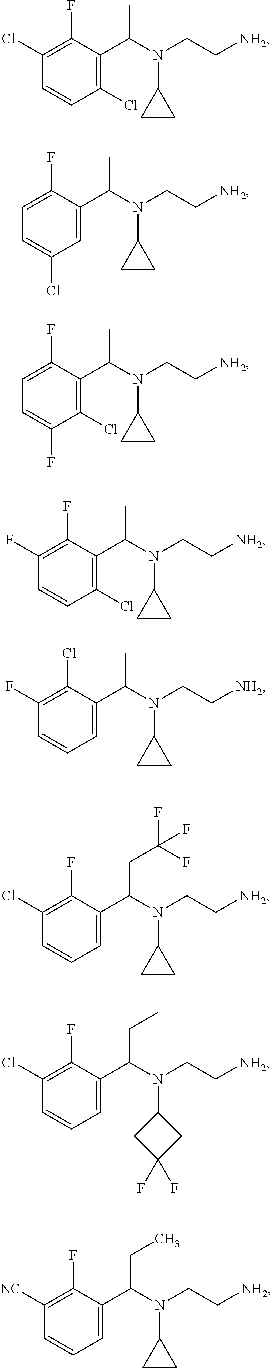

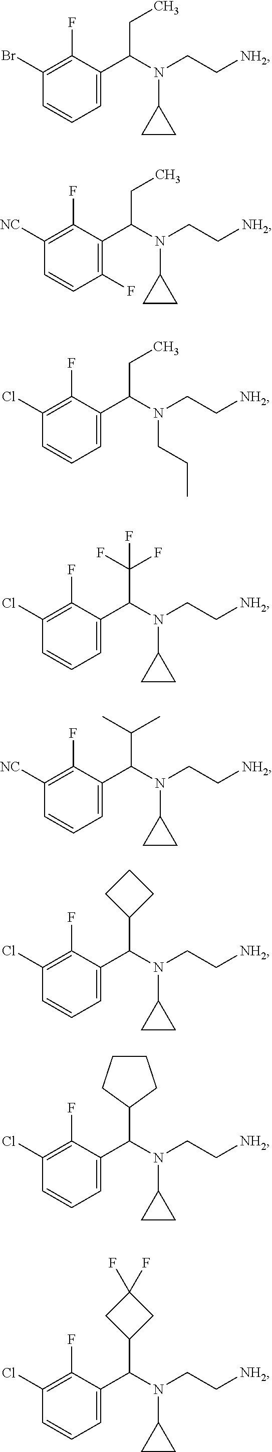

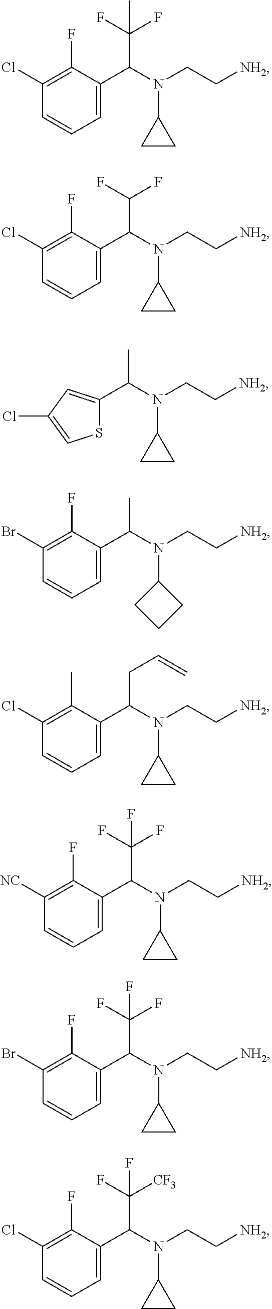

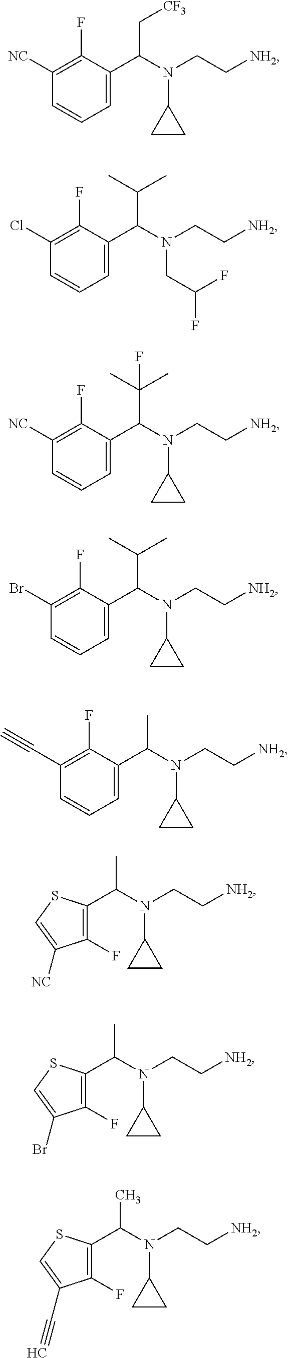

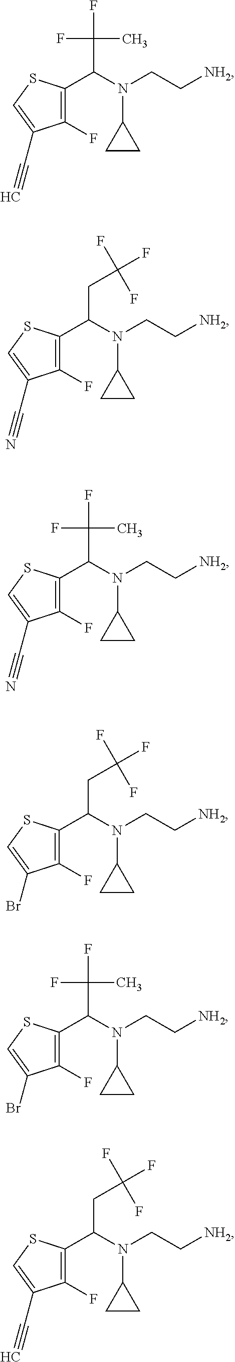 [SMILES: C#Cc1csc(C(N(CCN)C2CC2)C(C)(F)F)c1F ; N#Cc1csc(C(CC(F)(F)F)N(CCN)C2CC2)c1F ; CC(F)(F)C(c1scc(C#N)c1F)N(CCN)C1CC1 ; NCCN(C1CC1)C(CC(F)(F)F)c1scc(Br)c1F ; CC(F)(F)C(c1scc(Br)c1F)N(CCN)C1CC1 ; C#Cc1csc(C(CC(F)(F)F)N(CCN)C2CC2)c1F]

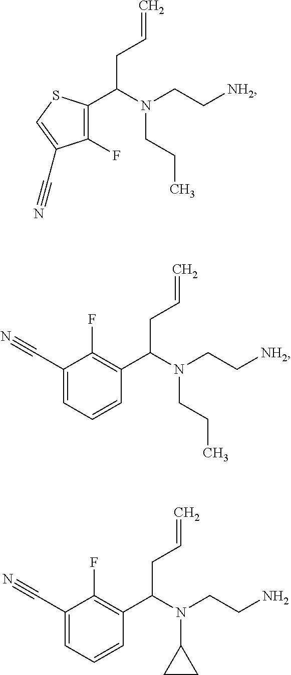 [SMILES: C=CCC(c1scc(C#N)c1F)N(CCC)CCN ; C=CCC(c1cccc(C#N)c1F)N(CCC)CCN ; C=CCC(c1cccc(C#N)c1F)N(CCN)C1CC1]

and a pharmaceutically acceptable salt, racemate, (R)- or (S)-enantiomer, hydrate, and/or isotope of any of these compounds. Most preferred is a pharmaceutically acceptable salt of any of the above-mentioned compounds.

In some embodiments, the compound according to the first aspect of the invention is a compound according to the formula Ia:

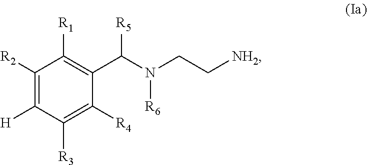

wherein:
R₁, R₂, R₃ and R₄ are each independently selected from H, F, Cl, Br, I, —CN and ethynyl, in particular from H, F, Cl, Br, I and —CN, and wherein at least one of R₁, R₂, R₃ and R₄ is selected from: F, Cl, Br, I, —CN and ethynyl, in particular from F, Cl, Br, I and —CN;
R₅ is selected from H, unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl; in particular from H, unsubstituted branched or linear C₁-C₄ alkyl, fluoro-substituted branched or linear C₁-C₄ alkyl, unsubstituted C₃-C₆ cycloalkyl, and fluoro-substituted C₃-C₆ cycloalkyl;
R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the proviso that if R₅ is methyl, one of R₂ and R₃ is H and the other is Cl and R₁ and R₄ are H, then R₆ is selected from unsubstituted linear C₃-C₆ alkyl, unsubstituted branched C₄-C₆ alkyl, substituted branched or linear C₂-C₆ alkyl, unsubstituted C₄-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the further proviso that if R₅ is methyl, two of R₁, R₂, R₃ and R₄ are Cl, while the other two are H, wherein either R₁ and R₂, R₃ and R₄, R₁ and R₃ or R₂ and R₄ are Cl, then R₆ is selected from unsubstituted branched or linear C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the further proviso that if R₅ is ethyl, one of R₂ and R₃ is H and the other is Cl and R₁ and R₄ are H, then R₆ is selected from unsubstituted linear C₃-C₆ alkyl, unsubstituted branched C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the further proviso that if R₅ is H, then the compound has one of the following formulas:

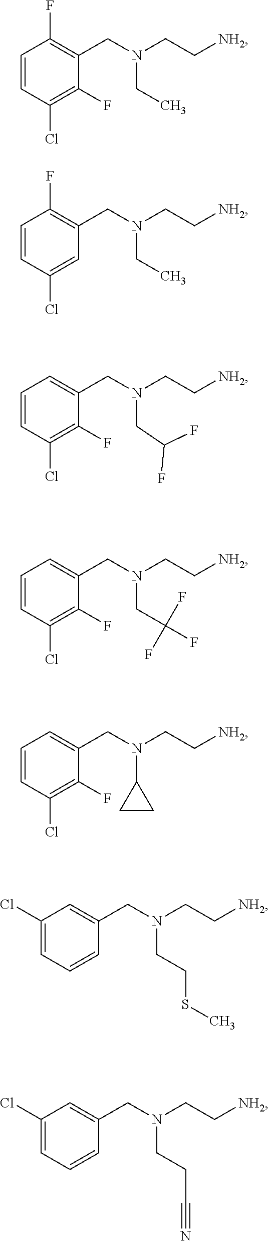

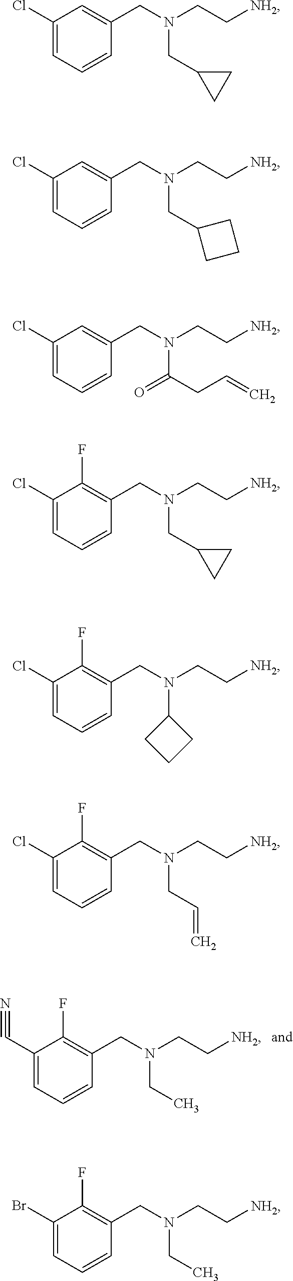

and a pharmaceutically acceptable salt, racemate, (R)- or (S)-enantiomer, hydrate, and/or isotope of any of these compounds. Most preferred is a pharmaceutically acceptable salt of any of the above-mentioned compounds.

In preferred embodiments of the invention, R₅ of the inventive compounds according to formula Ia is selected from unsubstituted branched or linear C₁-C₄ alkyl, fluoro-substituted branched or linear C₁-C₄ alkyl, unsubstituted C₃-C₆ cycloalkyl, and fluoro-substituted C₃-C₆ cycloalkyl. In particular in such scenarios, R₁, R₂, R₃ and R₄ are preferably each independently selected from H, F, Cl, Br and —CN. Similarly, (in particular where R₅ is not H), it is also preferred that at least two of R₁, R₂, R₃ and R₄ are H and one or two, preferably one of R₂ and R₃ is Cl or Br. For example, R₁ may be F, R₂ may be Cl and R₃ and R₄ are H. Another example is where R₁ and R₂ are H, R₃ is Cl and R₄ is F. In particularly preferred embodiments, R₅ is selected from unsubstituted branched or linear C₁-C₄ alkyl, fluoro-substituted branched or linear C₁-C₄ alkyl. Even more preferably, R₅ of the compounds according to the present invention is selected from unsubstituted branched C₃-C₄ or linear C₁-C₄ alkyl, preferably from linear C₁-C₄ alkyl. In some embodiments, R₅ is methyl.

R₆ of the compounds of the present invention according to formula Ia may be selected from substituted branched or linear C₂-C₆ alkyl, substituted C₃-C₆ cycloalkyl, substituted C₄-C₈ bicycloalkyl, substituted C₄-C₇ alkylcycloalkyl, and substituted C₃-C₆ alkenyl, and the substituents of substituted branched or linear C₂-C₆ alkyl, substituted C₃-C₆ cycloalkyl, substituted bicycloalkyl, substituted C₄-C₇ alkylcycloalkyl, and substituted C₃-C₆ alkenyl are each independently selected from halogen, CN, OH, alkylthio, and alkoxy. Preferably, the substituents of substituted branched or linear C₂-C₆ alkyl, substituted C₃-C₆ cycloalkyl, substituted bicycloalkyl, substituted C₄-C₇ alkylcycloalkyl, and substituted C₃-C₆ alkenyl are each independently selected from F, Cl, CN, —SCH₃ and OH. Particularly preferred embodiments of the compounds according to the present invention are characterised by R₆ being selected from cyclopropylmethyl, cyclobutylmethyl, cyclopropyl, cyclobutyl, cyclopentyl, bicyclo[1.1.1]pentan-1-yl-, allyl, —CH₂CH₂—S—CH₃, —CH₂CF₂H, —CH₂CF₃, and —CH₂CH₂CN. Most preferably, R₆ is selected from cyclopropyl, cyclobutyl, cyclopentyl, bicyclo[1.1.1]pentan-1-yl-, and allyl. Preferably, the substituent is not present on the carbon atom forming the point of attachment of R₆ to the nitrogen of formula I, Ia or Ib.

Possible combinations of R₅ and R₆ for compounds of the present invention according to formula Ia are those where R₅ is methyl and R₆ is selected from cyclopropyl, cyclobutyl, cyclopentyl, bicyclo[1.1.1]pentan-1-yl-, and allyl.

In embodiments where the compound according to the present invention is a pharmaceutically acceptable salt, the pharmaceutically acceptable salt is preferably a salt formed with an inorganic or organic acid. Pharmaceutically acceptable salts of a compound according to the invention may be salts of the compounds according to the first aspect of the invention with mineral acids, carboxylic acids or sulphonic acids. Particularly preferred are, for example, salts with hydrochloric acid, hydrobromic acid, sulphuric acid, phosphoric acid, methanesulphonic acid, ethanesulphonic acid, p-toluenesulphonic acid, benzenesulphonic acid, naphthalenedisulphonic acid, formic acid, acetic acid, trifluoroacetic acid, propionic acid, lactic acid, tartaric acid, citric acid, fumaric acid, maleic acid or benzoic acid. Preferred salts are selected from halides, formiates and trifluoroacetates.

Example for an inventive enantiomer is a compound selected from the following structures:

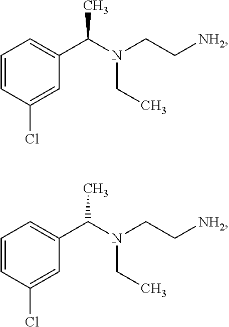

or a pharmaceutically acceptable salt, hydrate, and/or isotope of any of these compounds.

A compound according to the first aspect of the invention is preferably capable of inhibiting extrasynaptic toxic NMDA receptor activity. Suitable tests for assessing NMDA receptor activity are provided in the examples section of this application. A preferred test of assessing inhibition of extrasynaptic toxic NMDA receptor activity is to study said activity in primary neuronal cultures as set out further down below. Preferably, a compound according to the present invention achieves at a concentration of 10 μM least the same level of inhibitory activity (i.e. the same index rating) as (2-aminoethyl)[(3-chlorophenyl)methyl]ethylamine (compound P401 of WO 2020/079244) at 1 μM. Preferably, the inhibitory activity is even greater than the one of compound P401. This is in particular the case where a compound of the first aspect of the invention achieves the same inhibitory activity at a lower concentration than compound P401 (e.g. at 3.0 μM or lower, e.g. at a concentration of 1.0 μM, 0.3 μM, 0.1 μM, or even 0.03 μM). It is also preferred if a compound according to the first aspect of the invention interferes with NMDA receptor/TRPM4 complex formation. A suitable method to assess the capability of disrupting the complex is the co-immunoprecipitation and Western Blot detection method as set out in the examples section of this application.

A compound according to the present invention may be part of a composition according to the present invention. A composition according the present invention comprises at least one compound according to the first aspect of the invention and a suitable pharmaceutical carrier, excipient or diluent.

In a second aspect, the present invention relates to a compound for use in a method for treating or preventing a disease of the human or animal body, wherein the compound is a compound according to the following general formula I:

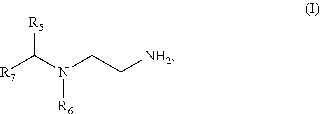

wherein:
R₇ is selected from and

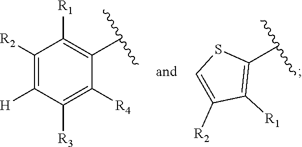

and wherein
R₁, R₂, R₃ and R₄ are each independently selected from H, F, Cl, Br, I, —CN and ethynyl;
R₅ is selected from H, unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl;
R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the proviso that if one of R₁ and R₄ is H and the other is Cl, and R₂, R₃, and R₅ are H, then R₆ is selected from unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if one of R₂ and R₃ is H and the other is Br, Cl or I, and R₁, R₄, and R₅ are H, then R₆ is selected from unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
or wherein the compound is a pharmaceutically acceptable salt, racemate, (R)- or (S)-enantiomer of a compound according to formula I.

The compound for use according to the second aspect of the invention may be compounds according to formulas Ta or Tb:

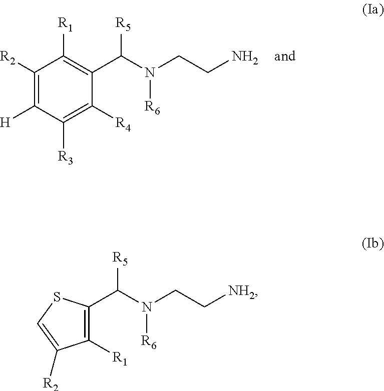

wherein R₁, R₂, R₃, R₄, R₅ and R₆ are as defined herein for formula I, Ia or Ib, respectively.

In some of the embodiments, where the compound for use of the second aspect of the invention is a compound according to formulas Ia,

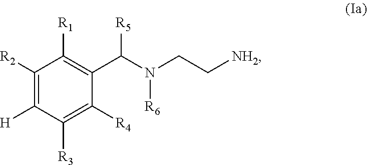

the following applies:
R₁, R₂, R₃ and R₄ are each independently selected from H, F, Cl, Br, I and —CN;
R₅ is selected from H, unsubstituted branched or linear C₁-C₄ alkyl, fluoro-substituted branched or linear C₁-C₄ alkyl, unsubstituted C₃-C₆ cycloalkyl, and fluoro-substituted C₃-C₆ cycloalkyl;
R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the proviso that if one of R₁ and R₄ is H and the other is Cl, and R₂, R₃, and R₅ are H, then R₆ is selected from unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if one of R₂ and R₃ is H and the other is Cl or I, and R₁, R₄, and R₅ are H, then R₆ is selected from unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl; or
wherein the compound is a pharmaceutically acceptable salt, racemate, (R)- or (S)-enantiomer, hydrate, and/or isotope of a compound according to formula I. Most preferred is a pharmaceutically acceptable salt of a compound according to formula I.

In a third aspect, the present invention relates to a method of treating a disease in a subject, the method comprising administering an effective amount of a compound to a subject in need thereof, wherein the compound is a compound according to the following general formula I:

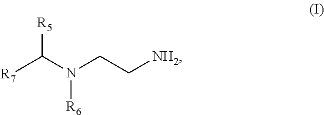

wherein:
R₇ is selected from

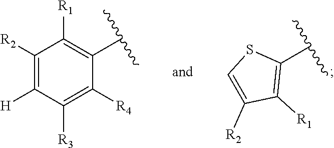

and wherein
R₁, R₂, R₃ and R₄ are each independently selected from H, F, Cl, Br, I, —CN and ethynyl;
R₅ is selected from H, unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl;
R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the proviso that if one of R₁ and R₄ is H and the other is Cl, and R₂, R₃, and R₅ are H, then R₆ is selected from unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if one of R₂ and R₃ is H and the other is Br, Cl or I, and R₁, R₄, and R₅ are H, then R₆ is selected from unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
or wherein the compound is a pharmaceutically acceptable salt, racemate, (R)- or (S)-enantiomer of a compound according to formula I.

The compound used in the method of the third aspect of the invention may be a compound according to formula Ia or Ib,
i.e.

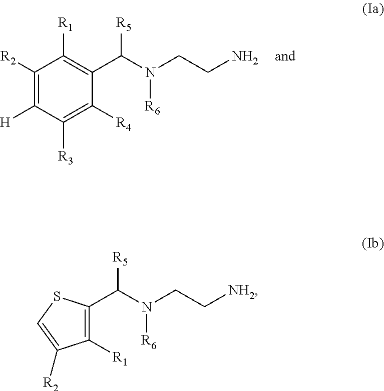

wherein R₁, R₂, R₃, R₄, R₅ and R₆ are as defined herein for formula I, Ia and/or Ib.

In some of the embodiments, the compound used in the method of the third aspect of the invention may be a compound according to formula Ta,

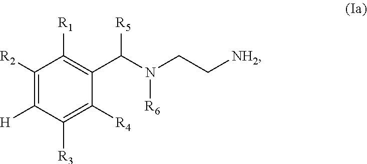

wherein:
R₁, R₂, R₃ and R₄ are each independently selected from H, F, Cl, Br, I and —CN;
R₅ is selected from H, unsubstituted branched or linear C₁-C₄ alkyl, fluoro-substituted branched or linear C₁-C₄ alkyl, unsubstituted C₃-C₆ cycloalkyl, and fluoro-substituted C₃-C₆ cycloalkyl;
R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the proviso that if one of R₁ and R₄ is H and the other is Cl, and R₂, R₃, and R₅ are H, then R₆ is selected from unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if one of R₂ and R₃ is H and the other is Cl or I, and R₁, R₄, and R₅ are H, then R₆ is selected from unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl; or
wherein the compound is a pharmaceutically acceptable salt, racemate, (R)- or (S)-enantiomer, hydrate, and/or isotope of any of these compounds. Most preferred is a pharmaceutically acceptable salt of a compound according to formula I.

The compound for use according to the second aspect of the invention, just as the compound used in the method of the third aspect of the invention, may be a compound according to formula I (or a pharmaceutically acceptable salt, racemate, (R)- or (S)-enantiomer thereof), wherein R₇ is

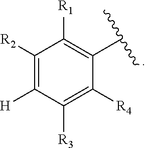

### Alternatively, R₇ is

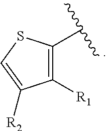

In some embodiments, R₁, R₂, R₃ and R₄ are each independently selected from H, F, Cl, Br and —CN. In some embodiments, at least one of R₁, R₂, R₃ and R₄ is ethynyl, preferably wherein R₂ is ethynyl. In some embodiments, two of R₁, R₂, R₃ and R₄ are each independently selected from H, F, Cl, Br, I, —CN and ethynyl. In some embodiments where R₇ is

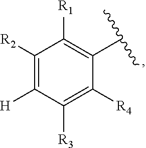

one of R₂ and R₃ is selected from H, F, Cl, Br, I, —CN and ethynyl, while the other is H. In other embodiments where R₇ is

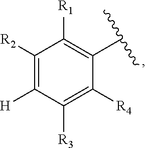

at least two of R₁, R₂, R₃ and R₄ are H and one of R₂ and R₃ is Cl. In some embodiments, R₁ is H or F, preferably F, and R₂ is selected from F, Cl, Br, I, CN and ethynyl, preferably from Cl, Br, CN and ethynyl. In some embodiments of formula Ia, R₄ is H or F, preferably F, and R₃ is selected from F, Cl, Br, I, CN and ethynyl, preferably from Cl, Br, CN and ethynyl. In some embodiments where R₇ is

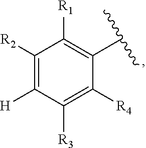

R₁ is F, R₂ is Cl and R₃ and R₄ are H; or R₁ and R₂ are H, R₃ is Cl and R₄ is F.

The compound for use according to the second aspect of the invention or the compound used in the method of the third aspect of the invention may be a compound according to formula I, Ia or Ib (or a pharmaceutically acceptable salt, racemate, (R)- or (S)-enantiomer thereof), wherein R₅ is selected from unsubstituted branched or linear C₁-C₄ alkyl, fluoro-substituted branched or linear C₁-C₄ alkyl, unsubstituted C₃-C₆ cycloalkyl, and fluoro-substituted C₃-C₆ cycloalkyl. In some embodiments, R₅ is H or methyl, as exemplified in the examples. In other embodiments, R₅ is selected from ethyl, isopropyl, —CH₂CF₃, —CF₂CF₃, —CF₂CH₃, —CHF₂, —CF₃, cyclopropyl, fluoro-substituted isopropyl, propenyl, cyclopropyl, cyclobutyl, fluoro-substituted cyclobutyl, and cyclopentyl, as also exemplified in the examples.

The compound for use according to the second aspect of the invention or the compound used in the method of the third aspect of the invention may be a compound according to formula I, Ia or Ib (or a pharmaceutically acceptable salt, racemate, (R)- or (S)-enantiomer thereof), wherein R₆ is selected from substituted branched or linear C₂-C₆ alkyl, substituted C₃-C₆ cycloalkyl, substituted C₄-C₈ bicycloalkyl, substituted C₄-C₇ alkylcycloalkyl, and substituted C₃-C₆ alkenyl, and wherein the substituents of substituted branched or linear C₂-C₆ alkyl, substituted C₃-C₆ cycloalkyl, substituted bicycloalkyl, substituted C₄-C₇ alkylcycloalkyl, and substituted C₃-C₆ alkenyl are each independently selected from F, Cl, CN, OH, alkylthio, and alkoxy, preferably are each independently selected from selected from F, Cl, CN, SCH₃ and OH. In particularly preferred embodiments, R₆ is selected from cyclopropylmethyl, cyclobutylmethyl, cyclopropyl, cyclobutyl, cyclopentyl, bicyclo[1.1.1]pentan-1-yl-, allyl, —CH₂CH₂—S—CH₃, —CH₂CF₂H, —CH₂CF₃, and —CH₂CH₂CN. Most preferably, R₆ is selected from cyclopropyl, cyclobutyl, cyclopentyl, bicyclo[1.1.1]pentan-1-yl-, and allyl.

Possible combinations of R₅ and R₆ for the second and third aspect of the invention are those where R₅ is methyl and R₆ is selected from cyclopropyl, cyclobutyl, cyclopentyl, bicyclo[1.1.1]pentan-1-yl-, and allyl.

For example: compounds according to the first aspect of the invention are compounds which qualify as compound for use according to the second aspect of the invention or which can be used in the method of the third aspect of the invention. Other suitable examples are compounds selected from the group of compounds consisting of:

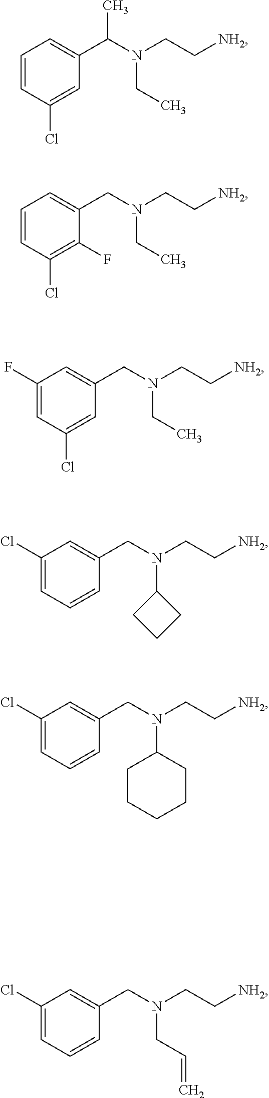 [SMILES: CCN(CCN)C(C)c1cccc(Cl)c1 ; CCN(CCN)Cc1cccc(Cl)c1F ; CCN(CCN)Cc1cc(F)cc(Cl)c1 ; NCCN(Cc1cccc(Cl)c1)C1CCC1 ; NCCN(Cc1cccc(Cl)c1)C1CCCCC1 ; C=CCN(CCN)Cc1cccc(Cl)c1]

and a pharmaceutically acceptable salt, racemate, (R)- or (S)-enantiomer, hydrate, and/or isotope of any of these compounds. Most preferred is a pharmaceutically acceptable salt of any of any of these compounds.

As already mentioned above for the compounds according to the first aspect of the invention: In embodiments where the compound for use according to the second aspect of the invention or the compound to be used in the method of the third aspect of the invention is a pharmaceutically acceptable salt, then the pharmaceutically acceptable salt is preferably a salt formed with an inorganic or organic acid. Pharmaceutically acceptable salts of a compound according to the invention may be salts of the compounds according to the first aspect of the invention with mineral acids, carboxylic acids or sulphonic acids. Particularly preferred are, for example, salts with hydrochloric acid, hydrobromic acid, sulphuric acid, phosphoric acid, methanesulphonic acid, ethanesulphonic acid, p-toluenesulphonic acid, benzenesulphonic acid, naphthalenedisulphonic acid, formic acid, acetic acid, trifluoroacetic acid, propionic acid, lactic acid, tartaric acid, citric acid, fumaric acid, maleic acid or benzoic acid. Most preferably the salt is selected from halides, formiates and trifluoroacetates.

Just like a compound according to the first aspect of the invention, a compound for use according the second aspect of the invention or any compound to be used in the method of the third aspect of the invention is preferably capable of inhibiting extrasynaptic toxic NMDA receptor activity and/or interferes with NMDA receptor/TRPM4 complex formation. For suitable tests reference is made to the corresponding passage above for the compounds according to the first aspect of the invention.

Compounds according to the first aspect of the invention or for use according to the second aspect of the invention or used in the context of the third aspect of the invention can be produced for example, and without being limited thereto, as set out in the examples and figures of the present application. tert-butyl-N-[2-(alkylamino)ethyl]carbamates F such as e.g. tert-butyl N-[2-(cyclopropylamino)ethyl]carbamate (CAS578706-31-7) are known from the literature and can be prepared in known ways, for example by alkylating an alkylamine with 2-(tert-butoxycarbonylamino)ethyl bromide (cf. WO2013062065), by reductive amination of N-Boc-2-aminoacetaldehyde (CAS89711-08-0) analogous to WO2003066621, or of ketones or aldehydes with tert-butyl-N-(2-aminoethyl)carbamate (CAS 57260-73-8) similar to JP2010064982. Tertiary amines C are obtained by reductive amination of ketones A with the resulting secondary amines F. Aldehydes R_{6c}—CHO or ketones R₆ₐ(R_{6b})CO can be converted into N-substituted benzylic amines B by reductive amination with benzylic amines E. (FIG. 1 C). The order of the reaction sequence is also interchangeable. In this case, E first gives D and then C (FIG. 1C). N-substituted benzylic amines B are also obtained by reductive amination of aryl ketones A with amines R₆—NH₂. (FIG. 1 A) By renewed reductive amination with N-Boc-2-aminoacetaldehyde (CAS89711-08-0), for example analogous to WO2003066621 or by alkylation with halides such as tert-butyl (2-bromoethyl)carbamate (CAS 39684-80-5) or reagents such as tert-butyl 2,2-dioxo-1,2λ6,3-oxathiazolidine-3-carboxylate, tertiary amines C are obtained, from which the compound according to formula I, in particular Ia according to the invention can be obtained after the BOC protective group has been eliminated. (FIG. 1A). The order of the reaction sequence is also interchangeable. In this case, A first gives D and then C (FIG. 1A). For the removal of the BOC group, all usual methods such as HCl/MeOH, HCl/EtOAc, TFA/DCM, hexafluoroisopropanol or all other acids are suitable. As reducing agents and catalysts for reductive aminations there are various options, e.g. NaBH₄, NaBH₃CN, NaBH(OAc)₃/TFA, PMHS/SnCl₂×2H₂O, Pd/C/Et₃SiH, PhSiH₃/Bu₂SnCl₂, Ph₂SiH₂/[RuCl₂ (p-cymene)]₂/MS4A, PhSiH₃/Cu(OAc)₂, Et₃SiH/InCl₃, NaBH₄/Ti(i-PrO)₄, HCOOH/Pd—C, H₂/Pd—C, or B₁₀H₁₄. Ethers such as THF, 2-methyl-THF, dioxane or Bu₂O, alcohols such as MeOH, EtOH, trifluoroethanol, ethylene glycol, TAME, diglyme, propanol or isopropanol, acetonitrile, butyronitrile, dichloromethane, 1,2-dichloroethane, 1,1,2,2-tetrachloroethane, acetic acid, DMF, DMAC, water or mixtures thereof may be used as solvents. Alkylations can be carried out, for example, with mesylates, tosylates, trifluoromesylates or halides, like for example, 1-chloro-2-methylsulfanyl-ethane, or of tert-butyl(ethyl)carbamate, preferably tert-butyl (2-bromoethyl)carbamate or with tert-butyl 2,2-dioxo-1,2a6,3-oxathiazolidine-3-carboxylate in solvents such as. e.g. THF, 2-methyl-THF, dioxane, DMF, acetonitrile, butyronitrile, dichloromethane, 1,2-dichloroethane, 1,1,2,2-tetrachloroethane, DMF, DMAC, diglyme, optionally in the presence of a base such as NaH, sodium carbonate, potassium carbonate, sodium methylate, KOtBut, triethylamine or DIPEA. Acylations can be carried out with the appropriate acid chlorides and an inorganic or organic base, with the corresponding acid anhydrides or with organic acids and dehydrating agents such as EDCI/DMAP/DCM. Instead of BOC, all other known protecting groups for amino groups are also contemplated, such as: Cbz, Fmoc, Alloc or phthalimide, which can be cleaved by known methods. These include e.g. H₂/Pd—C, HBr/AcOH, piperidine/DMF, Pd(PPh₃)₄/morpholine/DCM or hydrazine hydrate. Further routes for synthesis of compounds according to the present invention are set out in FigurelD. The required ketones A (FIG. 1E) can be prepared from the corresponding aldehydes O via Grignard reaction with R₅-magnesiumhalides in ethers like diethylether, THF, 2-methyl-THF, dioxane, MTBE to the alcohols P and their subsequent oxidation to aryl ketones A with common oxidants like e.g., Dess Martin periodinane (DMP), Pyridinium chlorochromate or activated MnO₂ in solvents like DCM, toluene, DMF, DMAC. Ketones A can also be prepared by ortho-metalation (cf. Santos et al., Org. Lett. 2021, 23, 7396) of substituted aryl compounds S, which are preferentially substituted with R₁=F and R₂=Cl, Br or CN using strong bases like LDA or TMP(2,2,6,6-Tetramethylpiperidyl) MgCl*LiCl in solvents like THF, 2-methyl-THF or 1,4-dioxane and subsequent reaction of the resulting anion with R5-esters (R5-CO2Et). On the other hand, these intermediate anions can also be reacted with R5-aldehydes (R5-CHO) to give the carbinol P. Trifluormethyl-groups can be introduced to benzaldehydes by reaction with TMSCF3/TBAF to give the trimethylsilylated carbinol T which can be hydrolyzed to the free carbinol P (R5=CF3) which can be oxidised to ketones A (R=CF3) like described above. Trifluormethyl-groups can be introduced to acetophenones by first converting them to the respective triethylsilylenolether U using TESCI/DIEA/n-BuLi in ether solvents like THF and then reacting it with Togni's reagent II in the presence of CuSCN in solvents like DMF, DMAC or NMP to give the trifluoroethyl-phenylketone A (R5=CH2CF3). Acetophenones A (R5=CH₃) can be accessed by reaction of the corresponding arylhalide R with tributyl(1-ethoxyvinyl)stannane, catalyzed by Pd-catalysts like Pd(PPh3)4, Palladium diacetate/2-Dicyclohexylphosphino-2′,6′-dimethoxybiphenyl/TEA, Tris(dibenzylideneacetone)dipalladium/Tri-tert-butylphosphine/Cesium fluoride or Pd(PPh₃)₂Cl₂ in solvents like dioxane, THF, 2-methyl-THF, toluene, benzene, NMP. Amines B can be prepared from benzaldehydes O by first reacting with an amine R6-NH2 in solvents like MeOH using acid catalysis like AcOH to give the Schiff' base imine V. This can be used to introduce R5 groups by reacting with the corresponding grignard reagent like R5-MgBr or other appropriate organometallics. Amines B can be reacted with halogenoacetamide like e.g. bromoacetamide in solvents like MeCN, Butyronitrile, DMF, DMAC, TAME in the presence of a base like potash, soda, TEA or DIPEA to give substituted aminoacetamides G which can be reduced by appropriate reduction reagents like LiAlH4, BH3*Me2S, BH3*THF in solvents like THF or 2-methyl-THF to the desired compound (I). Another route goes by reacting amine B with haloacylhalides like bromoacetylbromide or chloroacetylchloride to give haloacetamides H. These can be aminated either by using potassiumphthalamide yielding protected amines K that can be deprotected to give substituted aminoacetamides M with hydrazine hydrate or methylamine in methanol, ethanol and/or water or using NaBH₄/2-propanol, then acetic acid (J. O. Osby, M. G. Martin, B. Ganem, Tetrahedron Lett., 1984, 25, 2093-2096). Haloacetamides H can also be converted to the corresponding azidoacetamides L using sodiumazide in DMF or DMAC. Azides L can then be reduced to acetamides M using triphenylphosphine in THF/water. These compounds M can be reduced by appropriate reduction reagents like LiAlH4, BH3*Me2S, BH3*THF in solvents like THF or 2-methyl-THF, to the desired compound (I). It is understood that while FIG. 1 illustrates synthesis routes for compounds according to formula Ia, such procedures can be used in analogous manner to generate compounds according to formula Ib as used herein.

The disease to be treated according to the second or third aspect of the invention is preferably a neurological disease, in particular a neurodegenerative disease, or diseases potentially leading to or involving neurodegenerative events, for example infections leading to neurodegenerative events, in particular in the brain. The neurological or neurodegenerative disease may in some embodiments have an inflammatory component, i.e., is a neuroinflammatory disease. The neurodegenerative disease may by a progressive neurodegenerative disease. Preferably, the disease or disorder is selected from the group consisting of stroke, in particular ischemic stroke and hemorrhagic stroke, Alzheimer's disease (AD), amyotrophic lateral sclerosis (ALS), Huntington's disease (HD), traumatic brain injury, post traumatic brain injury, absent-mindedness, age-related loss of memory, aging-related memory decline, progressive nuclear palsy, multiple sclerosis, thalamic degeneration, glutamate induced excitotoxicity, dystonia, epilepsy, optic nerve disease, diabetic retinopathy, glaucoma, pain, particularly neuropathic pain, anti-NMDA receptor encephalitis, dementia, such as post stroke dementia, HIV dementia, Creutzfeldt-Jakob dementia, dementia with Lewy bodies (DLB), dementia with degeneration of the frontal lobes including Pick's disease, dementia with corticobasal degeneration, vascular dementia, microangiopathy, Binswanger's disease, cerebral ischemia, hypoxia, Parkinson's disease, Batten disease, schizophrenia, in particular schizophrenia with dementia, Korsakoff's psychosis, depression, cerebral malaria, toxoplasmosis (due to the risk of toxoplasmosis-associated brain damage), HIV infection/AIDS (due to the risk of HIV)-associated brain damage, and Zika virus infection (due to the possibility of Zika virus-associated brain damage), or any other viral infection potentially leading to neurodegenerative events and corresponding neuronal or brain damage, respectively, such as viral encephalopathy, viral meningitis or SARS-COV2 virus induced encephalitis. In a further embodiment the disease may be a brain tumour, in particular a glioblastoma. Three papers published recently in Nature (see Nature, 2019, Vol 573 pages 499-501) show that glioblastoma cells express NMDA receptors and that their growth is enhanced/stimulated by the activation of NMDA receptors. Therefore, the growth of glioblastoma cells may be inhibited when NMDA receptor signalling is blocked, e.g. by compounds as described herein. In contrast thereto, conventional blockers of NMDA receptors cannot be used in this case because they interfere with the physiological role of NMDA receptors in normal synaptic transmission and cognitive functions such as memory. Due to the general relevance of extrasynaptic NMDA receptor signalling, the compounds disclosed herein are also suitable to treat diseases of the central nervous system such as states of anxiety, tension and depression, sexual dysfunction disorders, and sleep disorders. They may also be used for controlling pathological disturbances of the intake of food, stimulants and addictive substances.

Typically, the method of treatment (in the context of the second or third aspect of the invention) will focus on stopping or slowing down the progression of the disorder. In the alternative, such compound can also be administered in a preventive manner, e.g. in situations where the subject is at (an increased) risk of suffering from a neurological and/or neurodegenerative disease. This includes an acute (increase in) risk (e.g. a thrombotic stroke after surgery) as well as a continuous risk (e.g. due to a genetic and/or familial predisposition for a given neurological and/or neurodegenerative disorder).

The subject to be treated is preferably a mammal, preferably selected from the group consisting of human, mouse, rat, dog, cat, cow, monkey, horse, hamster, guinea pig, pig, sheep, goat, rabbit etc. Most preferably, the subject is a human being.

For the purposes of the second and third aspect of the invention, the person skilled in the art will be readily capable of selecting an appropriate route of administration, depending on the specific disease to be treated or prevented and/or body part to be treated. The route of administration may be, for example, oral, topical, intranasal, parenteral, intravenous, rectal, pulmonal, sublingual, lingual, buccal, transdermal, conjunctival or any other route of administration suitable in the specific context. The compound can also be administered by using an implant releasing the compound over time. For example, if the disease is a cerebrovascular disease, e.g. stroke, then intranasal administration is a preferred route of administration. Intranasal administration is known to the skilled person as being particularly suitable for administering neuroprotective compounds in general, for example in the context of treatment of stroke and stroke induced brain damage. For oral administration, known forms of administration that deliver the active substance rapidly and/or in a modified form are suitable, such as tablets (uncoated and coated tablets, e.g. tablets with enteric coatings or film-coated tablets), capsules, granules, pellets, powders, emulsions, suspensions, solutions and aerosols. Parenteral administration can be carried out bypassing a resorption step (intravenous, intra-arterial, intra-cardiac, intraspinal or intralumbar) or involving resorption (intramuscular, subcutaneous, intracutaneous, percutaneous or intraperitoneal). For parenteral administration, suitable forms of administration include injection and infusion preparations in the form of solutions, suspensions, emulsions, lyophilizates and sterile powders. For the other routes of administration, suitable forms include inhalation medicines (e.g. powder inhalers, nebulisers), nasal drops/solutions, sprays, lingual, sublingual or buccal tablets or capsules, suppositories, ear and eye preparations, vaginal capsules, aqueous suspensions (lotions, shaking mixtures), lipophilic suspensions, ointments, creams, pastes, powder or implants such as stents. The active substances can be transferred to the above-mentioned forms of application in a manner known per se. This is done using inert, non-toxic, pharmaceutically suitable excipients. These include excipients (e.g. microcrystalline cellulose), solvents (e.g. polyethylene glycols), emulsifiers (e.g. sodium dodecyl sulphate), dispersants (e.g. polyvinylpyrrolidone), synthetic and natural biopolymers (e.g. albumin), stabilisers (e.g. antioxidants such as ascorbic acid), colourants (e.g. inorganic pigments such as iron oxides) or taste and/or odour correctors. The active ingredient may also be present in microencapsulated form in one or more of the excipients listed above, if desired. In general, in both human and veterinary medicine, it has been found advantageous to administer the active ingredient of the invention in total amounts of about 0.001 preferably up to about 60, 0.001 up to 40 mg/kg body weight per 24 hours, optionally in the form of several single administrations, to achieve the desired results. A single dose preferably contains the active ingredient of the invention in amounts of about 0.001 to about 30, in particular 0.001 to 20 mg/kg body weight.

In a fourth aspect, the present invention relates to a compound (intermediate) according to the following general formula II:

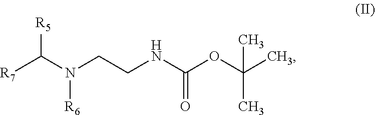

wherein:
R₇ is selected from

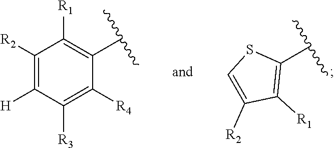

R₁, R₂, R₃ and R₄ are each independently selected from H, F, Cl, Br, I, —CN and ethynyl, and wherein at least one of R₁, R₂, R₃ and R₄ is selected from: F, Cl, Br, I, —CN and ethynyl;
R₅ is selected from H, unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl;
R₆ is selected from H, unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the proviso that if Rₛ is methyl, R₇ is

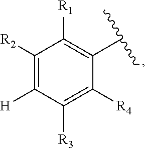

one of R₂ and R₃ is H and the other is Cl and R₁ and R₄ are H, then R₆ is selected from unsubstituted linear C₃-C₆ alkyl, unsubstituted branched C₄-C₆ alkyl, substituted branched or linear C₂-C₆ alkyl, unsubstituted C₄-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if R₅ is methyl, R₇ is

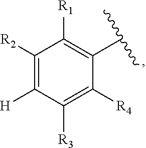

two of R₁, R₂, R₃ and R₄ are Cl, while the other two are H, wherein either R₁ and R₂, R₃ and R₄, R₁ and R₃ or R₂ and R₄ are Cl, then R₆ is selected from unsubstituted branched or linear C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further optional proviso that if R₅ is methyl, R₇ is

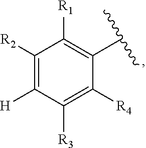

R₁ and R₄ are Cl, one of R₂ and R₃ is H and the other is F, then R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if R₅ is methyl, R₇ is

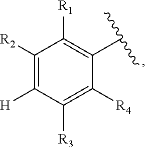

one of R₁ and R₄ is H and the other is F, and R₂ and R₃ are Cl, then R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further optional proviso that if R₅ is methyl, one of R₁ and R₄ is H and the other is Cl, and R₂ and R₃ are H, then R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if R₅ is ethyl, one of R₂ and R₃ is H and the other is Cl and R₁ and R₄ are H, then R₆ is selected from unsubstituted linear C₃-C₆ alkyl, unsubstituted branched C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the further proviso that if R₅ is H, and R₇ is

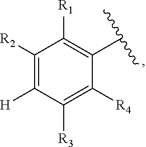

then the compound has one of the following formulas:

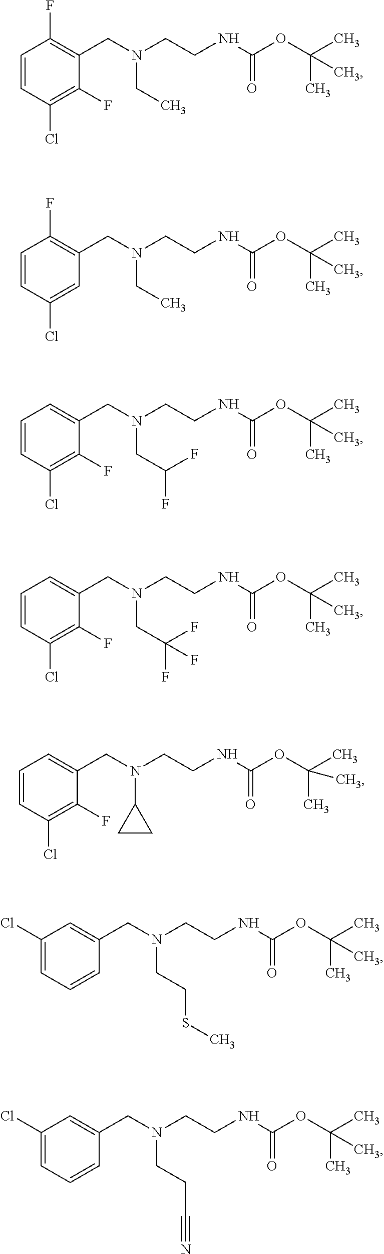

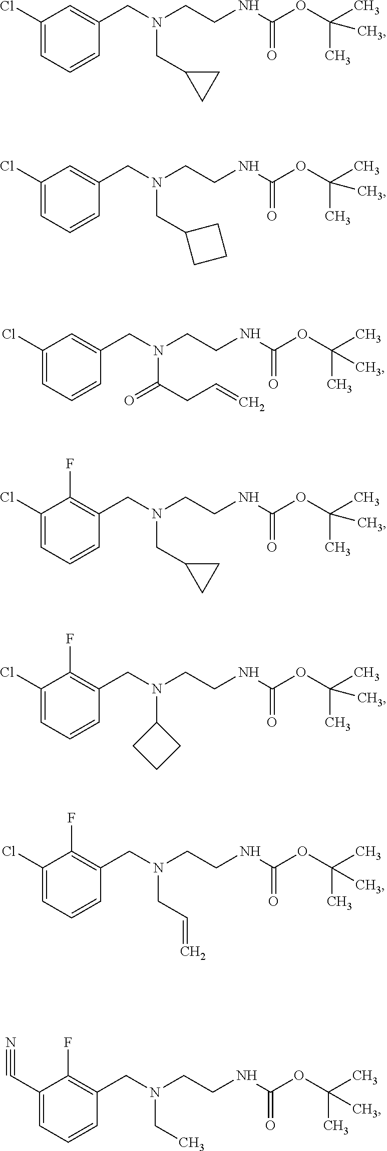

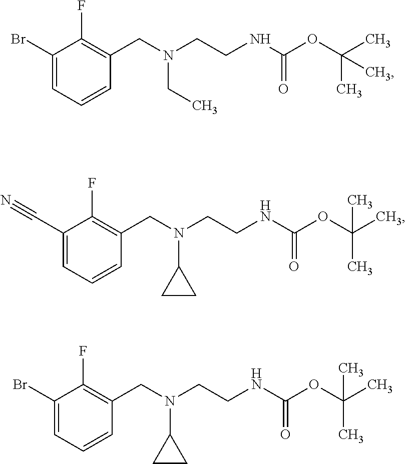 [SMILES: CCN(CCNC(=O)OC(C)(C)C)Cc1cccc(Br)c1F ; CC(C)(C)OC(=O)NCCN(Cc1cccc(C#N)c1F)C1CC1 ; CC(C)(C)OC(=O)NCCN(Cc1cccc(Br)c1F)C1CC1]

and a salt, racemate, (R)- or (S)-enantiomer, hydrate or isotope of any of these compounds

The compound according to the forth aspect of the invention may be a compound according to formula IIa or IIb,
i.e.

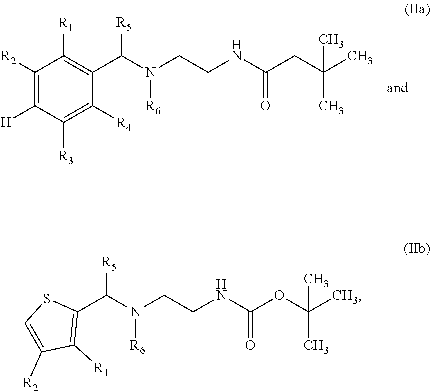

wherein R₁, R₂, R₃, R₄, R₅ and R₆ are as defined herein for formula II, or for formula I, Ia or Ib, respectively.

In some of the embodiments, the compound according to the fourth aspect of the invention may be a corresponding Boc protected compound illustrated in the examples section as direct precursor of a compound according to the first aspect of the invention or for use according to the second or third aspect or the invention.

In some of the embodiments, the compound according to the fourth aspect of the invention is a compound according to formula IIa,

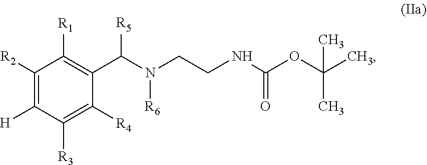

wherein:
R₁, R₂, R₃ and R₄ are each independently selected from H, F, Cl, Br, I and —CN and wherein at least one of R₁, R₂, R₃ and R₄ is selected from: F, Cl, Br, I and —CN;
R₅ is selected from H, unsubstituted branched or linear C₁-C₄ alkyl, fluoro-substituted branched or linear C₁-C₄ alkyl, unsubstituted C₃-C₆ cycloalkyl, and fluoro-substituted C₃-C₆ cycloalkyl;
R₆ is selected from H, unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the proviso that if R₅ is methyl, one of R₂ and R₃ is H and the other is Cl and R₁ and R₄ are H, then R₆ is selected from unsubstituted linear C₃-C₆ alkyl, unsubstituted branched C₄-C₆ alkyl, substituted branched or linear C₂-C₆ alkyl, unsubstituted C₄-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the further proviso that if R₅ is methyl, two of R₁, R₂, R₃ and R₄ are Cl, while the other two are H, wherein either R₁ and R₂, R₃ and R₄, R₁ and R₃ or R₂ and R₄ are Cl, then R₆ is selected from unsubstituted branched or linear C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the further proviso that if R₅ is methyl, one of R₁ and R₄ is H and the other is F, and R₂ and R₃ are Cl, then R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl, with the further proviso that if R₅ is ethyl, one of R₂ and R₃ is H and the other is Cl and R₁ and R₄ are H, then R₆ is selected from unsubstituted linear C₃-C₆ alkyl, unsubstituted branched C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the further proviso that if R₅ is H, then the compound has one of the following formulas:

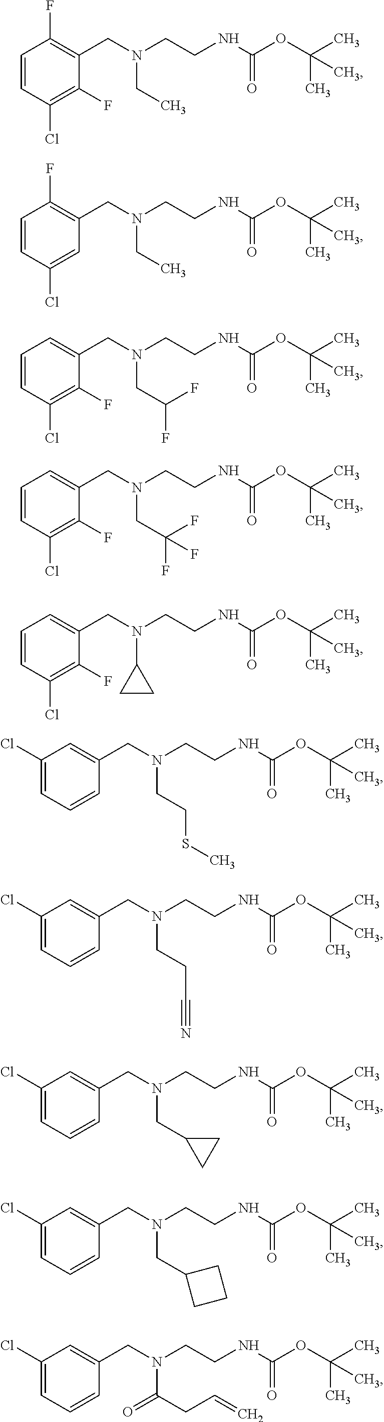

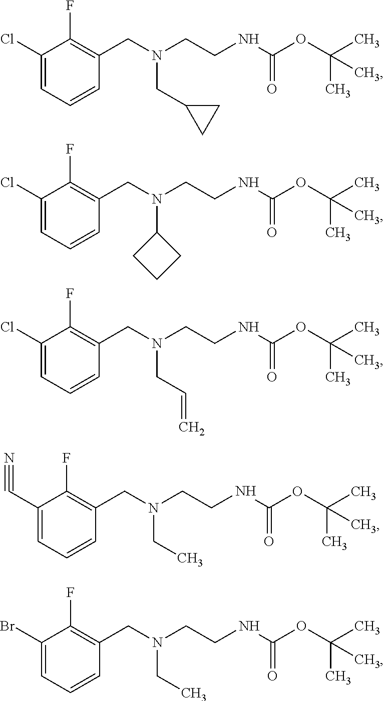

and a salt, racemate, (R)- or (S)-enantiomer, hydrate or isotope of any of these compounds.

The above-mentioned compounds of general formula II, IIa and IIb (intermediates in the manufacture of compounds according to general formula I, Ia and Ib) are particularly useful to manufacture compounds according to the first aspect of the invention or for manufacture of compounds for use according to the second aspect of the invention (or for the method according to the third aspect of the invention). As mentioned above, for the removal of the BOC group, all usual methods such as HCl/MeOH, HCl/EtOAc, TFA/DCM, hexafluoroisopropanol or all other acids are suitable. Preferred embodiments exemplified above for the inventive compounds according to the first aspect of the invention apply likewise to the compounds of the fourth aspect of the invention. Embodiments, which are particularly preferred are those where R₅ is selected from unsubstituted branched or linear C₁-C₄ alkyl, preferably from linear C₁-C₄ alkyl alkyl. Most preferably, R₅ of the intermediates is methyl. Particularly preferred embodiments of the intermediates according to the present invention are characterised by R₆ being selected from cyclopropylmethyl, cyclobutylmethyl, cyclopropyl, cyclobutyl, cyclopentyl, bicyclo[1.1.1]pentan-1-yl-, allyl, —CH₂CH₂—S—CH₃, —CH₂CF₂H, —CH₂CF₃, and —CH₂CH₂CN. Most preferably, R₆ of the intermediates is selected from cyclopropyl, cyclobutyl, cyclopentyl, bicyclo[1.1.1]pentan-1-yl-, and allyl. In the intermediates, R₁, R₂, R₃ and R₄ are preferably each independently selected from H, F, Cl, and —CN. It is also preferred that at least two of R₁, R₂, R₃ and R₄ are H and one or two, preferably one of R₂ and R₃ is Cl. Particularly preferred combinations of R₅ and R₆ are those where R₅ is methyl and R₆ is selected from cyclopropyl, cyclobutyl, cyclopentyl, bicyclo[1.1.1]pentan-1-yl-, and allyl.

In a preferred embodiment, the intermediate compound according to the fourth aspect of the invention is selected from one of the following formulas:

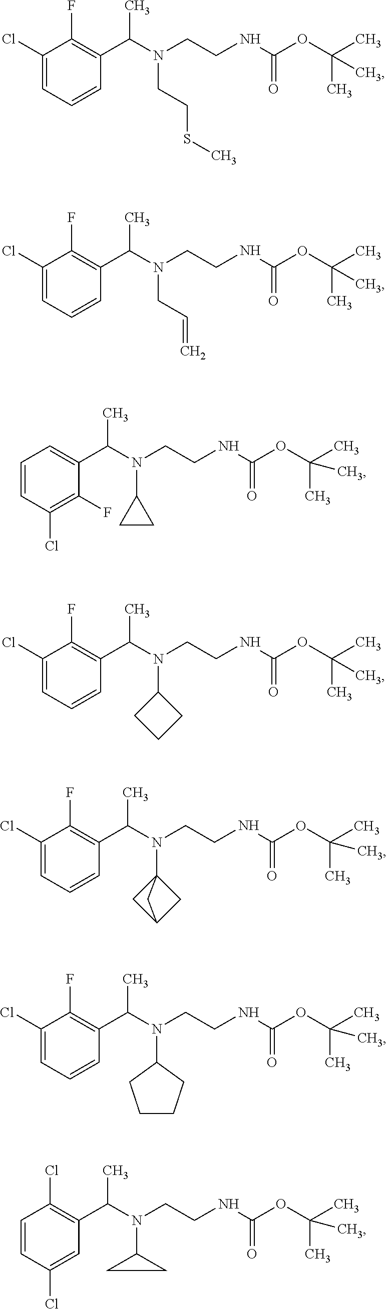

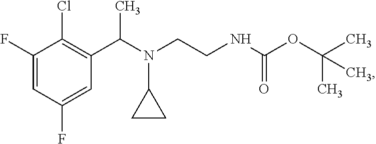 [SMILES: CC(c1cc(F)cc(F)c1Cl)N(CCNC(=O)OC(C)(C)C)C1CC1]

and a salt, racemate, (R)- or (S)-enantiomer, hydrate or isotope of any of these compounds.

In a fifth aspect, the present invention relates to further compounds (intermediates) which are not a Boc protected compound according to formula II, IIa or IIb, but are compounds according to the following general formula III:

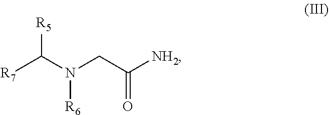

wherein:
R₇ is selected from

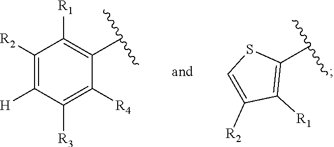

R₁, R₂, R₃ and R₄ are each independently selected from H, F, Cl, Br, I, —CN and ethynyl, and wherein at least one of R₁, R₂, R₃ and R₄ is selected from: F, Cl, Br, I, —CN and ethynyl;
R₅ is selected from H, unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl;
R₆ is selected from H, unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
and a salt, racemate, (R)- or (S)-enantiomer, hydrate or isotope thereof.

The compound according to the fifth aspect of the invention may be a compound according to formula IIIa or IIIb,
i.e.

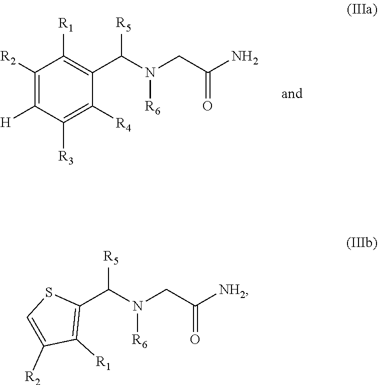

wherein R₁, R₂, R₃, R₄, Rₛ and R₆ are as defined herein for formula III, or for formula I, Ia or Ib, respectively.

In a sixth aspect, the present invention relates to even further intermediates, which do not fall under formula II or III, but which are compounds according to the following general formula IV:

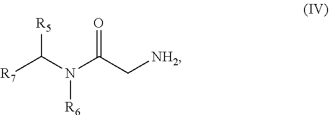

wherein:
R₇ is selected from

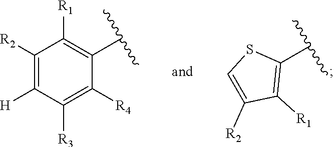

R₁, R₂, R₃ and R₄ are each independently selected from H, F, Cl, Br, I, —CN and ethynyl, and wherein at least one of R₁, R₂, R₃ and R₄ is selected from: F, Cl, Br, I, —CN and ethynyl;
R₅ is selected from H, unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl;
R₆ is selected from H, unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
and a salt, racemate, (R)- or (S)-enantiomer, hydrate or isotope thereof.

The compound according to the sixth aspect of the invention may be a compound according to formula IVa or IVb,
i.e.

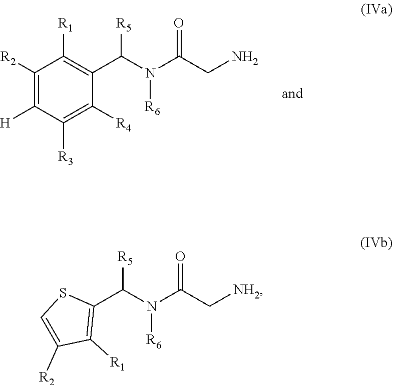

wherein R₁, R₂, R₃, R₄, R₅ and R₆ are as defined herein for formula IV, or for formula I, Ia or Ib, respectively.

In the examples section of the present invention, various direct precursors in the synthesis of a compound according to the first aspect of the invention or of a compound for use according to the second or third aspect of the invention are illustrated in more detail, which are compounds according to formula III or IV. All these intermediates of formula III and IV are specifically contemplated as preferred embodiments of the fifth and sixth aspect of the present invention, respectively.

The term “comprising”, as used herein, shall not be construed as being limited to the meaning “consisting of” (i.e. excluding the presence of additional other matter). Rather, “comprising” implies that optionally additional matter may be present. The term “comprising” encompasses as particularly envisioned embodiments falling within its scope “consisting of” (i.e. excluding the presence of additional other matter) and “comprising but not consisting of” (i.e. requiring the presence of additional other matter), with the former being more preferred.

### FIGURES

In the following a brief description of the appended figures will be given. The figures are intended to illustrate aspects of the present invention in more detail. However, they are not intended to limit the scope of the invention.

FIG. 1 provides various general reaction schemes to illustrate synthesis of compounds used in the present application. Different educts lead to tert-butyloxycarbonyl protected compounds (intermediates C) which are then converted into the final product (formula I); R₆ₐ and R_{6b} are those chains or ring members that form parts of R₆ after reductive amination; analogously, R_{6c}—CHO form the R₆ moiety; 1A) Access to compounds according to the invention starting from aryl ketones or benzaldehydes; 1B) convergent route to compounds of formula I, in particular Ia; 1C) access to compounds according to the invention starting from benzylic amines; 1D) further possible synthesis routes for compounds according to the present invention; 1E) synthesis of ketones A and alcohols P; 1F) introduction of trifluoromethyl groups to benzaldehydes and acetophenones.

FIG. 2 illustrates a quantification of cell survival after glutamate/NMDA treatment in primary cultured neurons. The area above the curve (AAC, shadow region in Vehicle and compound P401 group) were quantified to determine the protection index of other compounds. Compound P401 of WO 2020/079244 (10 μM) provides ˜60% protection and the protection index is defined as 6.0.

FIG. 3 illustrates a quantification of cell survival after H₂O₂ treatment in primary cultured neurons. A) Compound P401 (see WO 2020/079244) provides better protection against H₂O₂ insult compared to FDA-approved ALS drug Riluzole and Edaravone; B) Compound 220 provides better protection against H₂O₂ insult compared to P401 at 10 μM, which is similar to 0.1 μM of compound 220.

FIG. 4 illustrates a quantification of cell survival (%) after glutamate/NMDA treatment in human iPSC-derived prefrontal cortex organoids. Compound 120 provided a superior protection over compound P401 (see WO 2020/079244)

FIG. 5 provides the results of a co-immunoprecipitation of TRPM4 and subunits of NMDA receptor. Lysates from human iPSC-derived brain organoids were precipitated with anti-TRPM4. Both input (5%) and immune-precipitates were separated in SDS-PAGE, transferred, and blotted with antibodies against GluN2A, GluN2B, GluA2, TRPM4, and Tubulin. In presence of Compound 120, the NMDAR/TRPM4 complex was disrupted.

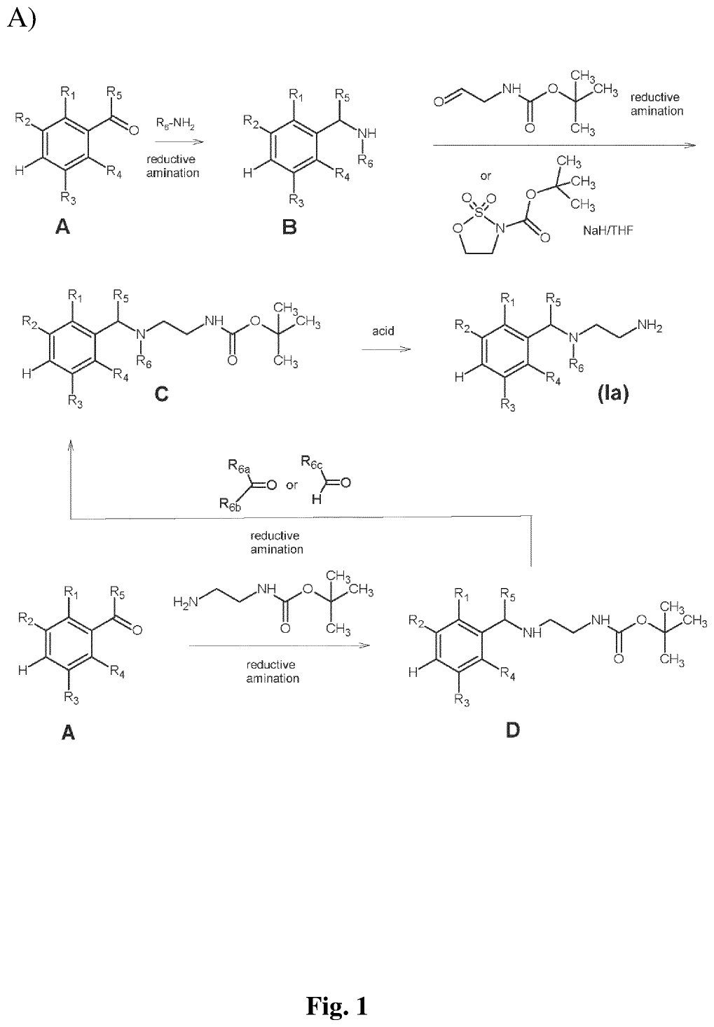

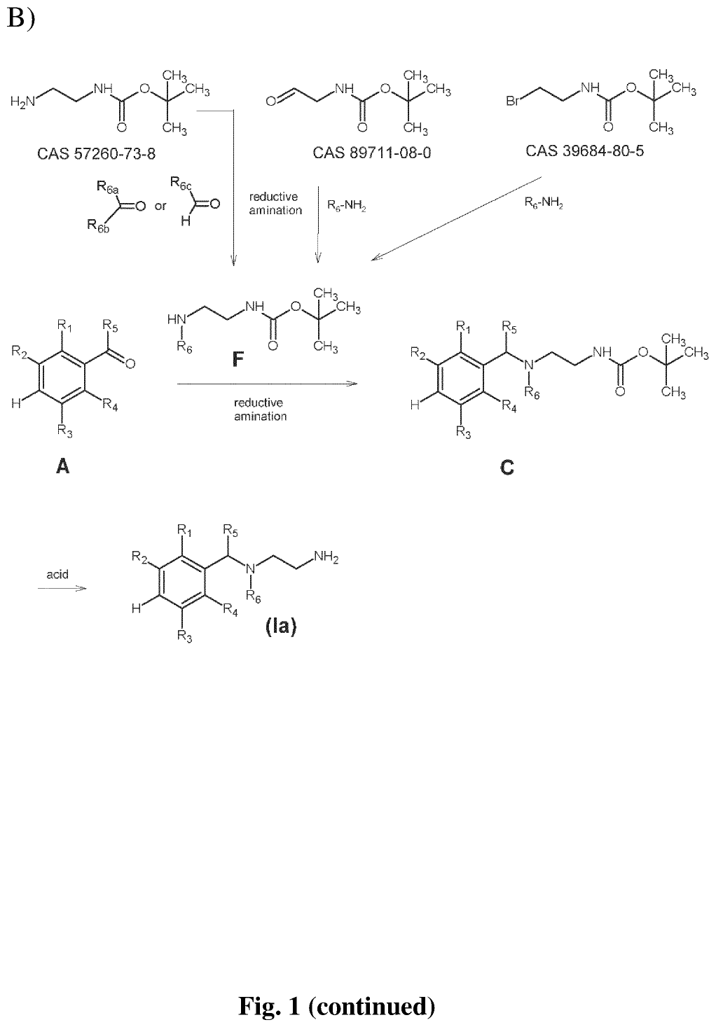

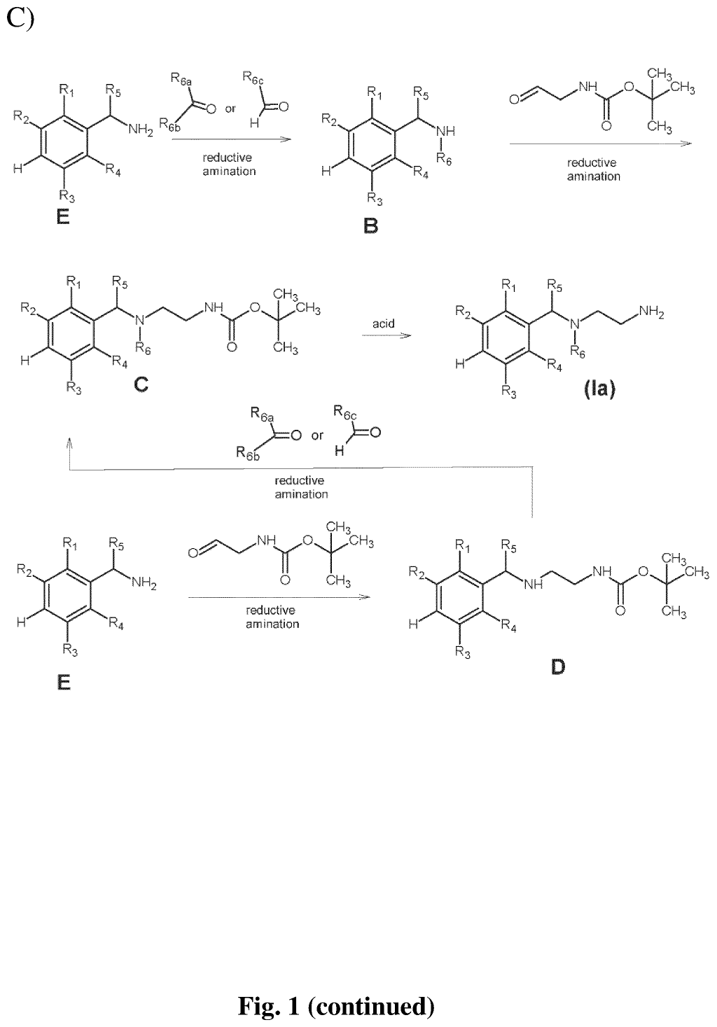

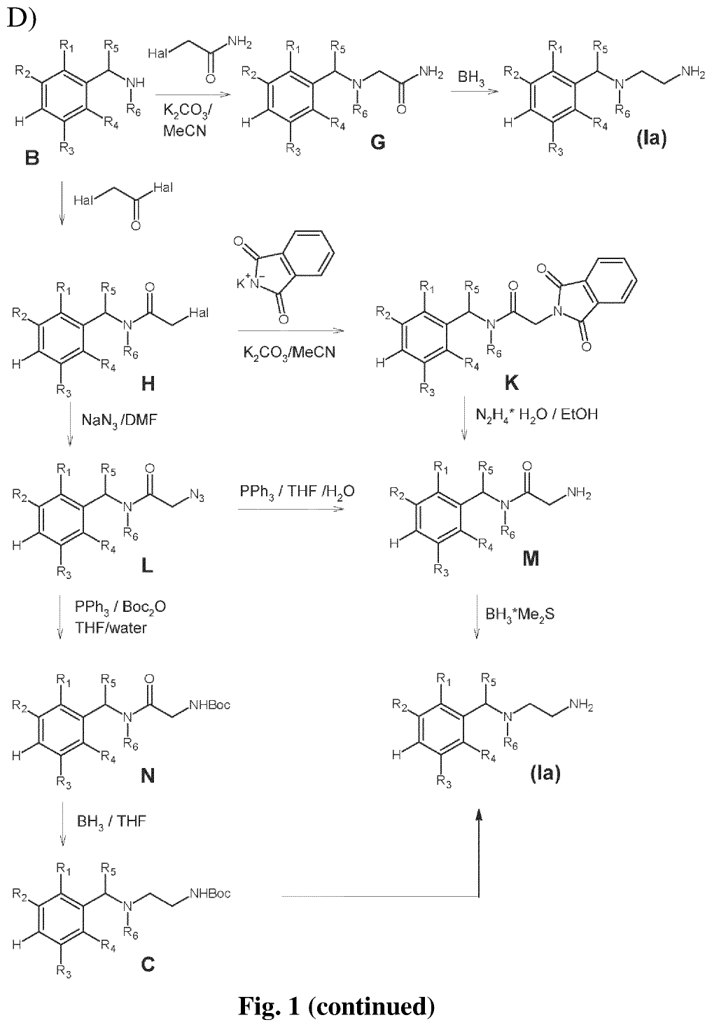

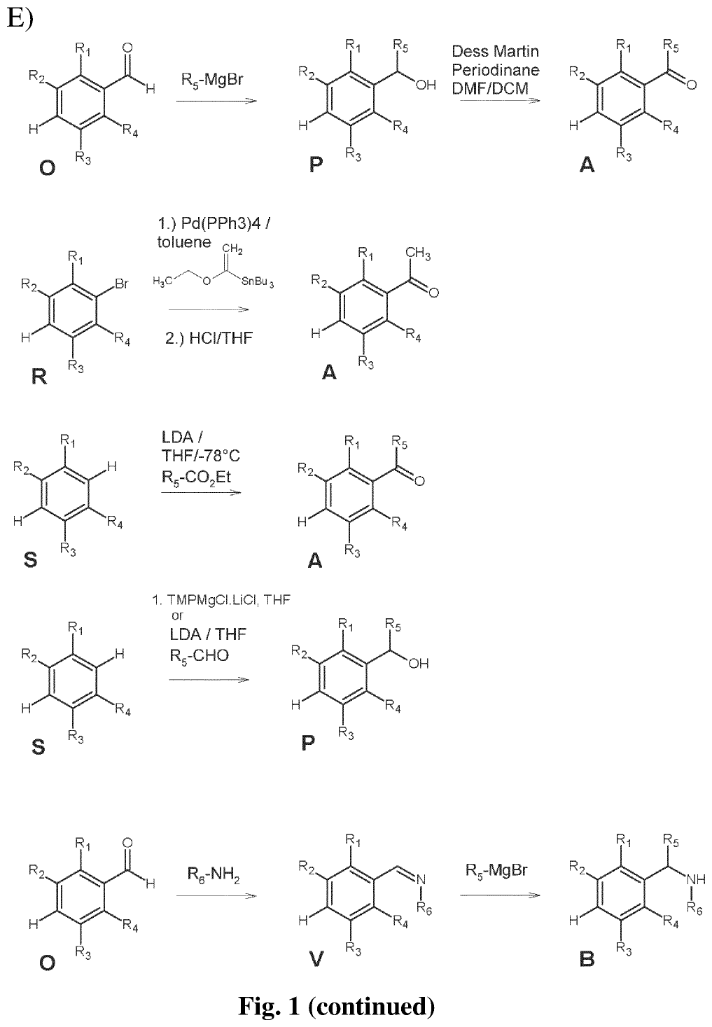

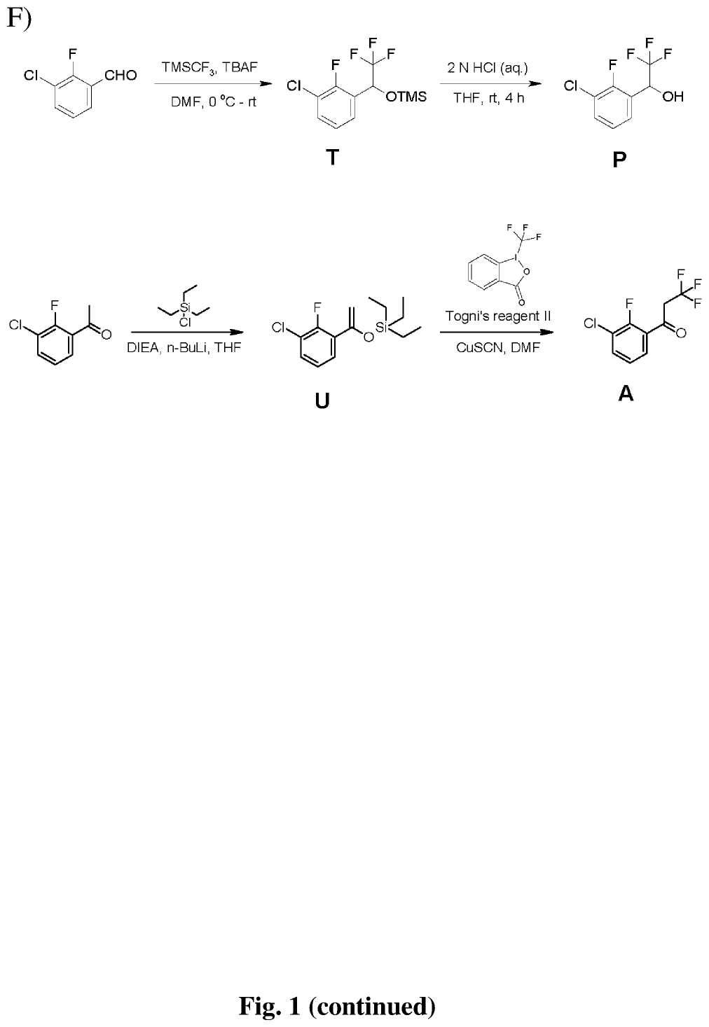

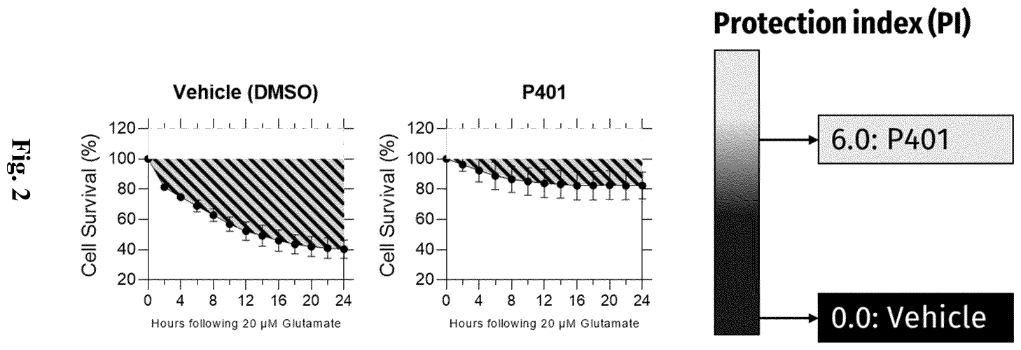

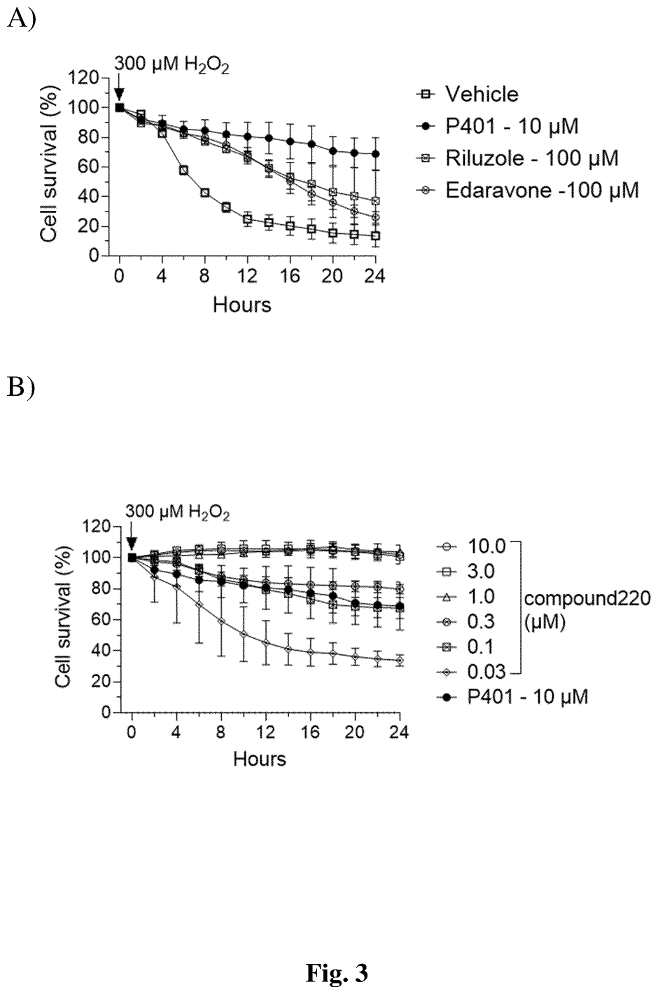

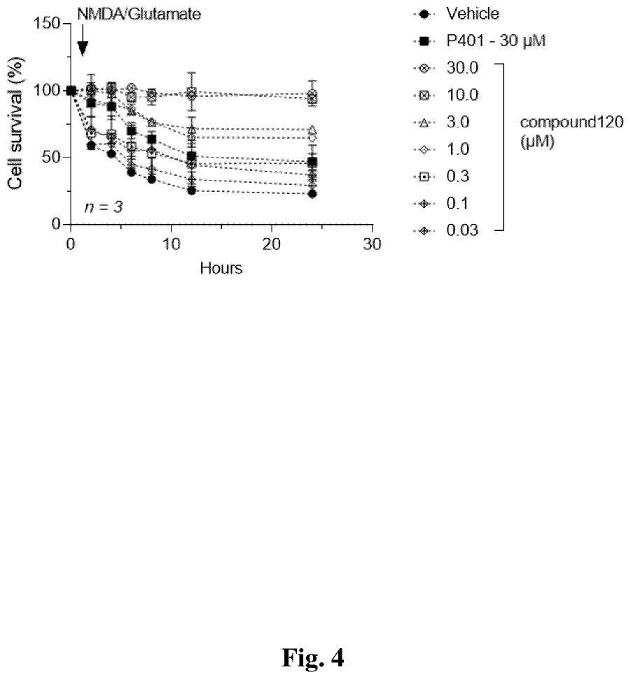

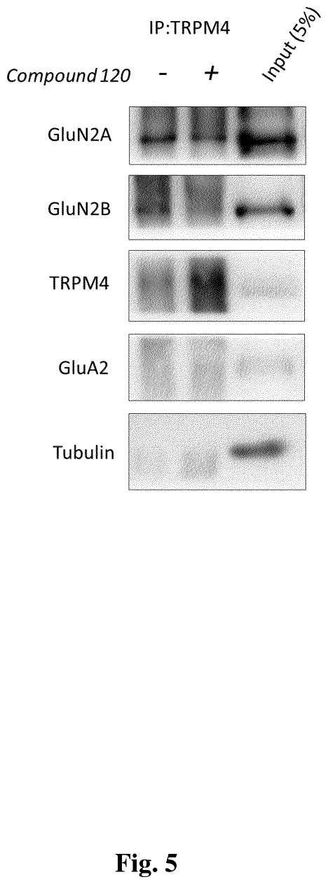

### EXAMPLES

In the following, specific examples illustrating embodiments and aspects of the invention are presented. However, the present invention shall not to be limited in scope by the specific examples described herein. Indeed, various modifications of the invention in addition to those described herein will become readily apparent to those skilled in the art from the foregoing description and the example below. All such modifications fall within the scope of the appended claims.

### Example 1: General Procedure

Compounds used in the context of the present invention for attenuation of extrasynaptic toxic NMDA receptor activity have been prepares in general as follows.

### Reductive Aminations—Preparation of BOC Protected Intermediates C

To a solution of tert-butyl N-(2-aminoethyl)carbamate (100 mg, 624.2 μmol, 98 μL, 1 eq) in MeOH (3 mL) the appropriate benzaldehyde or acetophenone (for expl. Compound 109) derivative (1 eq) was added. The mixture was stirred at 65° C. for 12 h. Then NaBH₃CN (196 mg, 3.1 mmol, 5 eq) was added, and the mixture was stirred at 65° C. for 2 h. Then acetaldehyde (344 mg, 3.1 mmol, 438 μL, 40% purity, 5 eq) (for expl. see Compounds 91, 92, 94, 95, 109) or the appropriate cyclopropancarbaldehyde derivative (for expl. see Compounds 153 and 157) was added and the mixture was stirred at 65° C. for 4 h. The reaction mixture was concentrated, and then diluted with 5 mL EtOAc and washed with H₂O (5 mL×3). The combined organic layers were dried over Na₂SO₄ and concentrated under reduced pressure to give a residue (intermediate C).

### Removal of BOC Group

A solution of intermediate C (637 μmol, 1 eq) in HCl/EtOAc (2 mL, 4 M) was stirred at 20° C. for 0.5 h and LC-MS showed consumption of starting material. The reaction mixture was concentrated to give a residue. The residue was purified by preparative HPLC (HCl condition, column: Phenomenex Luna 80×30 mm×3 um; mobile phase: [water(HCl)-ACN]; B %: 1%-1%, 8 min) to give after evaporation in vacuo the final product (Compound according to formula I) as shown in the table.

According to the general procedure the following compounds have been prepared:

| Compound | Structure | LCMS | H NMR |
|---|---|---|---|
| 91 | 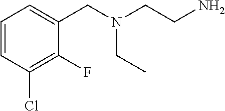 | 231.1 [M + H]⁺ Purity: 100% retention time: 0.849 min method: F | ¹HNMR (400 MHz, DMSO-d₆) δ = 11.67- 11.21 (m, 1H), 8.66-8.14 (m, 3H), 7.94-7.61 (m, 2H), 7.25 (s, 1H), 4.60- 4.31 (m, 2H), 3.31 (br s, 4H), 3.25-3.10 (m, 2H), |
| | 2 HCl | | 1.32 (br t, J = 6.8 Hz, 3H) |
| 92 | 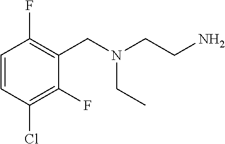 [SMILES: CCN(CCN)Cc1c(F)ccc(Cl)c1F] | 249.1 [M + H]⁺ Purity: 99.15% retention time: 1.260 min method: F | ¹HNMR (400 MHz, Methanol-d₄) δ = 7.71 (dt, J = 5.8, 8.8 Hz, 1H), 7.19 (dt, J = 1.7, 9.0 Hz, 1H), 4.56 (s, 2H), 3.55-3.41 (m, 4H), 3.35 (q, J = 7.2 Hz, 2H), 1.44 (t, J = 7.2 Hz, 3H) |
| | 2 HCl | | |
| 94 | 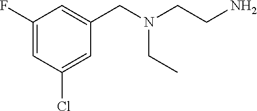 | 231.1 [M + H]⁺ Purity: 100% retention time: 1.110 min method: F | ¹HNMR (400 MHz, DMSO-d₆) δ = 11.81- 11.40 (m, 1H), 8.60-8.18 (m, 3H), 7.52 (br d, J = 3.4 Hz, 3H), 4.52-4.33 (m, 2H), 3.28 (br s, 4H), 3.20- 3.02 (m, 2H), 1.28 (br t, |
| | 2 HCl | | J = 7.1 Hz, 3H) |
| 95 | 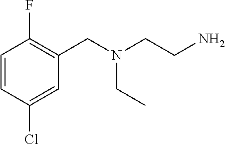 | 231.1 [M + H]⁺ Purity: 100% retention time: 1.639 min method: K | ¹HNMR (400 MHz, DMSO-d₆) δ = 11.51- 11.16 (m, 1H), 8.32 (br s, 3H), 7.97 (br d, J = 2.9 Hz, 1H), 7.67-7.54 (m, 1H), 7.41 (t, J = 9.2 Hz, 1H), 4.41 (br s, 2H), 3.39- 3.05 (m, 6H), 1.30 (br t, J = 6.2 Hz, 3H) |
| | 2 HCl | | |
| 109 | 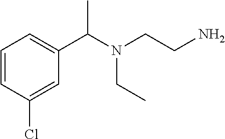 | 227.1 [M + H]⁺ Purity: 100% retention time: 1.222 min method: I | 1H NMR (400 MHz, DMSO-d₆) δ = 11.62- 11.37 (m, 1H), 8.67-8.28 (m, 3H), 7.86 (s, 1H), 7.72 (br d, J = 4.2 Hz, 1H), 7.50 (br s, 2H), 4.69 (br d, J = 6.0 Hz, 1H), 3.49 (br s, 2H), 3.30 (br s, 2H), 3.20- 3.01 (m, 2H), 1.68 (br d, J = |
| | 2 HCl | | 6.5 Hz, 3H), 1.34-1.15 |
| | | | (m, 3H) |
| 153 | 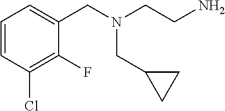 [SMILES: NCCN(Cc1cccc(Cl)c1F)CC1CC1] | 257.0 [M + H]⁺ Purity: 100% retention time: 1.471 min method: B | ¹H NMR (400 MHz, DMSO-d₆) δ = 11.66- 11.48 (m, 1H), 8.50 (br s, 3H), 7.86 (br t, J = 6.7 Hz, 1H), 7.71 (br t, J = 7.3 Hz, 1H), 7.33 (t, J = 7.9 Hz, 1H), 4.68-4.42 (m, 2H), |
| | 2 HCl | | 3.67-3.51 (m, 1H), 3.34 |
| | | | (br s, 3H), 3.12 (br s, 2H), |
| | | | 1.24 (br s, 1H), 0.66 (br d, |
| | | | J = 7.7 Hz, 2H), 0.46 (br s, |
| | | | 2H) |
| 157 | 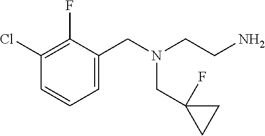 [SMILES: NCCN(Cc1cccc(Cl)c1F)CC1(F)CC1] | 275.0 [M + H]⁺ Purity: 100% retention time: 1.867 min method: K | ¹H NMR (400 MHz, DMSO-d₆) δ = 12.23- 11.54 (m, 1H), 8.75-8.06 (m, 3H), 7.91-7.54 (m, 2H), 7.31 (br t, J = 7.7 Hz, 1H), 4.81-4.43 (m, 6H), 3.57 (br d, J = 4.4 Hz, 2H), 1.41-0.86 (m, 4H) |
| | HCl | | |

### Example 2: Synthesis of Ni-(3-chlorobenzyl)-N1-cyclobutylethane-1,2-diamine hydro-chloride (Compound 123)

### 1) tert-butyl N-[2-[(3-chlorophenyl)methylamino]ethyl]carbamate

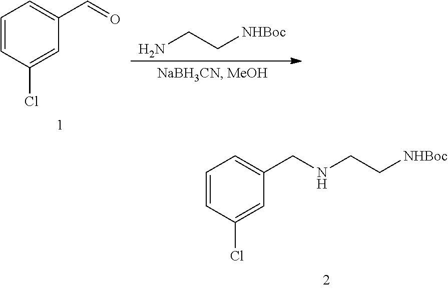 [SMILES: O=Cc1cccc(Cl)c1 ; CC(C)(C)OC(=O)NCCN ; CC(C)(C)OC(=O)NCCNCc1cccc(Cl)c1]

To a solution of tert-butyl N-(2-aminoethyl)carbamate (2.28 g, 14.23 mmol, 2.23 mL, 1 eq) in MeOH (30 mL) was added 3-chlorobenzaldehyde (2 g, 14.23 mmol, 1.61 mL, 1 eq). The mixture was stirred at 25° C. for 12 h. NaBH₃CN (4.47 g, 71.14 mmol, 5 eq) was added to the mixture. The mixture was stirred at 25° C. for 12 h. LC-MS showed the desired compound was detected. The reaction mixture was concentrated to dryness. The reaction was quenched by cold addition of 100 mL of aqueous NH₄Cl. The residue was partitioned between H₂O (100 mL) and ethyl acetate (100 mL). The mixture was extracted with ethyl acetate (100 mL×3). The separated organic layer was washed with brine (100 mL×3), dried over Na₂SO₄ and evaporated to dryness. The residue was purified by flash silica gel chromatography (ISCO®; 20 g SepaFlash® Silica Flash Column, Eluent of 0˜100% ethyl acetate/petroleum ether gradient @150 mL/min) to give compound tert-butyl N-[2-[(3-chlorophenyl)methylamino]ethyl]carbamate (2.3 g) as a yellow oil. 1H NMR (400 MHz, CHLOROFORM-d₆) δ=7.60 (dd, J=1.1, 2.1 Hz, 5H), 4.11 (q, J=7.2 Hz, 6H), 2.03 (s, 9H), 1.24 (t, J=7.2 Hz, 13H).

### 2) tert-butyl N-[2-[(3-chlorophenyl)methyl-cyclobutyl-amino]ethyl]carbamate

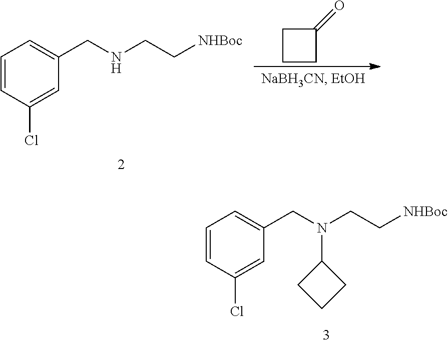 [SMILES: CC(C)(C)OC(=O)NCCNCc1cccc(Cl)c1 ; O=C1CCC1 ; CC(C)(C)OC(=O)NCCN(Cc1cccc(Cl)c1)C1CCC1]

To a solution of tert-butyl N-[2-[(3-chlorophenyl)methylamino]ethyl]carbamate (100 mg, 351 μmol, 1 eq) in EtOH (2 mL) was added dropwise cyclobutanone (246 mg, 3.51 mmol, 262 μL, 10 eq) at 20° C. Then CH₃COOH was added to adjust pH=4-5, the mixture was stirred at this temperature for 1 h, and then NaBH₃CN (66.20 mg, 1.05 mmol, 3 eq) was added dropwise. The resulting mixture was stirred at 40° C. for 12 h. LCMS showed the starting material was consumed completely and desired mass was detected. The reaction mixture was concentrated under reduced pressure to remove solvent. The residue was diluted with H₂O (3 mL) and extracted with ethyl acetate (3 mL×3). The combined organic layers were dried over Na₂SO₄, filtered and concentrated under reduced pressure to give tert-butyl N-[2-[(3-chlorophenyl)methyl-cyclobutyl-amino]ethyl]carbamate (100 mg, crude) as colourless oil.

### 3) N1-(3-chlorobenzyl)-N1-cyclobutylethane-1,2-diamine hydrochloride (Compound 123)

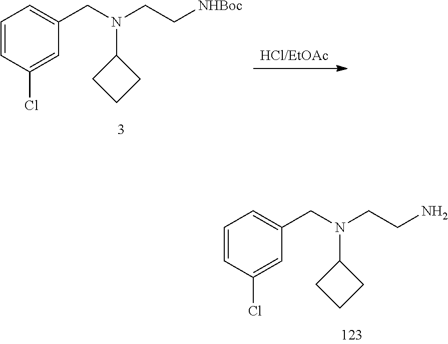 [SMILES: CC(C)(C)OC(=O)NCCN(Cc1cccc(Cl)c1)C1CCC1 ; NCCN(Cc1cccc(Cl)c1)C1CCC1]

A solution of tert-butyl N-[2-[(3-chlorophenyl)methyl-cyclobutyl-amino]ethyl]carbamate (100 mg, 295 μmol, 1 eq) in HC/EtOAc (2 mL, 4 M) was stirred at 20° C. for 1 h. LCMS showed the starting material was consumed completely. The reaction mixture was concentrated under reduced pressure to give a residue which was purified by prep-HPLC (HCl condition, column: Phenomenex Luna 80×30 mm×3 um; mobile phase: [water(HCl)-ACN]; B %: 1%-1%, 8 min) to give compound N¹-(3-chlorobenzyl)-N¹-cyclobutylethane-1,2-diamine hydrochloride (compound 123; 29 mg, 103 μmol, 35% yield, 98.7% purity, HCl) as a white solid. MS (ESI): m/z=239.1 [M+H]⁺; retention time: 1.542 min, method: A; ¹HNMR (400 MHz, METHANOL-d₄) δ=7.65 (br s, 1H), 7.54 (br d, J=4.0 Hz, 1H), 7.39 (br s, 2H), 4.31 (br s, 2H), 3.99-3.78 (m, 1H), 3.20 (br s, 1H), 2.59 (br s, 2H), 2.52-1.86 (m, 4H), 1.82-1.56 (m, 2H).

### Example 3: Synthesis of Compounds 124, 134 and 135

In analogous manner as set out above for Compound 123 (example 2), the following further compounds have been prepared:

| Compound | structure | LCMS | H NMR |
|---|---|---|---|
| 124 | 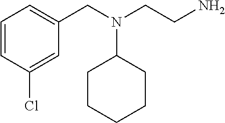 [SMILES: NCCN(Cc1cccc(Cl)c1)C1CCCCC1] | 267.1 [M + H]⁺ Purity: 100% retention time: 1.657 min method: B | 1H NMR (400 MHz, METHANOL-d₄) δ = 7.68 (s, 1H), 7.55 (br d, J = 7.1 Hz, 1H), 7.47-7.37 (m, 2H), 4.57-4.45 (m, 1H), 4.34-4.23 (m, 1H), 3.51 (br d, J = 9.8 Hz, 1H), 3.37- 3.25 (m, 3H), 2.20 (br d, J = |
| | HCl | | 1.3 Hz, 1H), 2.16-2.06 |
| | | | (m, 1H), 1.87 (br d, J = 12.6 Hz, |
| | | | 2H), 1.69-1.45 (m, |
| | | | 4H), 1.37-1.10 (m, 3H). |
| 134 | 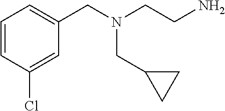 [SMILES: NCCN(Cc1cccc(Cl)c1)CC1CC1] | 239.1 [M + H]⁺ Purity: 100% retention time: 1.440 min method: B | 1H NMR (400 MHz, METHANOL-d₄) δ = 7.66 (d, J = 1.6 Hz, 1H), 7.56- 7.51 (m, 1H), 7.48-7.39 (m, 2H), 4.65-4.36 (m, 2H), 3.64-3.31 (m, 4H), 3.18-3.01 (m, 2H), 1.23- |
| | HCl | | 1.08 (m, 1H), 0.82-0.67 |
| | | | (m, 2H), 0.49-0.33 (m, |
| | | | 2H). |
| 135 | 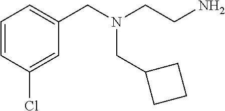 [SMILES: NCCN(Cc1cccc(Cl)c1)CC1CCC1] | 253.1 [M + H]⁺ Purity: 100% retention time: 1.640 min method: B | 1H NMR (400 MHz, METHANOL-d₄) δ = 7.66 (s, 1H), 7.53 (br d, J = 7.1 Hz, 1H), 7.48-7.38 (m, 2H), 4.36 (s, 2H), 3.36 (br s, 4H), 2.91-2.78 (m, 1H), 2.22-2.07 (m, 2H), 2.04- |
| | HCl | | 1.89 (m, 1H), 2.01-1.73 |
| | | | (m, 1H), 2.00-1.73 (m, |
| | | | 1H), 2.05-1.66 (m, 3H). |

### Example 4: Synthesis of N-(2-aminoethyl)-N-[(3-chlorophenyl)methyl]but-3-enamide hydrochloride (Compound 137)

### 1) tert-butyl N-[2-[but-3-enoyl-[(3-chlorophenyl)methyl]amino]ethyl]carbamate

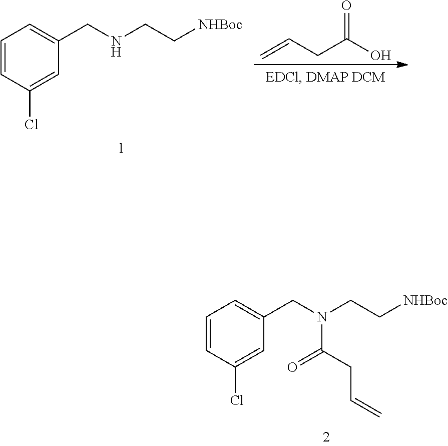

To a solution of tert-butyl N-[2-[(3-chlorophenyl)methylamino]ethyl]carbamate (100 mg, 351.2 μmol, 1 eq) in DCM (2 mL) was added EDCI (87.5 mg, 456.5 μmol, 1.3 eq) and DMAP (5.6 mg, 45.7 μmol, 0.13 eq). The mixture was stirred at 20° C. for 20 min. Then, but-3-enoic acid (39.3 mg, 456.5 μmol, 1.3 eq) was added and the mixture was stirred at 20° C. for 12 h. LCMS showed the starting material was consumed completely and the desired product was obtained. The reaction mixture was partitioned between DCM (2 mL) and H₂O (3 mL×2). The organic phase was separated, dried over Na₂SO₄, filtered and concentrated under reduced pressure to give tert-butyl N-[2-[but-3-enoyl-[(3-chlorophenyl)methyl]amino]ethyl]carbamate (60 mg, crude) as colourless oil.

### 2)N-(2-aminoethyl)-N-[(3-chlorophenyl)methyl]but-3-enamide hydrochloride (compound 137)

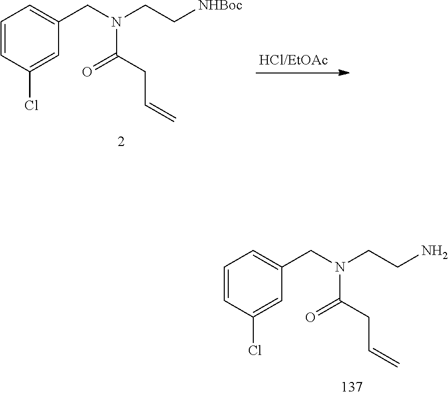 [SMILES: C=CCC(=O)N(CCNC(=O)OC(C)(C)C)Cc1cccc(Cl)c1 ; C=CCC(=O)N(CCN)Cc1cccc(Cl)c1]

A solution of tert-butyl N-[2-[but-3-enoyl-[(3-chlorophenyl)methyl]amino]ethyl]carbamate (60 mg, 170 μmol, 1 eq) in HCl/EtOAc (2 mL, 4 M) was stirred at 20° C. for 1 h. LCMS showed the starting material was consumed completely. The reaction mixture was concentrated under reduced pressure to give a residue. The residue was purified by prep-HPLC (HCl condition, column: Phenomenex Luna 80×30 mm×3 m; mobile phase: [water(HCl)-ACN]; B %: 10%-45%, 8 min) to give compound N-(2-aminoethyl)-N-[(3-chlorophenyl)methyl]but-3-enamide hydrochloride (19 mg, 62.4 μmol, 37% yield, 97.5% purity, HCl) as a white solid. MS (ESI): m/z=253.1 [M+H]⁺; retention time: 1.778 min, method: B; ¹H NMR (400 MHz, DMSO-d₆) δ=8.25-7.95 (m, 2H), 7.43-7.28 (m, 3H), 7.22 (br d, J=7.3 Hz, 1H), 6.02-5.84 (m, 1H), 5.23-5.03 (m, 2H), 4.69-4.48 (m, 5H), 3.63-3.51 (m, 2H), 2.96 (br s, 2H).

### Example 5: Synthesis of N1-(3-chloro-2-fluorobenzyl)-N1-(2,2-difluoroethyl)ethane-1,2-diamine (Compound 118)

### 1)N-[(3-chloro-2-fluoro-phenyl)methyl]-2,2-difluoro-ethanamine

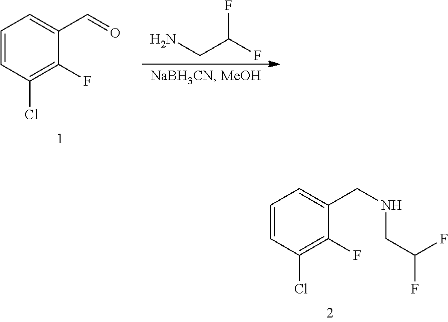 [SMILES: O=Cc1cccc(Cl)c1F ; NCC(F)F ; Fc1c(Cl)cccc1CNCC(F)F]

To a solution of 3-chloro-2-fluoro-benzaldehyde (500 mg, 3.15 mmol, 1 eq), 2,2-difluoroethanamine (384 mg, 4.7 mmol, 1.5 eq) in MeOH (15 mL) was added AcOH to adjust the pH to 4-5. After addition, the mixture was stirred at 25° C. for 2 h and then NaBH₃CN (793 mg, 12.6 mmol, 4 eq) was added at 25° C. The resulting mixture was stirred at 25° C. for 12 h. LC-MS showed the desired compound was detected. The reaction mixture was quenched by addition of satur. NaHCO₃ solution (10 mL) at 25° C. and then extracted with ethyl acetate (10 mL×3). The combined organic layers were washed with saline solution (15 mL×2), dried over Na₂SO₄, filtered and concentrated under reduced pressure to give compound N-[(3-chloro-2-fluoro-phenyl)methyl]-2,2-difluoro-ethanamine (712 mg, crude) as a yellow liquid.

### 2)N-[2-[(3-chloro-2-fluoro-phenyl)methyl-(2,2-difluoroethyl)amino]ethyl]carbamate

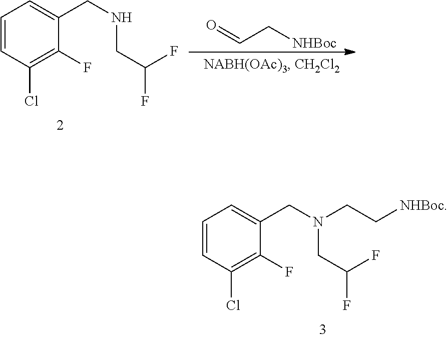

To N-[(3-chloro-2-fluoro-phenyl)methyl]-2,2-difluoro-ethanamine (200 mg, 894.4 μmol, 1 eq) and tert-butyl N-(2-oxoethyl)carbamate (142.37 mg, 894.37 μmol, 1 eq) in CH₂Cl₂ (10 mL) was added AcOH to adjust the pH to 4-5. The mixture was stirred at 25° C. for 2 h. Then NaBH(OAc)₃ (190 mg, 894.4 μmol, 1 eq) was added. The final mixture was stirred for 12 h. LC-MS showed the desired compound was detected. The reaction mixture was quenched by addition of satur. NaHCO₃ solution (15 mL) at 25° C., and extracted with CH₂Cl₂ (10 mL×3). The combined organic layers were washed with saline solution (15 mL×2), dried over Na₂SO₄, filtered and concentrated under reduced pressure to give compound N-[2-[(3-chloro-2-fluoro-phenyl)methyl-(2,2-difluoroethyl)amino]ethyl]carbamate (283 mg, crude) as colourless oil.

### 3) N1-(3-chloro-2-fluorobenzyl)-N1-(2,2-difluoroethyl)ethane-1,2-diamine (Compound 118)

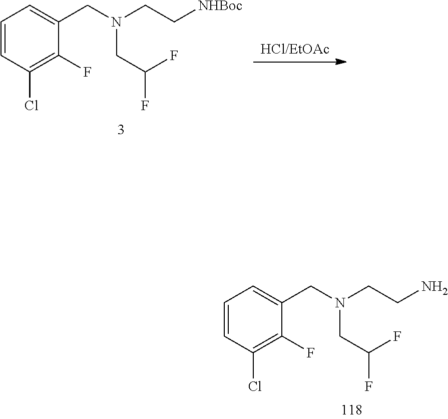 [SMILES: CC(C)(C)OC(=O)NCCN(Cc1cccc(Cl)c1F)CC(F)F ; NCCN(Cc1cccc(Cl)c1F)CC(F)F]

A mixture of tert-butyl N-[2-[(3-chloro-2-fluoro-phenyl)methyl-(2,2-difluoroethyl)amino]ethyl]carbamate (160 mg, 436.5 μmol, 1 eq), and HCl/EtOAc (3 mL 4M) was stirred 25° C. for 1 h. LC-MS showed the desired compound was detected. The residue was purified by prep-HPLC (HCL condition: column: Phenomenex Luna C18 100×30 mm×5 um; mobile phase: [water(HCl)-ACN]; B %: 45%-75%, 10 min) to give compound N1-(3-chloro-2-fluorobenzyl)-N1-(2,2-difluoroethyl)ethane-1,2-diamine hydrochloride (15 mg, 49.5 μmol, 11% yield, HCl) as a yellow liquid. MS (ESI): m/z=267.1 [M+H]⁺; retention time: 1.899 min, method: A. ¹H NMR (400 MHz, DMSO-d₆) δ=8.11-7.78 (m, 3H), 7.51 (q, J=7.7 Hz, 2H), 7.23 (t, J=7.8 Hz, 1H), 6.32-5.96 (m, 1H), 3.87 (s, 2H), 3.00-2.78 (m, 6H).

### Example 6: Synthesis of N1-(3-chloro-2-fluorobenzyl)-N1-(2,2,2-trifluoroethyl)ethane-1,2-diamine (Compound 119)

In analogous manner as set out above for Compound 118 (example 5), the following further compound has been prepared:

| Compound | Structure | LCMS | H NMR |
|---|---|---|---|
| 119 | 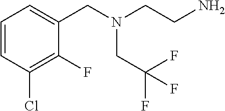 [SMILES: NCCN(Cc1cccc(Cl)c1F)CC(F)(F)F] | 267.0 [M + H]+ Purity: 100% retention time: 1.953 min method: G | ¹H NMR (400 MHz, DMSO-d₆) δ = 7.95 (br s, 3H), 7.45 (s, 1H), 7.42- 7.30 (m, 3H), 6.11-5.77 (m, 3H), 3.84 (s, 2H), 3.40 (q, J = 10.1 Hz, 2H), 2.87 (s, 4H). |
| | HCl | | |

### Example 7: Synthesis of N1-(3-chloro-2-fluorobenzyl)-N1-(2-(methylthio)ethyl)ethane-1,2-diamine hydrochloride (Compound 168)

### 1) tert-butyl N-[2-[(3-chloro-2-fluorophenyl)methylamino]ethyl]carbamate

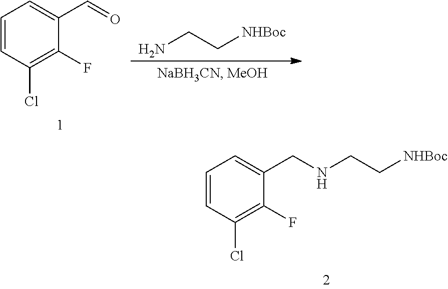 [SMILES: O=Cc1cccc(Cl)c1F ; CC(C)(C)OC(=O)NCCN ; CC(C)(C)OC(=O)NCCNCc1cccc(Cl)c1F]

To a solution of 3-chloro-2-fluoro-benzaldehyde (10 g, 63 mmol, 1 eq) in MeOH (150 mL) was added tert-butyl N-(2-aminoethyl)carbamate (10.1 g, 63 mmol, 9.91 mL, 1 eq). The mixture was stirred at 65° C. for 12 h. Then NaBH₃CN (19.82 g, 315.3 mmol, 5 eq) was added at 20° C. The mixture was stirred at 65° C. for 2 h. The reaction mixture was concentrated under reduced pressure to remove solvent. The residue was diluted with H₂O 150 mL and extracted with ethyl acetate (100 mL×3). The combined organic layers were washed with H₂O (100 mL×2), dried over Na₂SO₄, filtered and concentrated under reduced pressure to give a residue which was purified by flash silica gel chromatography (ISCO®; 40 g SepaFlash® Silica Flash Column, eluent of 0˜36% ethyl acetate/petroleum ether gradient @120 mL/min) to give compound tert-butyl N-[2-[(3-chloro-2-fluoro-phenyl)methylamino]ethyl]carbamate (10 g, 33 mmol, 52.4% yield) as a yellow oil.

### 2)N-[2-[(3-chloro-2-fluoro-phenyl)methyl-(2-methylsulfanylethyl)amino]ethyl]carbamate

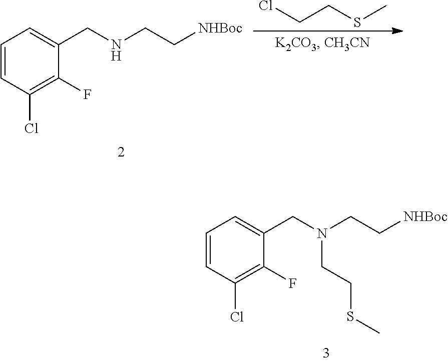

A mixture of tert-butyl N-[2-[(3-chloro-2-fluoro-phenyl)methylamino]ethyl]carbamate (500 mg, 1.65 mmol, 1 eq), 1-chloro-2-methylsulfanyl-ethane (182 mg, 1.65 mmol, 163 μL, 1 eq), K₂CO₃ (685 mg, 4.95 mmol, 3 eq) in CH₃CN (10 mL) was stirred at 80° C. for 12 h. LC-MS showed desired compound was detected. The mixture was filtered to remove the K₂CO₃, and then the mixture was concentrated under reduced pressure. The residue was purified by flash silica gel chromatography (ISCO®; 12 g SepaFlash® Silica Flash Column, eluent of 0˜100% ethyl acetate/petroleum ether gradient @80 mL/min) to give compound N-[2-[(3-chloro-2-fluoro-phenyl)methyl-(2-methylsulfanylethyl)amino]ethyl]carbamate (200 mg, 500.7 μmol, 30% yield, 94.4% purity) as a colourless oil.

### 3) N1-(3-chloro-2-fluorobenzyl)-N1-(2-(methylthio)ethyl)ethane-1,2-diamine hydrochloride (Compound 168)

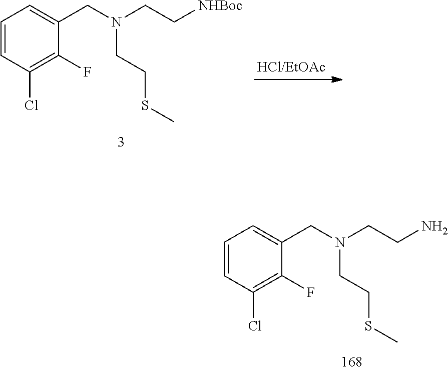 [SMILES: CSCCN(CCNC(=O)OC(C)(C)C)Cc1cccc(Cl)c1F ; CSCCN(CCN)Cc1cccc(Cl)c1F]

A solution of tert-butyl N-[2-[(3-chloro-2-fluoro-phenyl)methyl-(2-methylsulfanylethyl)amino]ethyl]carbamate (50 mg, 133 μmol, 1 eq) in HCl/EtOAc (1 mL, 4M) was stirred at 25° C. for 1 h. LC-MS showed that the desired compound was detected. The mixture was concentrated under reduce pressure. The residue was purified by prep-HPLC (HCl condition: column: Phenomenex Luna 80×30 mm×3 um; mobile phase: [water(HCl)-ACN]; B %: 1%-20%, 8 min) to give compound N1-(3-chloro-2-fluorobenzyl)-N1-(2-(methylthio)ethyl)ethane-1,2-diamine hydrochloride (7.5 mg, 26.7 μmol, 20% yield, 98.5% purity) as a white solid. MS (ESI): m/z=277.1 [M+H]⁺; retention time: 1.777 min, method: A ¹H NMR (400 MHz, DMSO-d₆) δ=8.20 (br s, 3H), 7.79-7.62 (m, 2H), 7.32 (br t, J=7.8 Hz, 1H), 4.60-4.07 (m, 2H), 3.27-2.80 (m, 8H), 2.09 (s, 3H).

### Example 8: Synthesis of Compounds 126, 136 and 160

In analogous manner as set out above for Compound 168 (example 7), the following further compounds have been prepared:

| Compound | Structure | LCMS | H NMR |
|---|---|---|---|
| 126 | 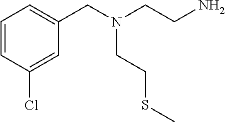 [SMILES: CSCCN(CCN)Cc1cccc(Cl)c1] | 259.0 [M + H]⁺ Purity: 100% retention time: 1.736 min method: B | 1H NMR (400 MHz, DMSO-d₆) δ = 11.94- 11.64 (m, 1H), 8.51- 8.25 (m, 3H), 7.84 (br s, 1H), 7.73-7.59 (m, 1H), 7.57-7.44 (m, 2H), 4.57- 4.31 (m, 2H), 3.28 (br s, 6H), 2.94 (br s, 2H), |
| | 2 HCl | | 2.07 (s, 3H). |
| 136 | 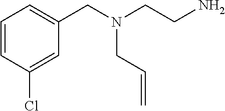 | 225.0 [M + H]⁺ Purity: 100% retention time: 1.411 min method: A | 1H NMR (400 MHz, DMSO-d₆) δ = 12.07- 11.65 (m, 1H), 8.47- 8.21 (m, 3H), 7.82 (br s, 1H), 7.64 (br d, J = 6.8 Hz, 1H), 7.56-7.43 (m, 2H), 6.17-6.00 (m, 1H), |
| | 2 HCl | | 5.66-5.45 (m, 2H), 4.53- |
| | | | 4.23 (m, 2H), 3.82- |
| | | | 3.65 (m, 2H), 3.30 (br s, |
| | | | 4H). |
| 160 | 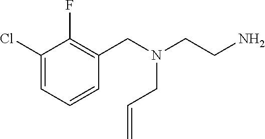 [SMILES: C=CCN(CCN)Cc1cccc(Cl)c1F] | 243.0 [M + H]+ Purity: 100% retention time: 1.525 min method: A | 1H NMR (400 MHz, DMSO-d₆) δ = 12.14- 11.49 (m, 1H), 8.59- 8.03 (m, 3H), 7.96-7.57 (m, 2H), 7.46-7.08 (m, 1H), 6.29-5.93 (m, 1H), 5.77-5.25 (m, 2H), 4.70- 4.17 (m, 2H), 3.89 (br s, 2H), 3.45-3.13 (m, |
| | 2 HCl | | 4H). |

### Example 9: Synthesis of 3-(((2-aminoethyl)(ethyl)amino)methyl)-2-fluorobenzonitrile TFA salt (Compound 175)

### 1) tert-butyl N-[2-[(3-bromo-2-fluoro-phenyl)methylamino]ethyl]carbamate

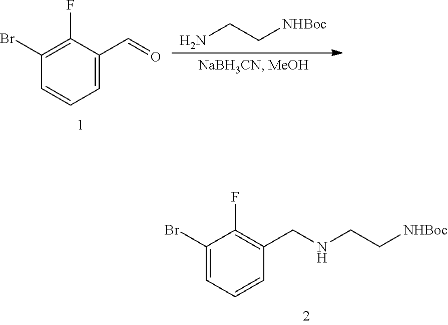

To a solution of 3-bromo-2-fluoro-benzaldehyde (400 mg, 1.97 mmol, 1 eq) and tert-butyl N-(2-aminoethyl)carbamate (316 mg, 2 mmol, 310 μL, 1 eq) in MeOH (5 mL) was added AcOH to adjust pH to 4-5, the mixture was stirred at 40° C. for 12 h, then NaBH₃CN (372 mg, 5.9 mmol, 3 eq) was added, the mixture was stirred at 25° C. for 2 h. The mixture was concentrated to get a residue, and the residue was poured into water and extracted by ethyl acetate (5 mL×3). Then the combined organic layers were washed by brine (5 mL×2) and then dried by Na₂SO₄, then filtered and the filtrate was concentrated in vacuo to get compound tert-butyl N-[2-[(3-bromo-2-fluoro-phenyl)methylamino]ethyl]carbamate (600 mg, crude) as a colourless oil.

### 2) tert-butyl N-[2-[(3-bromo-2-fluoro-phenyl)methyl-ethyl-amino]ethyl]carbamate

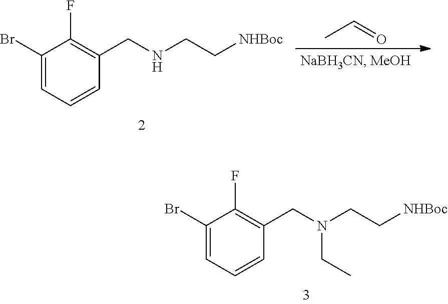

To a solution of tert-butyl N-[2-[(3-bromo-2-fluoro-phenyl)methylamino]ethyl]carbamate (600 mg, 1.73 mmol, 1 eq) and acetaldehyde (761 mg, 6.9 mmol, 970 μL, 40% purity, 4 eq) in MeOH (5 mL) was added AcOH to adjust pH to 4-5, the mixture was stirred at 25° C. for 12 h, then NaBH₃CN (326 mg, 5.18 mmol, 3 eq) was added and the mixture was stirred at 25° C. for 12 h. The mixture was concentrated in vacuo to get a residue. The residue was purified by column chromatography (SiO2, petroleum ether/ethyl acetate=10/1 to 1/1) to give compound tert-butyl N-[2-[(3-bromo-2-fluoro-phenyl)methyl-ethyl-amino]ethyl]carbamate (300 mg, 799.4 μmol, 46% yield) as a colourless oil.

### 3) tert-butyl N-[2-[(3-cyano-2-fluoro-phenyl)methyl-ethyl-amino]ethyl]carbamate

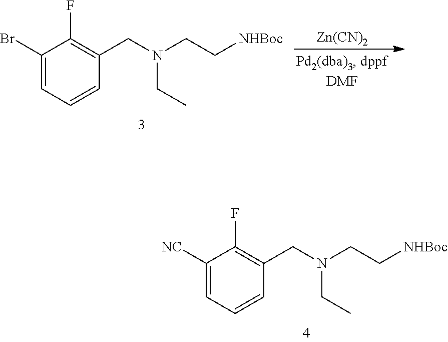

A mixture of tert-butyl N-[2-[(3-bromo-2-fluoro-phenyl)methyl-ethyl-amino]ethyl]carbamate (200 mg, 533 μmol, 1 eq), Zn(CN)₂ (188 mg, 1.6 mmol, 101.5 μL, 3 eq), Pd₂(dba)₃ (49 mg, 53.3 μmol, 0.1 eq) and dppf (59 mg, 106.6 μmol, 0.2 eq) in DMF (3 mL) was degassed and purged with N₂ for 3 times, and then the mixture was stirred at 100° C. for 12 h under N₂ atmosphere. The mixture was filtered and the filtrate was poured into water and extracted by ethyl acetate (3 mL×3). The combined organic layers were washed by brine (3 mL×2), then dried by Na₂SO₄, filtered and the filtrate was concentrated in vacuo to get compound tert-butyl N-[2-[(3-cyano-2-fluoro-phenyl)methyl-ethyl-amino]ethyl]carbamate (150 mg, crude) as a colourless oil.

### 4) 3-(((2-aminoethyl)(ethyl)amino)methyl)-2-fluorobenzonitrile TFA salt (Compound 175)

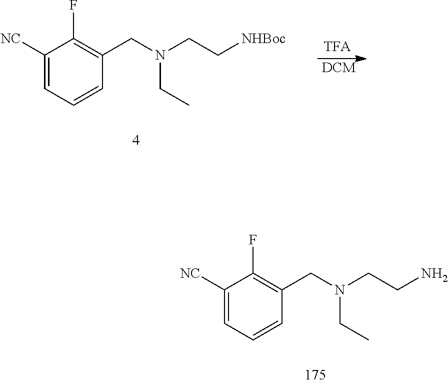

To a solution of tert-butyl N-[2-[(3-cyano-2-fluoro-phenyl)methyl-ethyl-amino]ethyl]carbamate (150 mg, 467 μmol, 1 eq) in DCM (2 mL) was added TFA (1.54 g, 13.5 mmol, 1 mL, 29 eq). The mixture was stirred at 25° C. for 12 h and the mixture was concentrated in vacuo to get a residue. The residue was purified by prep-HPLC (TFA condition; column: C18-1 150×30 mm×5 um; mobile phase: [water(TFA)-ACN]; B %: 1%-25%, 8 min) then purified by Prep-HPLC TFA condition column: C18-1 150×30 mm×5 um; mobile phase: [water(TFA)-ACN]; B %: 1%-25%, 8 min) to give compound 3-[[2-aminoethyl(ethyl)amino]methyl]-2-fluoro-benzonitrile TFA salt (145 mg, 413.4 μmol, 88.6% yield, 95.5% purity, TFA) as a colourless gum. MS (ESI): m/z=222.1 [M+H]⁺; retention time: 0.497 min, method: I; ¹H NMR (400 MHz, DMSO-d₆) δ=8.00-7.85 (m, 4H), 7.47 (t, J=7.8 Hz, 1H), 4.24-3.85 (m, 2H), 3.12-2.67 (m, 6H), 1.13 (br s, 3H).

### Example 10: Synthesis of Compound 175A

In analogous manner as set out above for Compound 175 (example 9), however without step 3 and using HCl instead of TFA in the deprotection step, Compound 175A has been prepared:

| Compound | Structure | LCMS | H NMR |
|---|---|---|---|
| 175A | 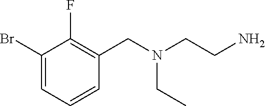 | 277.0 [M + H]⁺ Purity: 100% retention time: 2.282 min method: E | ¹H NMR (400 MHz, DMSO-d₆) δ = 11.77- 11.37 (m, 1H), 8.66-8.31 (m, 3H), 8.02-7.75 (m, 2H), 7.27 (br t, J = 7.8 Hz, 1H), 4.48 (br s, 2H), 3.54- 3.08 (m, 6H), 1.32 (br s, |
| | HCl | | 3H). |

### Example 11: Synthesis of N1-(3-chloro-2-fluorobenzyl)-N1-cyclobutylethane-1,2-diamine hydrochloride (Compound 159)

### 1) tert-butyl (2-((3-chloro-2-fluorobenzyl)(cyclobutyl)amino)ethyl)carbamate

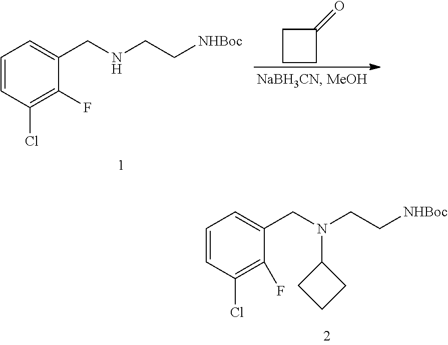 [SMILES: CC(C)(C)OC(=O)NCCNCc1cccc(Cl)c1F ; O=C1CCC1 ; CC(C)(C)OC(=O)NCCN(Cc1cccc(Cl)c1F)C1CCC1]

To a solution of tert-butyl N-[2-[(3-chloro-2-fluoro-phenyl)methylamino]ethyl]carbamate (150 mg, 495.4 μmol, 1 eq) and cyclobutanone (69.5 mg, 991 μmol, 74 μL, 2 eq) in MeOH (2 mL) was added AcOH to adjust pH to 4-5. The mixture was stirred at 25° C. for 1 h, then NaBH₃CN (93.4 mg, 1.49 mmol, 3 eq) was added. The mixture was stirred at 25° C. for 12 h. The mixture was poured into water 5 mL and then extracted by ethyl acetate (3 mL×3). Then the organic layers were washed by brine (2 mL×2) and then filtered and the filtrate was concentrated in vacuo to get compound tert-butyl N-[2-[(3-chloro-2-fluoro-phenyl)methyl-cyclobutyl-amino]ethyl]carbamate (170 mg, crude) as a white solid.

### 2) N1-(3-chloro-2-fluorobenzyl)-N1-cyclobutylethane-1,2-diamine dihydrochloride (Compound 159)

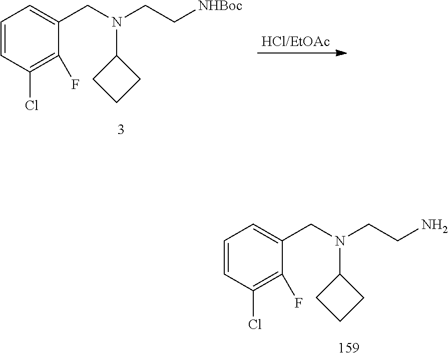 [SMILES: CC(C)(C)OC(=O)NCCN(Cc1cccc(Cl)c1F)C1CCC1 ; NCCN(Cc1cccc(Cl)c1F)C1CCC1]

To a solution of tert-butyl N-[2-[(3-chloro-2-fluoro-phenyl)methyl-cyclobutyl-amino]ethyl]carbamate (170 mg, 476 μmol, 1 eq) in HCl/EtOAc (1 mL, 4M) was added EtOAc (2 mL) and the mixture was stirred at 25° C. for 12 h. The mixture was concentrated in vacuo to get a residue which was purified by prep-HPLC (HCl condition; column: Phenomenex luna C18 80*40 mm*3 m; mobile phase: [water(HCl)-ACN]; B %: 1%-30%, 7 min) to afford N1-(3-chloro-2-fluorobenzyl)-N1-cyclobutylethane-1,2-diamine dihydrochloride (99 mg, 337.3 μmol, 71% yield, 100% purity, 2 HCl) as a white gum. MS (ESI): m/z=257.0 [M+H]⁺; retention time: 1.337 min, method: A; ¹H NMR (400 MHz, DMSO-d₆) δ=12.20-11.89 (m, 1H), 8.66-8.27 (m, 3H), 7.89-7.67 (m, 2H), 7.32 (t, J=7.9 Hz, 1H), 4.52-4.30 (m, 2H), 4.19-3.70 (m, 4H), 3.49-3.32 (m, 1H), 2.62-2.51 (m, 1H), 2.47-2.30 (m, 1H), 2.26-1.93 (m, 2H), 1.78-1.56 (m, 2H).

### Example 12: Synthesis of (R)—N1-(1-(3-chlorophenyl)ethyl)-N1-ethylethane-1,2-diamine dihydrochloride (Compound 176)

### 1) (R)-tert-butyl (2-((1-(3-chlorophenyl)ethyl)amino)ethyl)carbamate

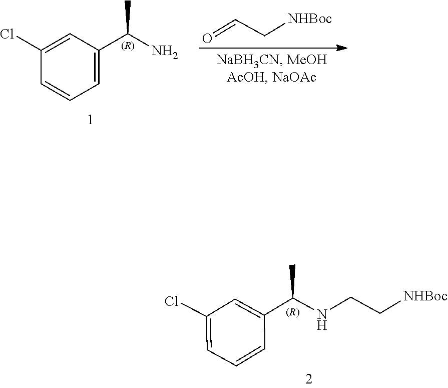

To a solution of tert-butyl N-(2-oxoethyl)carbamate (198.89 mg, 1.25 mmol, 1.2 eq) and (1R)-1-(3-chlorophenyl)ethanamine; hydrochloride (200 mg, 1.04 mmol, 1 eq) in MeOH (3 mL) was added AcOH (420 mg, 7 mmol, 400 μL, 6.72 eq) and NaOAc (85.4 mg, 1.04 mmol, 1 eq), then NaBH₃CN (78.5 mg, 1.25 mmol, 1.2 eq) was added. The mixture was stirred at 20° C. for 12 h. The reaction mixture was concentrated under reduced pressure to remove solvent. The residue was diluted with satur. NaHCO₃ (3 mL) and extracted with ethyl acetate (5 mL×3). The combined organic layers were washed with brine (5 mL×2), dried over Na₂SO₄, filtered and concentrated under reduced pressure to give (R)-tert-butyl (2-((1-(3-chlorophenyl)ethyl)amino)ethyl)carbamate (300 mg, 1 mmol, 96.4% yield) as a colourless oil.

### 2) (R)-tert-butyl (2-((1-(3-chlorophenyl)ethyl)(ethyl)amino)ethyl)carbamate

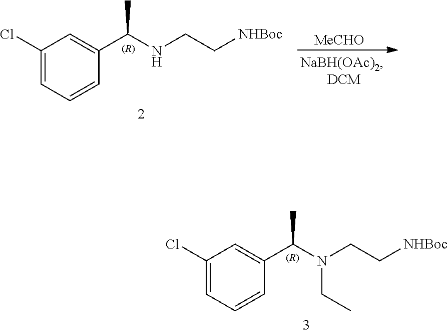

To a solution of tert-butyl N-[2-[[(1R)-1-(3-chlorophenyl)ethyl]amino]ethyl]carbamate (260 mg, 870 μmol, 1 eq) and acetaldehyde (192 mg, 4.35 mmol, 244 μL, 5 eq) in DCM (4 mL) was added AcOH to adjust pH to 4-5, the mixture was stirred at 20° C. for 2 h, then NaBH(OAc)₃ (369 mg, 1.74 mmol, 2 eq) was added and the mixture was stirred at 20° C. for 2 h. LC-MS showed reactant 2 was consumed completely and ˜49% of desired compound was detected. The mixture was concentrated to give a residue. The reaction mixture was quenched by addition of satur. NaHCO₃ solution to pH=8, and then extracted with EtOAc (10 ml×3). The combined organic phases were dried with anhydrous Na₂SO₄. The mixture was filtered and the filtrate was concentrated under vacuum. The residue was purified by prep-TLC (SiO₂, petroleum ether/ethyl acetate=1/1, Rf=0.44) to give (R)-tert-butyl (2-((1-(3-chlorophenyl)ethyl)(ethyl)amino)ethyl)carbamate (100 mg, 306 μmol, 35% yield) as a colourless oil.

### 3) (R)—N1-(1-(3-chlorophenyl)ethyl)-N1-ethylethane-1,2-diamine dihydrochloride (Compound 176)

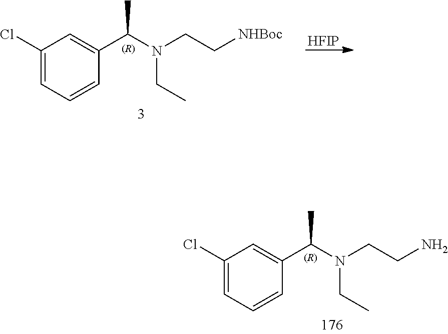

A solution of tert-butyl N-[2-[[(1R)-1-(3-chlorophenyl)ethyl]-ethyl-amino]ethyl]carbamate (70 mg, 214 μmol, 1 eq) in hexafluoroisopropanol (0.5 mL) was stirred at 80° C. for 3 h. LC-MS showed reactant 3 was consumed completely and ˜94% of desired compound was detected. The mixture was concentrated to give a residue. The residue was purified by prep-HPLC (HCl condition, column: Phenomenex luna C18 80×40 mm×3 μm; mobile phase: [water(HCl)-ACN]; B %: 1%-30%, 7 min) to give (R)—N1-(1-(3-chlorophenyl)ethyl)-N1-ethylethane-1,2-diamine dihydrochloride (13 mg, 48 μmol, 22% yield, 97.3% purity, 2 HCl) as a white solid. MS(ESI): m/z=227.1 [M+H]⁺; retention time: 1.799 min, method: K; ¹H NMR (400 MHz, DMSO-d₆) δ 10.99-12.28 (m, 1H), 8.26-8.86 (m, 3H), 7.87 (s, 1H), 7.67-7.77 (m, 1H), 7.41-7.53 (m, 2H), 4.69 (q, J=6.72 Hz, 1H), 3.77 (br s, 3H), 3.48 (br s, 1H), 2.82-3.22 (m, 2H), 1.74 (d, J=6.85 Hz, 3H), 1.17-1.41 (m, 3H).

### Example 13: Synthesis of (S)—N1-(1-(3-chlorophenyl)ethyl)-N1-ethylethane-1,2-diamine dihydrochloride (Compound 177)

In analogous manner as set out above for Compound 176 (example 12), Compound 177 has been prepared:

| Compound | Structure | LCMS | H NMR |
|---|---|---|---|
| 177 | 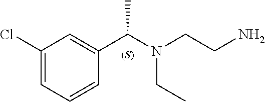 | 227.1 [M + H]⁺ Purity: 100% retention time: 1.253 min method: A | ¹H NMR (400 MHz, DMSO-d₆) δ = 11.95- 11.31 (m, 1H), 8.82 (s, 3H), 7.90-7.79 (m, 1H), 7.71 (br s, 1H), 7.57-7.45 (m, 2H), 4.83-4.50 (m, 1H), |
| | 2 HCl | | 3.61-2.74 (m, 6H), |
| | | | 1.86-1.60 (m, 3H), |
| | | | 1.46-1.13 (m, 3H). |

### Example 14: Synthesis of N1-(1-(3-chloro-2-fluorophenyl)ethyl)-N1-cyclopentylethane-1,2-diamine dihydrochloride (Compound 184)

### 1)N-(1-(3-chloro-2-fluorophenyl)ethyl)cyclopentanamine

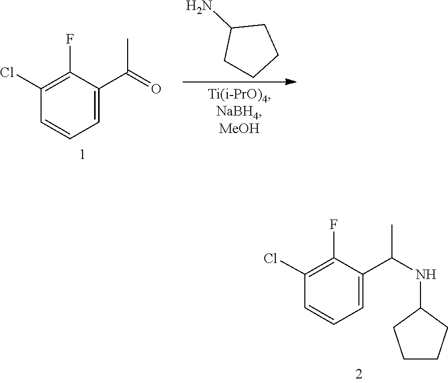 [SMILES: CC(=O)c1cccc(Cl)c1F ; NC1CCCC1 ; CC(NC1CCCC1)c1cccc(Cl)c1F]

A solution of 1-(3-chloro-2-fluoro-phenyl)ethanone (500 mg, 2.90 mmol, 1 eq) and cyclopentanamine (493 mg, 5.79 mmol, 571.7 μL, 2 eq) in tetraisopropoxytitanium (1.07 g, 3.8 mmol, 1.1 mL, 1.3 eq) was stirred at 20° C. for 1 h. Then MeOH (10 mL) and NaBH₄ (186 mg, 4.93 mmol, 1.7 eq) was added at 0° C., the mixture was stirred at 20° C. for 2 h. LC-MS showed reactant 1 was consumed completely and ˜70% of desired compound was detected. The reaction mixture was diluted with 10 mL H₂O and extracted with EtOAc (10 mL×3). The combined organic phase was dried with anhydrous Na₂SO₄, the mixture was filtered and the filtrate was concentrated under vacuum to give N-(1-(3-chloro-2-fluorophenyl)ethyl)cyclopentanamine (400 mg, crude) as a yellow oil.

### 2. tert-butyl (2-((1-(3-chloro-2-fluorophenyl)ethyl)(cyclopentyl)amino)ethyl)carbamate

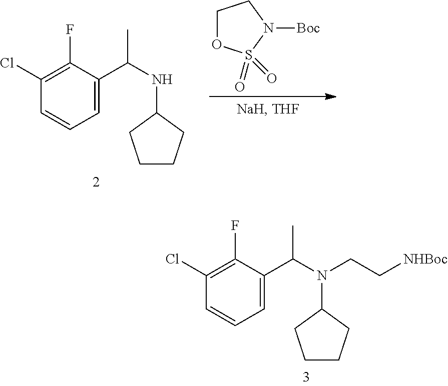 [SMILES: CC(NC1CCCC1)c1cccc(Cl)c1F ; CC(C)(C)OC(=O)N1CCOS1(=O)=O ; CC(c1cccc(Cl)c1F)N(CCNC(=O)OC(C)(C)C)C1CCCC1]

To a solution of N-(1-(3-chloro-2-fluorophenyl)ethyl)cyclopentanamine (280 mg, 1.16 mmol, 1 eq) and tert-butyl 2,2-dioxooxathiazolidine-3-carboxylate (259 mg, 1.16 mmol, 1 eq) in THF (6 mL) was added NaH (56 mg, 1.39 mmol, 60% purity, 1.2 eq) at 0° C., the mixture was stirred at 80° C. for 2 h under N₂ atmosphere. LC-MS showed ˜42% of reactant 2 was remained and ˜21% of desired compound was detected. The reaction mixture was diluted with 10 mL H₂O and extracted with EtOAc (8 mL×3). The combined organic phase was dried with anhydrous Na₂SO₄, the mixture was filtered and the filtrate was concentrated under vacuum. The residue was purified by prep-HPLC (HCl condition; column: Phenomenex Luna 80×30 mm×3 um; mobile phase: [water(HCl)-ACN]; B %: 20%-45%, 8 min) to give tert-butyl (2-((1-(3-chloro-2-fluorophenyl)ethyl)(cyclopentyl)amino)ethyl)carbamate (240 mg, 624 mol, 54% yield) as a white solid.

### 3. N1-(1-(3-chloro-2-fluorophenyl)ethyl)-N1-cyclopentylethane-1,2-diamine dihydrochloride (Compound 184)

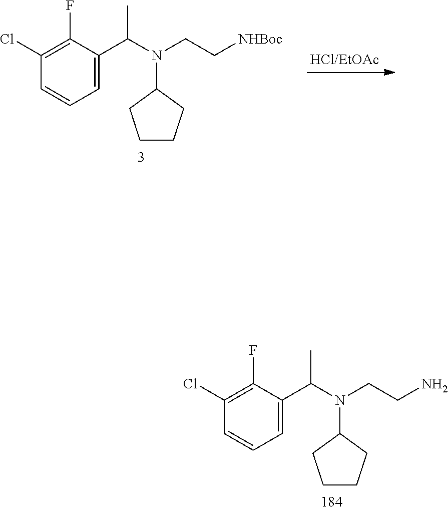 [SMILES: CC(c1cccc(Cl)c1F)N(CCNC(=O)OC(C)(C)C)C1CCCC1 ; CC(c1cccc(Cl)c1F)N(CCN)C1CCCC1]

A solution of tert-butyl (2-((1-(3-chloro-2-fluorophenyl)ethyl)(cyclopentyl)amino)ethyl) carbamate (40 mg, 103.9 μmol, 1 eq) and HCl/EtOAc (4 M, 1.5 mL, 57.74 eq) in EtOAc (1 mL) was stirred at 20° C. for 2 h. LC-MS showed reactant 3 was consumed completely and ˜23% of desired compound was detected. The reaction mixture was concentrated under reduced pressure to remove solvent. The residue was purified by prep-HPLC (HCl condition; column: Phenomenex Luna 80×30 mm×3 μm; mobile phase: [water(HCl)-ACN]: B %: 5%-25%, 8 min) to give N1-(1-(3-chloro-2-fluorophenyl)ethyl)-N1-cyclopentylethane-1,2-diamine dihydrochloride (10.24 mg, 33.19 μmol, 31.9% yield, 92.3% purity, 2 HCl) as a colourless gum. MS (ESI): m/z=285.1 [M+H]⁺; retention time: 1.641 min, method: A ¹H NMR (400 MHz, DMSO-d₆) δ 11.2-11.8 (m, 1H), 8.3-8.7 (m, 3H), 8.0-8.2 (m, 1H), 7.7-7.8 (m, 1H), 7.3-7.4 (m, 1H), 4.9-5.1 (m, 1H), 3.5-3.8 (m, 3H), 3.0-3.4 (m, 2H), 1.9-2.2 (m, 2H), 1.6-1.9 (m, 7H), 1.4-1.6 (m, 2H).

### Example 15: Synthesis of N′-[1-(3-chloro-2-fluoro-phenyl)ethyl]-N′-cyclopropyl-ethane-1,2-diamine hydrochloride (Compound 220) and enantiomers thereof

### 1)N-(1-(3-chloro-2-fluorophenyl)ethyl)cyclopropanamine

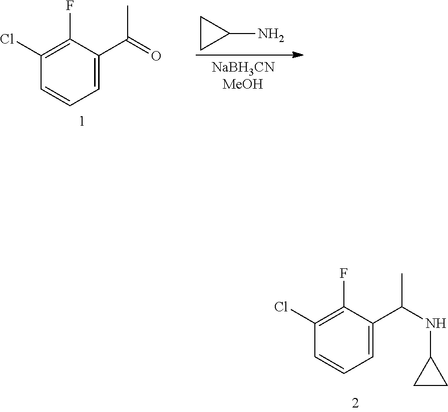

To a solution of 1-(3-chloro-2-fluoro-phenyl)ethanone (200 mg, 1.16 mmol, 1 eq) and cyclopropanamine (99.3 mg, 1.74 mmol, 120.4 μL, 1.5 eq) in MeOH (2 mL) was added AcOH to adjust pH to 4-5 and stirred at 40° C. for 12 h. Then NaBH₃CN (218.47 mg, 3.48 mmol, 3 eq) was added and the mixture was stirred at 40° C. for 2 h. The mixture was concentrated to get a residue which was purified by column chromatography (SiO₂, petroleum ether/ethyl acetate=10/1 to 3/1) to give compound N-[1-(3-chloro-2-fluoro-phenyl)ethyl]cyclopropanamine (200 mg, 936 μmol, 81% yield) as a colourless oil.

### 2) tert-butyl (2-((1-(3-chloro-2-fluorophenyl)ethyl)(cyclopropyl)amino)ethyl)carbamate

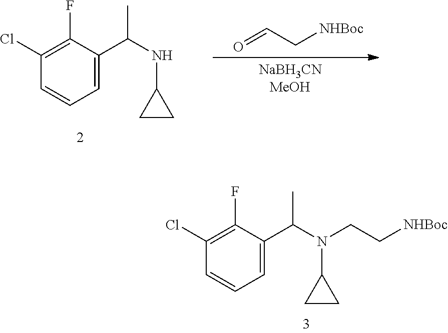 [SMILES: CC(NC1CC1)c1cccc(Cl)c1F ; CC(C)(C)OC(=O)NCC=O ; CC(c1cccc(Cl)c1F)N(CCNC(=O)OC(C)(C)C)C1CC1]

To a solution of N-[1-(3-chloro-2-fluoro-phenyl)ethyl]cyclopropanamine (200 mg, 936 mol, 1 eq) and tert-butyl N-(2-oxoethyl)carbamate (149 mg, 936 μmol, 1 eq) in MeOH (3 mL) was adjusted pH to 4-5 by AcOH, the mixture was stirred at 40° C. for 2 h, then NaBH₃CN (177 mg, 2.8 mmol, 3 eq) was added and the mixture was stirred at 40° C. for 12 h. The mixture was concentrated in vacuo to get compound tert-butyl N-[2-[1-(3-chloro-2-fluoro-phenyl)ethyl-cyclopropyl-amino]ethyl]carbamate (250 mg, crude) as a white solid.

### 3) N1-(1-(3-chloro-2-fluorophenyl)ethyl)-N1-cyclopropylethane-1,2-diamine hydrochloride (Compound 220)

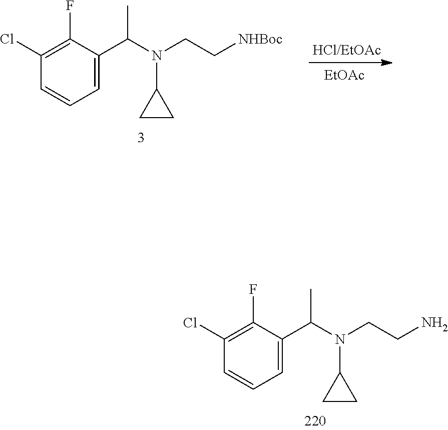 [SMILES: "EtOAc" ; CC(c1cccc(Cl)c1F)N(CCNC(=O)OC(C)(C)C)C1CC1 ; CC(c1cccc(Cl)c1F)N(CCN)C1CC1]

To a solution of tert-butyl N-[2-[1-(3-chloro-2-fluoro-phenyl)ethyl-cyclopropyl-amino]ethyl]carbamate (200 mg, 560.4 μmol, 1 eq) in EtOAc (2 mL) was added HCl/EtOAc (1 mL, 4M). The mixture was stirred at 25° C. for 1 h. The mixture was concentrated to get a residue and the residue was purified by prep-HPLC (HCl condition; column: Phenomenex Luna 80*30 mm*3 m; mobile phase: [water(HCl)-ACN]; B %: 5%-30%, 8 min) to give compound N′-[1-(3-chloro-2-fluoro-phenyl)ethyl]-N′-cyclopropyl-ethane-1,2-diamine hydrochloride (117 mg, 399 μmol, 71% yield, 100% purity, HCl) as a white solid. MS (ESI): m/z=257.0 [M+H]⁺; retention time: 1.744 min, method: K; ¹H NMR (400 MHz, DMSO-d₆) δ=8.44-8.17 (m, 3H), 7.75-7.65 (m, 1H), 7.60 (br t, J=7.4 Hz, 1H), 7.29 (t, J=7.9 Hz, 1H), 4.91-4.65 (m, 1H), 3.47-3.29 (m, 1H), 3.15 (br d, J=2.0 Hz, 3H), 2.43-2.26 (m, 1H), 1.67 (br d, J=6.7 Hz, 3H), 1.03-0.80 (m, 2H), 0.69 (br t, J=6.1 Hz, 2H).

### 4) Enantiomers of N1-(1-(3-chloro-2-fluorophenyl)ethyl)-N1-cyclopropylethane-1,2-diamine

200 mg of compound 220 was further separated by SFC (instrument: Waters SFC80 preparative SFC; column: Daicel ChiralPak IH, 250*30 mm, 10 μm; Flow rate: 60 g/min; Wavelength: 220 nm; Column temperature: 40° C.; System back pressure: 100 bar; mobile phase: A: CO2; B: [0.1% NH₃*H₂O in MeOH]; B %: 30%-30%, 3 min) (P₁ Ret.time=1.245, P₂ Ret.time=1.506) then the product with Ret.time=1.245 was further purified by Prep-HPLC (neutral condition column: Waters Xbridge Prep OBD C18 150*40 mm*10 μm; mobile phase: A: water (NH₃*H₂O+NH₄HCO₃); B: MeCN; B %: 5%-70%, 8 min) to give compound 220A (+)-N1-(1-(3-chloro-2-fluorophenyl)ethyl)-N1-cyclopropylethane-1,2-diamine (Ret. time=1.245; 20.2 mg, 76.88 μmol, 20% yield, 97.72% purity) as a brown oil ((+) rotation in DMSO @589 nm/19.9° C.; ee %=100%). MS (ESI): m/z=257.0 [M+H]⁺; ¹H NMR (400 MHz, DMSO-d₆) δ=7.50-7.45 (m, 1H), 7.43-7.37 (m, 1H), 7.26-7.15 (m, 1H), 4.34 (q, J=7.0 Hz, 1H), 2.71-2.53 (m, 3H), 2.42-2.28 (m, 1H), 1.80-1.66 (m, 1H), 1.38 (d, J=7.0 Hz, 3H), 0.48-0.41 (m, 2H), 0.40-0.24 (m, 2H).

As second fraction compound 220B (−)-N1-(1-(3-chloro-2-fluorophenyl)ethyl)-N1-cyclopropylethane-1,2-diamine ((Ret.time=1.506; 26.8 mg, 101.79 μmol, 27% yield, 97.52% purity) was obtained as a brown oil ((−)-rotation in DMSO @589 nm/19.9° C., ee %=97.86%). MS (ESI): m/z=257.0 [M+H]⁺, ¹H NMR (400 MHz, DMSO-d₆) δ=7.54-7.48 (m, 1H), 7.45-7.38 (m, 1H), 7.22 (t, J=7.9 Hz, 1H), 6.35-5.65 (m, 2H), 4.36 (q, J=7.0 Hz, 1H), 2.86-2.68 (m, 3H), 2.48-2.41 (m, 1H), 1.78-1.67 (m, 1H), 1.41 (d, J=7.0 Hz, 3H), 0.54-0.38 (m, 3H), 0.33-0.25 (m, 1H).

### Example 16: Synthesis of N′-[1-(2-chloro-3,5-difluoro-phenyl)ethyl]-N′-cyclopropyl-ethane-1,2-diamine hydrochloride (Compound 188)

In analogous manner as set out above for compound 220 (example 15), Compound 188 has been prepared:

| Compound | Structure | LCMS | H NMR |
|---|---|---|---|
| 188 | 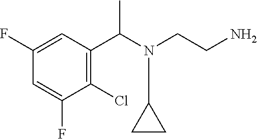 [SMILES: CC(c1cc(F)cc(F)c1Cl)N(CCN)C1CC1] | 275.0 [M + H]⁺ Purity: 97% retention time: 2.433 min method: A | ¹H NMR (400 MHz, DMSO-d₆) δ 7.50-7.97 (m, 3 H), 7.17-7.45 (m, 2 H), 4.42-4.53 (m, 1 H), 2.76- 3.00 (m, 4 H), 1.92- 2.08 (m, 1 H), 1.44 (d, J = 6.85 Hz, |
| | HCl | | 3 H), 0.39-0.58 |
| | | | (m, 4 H) |

### Example 17: Synthesis of N1-(bicyclo[1.1.1]pentan-1-yl)-N1-(1-(3-chloro-2-fluorophenyl) ethyl)ethane-1,2-diamine hydrochloride (Compound 182)

### 1)N-(1-(3-chloro-2-fluorophenyl)ethyl)bicyclo[1.1.1]pentan-1-amine

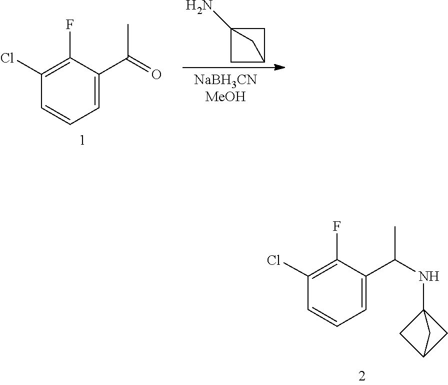 [SMILES: CC(=O)c1cccc(Cl)c1F ; NC12CC(C1)C2 ; CC(NC12CC(C1)C2)c1cccc(Cl)c1F]

To a solution of 1-(3-chloro-2-fluoro-phenyl)ethanone (400 mg, 2.3 mmol, 1 eq) and bicyclo[1.1.1]pentan-i-amine (289 mg, 3.48 mmol, 1.5 eq) in MeOH (5 mL) was added AcOH to adjust pH to 4-5 and stirred at 40° C. for 12 h. Then NaBH₃CN (437 mg, 7 mmol, 3 eq) was added at 20° C. The mixture was stirred at 40° C. for 2 h. LC-MS showed reactant 1 was consumed completely and ˜30% of desired compound was detected. The reaction mixture was quenched by addition of satur. NaHCO₃ solution (10 ml) at 0° C. to pH=7, and then diluted with H₂O (5 ml) and extracted with EtOAc (10 ml×3). The combined organic phase was dried with anhydrous Na₂SO₄, the mixture was filtered and the filtrate was concentrated under vacuum. The residue was purified by flash silica gel chromatography (ISCO®; 12 g SepaFlash® Silica Flash Column, Eluent of 0˜7% ethyl acetate/petroleum ether gradient @90 mL/min). (petroleum ether/ethyl acetate=1/1, Rf=0.62) to give N-(1-(3-chloro-2-fluorophenyl)ethyl)bicyclo[1.1.1]pentan-i-amine (300 mg, 1.25 mmol, 54% yield) as a colourless oil.

### 2) tert-butyl N-[2-[1-bicyclo[1.1.1]pentanyl-[1-(3-chloro-2-fluoro-phenyl)ethyl]amino]ethyl]carbamate

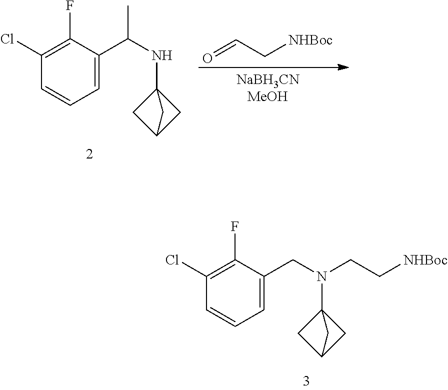 [SMILES: CC(NC12CC(C1)C2)c1cccc(Cl)c1F ; CC(C)(C)OC(=O)NCC=O ; CC(C)(C)OC(=O)NCCN(Cc1cccc(Cl)c1F)C12CC(C1)C2]

To a solution of N-[1-(3-chloro-2-fluoro-phenyl)ethyl]bicyclo[1.1.1]pentan-1-amine (270 mg, 1.13 mmol, 1 eq) and tert-butyl N-(2-oxoethyl)carbamate (179 mg, 1.13 mmol, 1 eq) in MeOH (4 mL) was added AcOH to adjust pH to 4-5 and stirred at 40° C. for 2 h. Then NaBH₃CN (212 mg, 3.38 mmol, 3 eq) was added and the mixture was stirred at 40° C. for 12 h. LC-MS showed ˜15% of reactant 2 was remained and ˜37% of desired compound was detected. The reaction mixture was quenched by addition of satur. NaHCO₃ solution (5 ml) at 0° C., and then diluted with H₂O (10 ml) and extracted with EtOAc (15 ml×3). The combined organic phase was dried with anhydrous Na₂SO₄, the mixture was filtered and the filtrate was concentrated under vacuum. The residue was purified by prep-TLC (SiO₂, petroleum ether/ethyl acetate=1/1, Rf=0.54) to give tert-butyl N-[2-[1-bicyclo[1.1.1]pentanyl-[1-(3-chloro-2-fluoro-phenyl)ethyl]amino]ethyl]carbamate (100 mg, 261 μmol, 23% yield) as a colourless oil.

### 3) N1-(bicyclo[1.1.1]pentan-1-yl)-N1-(1-(3-chloro-2-fluorophenyl)ethyl)ethane-1,2-diamine hydrochloride (Compound 182)

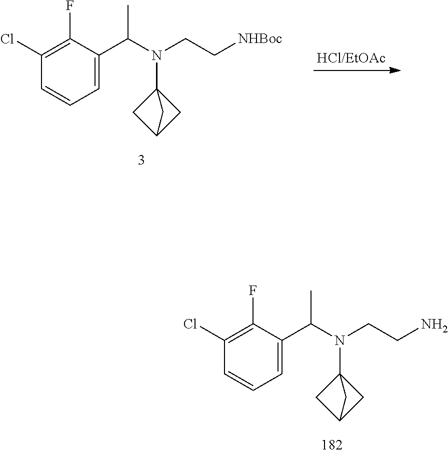 [SMILES: CC(c1cccc(Cl)c1F)N(CCNC(=O)OC(C)(C)C)C12CC(C1)C2 ; CC(c1cccc(Cl)c1F)N(CCN)C12CC(C1)C2]

To a solution of tert-butyl N-[2-[1-bicyclo[1.1.1]pentanyl-[1-(3-chloro-2-fluoro-phenyl)ethyl]amino]ethyl]carbamate (50 mg, 130.6 μmol, 1 eq) in EtOAc (1 mL) and HCl/EtOAc (0.5 mL, 4M) was stirred at 20° C. for 1 h. LC-MS showed reactant 1 was consumed completely and ˜64% of desired compound was detected. The mixture was concentrated to give a residue. The residue was purified by prep-HPLC (HCl condition, column: Phenomenex Luna 80×30 mm×3 um; mobile phase: [water(HCl)-ACN]; B %: 1%-40%, 8 min) to give N1-(bicyclo[1.1.1]pentan-1-yl)-N1-(1-(3-chloro-2-fluorophenyl)ethyl)ethane-1,2-diamine hydrochloride (33.5 mg, 118.5 μmol, 91% yield, 100% purity, HCl) as a white solid. MS (ESI): m/z=283.0 [M+H]⁺; retention time: 2.127 min, method:A ¹H NMR (400 MHz, DMSO-d₆) δ 8.16 (br s, 3H), 7.44-7.65 (m, 2H), 7.21 (t, J=7.76 Hz, 1H), 4.51 (q, J=6.93 Hz, 1H), 3.01-3.25 (m, 2H), 2.66-2.90 (m, 2H), 2.38 (s, 1H), 1.83-1.90 (m, 3H), 1.77-1.82 (m, 3H), 1.47 (d, J=6.97 Hz, 3H).

Example 18: Synthesis of N1-(cyclobutyl)-N1-(1-(3-chloro-2-fluorophen, l)ethyl)ethane-1,2-diamine hydrochloride (Compound 180) and N1-(cyclopropyl)-N1-(1-(2,5-dichlorophenyl)ethyl)ethane-1,2-diamine hydrochloride (Compound 185)

In analogous manner as set out above for Compound 182 (example 17), the following further compounds have been prepared:

| Compound | Structure | LCMS | H NMR |
|---|---|---|---|
| 180 | 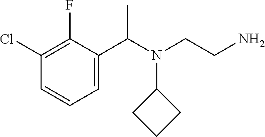 [SMILES: CC(c1cccc(Cl)c1F)N(CCN)C1CCC1] | 271.0 [M + H]⁺ Purity: 99.45% retention time: 1.912 min method: A | ¹HNMR (400 MHz, DMSO-d₆) δ 11.94 (br s, 1 H), 8.51 (br d, J = 17.39 Hz, 3 H), 7.65- 8.03 (m, 2 H), 7.27- 7.44 (m, 1 H), 4.74- 5.06 (m, 1 H), 3.56- 4.16 (m, 3 H), 3.40 (br d, J = 12.63 Hz, 1 H), 2.98- |
| | 2 HCl | | 3.19 (m, 1 H), 2.56- |
| | | | 2.83 (m, 1 H), 2.43 (br d, |
| | | | J = 8.38 Hz, 1 H), 2.07- |
| | | | 2.28 (m, 2 H), 1.50- |
| | | | 1.79 (m, 5 H). |
| 185 | 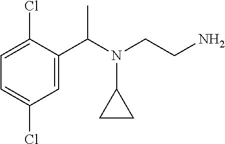 [SMILES: CC(c1cc(Cl)ccc1Cl)N(CCN)C1CC1] | 273.0 [M + H]⁺ Purity: 99.12% retention time: 1.998 min method: A | ¹HNMR (400 MHz, DMSO-d₆) δ 8.13-8.47 (m, 3 H), 7.93 (br s, 1 H), 7.50-7.56 (m, 1 H), 7.42-7.47 (m, 1 H), 4.77 (br s, 1 H), 3.02- 3.42 (m, 4 H), 2.43 (br s, 1 H), 1.62 (br d, J = 6.72 Hz, 3 H), 0.75-1.06 |
| | HC1 | | (m, 2 H), 0.55-0.74 (m, |
| | | | 2 H). |

### Example 19: Synthesis of N1-allyl-N1-(1-(3-chloro-2-fluorophenyl)ethyl)ethane-1,2-di-amine hydrochloride (Compound 172)

### 1) tert-butyl(2-((1-(3-chloro-2-fluorophenyl)ethyl)amino)ethyl)carbamate

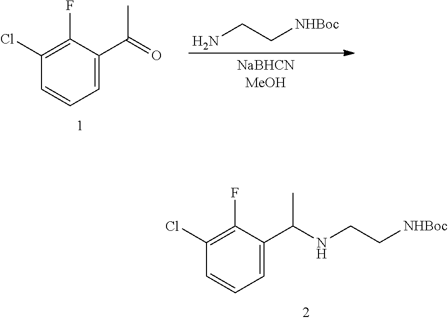 [SMILES: CC(=O)c1cccc(Cl)c1F ; CC(C)(C)OC(=O)NCCN ; CC(NCCNC(=O)OC(C)(C)C)c1cccc(Cl)c1F]

A solution of 1-(3-chloro-2-fluoro-phenyl)ethanone (2 g, 11.59 mmol, 1 eq) and tert-butyl N-(2-aminoethyl)carbamate (1.86 g, 11.59 mmol, 1.82 mL, 1 eq) in MeOH (20 mL) was adjusted to pH 4-5 by addition of AcOH. The mixture was stirred at 70° C. for 12 h, then NaBH₃CN (2.18 g, 34.77 mmol, 3 eq) was added and the mixture was stirred at 70° C. for 2 h. The reaction mixture was concentrated under reduced pressure to remove solvent to get a residue which was diluted with water (10 mL) and extracted with ethyl acetate (10 mL×3). The combined organic layers were washed with brine (10 mL×2), dried over Na₂SO₄, filtered and concentrated under reduced pressure to give a residue which was purified by column chromatography (SiO₂, petroleum ether/ethyl acetate=5/1 to 1/1) to give compound tert-butyl N-[2-[1-(3-chloro-2-fluoro-phenyl)ethylamino]ethyl]carbamate (1.8 g, 5.68 mmol, 49% yield) as a red oil.

### 2) tert-butyl (2-(allyl(1-(3-chloro-2-fluorophenyl)ethyl)amino)ethyl)carbamate

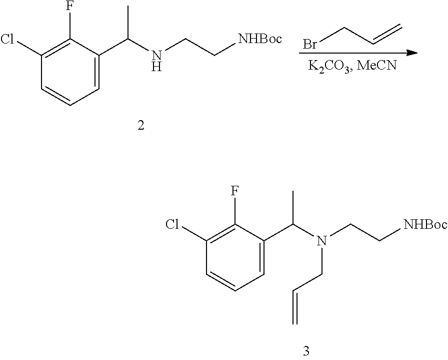

To a solution of tert-butyl N-[2-[1-(3-chloro-2-fluoro-phenyl)ethylamino]ethyl]carbamate (200 mg, 631.3 μmol, 1 eq) in MeCN (2 mL) was added K₂CO₃ (262 mg, 1.89 mmol, 3 eq) and 3-bromoprop-1-ene (115 mg, 947 μmol, 1.5 eq). The mixture was stirred at 70° C. for 12 h. Then the mixture was filtrated and the filtrate was concentrated to get compound tert-butyl N-[2-[allyl-[1-(3-chloro-2-fluoro-phenyl)ethyl]amino]ethyl]carbamate (200 mg, crude) as a yellow oil.

### 3) N1-allyl-N1-(1-(3-chloro-2-fluorophenyl)ethyl)ethane-1,2-diamine hydrochloride (Compound 172)

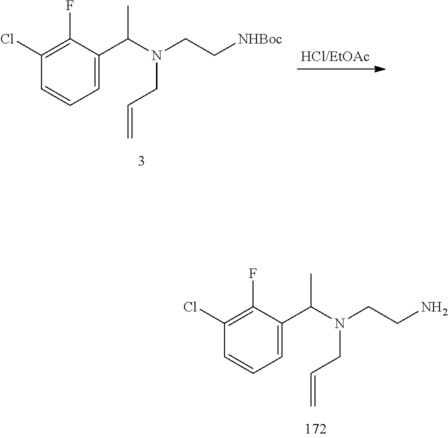 [SMILES: C=CCN(CCNC(=O)OC(C)(C)C)C(C)c1cccc(Cl)c1F ; C=CCN(CCN)C(C)c1cccc(Cl)c1F]

To a solution of tert-butyl N-[2-[allyl-[1-(3-chloro-2-fluoro-phenyl)ethyl]amino]ethyl]-carbamate (100 mg, 280.2 μmol, 1 eq) in EtOAc (2 mL) was added HCl/EtOAc (1 mL, 4M). The mixture was stirred at 25° C. for 12 h. The mixture was concentrated to get a residue. The residue was purified by prep-HPLC (HCl condition; column: Phenomenex Luna 80×30 mm×3 um; mobile phase: [water(HCl)-ACN]; B %: 15%-45%, 8 min) to give compound N1-allyl-N1-(1-(3-chloro-2-fluorophenyl)ethyl)ethane-1,2-diamine hydrochloride (58 mg, 196.4 μmol, 70% yield, 99.5% purity, HCl) as a colourless oil. MS (ESI): m/z=257.0 [M+H]⁺; retention time: 1.607 min, method: K; ¹H NMR (400 MHz, DMSO-d₆) δ=8.43-8.07 (m, 3H), 7.86-7.73 (m, 1H), 7.62 (br t, J=7.5 Hz, 1H), 7.32 (t, J=7.9 Hz, 1H), 6.10-5.92 (m, 1H), 5.48-5.35 (m, 2H), 4.75-4.54 (m, 1H), 3.84-2.96 (m, 6H), 1.66 (br d, J=4.8 Hz, 3H).

### Example 20: Synthesis of N1-(2-(methylthio)ethyl)-N1-(1-(3-chloro-2-fluorophenyl)ethyl) ethane-1,2-diamine (Compound 169)

In analogous manner as set out above for Compound 172 (example 19), the following further compound has been prepared:

| Compound | Structure | LCMS | H NMR |
|---|---|---|---|
| 169 | 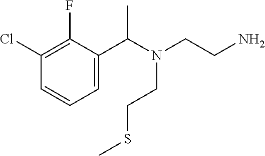 [SMILES: CSCCN(CCN)C(C)c1cccc(Cl)c1F] | 291.0 [M + H]⁺ Purity: 92.68% retention time: 1.905 min method: K | ¹H NMR (400 MHz, DMSO-d₆) δ = 8.36-8.16 (m, 3H), 7.86-7.71 (m, 1H), 7.61 (br t, J = 7.5 Hz, 1H), 7.32 (t, J = 8.0 Hz, 1H), 4.83-4.61 (m, 1H), 3.21-3.05 (m, 5H), 3.04- 2.74 (m, 4H), 2.06 (s, 3H), 1.64 (br d, J = 6.3 Hz, 3H). |
| | HCl | | |

### Example 21: Synthesis of N1-(3-chloro-2-fluorobenzyl)-N1-cyclopropylethane-1,2-diamine hydrochloride (Compound 120)

### 1)N-(3-chloro-2-fluorobenzyl)cyclopropanamine

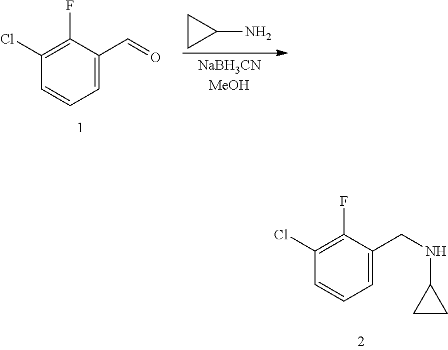

To a solution of 3-chloro-2-fluoro-benzaldehyde (500 mg, 3.15 mmol, 1 eq) and cyclopropanamine (360 mg, 6.31 mmol, 437 μL, 2 eq) in MeOH (20 mL) was added AcOH to adjust pH to 4-5 and then the mixture was stirred at 25° C. for 12 h. Then NaBH₃CN (595 mg, 9.46 mmol, 3 eq) was added, the mixture was stirred at 25° C. for 2 h. The reaction mixture was quenched by addition of satur. NaHCO₃ solution 5 mL at 25° C., and extracted with ethyl acetate (15 mL×3). The combined organic layers were washed with brine (5 mL×2), dried over Na₂SO₄, filtered and concentrated under reduced pressure to give compound N-[(3-chloro-2-fluoro-phenyl)methyl]cyclopropanamine (600 mg, crude) as a colourless oil.

### 2) tert-butyl (2-((3-chloro-2-fluorobenzyl)(cyclopropyl)amino)ethyl)carbamate

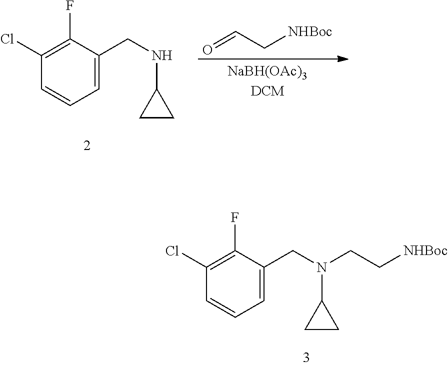 [SMILES: Fc1c(Cl)cccc1CNC1CC1 ; CC(C)(C)OC(=O)NCC=O ; CC(C)(C)OC(=O)NCCN(Cc1cccc(Cl)c1F)C1CC1]

To a solution of N-[(3-chloro-2-fluoro-phenyl)methyl]cyclopropanamine (200 mg, 1 mmol, 1 eq) in DCM (3 mL) was added tert-butyl N-(2-oxoethyl)carbamate (160 mg, 1 mmol, 1 eq), the mixture was adjusted to pH to 4-5 by addition of AcOH and stirred at 25° C. for 1 h. Then NaBH(OAc)₃ (637 mg, 3 mmol, 3 eq) was added and the mixture was stirred at 25° C. for 12 h. The mixture was concentrated in vacuo to get the crude product tert-butyl N-[2-[(3-chloro-2-fluoro-phenyl)methyl-cyclopropyl-amino]ethyl]carbamate (160 mg, crude) as a pale yellow oil.

### 3) N1-(3-chloro-2-fluorobenzyl)-N1-cyclopropylethane-1,2-diamine hydrochloride (Compound 120)

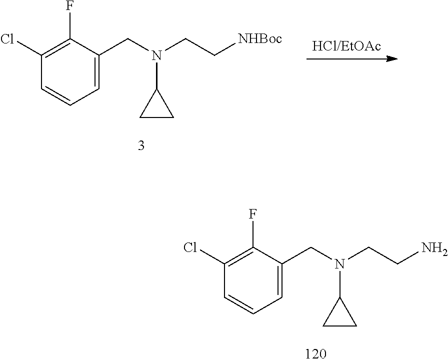 [SMILES: CC(C)(C)OC(=O)NCCN(Cc1cccc(Cl)c1F)C1CC1 ; NCCN(Cc1cccc(Cl)c1F)C1CC1]

A mixture of tert-butyl N-[2-[(3-chloro-2-fluoro-phenyl)methyl-cyclopropyl-amino]ethyl]carbamate (160 mg, 467 μmol, 1 eq) in HCl/EtOAc (2 mL, 4M) was stirred at 25° C. for 30 min. The mixture was concentrated in vacuo to get a residue which was purified by prep-HPLC (HCl condition column: Phenomenex luna C18 80*40 mm*3 μm; mobile phase: [water(HCl)-ACN]; B %: 1%-20%, 7 min) to give compound N1-(3-chloro-2-fluorobenzyl)-N1-cyclopropylethane-1,2-diamine hydrochloride (59 mg, 210 μmol, 45% yield, 99.7% purity, HCl) as a white solid. MS (ESI): m/z=243.0 [M+H]⁺; retention time: 1.654 min, method: A ¹H NMR (400 MHz, DMSO-d₆) δ 8.16 (br s, 3H), 7.44-7.65 (m, 2H), 7.21 (t, J=7.76 Hz, 1H), 4.51 (q, J=6.93 Hz, 1H), 3.01-3.25 (m, 2H), 2.66-2.90 (m, 2H), 2.38 (s, 1H), 1.83-1.90 (m, 3H), 1.77-1.82 (m, 3H), 1.47 (d, J=6.97 Hz, 3H).

### Example 22: Synthesis of Compound 130

In analogous manner as set out above for Compound 120 (example 21), Compound 130 has been prepared:

| Compound | Structure | LCMS | H NMR |
|---|---|---|---|
| 130 | HCl | 256.0 [M + H]+ | 1H NMR (400 MHz, |
| | | Purity: 98.16% | DMSO-d₆) δ = 7.99- |
| | | retention time: | 7.75 (m, 3H), 7.64-7.47 |
| | | 1.755 min | (m, 2H), 7.32-7.20 (m, |
| | | method: A | 1H), 3.85 (br s, 2H), |
| | | | 3.11-2.70 (m, 7H). |

### Example 23: Synthesis of Ni-(1-(3-bromo-2-fluorophenyl)ethyl)-N¹-cyclopropylethane-1,2-diamine hydrochloride (Compound 191)

### 1)N-(1-(3-bromo-2-fluorophenyl)ethyl)cyclopropanamine

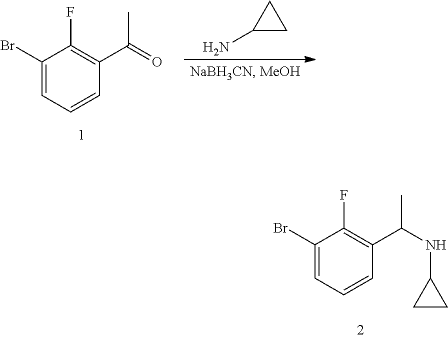

To a solution of 1-(3-bromo-2-fluoro-phenyl)ethenone (CAS161957-61-5) (500 mg, 2.3 mmol, 1 eq) in MeOH (5 mL) was added cyclopropanamine (197.3 mg, 3.5 mmol, 239.4 μL, 1.5 eq). Then the reaction was stirred at 60° C. for 12 h. Then NaBH₃CN (579.1 mg, 9.2 mmol, 4 eq) was added at 20° C. and the mixture was stirred at 60° C. for 2 h. LC-MS showed reactant 1 was consumed and desired compound was detected. The reaction mixture was concentrated under reduced pressure to remove solvent. The residue was diluted with H₂O 6 mL and extracted with Ethyl acetate (4 mL×3). The combined organic layers were washed with H₂O (8 mL×2), dried over Na₂SO₄, filtered and concentrated under reduced pressure to give a residue. The residue was purified by prep-TLC (SiO₂, Petroleum ether/Ethyl acetate=5/1). Compound N-[1-(3-bromo-2-fluoro-phenyl)ethyl]cyclopropanamine (320 mg, 1.2 mmol, 53.8% yield) was obtained as a white solid.

### 2) 2-((1-(3-bromo-2-fluorophenyl)ethyl)(cyclopropyl)amino)acetamide

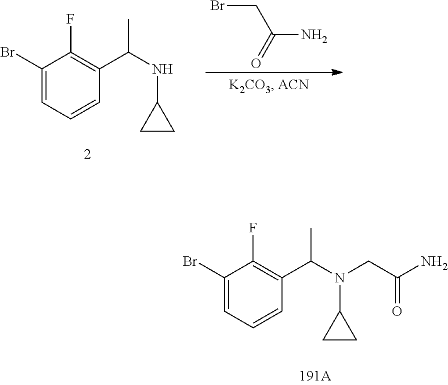 [SMILES: CC(NC1CC1)c1cccc(Br)c1F ; NC(=O)CBr ; CC(c1cccc(Br)c1F)N(CC(N)=O)C1CC1]

To a solution of N-[1-(3-bromo-2-fluoro-phenyl)ethyl]cyclopropanamine (290 mg, 1.1 mmol, 1 eq) in ACN (4 mL) was added 2-bromoacetamide (372.0 mg, 2.7 mmol, 2.4 eq) and K₂CO₃ (465.81 mg, 3.37 mmol, 3 eq). The mixture was stirred at 80° C. for 12 hr. LC-MS showed ˜50% of 2 remained and ˜24% of desired compound was detected. The reaction mixture was concentrated under reduced pressure to remove solvent. The residue was diluted with H₂O 6 mL and extracted with Ethyl acetate (4 mL×3). The combined organic layers were washed with H₂O (8 mL×2), dried over Na₂SO₄, filtered and concentrated under reduced pressure to give a residue. The residue was purified by prep-TLC (SiO₂, Petroleum ether/Ethyl acetate=1/1). Compound 2-[1-(3-bromo-2-fluoro-phenyl)ethyl-cyclopropyl-amino]acetamide (58 mg, 184.0 μmol, 16.4% yield) was obtained as a yellow oil.

### 3) N¹-(1-(3-bromo-2-fluorophenyl)ethyl)-N¹-cyclopropylethane-1,2-diamine hydrochloride (Compound 191)

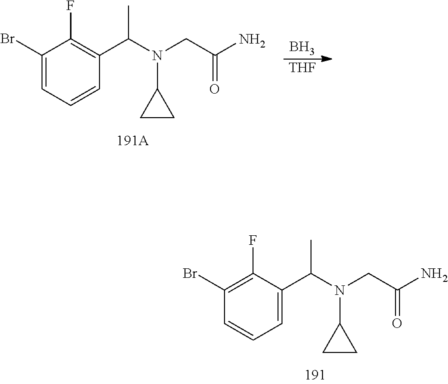

To a solution of 2-[1-(3-bromo-2-fluoro-phenyl)ethyl-cyclopropyl-amino]acetamide (58 mg, 184.0 μmol, 1 eq) in THF (1 mL) was added BH₃·THF (1 M, 736.1 μL, 4 eq). The mixture was stirred at 70° C. for 12 hr. LC-MS showed desired compound was detected. The residue was diluted with MeOH 5 mL and concentrated under reduced pressure to remove solvent. The residue was purified by prep-HPLC (HCl condition, column: Phenomenex luna C18 80×40 mm×3 um; mobile phase: [water(HCl)-ACN]; B %: 5%-35%, 7 min). Compound N¹-(1-(3-bromo-2-fluorophenyl)ethyl)-N¹-cyclopropylethane-1,2-diamine hydrochloride (4.3 mg, 14.3 μmol, 7.8% yield, HCl, 100% purity) was obtained as colourless oil. MS (ESI): m/z 301.3 [M+H]⁺; retention time: 1.838 min, method: B ¹H NMR (400 MHz, DMSO-d6) δ 8.41-7.91 (m, 3H), 7.90-7.56 (i, 2H), 7.31-7.16 (m, 1H), 5.24-4.52 (m, 1H), 3.31-2.98 (m, 4H), 2.54 (s, 1H), 1.76-1.51 (m, 3H), 1.14-0.44 (m, 4H).

### Example 24: Synthesis of Compounds 242, 243, 244, and 245

In analogous manner as set out above for Compound 191 (example 23), the following further compounds have been prepared:

| Compound | Structure | LCMS | H NMR |
|---|---|---|---|
| 242 | 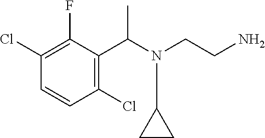 [SMILES: CC(c1c(Cl)ccc(Cl)c1F)N(CCN)C1CC1] | 291.2 [M + H]⁺ Purity: 100% retention time: 1.618 min, method: M | ¹H NMR (400 MHz, DMSO-d6) δ = 8.34- 7.86 (m, 3H), 7.76-7.57 (m, 1H), 7.43 (br d, J = 8.0 Hz, 1H), 4.76 (br d, J = 4.1 Hz, 5H), 3.49- 3.18 (m, 1H), 1.75-1.54 (m, 3H), 0.85-0.25 (m, 4H). |
| | HCl | | |
| 243 | 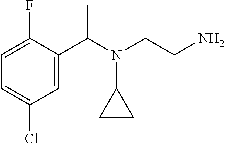 [SMILES: CC(c1cc(Cl)ccc1F)N(CCN)C1CC1] | 257.2 [M + H]⁺ Purity: 99.62% retention time: 1.429 min, method: M | ¹H NMR (400 MHz, DMSO-d6) δ = 8.59- 8.14 (m, 3H), 7.93 (br d, J = 1.6 Hz, 1H), 7.56 (br s, 1H), 7.43-7.31 (m, 1H), 5.06-4.79 (m, 1H), 3.59-3.02 (m, 4H), 2.81- 2.56 (m, 1H), 1.72 (br s, 3H), 1.34-0.57 (m, 4H). |
| | HCl | | |
| 244 | HCl | 275.2 [M + H]⁺ | ¹H NMR (400 MHz, |
| | | Purity: 100% | DMSO-d6) δ = 8.42- |
| | | retention | 8.21 (m, 3H), 7.58 (dt, J = |
| | | time: 1.515 min, | 4.5, 8.6 Hz, 1H), 7.39 |
| | | method: M | (dt, J = 3.9, 9.9 Hz, 1H), |
| | | | 4.95 (br d, J = 2.3 Hz, |
| | | | 1H), 3.51-3.17 (m, 4H), |
| | | | 2.48-2.30 (m, 1H), 1.77- |
| | | | 1.66 (m, 3H), 0.98- |
| | | | 0.55 (m, 4H). |
| 245 | HCl | 275.2 [M + H]⁺ | ¹H NMR (400 MHz, |
| | | Purity: 100.00% | DMSO-d6) δ = 8.31- |
| | | retention | 7.83 (m, 3H), 7.63-7.31 |
| | | time: 1.504 min | (m, 2H), 4.94-4.62 (m, |
| | | method: M | 1H), 3.46-2.78 (m, 4H), |
| | | | 2.34-2.01 (m, 1H), 1.65 |
| | | | (br s, 3H), 0.88-0.33 |
| | | | (m, 4H) |

### Example 25: Synthesis of Ni-(1-(3-chloro-2-fluorophenyl)propyl)-N1-cyclopropylethane-1,2-diamine hydrochloride (Compound 224)

### 1) 1-(3-chloro-2 fluorophenyl)propan-1-ol

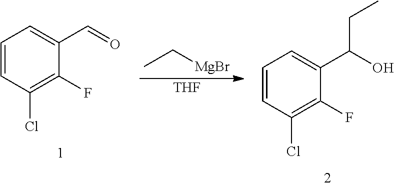

To a solution of 3-chloro-2-fluoro-benzaldehyde (1 g, 6.3 mmol, 1 eq) in THF (5 mL) was added bromo (ethyl) magnesium (3 M, 2.5 mL, 1.2 eq) at 0° C. under N₂. The mixture was stirred at 20° C. for 12 hr. TLC indicated Reactant 1 was consumed completely and one new spot formed. The reaction mixture was quenched by addition into sat. NH₄Cl solution (20 ml) at 20° C. and extracted with Ethyl acetate (10 mL×3). The combined organic layers were washed with NaCl (20 mL×2), dried over Na₂SO₄, and concentrated under reduced pressure to give crude product 1-(3-chloro-2-fluorophenyl)propan-1-ol (1 g, 5.3 mmol, 84.1% yield) as yellow oil.

### 2) 1-(3-chloro-2-fluorophenyl)propan-1-one

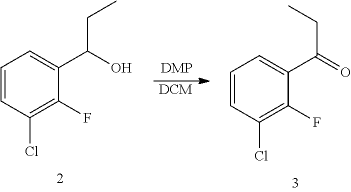

To a solution of 1-(3-chloro-2-fluoro-phenyl)propan-1-ol (1 g, 5.3 mmol, 1 eq) in DCM (20 mL) was added DMP (3.4 g, 8.0 mmol, 1.5 eq). The mixture was stirred at 20° C. for 12 hr. TLC indicated 2 was consumed and one major new spot with larger polarity was detected. The residue was quenched with sat. Na₂SO₃ 20 mL and extracted with DCM 30 mL (10 mL×3). The combined organic layers were dried over Na₂SO₄, filtered and concentrated under reduced pressure to give a residue. The residue was purified by flash silica gel chromatography (Silica Flash Column, Eluent of 0˜20% Ethyl acetate/Petroleum ether gradient @120 mL/min) to afford compound 1-(3-chloro-2-fluorophenyl)propan-1-one (790 mg, 4.23 mmol, 79.85% yield) as colourless oil.

### 3)N-(1-(3-chloro-2-fluorophenyl)propyl)cyclopropanamine

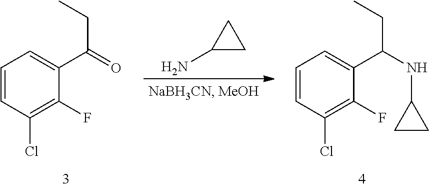

To a solution of 1-(3-chloro-2-fluoro-phenyl)propan-1-one (390 mg, 2.1 mmol, 1 eq) in MeOH (2 mL) was added cyclopropanamine (179.0 mg, 3.1 mmol, 217.2 uL, 1.5 eq). Then the reaction was stirred at 60° C. for 12 hr. Then NaBH₃CN (525.3 mg, 8.4 mmol, 4 eq) was added and the mixture was stirred at 60° C. for 2 h. LC-MS showed desired compound was detected. The reaction mixture was concentrated under reduced pressure to remove solvent. The residue was diluted with H₂O 6 mL and extracted with Ethyl acetate (4 mL×3). The combined organic layers were washed with H₂O (8 mL×2), dried over Na₂SO₄, filtered and concentrated under reduced pressure to give a residue. The residue was purified by prep-TLC (SiO₂, Petroleum ether/Ethyl acetate=3:1) to give compound N-(1-(3-chloro-2-fluorophenyl)propyl)cyclopropanamine (250 mg, 805.2 μmol, 38.5% yield, 73.3% purity) as yellow oil.

### 4) 2-((1-(3-chloro-2-fluorophenyl)propyl)(cyclopropyl)amino)acetamide

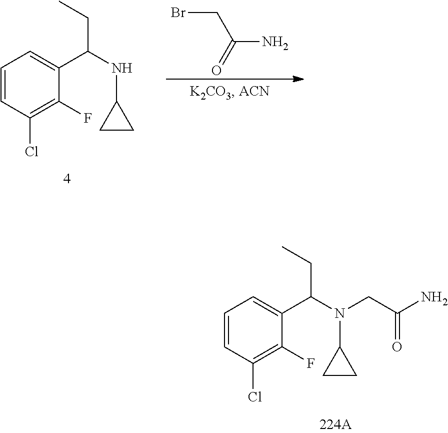 [SMILES: CCC(NC1CC1)c1cccc(Cl)c1F ; NC(=O)CBr ; CCC(c1cccc(Cl)c1F)N(CC(N)=O)C1CC1]

To a solution of 2-bromoacetamide (455.3 mg, 3.3 mmol, 3 eq) in ACN (5 mL) was added K₂CO₃ (456.09 mg, 3.30 mmol, 3 eq) and N-[1-(3-chloro-2-fluoro-phenyl)propyl]cyclopropanamine (250 mg, 1.1 mmol, 1 eq). The mixture was stirred at 80° C. for 12 hr. LC-MS showed desired compound was detected. The reaction mixture was concentrated under reduced pressure to remove solvent. The residue was diluted with H₂O 50 mL and extracted with EtOAc 150 mL (50 mL×3), dried over Na₂SO₄, filtered and concentrated under reduced pressure to give a residue. The residue was purified by prep-TLC (SiO₂, Petroleum ether/Ethyl acetate=0:1). The residue was purified by prep-HPLC (HCl condition). (column: Phenomenex Luna 80×30 mm×3 um; mobile phase: [water(HCl)-ACN]; B %: 10%-45%, 8 min) to give Compound 2-((1-(3-chloro-2-fluorophenyl)propyl)(cyclopropyl)amino) acetamide (110 mg, 353.6 μmol, 32.2% yield, 91.6% purity) as a white solid.

### 5) N1-(1-(3-chloro-2-fluorophenyl)propyl)-N1-cyclopropylethane-1,2-diamine hydrochloride (Compound 224)

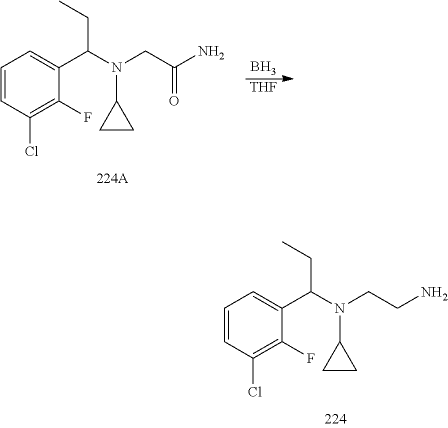

To a solution of 2-[1-(3-chloro-2-fluoro-phenyl)propyl-cyclopropyl-amino]acetamide (110 mg, 386.3 μmol, 1 eq) in THF (3 mL) was added BH₃·THF (1 M, 2.4 mL, 6 eq). The mixture was stirred at 70° C. for 12 h. LC-MS showed desired compound was detected. 2 mL MeOH was added to the reaction. The reaction mixture was concentrated under reduced pressure to remove solvent. The residue was purified by prep-HPLC (HCl condition). column: Phenomenex Luna 80×30 mm×3 um; mobile phase: [water(HCl)-ACN]; B %: 5%-35%, 8 min to afford compound N1-(1-(3-chloro-2-fluorophenyl)propyl)-N1-cyclopropylethane-1,2-diamine hydrochloride (37 mg, 136.7 μmol, 35.4% yield, 100% purity, HCl) as colorless oil. MS (ESI): m/z=271.1 [M+H]⁺; retention time: 1.558 min, method: M. ¹H NMR (400 MHz, DMSO-d6) δ=8.01 (br dd, J=1.8, 3.3 Hz, 3H), 7.61-7.50 (m, 2H), 7.33-7.24 (m, 1H), 4.33 (br dd, J=1.0, 2.0 Hz, 1H), 3.29-3.14 (m, 1H), 3.13-2.98 (m, 2H), 2.96-2.77 (m, 1H), 2.26-2.13 (m, 1H), 2.11-1.92 (m, 2H), 0.88-0.70 (m, 5H), 0.63 (br d, J=4.0 Hz, 2H).

### Example 26: Synthesis of Ni-(1-(3-chloro-2-fluorophenyl) propyl)-N1-propylethane-1,2-diamine hydrochloride (Compound 262)

### 1) 1-(3-chloro-2-fluorophenyl)propan-1-ol

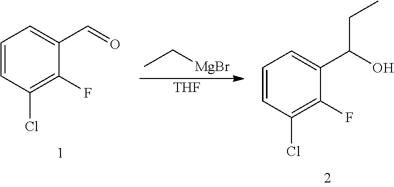

To a solution of 3-chloro-2-fluoro-benzaldehyde (1 g, 6.3 mmol, 1 eq) in THF (5 mL) was added bromo(ethyl)magnesium (3 M, 2.5 mL, 1.2 eq) at 0° C. under N₂. The mixture was stirred at 20° C. for 12 h. TLC indicated Reactant 1 was consumed completely and one new spot formed. The reaction mixture was quenched by addition into sat. NH₄Cl solution (20 ml) at 20° C. and extracted with Ethyl acetate (10 mL×3). The combined organic layers were washed with NaCl (20 mL×2), dried over Na₂SO₄, and concentrated under reduced pressure to give crude product 1-(3-chloro-2-fluorophenyl)propan-1-ol (1 g, 5.3 mmol, 84.1% yield) as yellow oil.

### 2) 1-(3-chloro-2-fluorophenyl)propan-1-one

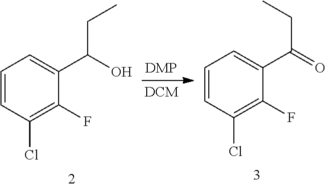

To a solution of 1-(3-chloro-2-fluoro-phenyl)propan-1-ol (1 g, 5.3 mmol, 1 eq) in DCM (20 mL) was added DMP (3.4 g, 8.0 mmol, 1.5 eq). The mixture was stirred at 20° C. for 12 hr. TLC indicated one major new spot with larger polarity was detected. The residue was diluted with sat. Na₂SO₃ 200 mL and extracted with DCM 300 mL (100 mL×3). The combined organic layers were dried over Na2SO4, filtered and concentrated under reduced pressure to give a residue. The residue was purified by flash silica gel chromatography (Silica Flash Column, Eluent of 0-20% Ethyl acetate/Petroleum ether gradient @120 m/min) to afford compound 1-(3-chloro-2-fluorophenyl)propan-1-one (790 mg, 4.23 mmol, 79.85% yield) as colourless oil.

### 3) 1-(3-chloro-2-fluorophenyl)-N-propylpropan-1-amine

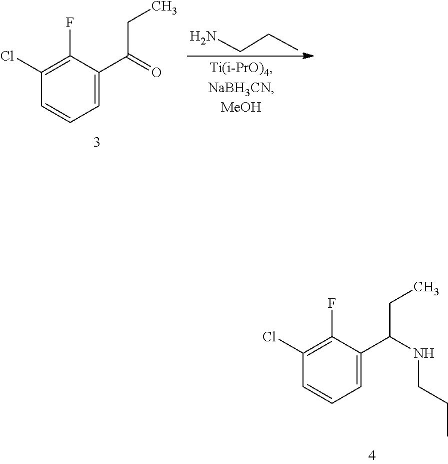

To a solution of 1-(3-chloro-2-fluoro-phenyl)propan-1-one (200 mg, 1.1 mmol, 1 eq) in MeOH (3 mL) was added propan-1-amine (95.0 mg, 1.6 mmol, 132.2 μL, 1.5 eq) and Ti(i-PrO)₄ (913.8 mg, 3.2 mmol, 948.9 μL, 3 eq). The mixture was stirred at 60° C. for 12 hr. NaBH₃CN (269.4 mg, 4.3 mmol, 4 eq) was added to the reaction at 20° C. and stirred at 60° C. for 2 hr. LC-MS showed 3 was consumed completely and one main peak with desired m/z was detected. The reaction mixture was concentrated under reduced pressure to remove solvent. The residue was diluted with H₂O 15 mL and extracted with EtOAc 30 mL (10 mL×3), dried over Na₂SO₄, filtered and concentrated under reduced pressure to give crude product 1-(3-chloro-2-fluorophenyl)-N-propylpropan-1-amine (210 mg, crude) as yellow oil.

### 4) tert-butyl (2-((1-(3-chloro-2-fluorophenyl)propyl)(propyl)amino)ethyl)carbamate

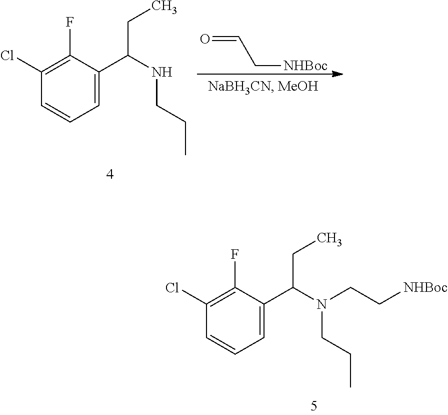 [SMILES: CCCNC(CC)c1cccc(Cl)c1F ; CC(C)(C)OC(=O)NCC=O ; CCCN(CCNC(=O)OC(C)(C)C)C(CC)c1cccc(Cl)c1F]

To a solution of 1-(3-chloro-2-fluoro-phenyl)-N-propyl-propan-1-amine (150 mg, 653.0 mol, 1 eq) in MeOH (2 mL) was added tert-butyl N-(2-oxoethyl)carbamate (727.6 mg, 4.6 mmol, 7 eq) and AcOH to adjust pH=5, the mixture was stirred at 60° C. for 12 hr. Then NaBH₃CN (164.1 mg, 2.6 mmol, 4 eq) was added at 20° C. The mixture was stirred at 60° C. for 2 hr. LC-MS showed 4 was consumed completely and one main peak with desired m/z was detected. The reaction mixture was concentrated under reduced pressure to remove solvent. The residue was diluted with H₂O 10 mL and extracted with EtOAc 30 mL (10 mL×3), dried over Na₂SO₄, filtered and concentrated under reduced pressure to give a residue. The residue was purified by prep-TLC (SiO₂, Petroleum ether/Ethyl acetate=3/1) to give compound tert-butyl (2-((1-(3-chloro-2-fluorophenyl)propyl)(propyl)amino)ethyl) carbamate (73 mg, 195.8 μmol, 30.0% yield) as a white solid.

### 5) N1-(1-(3-chloro-2-fluorophenyl)propyl)-N1-propylethane-1,2-diamine hydrochloride (Compound 262)

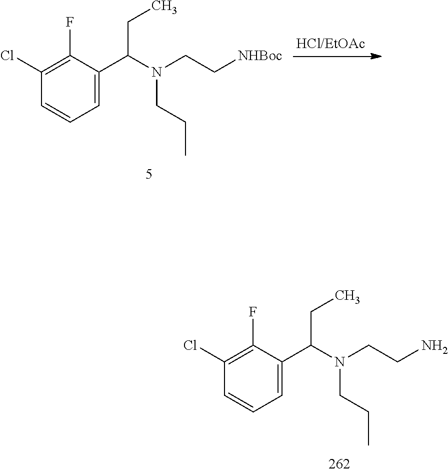 [SMILES: CCCN(CCNC(=O)OC(C)(C)C)C(CC)c1cccc(Cl)c1F ; CCCN(CCN)C(CC)c1cccc(Cl)c1F]

A solution of tert-butyl N-[2-[1-(3-chloro-2-fluoro-phenyl)propyl-propyl-amino]ethyl]carbamate (73 mg, 195.8 μmol, 1 eq) in HCl/EtOAc (5 mL) was stirred at 20° C. for 1 hr. LC-MS showed 5 was consumed completely and one main peak with desired m/z was detected. The reaction mixture was concentrated under reduced pressure to remove solvent. 2 mL MeOH was added to the reaction. The residue was purified by prep-HPLC (HCl condition). column: Phenomenex Luna 80×30 mm×3 um; mobile phase: [water(HCl)-ACN]; gradient: 10%-35% B over 8 min to give compound N1-(1-(3-chloro-2-fluorophenyl) propyl)-N1-propylethane-1,2-diamine hydrochloride (28.03 mg, 90.64 μmol, 46.30% yield, 100% purity, HCl) as yellow oil. MS (ESI): m/z=273.3[M+H]⁺; retention time: 1.368 min, method: M. Special ¹H NMR (400 MHz, DMSO-d6) δ=8.81-8.22 (m, 3H), 7.95-7.79 (m, 1H), 7.67 (br t, J=7.5 Hz, 1H), 7.36 (t, J=7.9 Hz, 1H), 4.70-4.44 (m, 1H), 3.34-3.09 (m, 4H), 3.05 (br s, 2H), 2.41-2.07 (m, 2H), 1.85-1.61 (m, 2H), 0.87 (br t, J=6.9 Hz, 3H), 0.72 (t, J=7.3 Hz, 3H).

### Example 27: Synthesis of Compounds 190, 258 and 248

In analogous manner as set out above for Compound 262 (example 26), the following further compounds have been prepared:

| Compound | Structure | LCMS | H NMR |
|---|---|---|---|
| 190 | 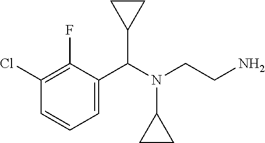 [SMILES: NCCN(C1CC1)C(c1cccc(Cl)c1F)C1CC1] | 283.0 [M + H]⁺ Purity: 99.34% retention time: 1.932 min method: M | ¹H NMR (400 MHz, DMSO-d6) δ ppm 8.12 (br d, J = 1.50 Hz, 3 H), 7.75 (br s, 1 H), 7.51- 7.60 (m, 1 H), 7.27 (t, J = 7.88 Hz, 1 H), 3.35 (br s, 1 H), 3.13 (br s, 4 H), 2.21-2.42 (m, 1 H), 1.61 (br s, 1 H), 0.59- |
| | HCl | | 0.87 (m, 6 H), 0.48- |
| | | | 0.55 (m, 1 H), 0.07 (dq, |
| | | | J = 9.77, 5.00 Hz, 1 H). |
| 258 | 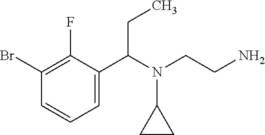 [SMILES: CCC(c1cccc(Br)c1F)N(CCN)C1CC1] | 315.2 [M + H]⁺ Purity: 99.25% retention time: 1.610 min method: M | Special NMR (400 MHz, DMSO-d6) δ = 8.28- 7.89 (m, 3H), 7.69 (br t, J = 7.2 Hz, 1H), 7.60 (br s, 1H), 7.22 (t, J = 7.9 Hz, 1H), 4.43-4.28 (m, 1H), 3.22-2.84 (m, 4H), 2.27-2.13 (m, 1H), 2.12- 1.92 (m, 2H), 0.76 (t, J = |
| | HCl | | 7.3 Hz, 5H), 0.69-0.59 |
| | | | (m, 2H). |
| 248 | 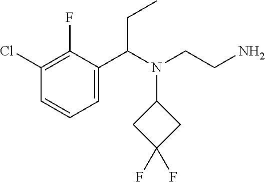 [SMILES: CCC(c1cccc(Cl)c1F)N(CCN)C1CC(F)(F)C1] | 321.3 [M + H]⁺ Purity: 98.00% retention time: 1.714 min method: M | ¹H NMR (400 MHz, DMSO-d6) 8 = 8.35 (br s, 3H), 7.84-7.58 (m, 2H), 7.37-7.28 (m, 1H), 3.74-3.54 (m, 1H), 3.30 (br dd, J = 2.3, 3.4 Hz, 1H), 3.24-2.60 (m, 8H), 2.27-1.98 (m, 2H), 0.70 (br t, J = 7.0 Hz, 3H) |
| | HCl | | |

### Example 28: Synthesis of Compounds 218 and 227

Starting from 3-Acetyl-2-fluorobenzonitrile (CAS 112279-89-7) instead of intermediate 3 in step 3 and using cyclopropylamine or cyclobutylamine resp., the following compounds were prepared according to the method described for the synthesis of Compound 262 (example 26):

| Compound | Structure | LCMS | H NMR |
|---|---|---|---|
| 218 | HCl | 248.2 [M + H]⁺ | Special ¹H NMR (400 |
| | | Purity: 100% | MHz, DMSO-d6) δ = |
| | | retention time: | 8.16-7.93 (m, 4H), |
| | | 1.302 min | 7.87 (br t, J = 6.9 Hz, |
| | | method: M | 1H), 7.44 (t, J = 7.8 Hz, |
| | | | 1H), 4.70-4.58 (m, |
| | | | 1H), 3.11-2.89 (m, |
| | | | 4H), 2.21-2.12 (m, |
| | | | 1H), 1.59 (br d, J = 6.8 |
| | | | Hz, 3H), 0.75-0.58 (m, |
| | | | 4H). |
| 227 | HCl | 262.3 [M + H]⁺ | ¹H NMR (400 MHz, |
| | | Purity: 100% | DMSO-d6) δ = 8.58- |
| | | retention time: | 8.23 (m, 4H), 8.04 (br s, |
| | | 1.128 min | 1H), 7.52 (br t, J = 7.6 |
| | | method: M | Hz, 1H), 4.99-4.78 (m, |
| | | | 1H), 3.91-3.78 (m, |
| | | | 1H), 3.31-2.85 (m, |
| | | | 4H), 2.45-2.07 (m, |
| | | | 3H), 1.83-1.45 (m, |
| | | | 6H). |

### Example 29: Synthesis of N′-(1-(3-chloro-2-fluorophenyl)-2,2-difluoroethyl)-N′-cyclopropylethane-1,2-diamine hydrochloride (Compound 275)

### 1) 1-(3-chloro-2-fluorophenyl)-2,2-difluoroethan-1-one

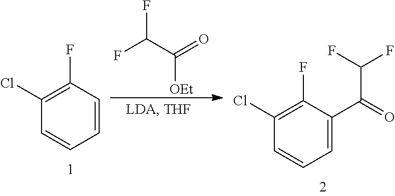 [SMILES: Fc1ccccc1Cl ; CCOC(=O)C(F)F ; O=C(c1cccc(Cl)c1F)C(F)F]

A solution of 1-chloro-2-fluoro-benzene (3 g, 23.0 mmol, 1 eq) in THF (30 mL) was degassed and purged with N₂ for 3 times, and then LDA (2 M, 23.0 mL, 2 eq) was added to the reaction at −78° C., the mixture was stirred at −78° C. for 15 min under N₂ atmosphere. And then ethyl 2,2-difluoroacetate (3.1 g, 25.3 mmol, 1.1 eq) was added to the reaction at −78° C., the mixture was stirred at 25° C. for 12 hr under N₂ atmosphere. TLC indicated reactant 1 was consumed completely and many new spots formed. The reaction mixture was quenched by addition sat. NH₄Cl 10 mL at 0° C., and then extracted with ethyl acetate 30 mL (10 mL×3). The combined organic layers were dried over Na₂SO₄, filtered and concentrated under reduced pressure to give a residue. The residue was purified by flash silica gel chromatography (10 g Silica Flash Column, Eluent of 0˜10% Ethyl acetate/Petroleum ether gradient at 80 mL/min) to afford compound 1-(3-chloro-2-fluoro-phenyl)-2,2-difluoro-ethanone (pure product 0.4 g, crude product 2.5 g) as a yellow oil.

¹H NMR (400 MHz, CHLOROFORM-d) δ=7.55-7.49 (m, 1H), 7.37-7.32 (m, 1H), 7.08 (t, J=7.9 Hz, 1H), 6.68 (s, 1H)

### 2)N-(1-(3-chloro-2-fluorophenyl)-2,2-difluoroethyl)cyclopropanamine

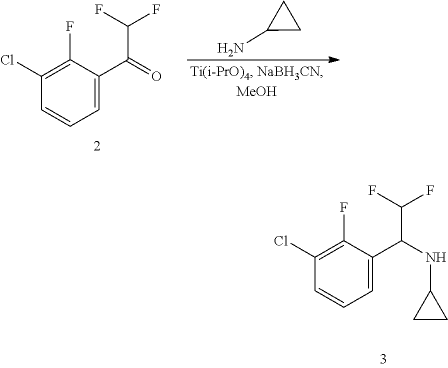

To a solution of 1-(3-chloro-2-fluoro-phenyl)-2,2-difluoro-ethanone (2.5 g, 11.8 mmol, 1 eq) in MeOH (2 mL) was added cyclopropanamine (1.0 g, 17.6 mmol, 1.2 mL, 1.5 eq) and Ti(i-PrO)₄ (10.0 g, 35.2 mmol, 10.4 mL, 3 eq). The mixture was stirred at 60° C. for 12 hr. And then NaBH₃CN (3.0 g, 47.0 mmol, 4 eq) was added and the resulting mixture was stirred at 60° C. for 3 hr. LC-MS showed ˜29% of desired compound was detected. The reaction mixture was filtered and concentrated under reduced pressure to give a residue. The residue was purified by flash silica gel chromatography (20 g Silica Flash Column, Eluent of 0˜5% ethyl acetate/petroleum ether gradient @80 m/min) to give compound N-(1-(3-chloro-2-fluorophenyl)-2,2-difluoroethyl)cyclopropanamine (2.0 g, 2.8 mmol, 23.9% yield, 35% purity) as a white solid.

### 3) 2-bromo-N-(1-(3-chloro-2-fluorophenyl)-2,2-difluoroethyl)-N-cyclopropylacetamide

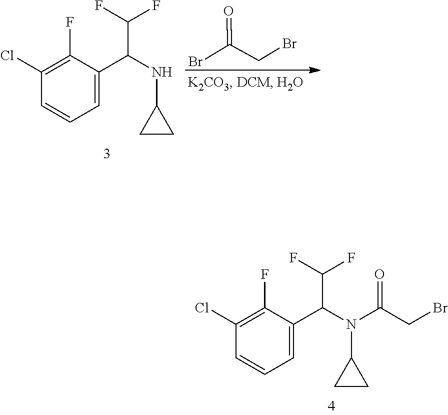

To a solution of N-[1-(3-chloro-2-fluoro-phenyl)ethyl]-3,3-difluoro-prop-2-en-1-amine (1.8 g, 7.4 mmol, 1 eq) in DCM (20 mL) was added K₂CO₃ (2.0 g, 14.7 mmol, 2 eq) in H₂O (20 mL) and 2-bromoacetyl bromide (1.8 g, 8.8 mmol, 768.1 μL, 1.2 eq) was added to the reaction at 0° C. The mixture was stirred at 25° C. for 12 hr. LC-MS showed reactant 3 was consumed completely and ˜65% of desired mass was detected. The reaction mixture was extracted with DCM 60 mL (20 mL×3). The combined organic layers were washed with sat. NaCl 20 mL, dried over Na₂SO₄, filtered and concentrated under reduced pressure to give a residue. The residue was purified by flash silica gel chromatography (10 g Silica Flash Column, Eluent of 0˜10% ethyl acetate/petroleum ether gradient at 80 mL/min) to give compound 2-bromo-N-(1-(3-chloro-2-fluorophenyl)-2,2-difluoroethyl)-N-cyclopropyl acetamide (1.5 g, 4.0 mmol, 54.0% yield) as a yellow oil.

### 4)N-(1-(3-chloro-2-fluorophenyl)-2,2-difluoroethyl)-N-cyclopropyl-2-(1,3-dioxoisoindolin-2-yl)acetamide

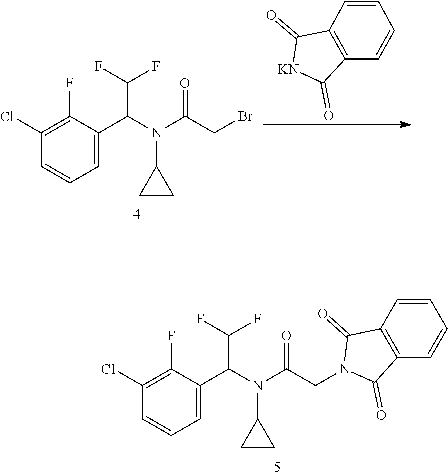

To a solution of 2-bromo-N-(1-(3-chloro-2-fluorophenyl)-2,2-difluoroethyl)-N-cyclopropylacetamide (1.5 g, 4.0 mmol, 1 eq) in DMF (15 mL) was added (1,3-dioxoisoindolin-2-yl)potassium (952.8 mg, 5.1 mmol, 1.3 eq). The mixture was stirred at 25° C. for 12 hr. LC-MS showed reactant 4 was consumed completely and ˜20% of desired mass was detected. The reaction mixture was quenched by addition water 20 mL at 25° C., and then extracted with ethyl acetate 60 mL (20 mL×3). The combined organic layers were washed with sat. NaCl 20 mL, dried over Na2SO4, filtered and concentrated under reduced pressure to give a residue. The residue was purified by flash silica gel chromatography (10 g Silica Flash Column, Eluent of 0-80% ethyl acetate/petroleum ether gradient at 80 mL/min) to give compound N-(1-(3-chloro-2-fluorophenyl)-2,2-difluoroethyl)-N-cyclopropyl-2-(1,3-dioxoisoindolin-2-yl)acetamide (0.6 g, 1.3 mmol, 33.8% yield) as an off-white solid.

### 5) 2-amino-N-(1-(3-chloro-2-fluorophenyl)-2,2-difluoroethyl)-N-cyclopropylacetamide

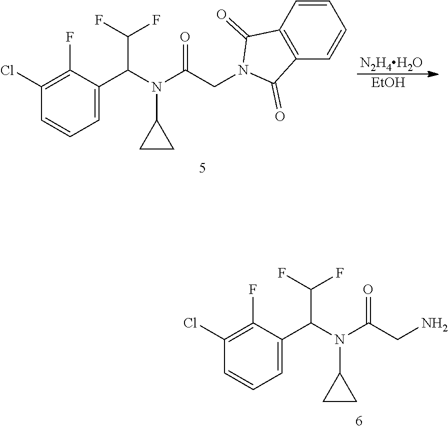

To a solution of N-(1-(3-chloro-2-fluorophenyl)-2,2-difluoroethyl)-N-cyclopropyl-2-(1,3-dioxoisoindolin-2-yl)acetamide (0.6 g, 1.3 mmol, 1 eq) in EtOH (10 mL) was added N₂H₄—H₂O (83.6 mg, 1.3 mmol, 81.0 μL, 80% purity, 1 eq). The mixture was stirred at 80° C. for 12 hr. LC-MS showed reactant 5 was consumed completely and ˜27% of desired mass was detected. The reaction mixture was filtered and concentrated under reduced pressure to give a residue. The residue was purified by flash silica gel chromatography (4 g Silica Flash Column, Eluent of 0-8% ethyl acetate/petroleum ether gradient at 40 mL/min) to give compound 2-amino-N-(1-(3-chloro-2-fluorophenyl)-2,2-difluoroethyl)-N-cyclopropylacetamide (0.4 g, 1.3 mmol, 97.7% yield) as a yellow oil.

### 6) N1-(1-(3-chloro-2-fluorophenyl)-2,2-difluoroethyl)-N1-cyclopropylethane-1,2-diamine hydrochloride (Compound 275)

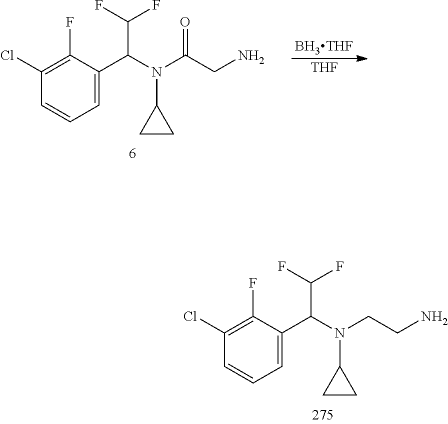 [SMILES: NCC(=O)N(C1CC1)C(c1cccc(Cl)c1F)C(F)F ; NCCN(C1CC1)C(c1cccc(Cl)c1F)C(F)F ; "BH3·THF" ; "THF"]

A mixture of 2-amino-N-(1-(3-chloro-2-fluorophenyl)-2,2-difluoroethyl)-N-cyclopropylacetamide (0.4 g, 1.1 mmol, 1 eq) in THF (10 mL) was degassed and purged with N₂ for 3 times, and then BH₃·THF (1 M, 11.4 mL, 10 eq) was added to the reaction at 0° C. and stirred at 0° C. for 30 min, the mixture was stirred at 60° C. for 12 under N₂ atmosphere. LC-MS showed reactant 6 was consumed completely and ˜66% of desired mass was detected. The reaction mixture was quenched by addition MeOH 2 mL at 0° C., and then diluted with water 5 mL and extracted with ethyl acetate 15 mL (5 mL×3). The combined organic layers were washed with sat. NaCl 10 mL, dried over Na₂SO₄, filtered and concentrated under reduced pressure to give a residue. The crude product was purified by reversed-phase HPLC (0.04% HCl condition, Column: Phenomenex Luna C18 75×30 mm×3 um; mobile phase: [H₂O (0.04% HCl)-ACN]; gradient: 15%-45% B over 8.0 min) to give compound N¹-(1-(3-chloro-2-fluorophenyl)-2,2-difluoroethyl)-N¹-cyclopropylethane-1,2-diamine hydrochloride (0.2 g, 616.4 μmol, 54.0% yield, 99.2% purity, HCl) as a white solid. MS (ESI): m/z=293.2 [M+H]⁺; retention time: 1.600 min, method:M ¹H NMR (400 MHz, DMSO-d₆) δ=8.09-7.84 (m, 3H), 7.67-7.61 (m, 1H), 7.58-7.52 (m, 1H), 7.30 (t, J=7.9 Hz, 1H), 6.91-6.59 (m, 2H), 4.49 (dt, J=6.0, 12.4 Hz, 1H), 3.09-2.81 (m, 3H), 2.73-2.63 (m, 1H), 1.85-1.77 (m, 1H), 0.68-0.40 (m, 4H).

### Example 30: Synthesis of Compounds 225 and 274

In analogous manner as set out above for Compound 275 (example 30), the following further compounds have been prepared:

| Compound | Structure | LCMS | H NMR |
|---|---|---|---|
| 225 | 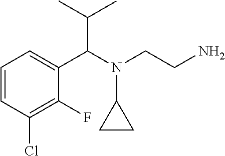 [SMILES: CC(C)C(c1cccc(Cl)c1F)N(CCN)C1CC1] | 285.3 [M + H]⁺ Purity: 99.51% retention time: 1.853 min method: M | ¹H NMR (400 MHz, DMSO-d6) δ = 7.92- 7.66 (m, 3H), 7.53-7.47 (m, 1H), 7.39-7.32 (m, 1H), 7.28-7.22 (m, 1H), 3.73 (br d, J = 10.8 Hz, 1H), 3.02-2.93 (m, 2H), 2.93-2.83 (m, 1H), 2.48- 2.27 (m, 2H), 1.52- 1.44 (m, 1H), 1.03 (d, J = |
| | HCl | | 6.3 Hz, 3H), 0.65 (d, J = |
| | | | 6.4 Hz, 3H), 0.64- |
| | | | 0.39 (m, 4H). |
| 274 | HCl | 307.2 [M + H]⁺ | ¹H NMR (400 MHz, |
| | | Purity: 97.09% | DMSO-d6) δ = 7.80 (br |
| | | retention | d, J = 1.0 Hz, 3H), 7.69- |
| | | time: 1.747 min, | 7.61 (m, 1H), 7.57 (br t, |
| | | method: M | J = 7.1 Hz, 1H), 7.35- |
| | | | 7.28 (m, 1H), 4.73-4.57 |
| | | | (m, 1H), 3.13-3.02 (m, |
| | | | 1H), 2.93 (br d, J = 5.8 Hz, |
| | | | 2H), 2.54 (br s, 1H), |
| | | | 1.80-1.67 (m, 4H), 0.71- |
| | | | 0.57 (m, 2H), 0.56- |
| | | | 0.47 (m, 2H) |

### Example 31: Synthesis of N′-[1-(3-chloro-2-fluoro-phenyl)-3,3,3-trifluoro-propyl]-N′-cyclopropyl-ethane-1,2-diamine hydrochloride (Compound 247)

### 1) ((1-(3-chloro-2-fluorophenyl)vinyl)oxy)triethylsilane

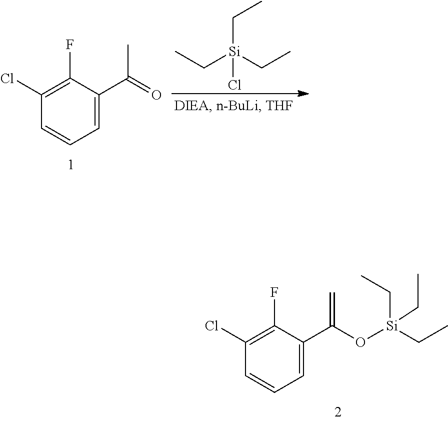

To a stirring solution of DIEA (1.9 g, 19.1 mmol, 2.7 mL, 1.1 eq) in THF (10 mL) at −78° C. was added n-BuLi (2.5 M, 7.7 mL, 1.1 eq) dropwise, and the solution was stirred for 30 min. To this solution was added the 1-(3-chloro-2-fluoro-phenyl)ethanone (3 g, 17.4 mmol, 1 eq) followed by chloro(triethyl)silane (2.9 g, 19.1 mmol, 3.3 mL, 1.1 eq), and the reaction mixture was stirred at 20° C. for 12 hr. TLC indicated 1 was consumed completely and many new spots formed. The reaction mixture was quenched by addition H₂O 2 mL at 0° C., and then diluted with H₂O 20 mL and extracted with EtOAc 45 mL (15 mL×3), dried over Na₂SO₄, filtered and concentrated under reduced pressure to give a residue. The residue was purified by flash silica gel chromatography (12 g Silica Flash Column, Eluent of 0˜0% Ethyl acetate/Petroleum ether gradient @80 mL/min) to give compound ((1-(3-chloro-2-fluorophenyl)vinyl)oxy)triethylsilane (2.0 g, 7.0 mmol, 40.1% yield) as a colorless oil.

### 2) 1-(3-chloro-2-fluorophenyl)-3,3,3-trifluoropropan-1-one

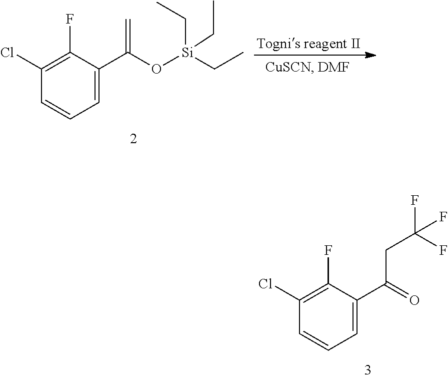 [SMILES: C=C(O[Si](CC)(CC)CC)c1cccc(Cl)c1F ; O=C(CC(F)(F)F)c1cccc(Cl)c1F]

1-(trifluoromethyl)-1λ3,2-benziodoxol-3-one (2.5 g, 7.8 mmol, 1.5 eq) and CuSCN (65.7 mg, 522.9 μmol, 0.1 eq) under N₂ atmosphere. To the mixture were added ((1-(3-chloro-2-fluorophenyl)vinyl)oxy)triethylsilane (1.5 g, 5.23 mmol, 1 eq) and DMF (15 mL). The mixture was stirred at 20° C. for 12 h. TLC indicated 2 was consumed completely and one major new spot formed. The reaction mixture was diluted with H₂O 25 mL and extracted with EtOAc 75 mL (25 mL×3). The combined organic layers were washed with brine 20 mL (10 mL×2), dried over Na₂SO₄, filtered and concentrated under reduced pressure to give a residue. The residue was purified by flash silica gel chromatography (12 g Silica Flash Column, Eluent of 0-0% Ethyl acetate/Petroleum ether gradient @80 mL/min) to give compound 1-(3-chloro-2-fluorophenyl)-3,3,3-trifluoropropan-1-one (0.8 g, 3.4 mmol, 64.4% yield) as a yellow oil. ¹H NMR (400 MHz, CHLOROFORM-d) δ=7.85-7.79 (m, 1H), 7.70-7.64 (m, 1H), 7.27-7.22 (m, 1H), 3.85 (dq, J=2.6, 9.8 Hz, 2H).

### 3)N-(1-(3-chloro-2-fluorophenyl)-3,3,3-trifluoropropyl)cyclopropanamine

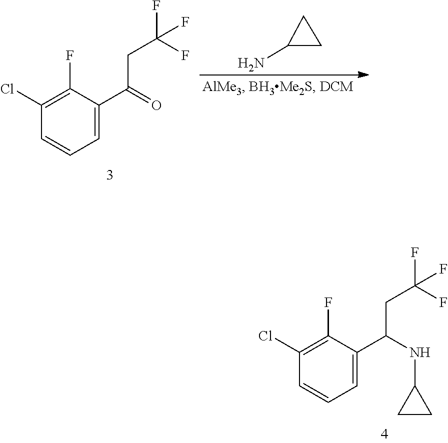

Dissolve the 1-(3-chloro-2-fluoro-phenyl)-3,3,3-trifluoro-propan-1-one (300 mg, 1.3 mmol, 1 eq) and the cyclopropanamine (106.8 mg, 1.9 mmol, 129.6 μL, 1.5 eq) under N₂ in dry DCM (6 mL). Add AlMe₃ (2 M, 935.2 μL, 1.5 eq) dropwise through syringe to the reaction mixture. Stir the solution at 20° C. for 15 hr. Add BH₃-Me₂S (10 M, 249.4 μL, 2 eq) dropwise to the reaction mixture. Stir the mixture at 20° C. for 2 hr. LC-MS showed one main peak with desired m/z was detected. Quench the reaction mixture by dropwise addition of 20% aqueous NaOH. Extract the aqueous layer with CH₂Cl₂ (3×30 mL). Dry the organic layer over Na₂SO₄. Remove the solvent under reduced pressure. The residue was purified by Prep-TLC (SiO₂, Petroleum ether/Ethyl acetate=5/1) to give compound N-(1-(3-chloro-2-fluorophenyl)-3,3,3-trifluoropropyl)cyclopropanamine (170 mg, 603.5 μmol, 48.4% yield) as a yellow oil.

### 4) 2-((1-(3-chloro-2-fluorophenyl)-3,3,3-trifluoropropyl)(cyclopropyl)amino)acetamide

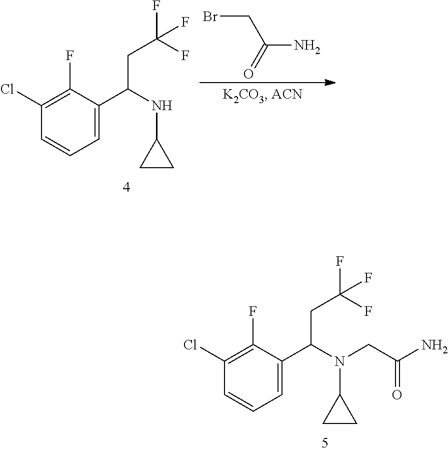

To a solution of N-(1-(3-chloro-2-fluorophenyl)-3,3,3-trifluoropropyl)cyclopropanamine (170 mg, 603.5 μmol, 1 eq) in ACN (3 mL) was added K₂CO₃ (417.06 mg, 3.02 mmol, 5 eq) and 2-bromoacetamide (499.59 mg, 3.62 mmol, 6 eq). The mixture was stirred at 80° C. for 12 hr. LC-MS showed 4 was consumed completely and one main peak with desired m/z was detected. The reaction mixture was concentrated under reduced pressure to remove solvent. The residue was diluted with H₂O 15 mL and extracted with EtOAc 30 mL (10 mL×3), dried over Na₂SO₄, filtered and concentrated under reduced pressure to give a residue. The residue was purified by prep-TLC (SiO₂, Petroleum ether/Ethyl acetate=5/1) to give compound 2-((1-(3-chloro-2-fluorophenyl)-3,3,3-trifluoropropyl)(cyclopropyl)amino)acetamide (101 mg, 298.2 μmol, 49.4% yield) as a yellow oil.

### 5) N¹-(1-(3-chloro-2-fluorophenyl)-3,3,3-trifluoropropyl)-N¹-cyclopropylethane-1,2-diamine hydrochloride (Compound 247)

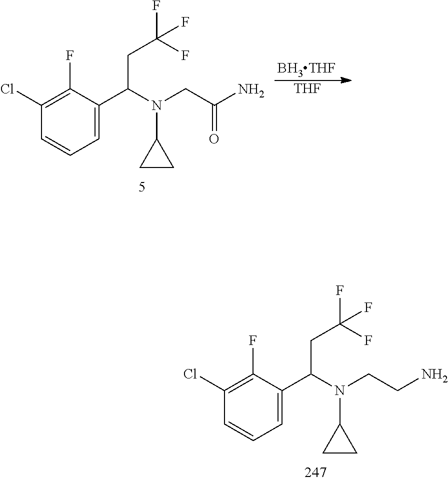 [SMILES: NC(=O)CN(C1CC1)C(CC(F)(F)F)c1cccc(Cl)c1F ; NCCN(C1CC1)C(CC(F)(F)F)c1cccc(Cl)c1F ; "THF" ; "BH3·THF"]

To a solution of 2-[[1-(3-chloro-2-fluoro-phenyl)-3,3,3-trifluoro-propyl]-cyclopropyl-amino]acetamide (101 mg, 298.2 μmol, 1 eq) in THF (10 mL) was added BH₃·THF (1 M, 3.0 mL, 10 eq). The mixture was stirred at 70° C. for 12 hr. LC-MS showed 5 was consumed completely and one peak with desired m/z was detected. The residue was diluted with NH₄C1 5 mL and extracted with EtOAc 15 mL (5 mL×3), dried over Na₂SO₄, filtered and concentrated under reduced pressure to give a residue. The residue was purified by prep-HPLC (HCl condition, column: Waters Xbridge BEH C18 100×30 mm×10 um; mobile phase: [water(HCl)-ACN]; B %: 25%-55%, 8 min) to give compound N′-[1-(3-chloro-2-fluoro-phenyl)-3,3,3-trifluoro-propyl]-N′-cyclopropyl-ethane-1,2-diamine hydrochloride (11 mg, 30.2 μmol, 10.1% yield, 99% purity, HCl) as a colorless oil. MS (ESI): m/z=325.3 [M+H]⁺; retention time: 1.790 min, method:M. ¹H NMR (400 MHz, DMSO-d6) δ=8.08-7.89 (m, 3H), 7.63-7.54 (m, 2H), 7.28 (br t, J=7.8 Hz, 1H), 4.60-4.52 (m, 1H), 3.22-2.94 (m, 4H), 2.89 (br s, 2H), 1.68-1.57 (m, 1H), 0.65-0.40 (m, 4H).

### Example 32: Synthesis of N¹-((3-chloro-2-fluorophenyl)(cyclobutyl)methyl)-N¹-cyclopropylethane-1,2-diamine hydrochloride (Compound 267)

### 1) (3-chloro-2-fluorophenyl)(cyclobutyl)methanol

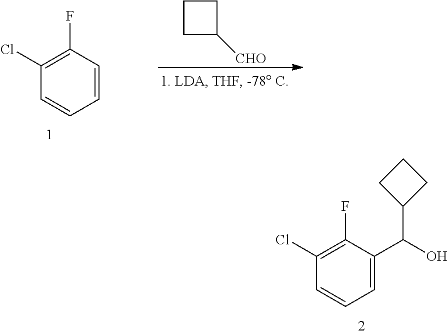 [SMILES: Fc1ccccc1Cl ; O=CC1CCC1 ; OC(c1cccc(Cl)c1F)C1CCC1]

To a solution of 1-chloro-2-fluoro-benzene (1 g, 7.7 mmol, leq) in THF (10 mL) was degassed and purged with N₂ for three times, LDA (2 M, 7.7 mL, 2 eq) was added to the reaction at −78° C. The mixture was stirred at −78° C. for 15 min. And then cyclobutanecarbaldehyde (708.8 mg, 8.4 mmol, 1.1 eq) was added to the reaction, the reaction was stirred at 25° C. for 12 hr. TLC indicated reactant 1 was consumed completely and new spots were detected. The reaction mixture was quenched by addition sat.NH₄Cl 10 mL at 0° C., and then extracted with ethyl acetate 30 mL (10 mL×3). The combined organic layers were dried over Na₂SO₄, filtered and concentrated under reduced pressure to give a residue. The residue was purified by flash silica gel chromatography (10 g Silica Flash Column, Eluent of 0˜5% ethyl acetate/petroleum ether gradient at 80 m/min) to give compound (3-chloro-2-fluorophenyl)(cyclobutyl)methanol (1.0 g, 4.8 mmol, 63.3% yield) as a yellow oil. H NMR (400 MHz, CHLOROFORM-d) δ=7.35-7.28 (m, 2H), 7.11-7.01 (m, 1H), 4.96 (dd, J=2.7, 7.4 Hz, 1H), 2.75-2.63 (m, 1H), 2.07-2.01 (m, 2H), 1.89-1.81 (m, 4H).

### 2) (3-chloro-2-fluorophenyl)(cyclobutyl)methanone

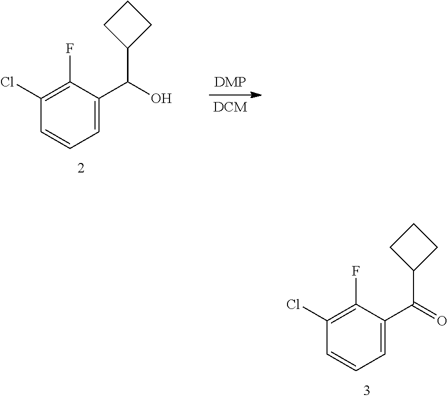 [SMILES: OC(c1cccc(Cl)c1F)C1CCC1 ; O=C(c1cccc(Cl)c1F)C1CCC1]

To a solution of (3-chloro-2-fluorophenyl)(cyclobutyl)methanol (1.0 g, 4.8 mmol, 1 eq) in DCM (10 mL) was added DMP (2.5 g, 5.8 mmol, 1.8 mL, 1.2 eq). The mixture was stirred at 25° C. for 12 hr. TLC indicated 2 was consumed completely and new spots formed. The reaction mixture was filtered and concentrated under reduced pressure to give a residue. The residue was purified by flash silica gel chromatography (10 g Silica Flash Column, Eluent of 0˜8% ethylacetate/petroleum ether gradient at 80 mL/min) to give compound (3-chloro-2-fluorophenyl)(cyclobutyl)methanone (0.5 g, 2.3 mmol, 46.7% yield) as a yellow oil. ¹H NMR (400 MHz, CHLOROFORM-d) δ=7.74 (ddd, J=1.8, 6.3, 7.9 Hz, 1H), 7.59-7.53 (m, 1H), 7.18 (t, J=7.9 Hz, 1H), 3.90 (dquin, J=2.8, 8.3 Hz, 1H), 2.40-2.27 (m, 4H), 2.12-1.99 (m, 1H), 1.95-1.84 (m, 1H).

### 3)N-((3-chloro-2-fluorophenyl)(cyclobutyl)methyl)cyclopropanamine

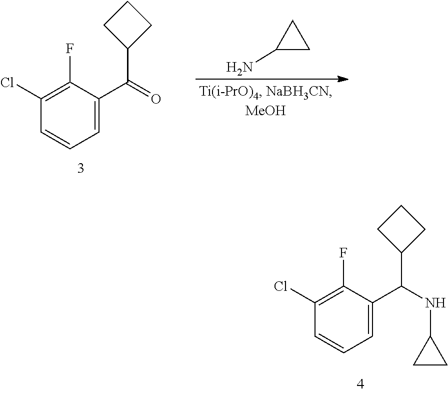

To a solution of (3-chloro-2-fluorophenyl)(cyclobutyl)methanone (0.5 g, 2.3 mmol, 1 eq) in MeOH (5 mL) was added cyclopropanamine (193.9 mg, 3.4 mmol, 235.3 μL, 1.5 eq) and Ti(i-PrO)₄ (1.9 g, 6.8 mmol, 2.0 mL, 3 eq). The mixture was stirred at 60° C. for 12 hr. And then NaBH₃CN (569.0 mg, 9.0 mmol, 4 eq) was added. The resulting mixture was stirred at 60° C. for 3 hr. LC-MS showed 3 was consumed completely and desired mass was detected. The reaction mixture was concentrated under reduced pressure to give a residue. The residue was purified by flash silica gel chromatography (4 g Silica Flash Column, Eluent of 0˜5% ethyl acetate/petroleum ether gradient at 40 mL/min) to give compound N-((3-chloro-2-fluorophenyl)(cyclobutyl)methyl)cyclopropanamine (0.3 g, 1.2 mmol, 52.2% yield) as a yellow oil.

### 4)N-((3-chloro-2-fluorophenyl)(cyclobutyl)methyl)cyclopropanamine

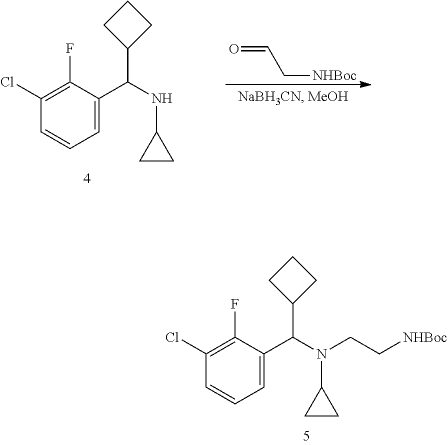 [SMILES: Fc1c(Cl)cccc1C(NC1CC1)C1CCC1 ; CC(C)(C)OC(=O)NCC=O ; CC(C)(C)OC(=O)NCCN(C1CC1)C(c1cccc(Cl)c1F)C1CCC1]

To a solution of N-((3-chloro-2-fluorophenyl)(cyclobutyl)methyl)cyclopropanamine (0.1 g, 512.3 μmol, 1 eq) in MeOH (1 mL) was added tert-butyl N-(2-oxoethyl)carbamate (489.3 mg, 3.1 mmol, 6 eq). The mixture was stirred at 60° C. for 12 hr, and then NaBH₃CN (128.8 mg, 2.1 mmol, 4 eq) was added to the reaction. The mixture was stirred at 60° C. for 3 hr. LC-MS showed 4 was consumed completely and desired mass was detected. The reaction mixture was concentrated under reduced pressure to give a residue. The crude product tert-butyl (2-(((3-chloro-2fluorophenyl)(cyclobutyl)methyl)(cyclopropyl)amino)ethyl) carbamate (0.2 g, 378.6 μmol, 73.8% yield, 37.5% purity) as a yellow oil.

### 5) N¹-((3-chloro-2-fluorophenyl)(cyclobutyl)methyl)-N¹-cyclopropylethane-1,2-diamine hydrochloride (Compound 267)

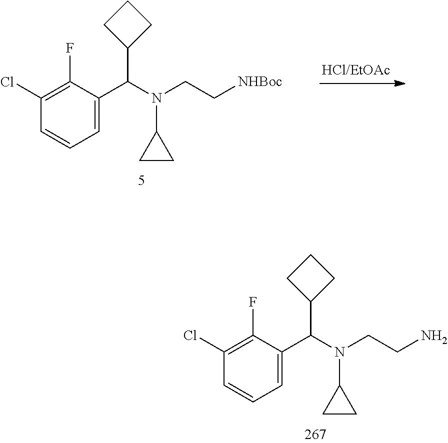 [SMILES: CC(C)(C)OC(=O)NCCN(C1CC1)C(c1cccc(Cl)c1F)C1CCC1 ; NCCN(C1CC1)C(c1cccc(Cl)c1F)C1CCC1]

To a solution of tert-butyl (2-(((3-chloro-2-fluorophenyl)(cyclobutyl)methyl)(cyclopropyl)amino)ethyl)carbamate (0.2 g, 377.9 μmol, 1 eq) in HCl/EtOAc (3 mL) was stirred at 25° C. for 1 hr. LC-MS showed ˜3% of reactant 5 remained. Several new peaks were shown on LC-MS and ˜40% of desired compound was detected. The reaction mixture was concentrated under reduced pressure to give a residue. The crude product was purified by reversed-phase HPLC (0.04% HCl condition, column: Phenomenex Luna C18 75×30 mm×3 um; mobile phase: [H2O (0.04% HCl)-ACN]; gradient: 15%-45% B over 8.0 min) to give compound N¹-((3-chloro-2-fluorophenyl)(cyclobutyl)methyl)-N¹-cyclopropylethane-1,2-diamine hydrochloride (0.08 g, 271.7 μmol, 71.9% yield, 99.6% purity, HCl) as a yellow oil. MS (ESI): m/z=297.1 [M+H]⁺; retention time: 2.021 min, method:M ¹H NMR (400 MHz, DMSO-d6) δ=8.07-7.75 (m, 3H), 7.52 (t, J=7.4 Hz, 1H), 7.43 (br s, 1H), 7.27-7.20 (m, 1H), 4.35-4.23 (m, 1H), 3.20-2.91 (m, 4H), 2.81-2.64 (m, 1H), 2.28-2.16 (m, 1H), 2.03-1.66 (m, 5H), 1.44-1.30 (m, 1H), 0.60 (br d, J=5.4 Hz, 4H).

### Example 33: Synthesis of Compounds 268 and 273

In analogous manner as set out above for Compound 267 (example 33), the following further compounds have been prepared:

| Compound | Structure | LCMS | H NMR |
|---|---|---|---|
| 268 | 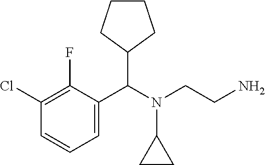 [SMILES: NCCN(C1CC1)C(c1cccc(Cl)c1F)C1CCCC1] | 311.2 [M + H]+ Purity: 100% retention time: 1.947 min, method: M | Special NMR (400 MHz, DMSO-d6) δ = 8.51-8.38 (m, 1H), 8.09 (br s, 3H), 7.55- 7.43 (m, 2H), 7.28- 7.22 (m, 1H), 4.02 (br d, J = 9.8 Hz, 1H), 3.19 (br dd, J = 7.0, 12.4 Hz, 1H), 2.99 (br s, 2H), 2.76-2.60 (m, 2H), 1.95-1.86 (m, 1H), |
| | 2 HCl | | 1.63-1.37 (m, 6H), |
| | | | 1.05-0.41 (m, 6H). |
| 273 | HCl | 333.1 [M + H]⁺ | ¹H NMR (400 MHz, |
| | | Purity: 99.77% | DMSO-d6) δ = 7.82 (br |
| | | retention | s, 3H), 7.56 (br t, J = |
| | | time: 2.082 min, | 7.4 Hz, 1H), 7.41 (br s, |
| | | method: M | 1H), 7.29-7.23 (m, |
| | | | 1H), 4.22-4.08 (m, |
| | | | 1H), 2.92 (br s, 4H), |
| | | | 2.81-2.52 (m, 2H), |
| | | | 2.40 (br dd, J = 7.4, |
| | | | 12.5 Hz, 2H), 1.99- |
| | | | 1.85 (m, 1H), 1.65- |
| | | | 1.44 (m, 1H), 0.66- |
| | | | 0.40 (m, 4H). |

### Example 34: Synthesis of 3-(1-((2-aminoethyl)(cyclopropyl)amino)propyl)-2-fluoro benzonitrile hydrochloride (Compound 257)

### 1) 2-fluoro-3-(1-hydroxypropyl)benzonitrile

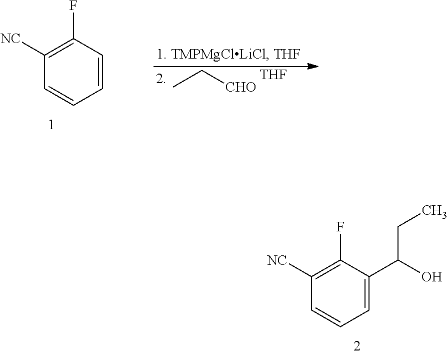

A dry round-bottom flask flushed with N₂ was charged with the desired 2-fluorobenzonitrile (1.5 g, 12.4 mmol, 1.3 mL, 1 eq) and THF (20 mL). The base, lithium; chloro-(2,2,6,6-tetramethyl-1-piperidyl)magnesium; chloride (1 M, 16.1 mL, 1.3 eq), was added dropwise, and the reaction was kept at 20° C. for 1 hr. The generated organomagnesium species were trapped with a propanal (791.3 mg, 13.6 mmol, 991.6 μL, 1.1 eq), and the mixture was allowed to react at 20° C. for 12 hr. TLC indicated Reactant 1 was consumed completely and one major new spot formed. The reaction was quenched with saturated aqueous NH₄Cl and extracted with ethyl acetate (3×10 mL). The combined organic phases were dried over Na₂SO₄, filtered and concentrated under reduced pressure. The residue was purified by flash silica gel chromatography (12 g Silica Flash Column, Eluent of 0˜5% Ethyl acetate/Petroleum ether gradient @60 mL/min) to give compound 2-fluoro-3-(1-hydroxypropyl)benzonitrile (876.9 mg, 4.9 mmol, 39.5% yield) as a yellow oil.

### 2) 2-fluoro-3-propionylbenzonitrile

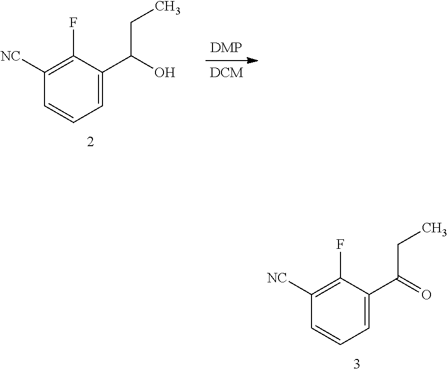

To a solution of 2-fluoro-3-(1-hydroxypropyl)benzonitrile (800 mg, 4.5 mmol, 1 eq) in DCM (35 mL) was added DMP (2.3 g, 5.36 mmol, 1.2 eq). The mixture was stirred at 20° C. for 12 hr. TLC indicated 2 was consumed completely and one major new spot formed. The reaction mixture was diluted with H₂O 6 mL and extracted with DCM 15 mL (5 mL×3), dried over Na₂SO₄, filtered and concentrated under reduced pressure to give a residue. The residue was purified by flash silica gel chromatography (12 g Silica Flash Column, Eluent of 0-3% Ethyl acetate/Petroleum ether gradient @60 m/min) to give compound 2-fluoro-3-propionylbenzonitrile (468 mg, 2.6 mmol, 59.2% yield) as a white solid.

### 3) 3-(1-(cyclopropylamino)propyl)-2-fluorobenzonitrile

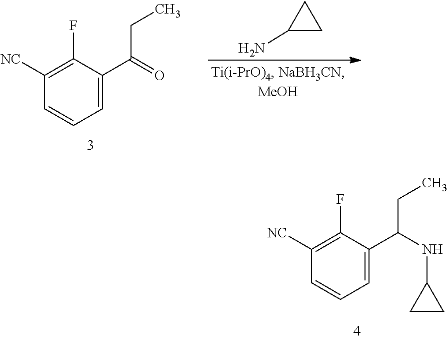

To a solution of 2-fluoro-3-propanoyl-benzonitrile (200 mg, 1.1 mmol, 1 eq) in MeOH (3 mL) was added cyclopropanamine (96.7 mg, 1.7 mmol, 117.3 μL, 1.5 eq) and Ti(i-PrO)₄ (962.5 mg, 3.4 mmol, 999.5 μL, 3 eq). The mixture was stirred at 60° C. for 12 hr. NaBH₃CN (283.6 mg, 4.5 mmol, 4 eq) was added to the reaction at 20° C. and stirred at 60° C. for 2 hr. LC-MS showed one main peak with desired m/z was detected. The reaction mixture was concentrated under reduced pressure to remove solvent. The residue was diluted with H₂O 15 mL and extracted with EtOAc 30 mL (10 mL×3), dried over Na₂SO₄, filtered and concentrated under reduced pressure to give compound 3-(1-(cyclopropylamino)propyl)-2-fluorobenzonitrile (263 mg, crude) as a yellow oil.

### 4) tert-butyl (2-((1-(3-cyano-2-fluorophenyl)propyl)(cyclopropyl)amino)ethyl)carbamate

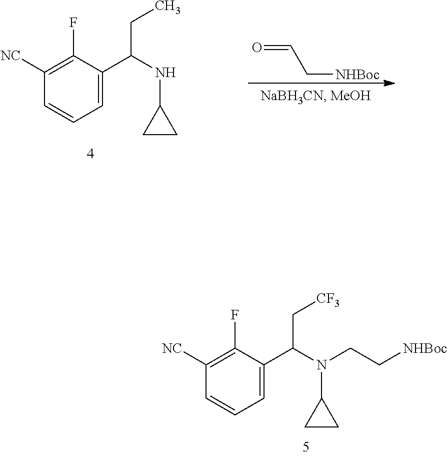 [SMILES: CCC(NC1CC1)c1cccc(C#N)c1F ; CC(C)(C)OC(=O)NCC=O ; CC(C)(C)OC(=O)NCCN(C1CC1)C(CC(F)(F)F)c1cccc(C#N)c1F]

To a solution of 3-[1-(cyclopropylamino)propyl]-2-fluoro-benzonitrile (150 mg, 687.2 μmol, 1 eq) in MeOH (3 mL) was added tert-butyl N-(2-oxoethyl)carbamate (656.4 mg, 4.1 mmol, 6 eq) and AcOH to adjust pH=5, the mixture was stirred at 60° C. for 12 hr. Then NaBH₃CN (172.8 mg, 2.8 mmol, 4 eq) was added at 20° C. The mixture was stirred at 60° C. for 2 hr. LC-MS showed 4 was consumed completely and one main peak with desired m/z was detected. The reaction mixture was concentrated under reduced pressure to remove solvent. The residue was diluted with H₂O 10 mL and extracted with EtOAc 30 mL (10 mL×3), dried over Na₂SO₄, filtered and concentrated under reduced pressure to give a residue. The residue was purified by prep-TLC (SiO₂, Petroleum ether/Ethyl acetate=5/1) to give compound tert-butyl (2-((1-(3-cyano-2-fluorophenyl)propyl)(cyclopropyl)amino) ethyl)carbamate (220 mg, 608.7 μmol, 88.6% yield) as a yellow oil.

### 5) 3-(1-((2-aminoethyl)(cyclopropyl)amino)propyl)-2-fluorobenzonitrile hydrochloride (Compound 257)

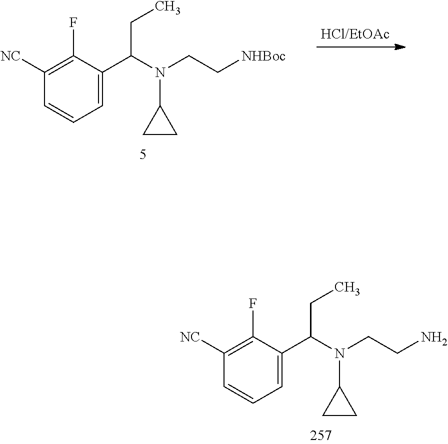 [SMILES: CCC(c1cccc(C#N)c1F)N(CCNC(=O)OC(C)(C)C)C1CC1 ; CCC(c1cccc(C#N)c1F)N(CCN)C1CC1]

A solution of tert-butyl N-[2-[1-(3-cyano-2-fluoro-phenyl)propyl-cyclopropyl-amino]ethyl]carbamate (220 mg, 608.7 μmol, 1 eq) in HCl/EtOAc (5 mL) was stirred at 20° C. for 3 hr. LC-MS showed 5 was consumed completely and one main peak with desired m/z was detected. The reaction mixture was concentrated under reduced pressure to remove solvent. The residue was purified by prep-HPLC (HCl condition). column: Phenomenex Luna C18 75×30 mm×3 um; mobile phase: [H2O (0.04% HCl)-ACN]; gradient: 10%-40% B over 8.0 min to give compound 3-(1-((2-aminoethyl) (cyclopropyl)amino)propyl)-2-fluorobenzonitrile hydrochloride (91.2 mg, 306.3 μmol, 50.3% yield, 100% purity, HCl) as a yellow oil. MS (ESI): m/z=262.4 [M+H]⁺; retention time: 1.496 min, method:M. Special ¹NMR (400 MHz, DMSO-d6) δ=8.36 (br s, 3H), 8.09 (br t, J=7.1 Hz, 1H), 7.94 (ddd, J=1.5, 6.3, 7.7 Hz, 1H), 7.52-7.45 (m, 1H), 4.56 (br dd, J=4.3, 10.4 Hz, 1H), 3.54-3.38 (m, 1H), 3.30-3.05 (m, 3H), 2.42-2.27 (m, 2H), 2.17-2.06 (m, 1H), 0.97 (br s, 2H), 0.73 (t, J=7.3 Hz, 5H).

### Example 35: Synthesis of Compound 259

In analogous manner as set out above for Compound 257 (example 35), the following further compound has been prepared:

| Compound | Structure | LCMS | H NMR |
|---|---|---|---|
| 259 | 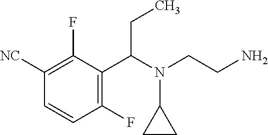 [SMILES: CCC(c1c(F)ccc(C#N)c1F)N(CCN)C1CC1] | 280.2 [M + H]⁺ Purity: 97.82% retention time: 1.522 min method: M | ¹H NMR (400 MHz, DMSO-d6) δ = 8.59- 8.42 (m, 3H), 8.22- 8.14 (m, 1H), 7.49 (br t, J = 9.3 Hz, 1H), 4.73 (br s, 1H), 3.71-3.18 (m, 4H), 2.28 (br d, J = 4.5 Hz, 2H), 1.21-0.91 (m, 2H), 0.75 (br t, J = |
| | HCl | | 7.1 Hz, 6H). |

### Example 36: Synthesis of 3-(((2-aminoethyl)(cyclopropyl)amino)methyl)-2-fluoro benzonitrile hydrochloride (Compound 219)

### 1) 3-((cyclopropylamino)methyl)-2-fluorobenzonitrile

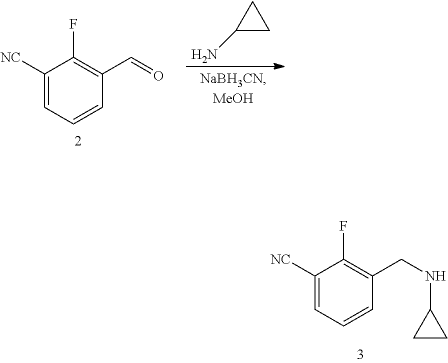

To a solution of 2-fluoro-3-formyl-benzonitrile (500 mg, 3.4 mmol, 1 eq) in MeOH (3 mL) was added cyclopropanamine (287.2 mg, 5.0 mmol, 348.5 uL, 1.5 eq). The mixture was stirred at 60° C. for 12 hr. NaBH₃CN (842.8 mg, 13.4 mmol, 4 eq) was added to the reaction at 20° C. and stirred at 60° C. for 2 hr. LC-MS showed Reactant 2 was consumed completely and one main peak with desired m/z was detected. The reaction mixture was concentrated under reduced pressure. The residue was diluted with H₂O 15 mL and extracted with EtOAc 45 mL (15 mL×3), dried over Na₂SO₄, filtered and concentrated under reduced pressure to give a residue. The residue was purified or by prep-TLC (SiO₂, Petroleum ether/Ethyl acetate=10/1) to give compound 3-((cyclopropylamino)methyl)-2-fluorobenzonitrile (363.0 mg, 1.9 mmol, 56.9% yield) as a yellow oil.

### 2) tert-butyl (2-((3-cyano-2-fluorobenzyl)(cyclopropyl)amino)ethyl)carbamate

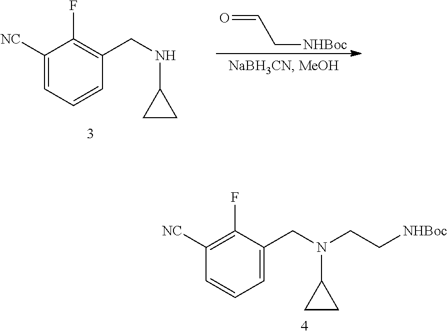 [SMILES: N#Cc1cccc(CNC2CC2)c1F ; CC(C)(C)OC(=O)NCC=O ; CC(C)(C)OC(=O)NCCN(Cc1cccc(C#N)c1F)C1CC1]

To a solution of 3-((cyclopropylamino)methyl)-2-fluorobenzonitrile (200.0 mg, 1.1 mmol, 1 eq) in MeOH (3 mL) was added tert-butyl N-(2-oxoethyl)carbamate (1.0 g, 6.3 mmol, 6 eq). The mixture was stirred at 60° C. for 12 hr. NaBH₃CN (264.3 mg, 4.2 mmol, 4 eq) was added to the reaction at 20° C. and stirred at 60° C. for 2 hr. LC-MS showed Reactant 3 was consumed completely and one main peak with desired m/z was detected. The reaction mixture was concentrated under reduced pressure to remove solvent. The residue was diluted with H₂O 15 mL and extracted with EtOAc 45 mL (15 mL×3), the combined organic layers were dried over Na₂SO₄, filtered and concentrated under reduced pressure to give a residue. The residue was purified by prep-TLC (SiO₂, Petroleum ether/Ethyl acetate=3/1) to give compound tert-butyl (2-((3-cyano-2-fluorobenzyl)(cyclopropyl)amino)ethyl)carbamate (262.6 mg, 787.6 μmol, 74.9% yield) as a yellow oil.

### 3) 3-(((2-aminoethyl)(cyclopropyl)amino)methyl)-2-fluorobenzonitrile hydrochloride (Compound 219)

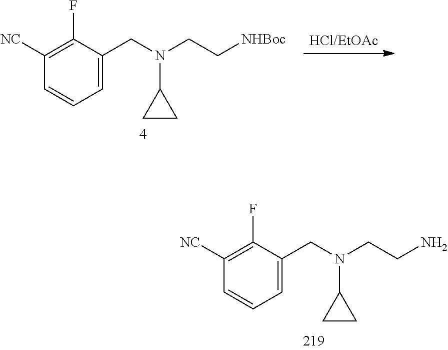 [SMILES: CC(C)(C)OC(=O)NCCN(Cc1cccc(C#N)c1F)C1CC1 ; N#Cc1cccc(CN(CCN)C2CC2)c1F]

To a solution of tert-butyl tert-butyl (2-((3-cyano-2-fluorobenzyl)(cyclopropyl)amino)ethyl)carbamate (262.6 mg, 787.6 μmol, 1 eq) in HCl/EtOAc (3 mL) was stirred at 20° C. for 1 hr. LC-MS showed Reactant 4 was consumed completely. Several new peaks were shown on LC-MS and 18% of desired compound was detected. The reaction mixture was concentrated under reduced pressure. The residue was purified by prep-HPLC (HCl condition, Column: Phenomenex Luna 80×30 mm×3 um; mobile phase: [water(HCl)-ACN]; B %: 1%-25%, 8 min) to give compound 3-(((2-aminoethyl)(cyclopropyl)amino)methyl)-2-fluorobenzonitrile hydrochloride (25.1 mg, 107.6 μmol, 13.7% yield, 100% purity, HCl) as a black-brown oil. MS (ESI): m/z=234.3[M+H]⁺, retention time: 1.253 min, method:M. ¹H NMR (400 MHz, DMSO-d6) δ=8.42 (br s, 3H), 8.13-7.92 (m, 2H), 7.42 (t, J=7.8 Hz, 1H), 4.44 (br s, 2H), 3.46-3.24 (m, 4H), 2.71 (br s, 1H), 0.99-0.59 (m, 4H).

### Example 37: Synthesis of Compounds 186, 187, 219B, 227B and 246

In analogous manner as set out above for Compound 219 (example 39), the following further compounds have been prepared:

| Compound | Structure | LCMS | H NMR |
|---|---|---|---|
| 186 | 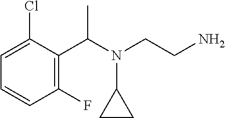 [SMILES: CC(c1c(F)cccc1Cl)N(CCN)C1CC1] | 257.1 [M + H]⁺ Purity: 96.86% retention time: 1.634 min method: M | 1H NMR (400 MHz, DMSO-d6) δ = 8.35- 7.87 (m, 3H), 7.51-7.39 (m, 1H), 7.38-7.32 (m, 1H), 7.27-7.18 (m, 1H), 4.82 (br d, J = 6.6 Hz, 1H), 3.41-2.98 (m, 4H), 2.29-2.09 (m, 1H), 1.67 |
| | HCl | | (dd, J = 1.7, 6.9 Hz, 3H), |
| | | | 0.88-0.39 (m, 4H). |
| 187 | 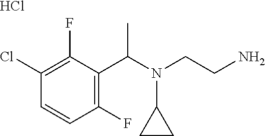 [SMILES: Cl ; CC(c1c(F)ccc(Cl)c1F)N(CCN)C1CC1] | 275.1 [M + H]⁺ Purity: 99.94% retention time: 1.852 min method: M | ¹H NMR (400 MHz, DMSO-d₆) δ = 8.59- 7.91 (m, 3H), 7.75-7.60 (m, 1H), 7.22 (br t, J = 9.2 Hz, 1H), 4.93-4.44 (m, 1H), 3.88-3.17 (m, 3H), 2.90-2.68 (m, 1H), 2.11-1.78 (m, 1H), 1.74- |
| | HCl | | 1.39 (m, 3H), 0.94- |
| | | | 0.20 (m, 4H). |
| 219B | 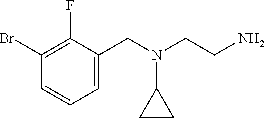 [SMILES: NCCN(Cc1cccc(Br)c1F)C1CC1] | 287.1 [M + H]⁺ Purity: 100% retention time: 1.427 min method: M | ¹H NMR (400 MHz, DMSO-d6) δ = 8.54 (br s, 3H), 7.85-7.74 (m, 2H), 7.23 (t, J = 7.9 Hz, 1H), 4.50 (br s, 2H), 3.51-3.33 (m, 4H), 2.79 (br s, 1H), 1.04-0.64 (m, 4H). |
| | HCl | | |
| 227B | 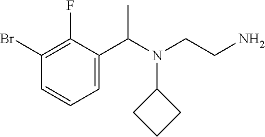 [SMILES: CC(c1cccc(Br)c1F)N(CCN)C1CCC1] | 315.2 [M + H]⁺ Purity: 100% retention time: 1.267 min method: M | Special ¹H NMR (400 MHz, DMSO-d6) δ = 8.64-8.27 (m, 3H), 7.91- 7.72 (m, 2H), 7.28- 7.21 (m, 1H), 4.86-4.68 (m, 1H), 3.73 (br s, 4H), 2.43-2.09 (m, 3H), 2.02- 1.85 (m, 1H), 1.80- 1.54 (m, 6H). |
| | HCl | | |
| 246 | 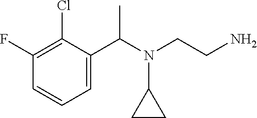 [SMILES: CC(c1cccc(F)c1Cl)N(CCN)C1CC1] | 257.2 [M + H]⁺ Purity: 99.76% retention time: 1.430 min method: M | Special ¹H NMR (400 MHz, DMSO-d6) δ = 8.08-7.78 (m, 3H), 7.59- 7.51 (m, 1H), 7.42 (dt, J = 5.8, 7.9 Hz, 1H), 7.38-7.31 (m, 1H), 4.70- 4.58 (m, 1H), 3.12- 2.95 (m, 4H), 2.24-2.12 |
| | HCl | | (m, 1H), 1.54 (br d, J = |
| | | | 6.7 Hz, 3H), 0.68-0.53 |
| | | | (m, 4H) |

### Example 38: Synthesis of 3-(1-((2-aminoethyl) (cyclopropyl)amino)-2-methylpropyl)-2-fluorobenzonitrile TFA salt (Compound 264)

### 1) 2-fluoro-3-(1-hydroxy-2-methylpropyl)benzonitrile

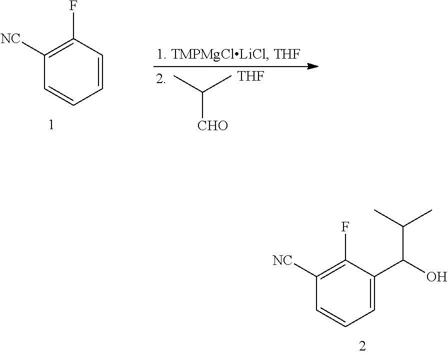

A dry round-bottom flask flushed with N₂ was charged with the desired 2-fluorobenzonitrile (2 g, 16.51 mmol, 1.76 mL, 1 eq) and THF (25 mL). The base, lithium; chloro-(2,2,6,6-tetramethyl-1-piperidyl)magnesium; chloride (1 M, 21.47 mL, 1.3 eq), was added dropwise, and the reaction was kept at 20° C. for 1 hr. The generated organomagnesium species were trapped with 2-methylpropanal (1.31 g, 18.17 mmol, 1.66 mL, 1.1 eq), and the mixture was allowed to react at 20° C. for 12 hr. TLC indicated Reactant 1 was consumed completely and one major new spot formed. The reaction was quenched with saturated aqueous NH₄Cl and extracted with ethyl acetate (3×10 mL). The combined organic phases were dried over Na₂SO₄, filtered and concentrated under reduced pressure. The residue was purified by flash silica gel chromatography (Silica Flash Column, Eluent of 0-6% Ethyl acetate/Petroleum ethergradient @60 mL/min) to give compound 2-fluoro-3-(1-hydroxy-2-methylpropyl)benzonitrile (1.2 g, 6.21 mmol, 37.61% yield) as a yellow oil. ¹H NMR (400 MHz, CHLOROFORM-d) δ=7.77-7.69 (m, 1H), 7.55-7.48 (m, 1H), 7.26-7.22 (m, 1H), 4.81 (d, J=6.3 Hz, 1H), 2.00-1.92 (m, 1H), 0.95 (d, J=6.8 Hz, 3H), 0.87 (d, J=6.8 Hz, 3H).

### 2) 2-fluoro-3-isobutyrylbenzonitrile

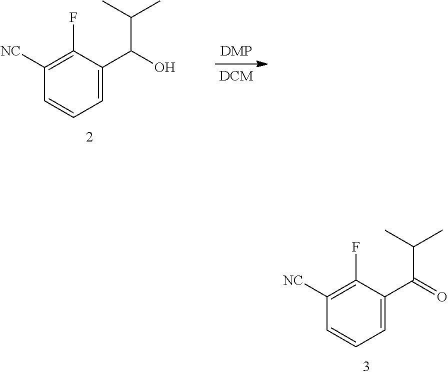

To a solution of 2-fluoro-3-(1-hydroxy-2-methyl-propyl)benzonitrile (1.2 g, 6.21 mmol, 1 eq) in DCM (30 mL) was added DMP (3.16 g, 7.45 mmol, 2.31 mL, 1.2 eq). The mixture was stirred at 20° C. for 12 hr. TLC indicated Reactant 2 was consumed completely and one major new spot formed. The reaction mixture was diluted with H₂O 10 mL and extracted with DCM 30 mL (10 mL×3), dried over Na₂SO₄, filtered and concentrated under reduced pressure to give a residue. The residue was purified by flash silica gel chromatography (Silica Flash Column, Eluent of 0-5% Ethyl acetate/Petroleum ether gradient @60 mL/min) to give compound 2-fluoro-3-isobutyrylbenzonitrile (840 mg, 4.39 mmol, 70.74% yield) as a yellow oil. ¹H NMR (400 MHz, CHLOROFORM-d) δ=8.07-8.00 (m, 1H), 7.80 (ddd, J=1.8, 5.9, 7.6 Hz, 1H), 7.38 (t, J=7.8 Hz, 1H), 3.42-3.32 (m, 1H), 1.22 (dd, J=0.8, 6.9 Hz, 6H).

### 3) 3-(1-(cyclopropylamino)-2-methylpropyl)-2-fluorobenzonitrile

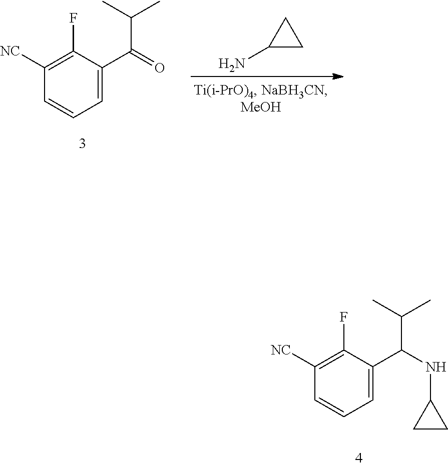

To a solution of 2-fluoro-3-(2-methylpropanoyl)benzonitrile (840 mg, 4.39 mmol, 1 eq) in MeOH (8 mL) was added cyclopropanamine (376.25 mg, 6.59 mmol, 456.61 μL, 1.5 eq) and Ti(i-PrO)₄ (3.75 g, 13.18 mmol, 3.89 mL, 3 eq). The mixture was stirred at 60° C. for 12 hr. NaBH₃CN (1.10 g, 17.57 mmol, 4 eq) was added to the reaction at 20° C. and stirred at 60° C. for 2 hr. LC-MS showed one main peak with desired m/z was detected. The reaction mixture was concentrated under reduced pressure to remove solvent. The residue was diluted with H₂O 10 mL and extracted with EtOAc 30 mL (10 mL×3), dried over Na₂SO₄, filtered and concentrated under reduced pressure to give a residue. The residue was purified by flash silica gel chromatography (Silica Flash Column, Eluent of 0-7% Ethyl acetate/Petroleum ether gradient @80 m/min) to give compound 3-(1-(cyclopropylamino)-2-methylpropyl)-2-fluorobenzonitrile (577 mg, 2.48 mmol, 56.54% yield) as a colorless oil.

### 4) 2-bromo-N-(1-(3-cyano-2-fluorophenyl)-2-methylpropyl)-N-cyclopropylacetamide

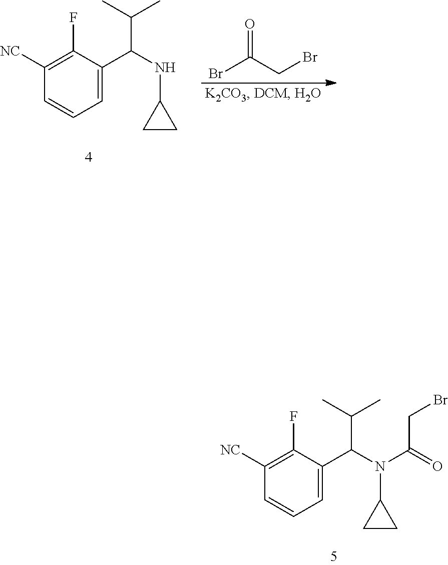

To a stirred cooled solution of K₂CO₃ (686.60 mg, 4.97 mmol, 2 eq) in H₂O (5 mL) was added a solution of 3-[1-(cyclopropylamino)-2-methyl-propyl]-2-fluoro-benzonitrile (577 mg, 2.48 mmol, 1 eq) in DCM (5 mL), followed by addition of 2-bromoacetyl bromide (601.63 mg, 2.98 mmol, 259.66 μL, 1.2 eq). The reaction mixture was stirred at 20° C. for 1 h. LC-MS showed Reactant 4 was consumed completely and one main peak with desired m/z was detected. The reaction mixture was diluted with H₂O 10 mL and extracted with DCM 30 mL (10 mL×3), dried over Na₂SO₄, filtered and concentrated under reduced pressure to give crude product 2-bromo-N-[1-(3-cyano-2-fluoro-phenyl)-2-methyl-propyl]-N-cyclopropyl-acetamide (820 mg, crude yellow oil.

### 5)N-(1-(3-cyano-2-fluorophenyl)-2-methylpropyl)-N-cyclopropyl-2-(1,3-dioxoisoindolin-2-yl)acetamide

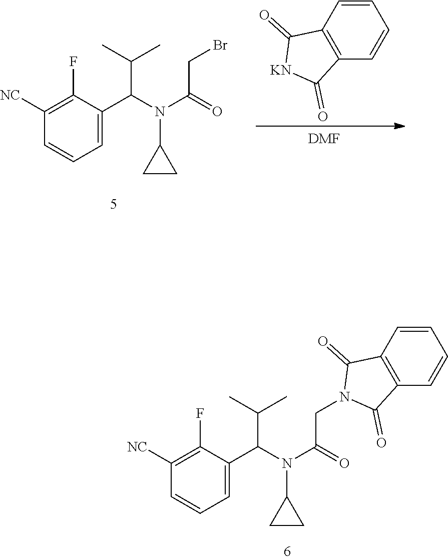

To a solution of 2-bromo-N-[1-(3-cyano-2-fluoro-phenyl)-2-methyl-propyl]-N-cyclopropyl-acetamide (820 mg, 2.32 mmol, 1 eq) in DMF (20 mL) was added (1,3-dioxoisoindolin-2-yl)potassium (515.98 mg, 2.79 mmol, 1.2 eq). The mixture was stirred at 20° C. for 12 hr. LC-MS showed Reactant 5 was consumed completely and one main peak with desired m/z was detected. The reaction mixture was diluted with H₂O 15 mL and extracted with EtOAc 45 mL (15 mL×3). The combined organic layers were washed with aqueous NaCl 20 mL (10 mL×2), dried over Na₂SO₄, filtered and concentrated under reduced pressure to give a residue. The residue was purified by flash silica gel chromatography (Silica Flash Column, Eluent of 0-20% Ethyl acetate/Petroleum ether gradient @80 m/min) to give crude product N-(1-(3-cyano-2-fluorophenyl)-2-methylpropyl)-N-cyclopropyl-2-(1,3-dioxoisoindolin-2-yl)acetamide (1.08 g, crude) yellow oil.

### 6) 2-amino-N-(1-(3-cyano-2-fluorophenyl)-2-methylpropyl)-N-cyclopropylacetamide

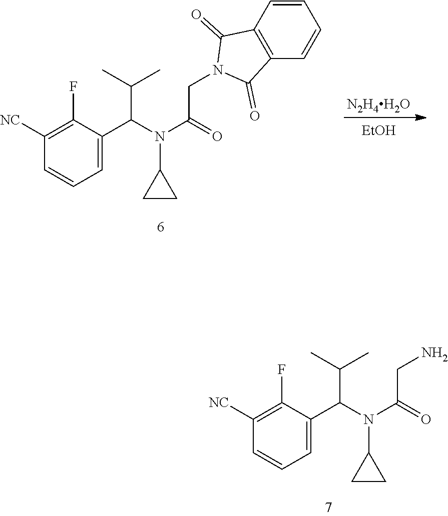 [SMILES: "EtOH" ; CC(C)C(c1cccc(C#N)c1F)N(C(=O)CN1C(=O)c2ccccc2C1=O)C1CC1 ; "N2H4·H2O" ; CC(C)C(c1cccc(C#N)c1F)N(C(=O)CN)C1CC1]

To a solution of N-[1-(3-cyano-2-fluoro-phenyl)-2-methyl-propyl]-N-cyclopropyl-2-(1,3-dioxoisoindolin-2-yl)acetamide (200 mg, 476.82 μmol, 1 eq) in EtOH (20 mL) was added N₂H₄·H₂O (59.67 mg, 953.64 μmol, 57.82 μL, 80% purity, 2 eq). The mixture was stirred at 80° C. for 3 hr. LC-MS showed Reactant 6 was consumed completely and one main peak with desired m/z was detected. The mixture was cooled to 4° C. and the phthalyl hydrazide removed by filtration. The ethanol was removed in vacuo. The solution was extracted with EtOAc 30 mL (10 mL×3) and the organic extract was dried over Na₂SO₄, filtered and concentrated under reduced pressure to give a residue. The crude product 2-amino-N-[1-(3-cyano-2-fluorophenyl)-2-methylpropyl]-N-cyclopropylacetamide (146 mg, crude) yellow oil.

### 7) 3-(1-((2-aminoethyl)(cyclopropyl)amino)-2-methylpropyl)-2-fluorobenzonitrile TFA salt

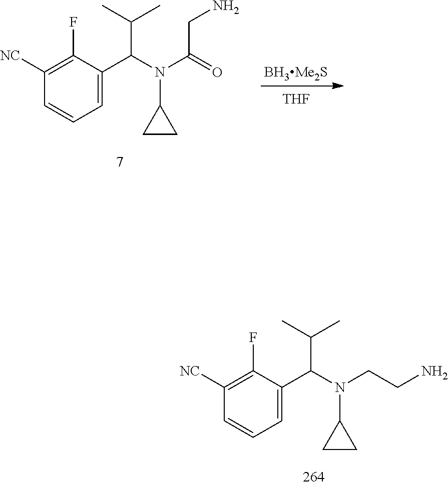 [SMILES: CC(C)C(c1cccc(C#N)c1F)N(C(=O)CN)C1CC1 ; CC(C)C(c1cccc(C#N)c1F)N(CCN)C1CC1 ; "THF"]

A dry round-bottom flask flushed with N₂ was charged with the desired 2-amino-N-[1-(3-cyano-2-fluoro-phenyl)-2-methyl-propyl]-N-cyclopropyl-acetamide (96 mg, 331.78 μmol, 1 eq) and THF (5 mL), BH₃-Me₂S (10 M, 66.36 μL, 2 eq) was added to the reaction at 0° C. and stirred at 20° C. for 4 hr. LC-MS showed Reactant 7 was consumed completely and one main peak with desired m/z was detected. The reaction mixture was quenched by addition MeOH 2 mL at 0° C. and stirred at 60° C. for 2 hr, and then diluted with H₂O 10 mL and extracted with EtOAc 30 mL (10 mL×3), dried over Na₂SO₄, filtered and concentrated under reduced pressure to give a residue. The residue was purified by prep-HPLC (TFAcondition). column: Phenomenex Luna C18 75*30 mm*3 um; mobile phase: [H2O (0.1% TFA)-ACN]; gradient: 15%-45% B over 8.0 min to give compound 264 (3-(1-((2-aminoethyl)(cyclopropyl)amino)-2-methylpropyl)-2-fluorobenzonitrile TFA salt) (10.85 mg, 26.75 μmol, 8.06% yield, 95.99% purity, TFA) as a yellow oil. MS (ESI): m/z=276.3[M+H]⁺, retention time: 1.702 min, method:B. ¹H NMR (400 MHz, DMSO-d6) δ=7.90 (dt, J=1.5, 6.9 Hz, 1H), 7.81-7.61 (m, 4H), 7.46 (t, J=7.8 Hz, 1H), 3.71 (d, J=11.1 Hz, 1H), 3.06-2.83 (m, 3H), 2.40-2.23 (m, 2H), 1.39-1.29 (m, 1H), 1.01 (d, J=6.4 Hz, 3H), 0.67-0.54 (m, 5H), 0.53-0.34 (m, 2H).

### Example 39: Synthesis of Ni-(1-(3-chloro-2-fluorophenyl)-2,2,2-trifluoroethyl)-N¹-cyclopropylethane-1,2-diamine hydrochloride (Compound 263)

### 1) 1-(3-chloro-2-fluorophenyl)-2,2,2-trifluoroethan-1-ol

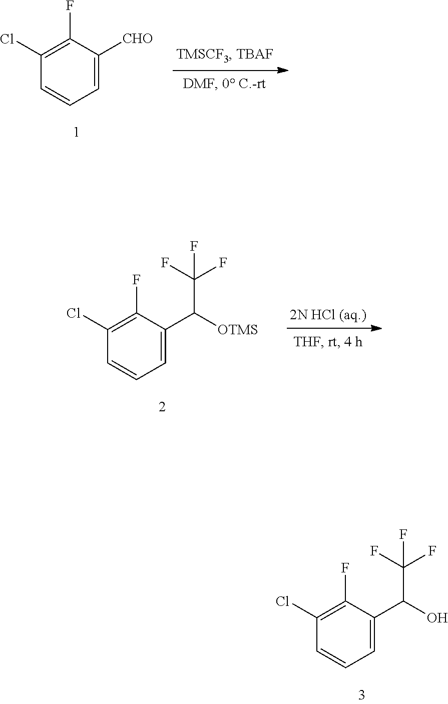

To a solution of 3-chloro-2-fluoro-benzaldehyde (4 g, 25.2 mmol, 1 eq) in DMF (20 mL) was added TMSCF₃ (4.7 g, 32.8 mmol, 1.3 eq) and stirred at 0° C. for 15 min, and then TBAF (1 M, 252.3 μL, 0.01 eq) was added into the reaction mixture. The mixture was stirred at 25° C. for 12 hr. And then HCl (2 M, 25.2 mL, 2 eq) was added to the mixture, and the mixture was stirred at 25° C. for 3 hr. TLC indicated Reactant 1 was consumed completely and many new spots formed. The reaction mixture was partitioned between Ethyl acetate 20 mL and reaction mixture. The organic phase was separated, washed with sat. NaCl 20 mL, dried over Na₂SO₄, filtered and concentrated under reduced pressure to give a residue. The residue was purified by flash silica gel chromatography (20 g Silica Flash Column, Eluent of 0-5% Ethyl acetate/Petroleum ether gradient @80 m/min) to give compound 1-(3-chloro-2-fluorophenyl)-2,2,2-trifluoroethan-1-ol (5.1 g, 22.2 mmol, 88.1% yield) as a white solid. ¹H NMR (400 MHz, CHLOROFORM-d) δ=7.54 (t, J=6.9 Hz, 1H), 7.50-7.44 (m, 1H), 7.22-7.16 (m, 1H), 5.49-5.41 (m, 1H)

### 2) 1-(3-chloro-2-fluorophenyl)-2,2,2-trifluoroethan-1-one

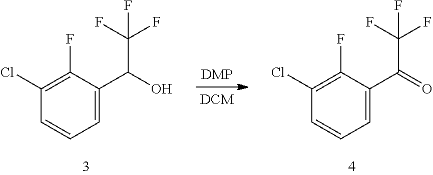 [SMILES: OC(c1cccc(Cl)c1F)C(F)(F)F ; O=C(c1cccc(Cl)c1F)C(F)(F)F]

To a solution of 1-(3-chloro-2-fluorophenyl)-2,2,2-trifluoroethan-1-ol (2.5 g, 10.9 mmol, 1 eq) in DCM (10 mL) was added DMP (5.6 g, 13.1 mmol, 4.1 mL, 1.2 eq). The mixture was stirred at 25° C. for 12 hr. TLC indicated Reactant 3 was consumed completely and two new spots formed. The reaction mixture was concentrated under reduced pressure to give a residue. The residue was purified by flash silica gel chromatography (10 g Silica Flash Column, Eluent of 0-10% Ethyl acetate/Petroleum ether gradient @80 m/min) to give compound 1-(3-chloro-2-fluorophenyl)-2,2,2-trifluoroethan-1-one (1.3 g, 5.9 mmol, 54.1% yield) as a white solid. 1H NMR (400 MHz, CHLOROFORM-d) δ=7.76-7.62 (m, 2H), 7.27-7.16 (m, 1H)

### 3) (E)-1-(3-chloro-2-fluorophenyl)-N-cyclopropyl-2,2,2-trifluoroethan-1-imine

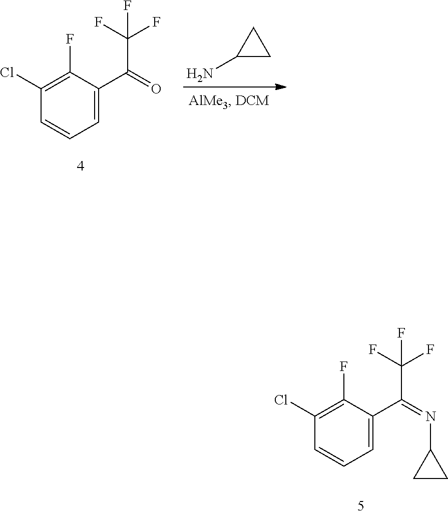

Dissolve the 1-(3-chloro-2-fluorophenyl)-2,2,2-trifluoroethan-1-one (1.3 g, 5.9 mmol, 1 eq) and cyclopropanamine (506.5 mg, 8.9 mmol, 614.7 μL, 1.5 eq) under N₂ in DCM (5 mL). Add AlMe₃ (2 M, 4.4 mL, 1.5 eq) dropwise to the reaction mixture. Stir the solution at 20° C. for 5 hr. LC-MS showed ˜23% of desired compound was detected. Quench the reaction mixture by dropwise addition of 20% aqueous NaOH. Extract the aqueous layer with CH₂Cl₂ (3×30 mL). Dry the organic layer over Na₂SO₄. Remove the solvent under reduced pressure. The residue was purified by flash silica gel chromatography (10 g Silica Flash Column, Eluent of 0˜10% Ethyl acetate/Petroleum ether gradient @80 mL/min) to give compound (E)-1-(3-chloro-2-fluorophenyl)-N-cyclopropyl-2,2,2-trifluoroethan-1-imine (0.58 g, 1.3 mmol, 22.5% yield, 61.4% purity) as a yellow oil.

### 4)N-(1-(3-chloro-2-fluorophenyl)-2,2,2-trifluoroethyl)cyclopropanamine

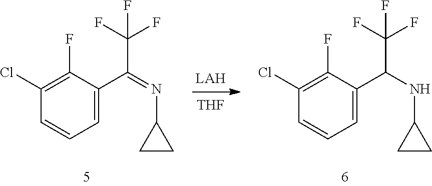 [SMILES: Fc1c(Cl)cccc1/C(=N\C1CC1)C(F)(F)F ; Fc1c(Cl)cccc1C(NC1CC1)C(F)(F)F ; "THF"]

A mixture of (E)-1-(3-chloro-2-fluorophenyl)-N-cyclopropyl-2,2,2-trifluoroethan-1-imine (0.58 g, 2.2 mmol, 1 eq) in THF (6 mL) was degassed and purged with N₂ for 3 times, and then LAH (2.5 M, 1.7 mL, 2.0 eq) was added to the reaction at 0° C., the mixture was stirred at 0° C. for 2 hr under N₂ atmosphere. LC-MS showed Reactant 5 was consumed completely and desired mass was detected. The reaction mixture was quenched by addition sat. MgSO₄ 10 mL at 0° C., and then extracted with Ethyl acetate 30 mL (10 mL×3). The combined organic layers were washed with water 30 mL, dried over Na₂SO₄, filtered and concentrated under reduced pressure to give a residue. The residue was purified by flash silica gel chromatography (10 g Silica Flash Column, Eluent of 0-10% Ethyl acetate/Petroleum ether gradient @80 mL/min) to give compound N-(1-(3-chloro-2-fluorophenyl)-2,2,2-trifluoroethyl)cyclopropanamine (0.5 g, 805.5 μmol, 37.1% yield, 41.5% purity) as a yellow oil.

### 5) 2-bromo-N-(1-(3-chloro-2-fluorophenyl)-2,2,2-trifluoroethyl)-N-cyclopropylacetamide

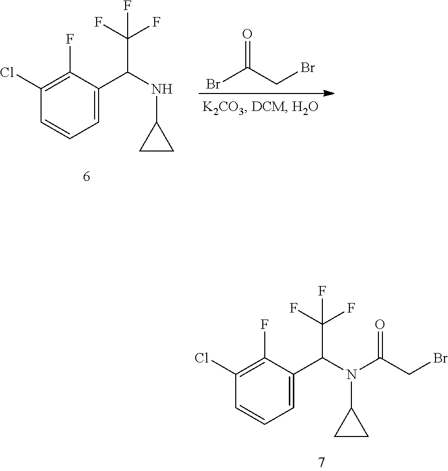

To a solution of N-(1-(3-chloro-2-fluorophenyl)-2,2,2-trifluoroethyl)cyclopropanamine (0.5 g, 1.9 mmol, 1 eq) in DCM (6 mL) was added K₂CO₃ (537.0 mg, 3.9 mmol, 2 eq) in H₂O (6 mL) and 2-bromoacetyl bromide (470.6 mg, 2.3 mmol, 203.1 μL, 1.2 eq) was added to the reaction at 0° C. The mixture was stirred at 25° C. for 12 hr. LC-MS showed Reactant 6 was consumed completely and ˜48% of desired mass was detected. The reaction mixture was extracted with Dichloromethane 15 mL (5 mL×3). The combined organic layers were washed with sat. NaCl 10 mL, dried over Na₂SO₄, filtered and concentrated under reduced pressure to give crude product 2-bromo-N-(1-(3-chloro-2-fluorophenyl)-2,2,2-trifluoroethyl)-N-cyclopropylacetamide (0.56 g, 682.6 μmol, 35.1% yield, 47.5% purity) as a yellow oil.

### 6) 2-azido-N-(1-(3-chloro-2-fluorophenyl)-2,2,2-trifluoroethyl)-N-cyclopropylacetamide

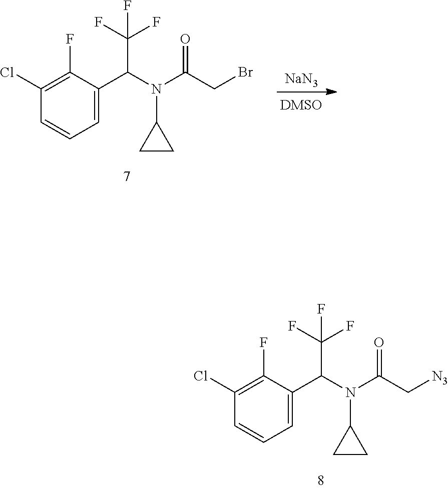

To a solution of 2-bromo-N-(1-(3-chloro-2-fluorophenyl)-2,2,2-trifluoroethyl)-N-cyclopropylacetamide (0.5 g, 1.2 mmol, 1 eq) in DMSO (1 mL) was added NaN₃ (82.8 mg, 1.3 mmol, 1.1 eq). The mixture was stirred at 20° C. for 12 hr. LC-MS showed Reactant 7 was consumed completely and ˜16% of desired mass was detected. The reaction mixture was quenched by addition water 10 mL at 0° C., and then extracted with Ethyl acetate 15 mL (5 mL×3). The combined organic layers were dried over Na₂SO₄, filtered and concentrated under reduced pressure to give a residue. The crude product was purified by reversed-phase HPLC (0.1% NH₃H₂O condition, Column: Waters Xbridge BEH C18 100*30 mm*10 um; mobile phase: [H₂O (10 mM NH₄HCO₃)-ACN]; gradient: 50%-80% B over 8.0 min to give compound 2-azido-N-(1-(3-chloro-2-fluorophenyl)-2,2,2-trifluoroethyl)-N-cyclopropylacetamide (90.4 mg, 245.1 μmol, 21.2% yield, 95.1% purity) as a white solid. ¹H NMR (400 MHz, DMSO-d6) δ=7.75-7.69 (m, 1H), 7.66-7.60 (m, 1H), 7.38-7.32 (m, 1H), 6.46-6.38 (m, 1H), 4.52-4.41 (m, 2H), 2.48-2.41 (m, 1H), 0.95-0.60 (m, 4H)

### 7) 2-amino-N-(1-(3-chloro-2-fluorophenyl)-2,2,2-trifluoroethyl)-N-cyclopropylacetamide

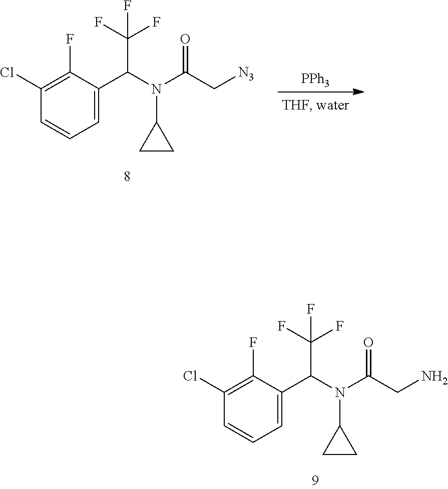 [SMILES: [N-]=[N+]=NCC(=O)N(C1CC1)C(c1cccc(Cl)c1F)C(F)(F)F ; "THF, water" ; NCC(=O)N(C1CC1)C(c1cccc(Cl)c1F)C(F)(F)F ; "PPh3"]

To a solution of 2-azido-N-(1-(3-chloro-2-fluorophenyl)-2,2,2-trifluoroethyl)-N-cyclopropylacetamide (0.08 g, 228.1 μmol, 1 eq) in THF (1.5 mL) and Water (0.5 mL) was added PPh₃ (89.8 mg, 342.2 μmol, 1.5 eq). The mixture was stirred at 25° C. for 12 hr. LC-MS showed Reactant 8 was consumed completely and ˜49% of desired mass was detected. The reaction mixture was filtered and concentrated under reduced pressure to give a residue. The residue was purified by column chromatography (SiO₂, DCM/MeOH=10/1) to give compound 2-amino-N-(1-(3-chloro-2-fluorophenyl)-2,2,2-trifluoroethyl)-N-cyclopropylacetamide (0.02 g, 61.6 μmol, 27.0% yield) as a colorless oil.

### 8) N¹-(1-(3-chloro-2-fluorophenyl)-2,2,2-trifluoroethyl)-N¹-cyclopropylethane-1,2-diamine hydrochloride

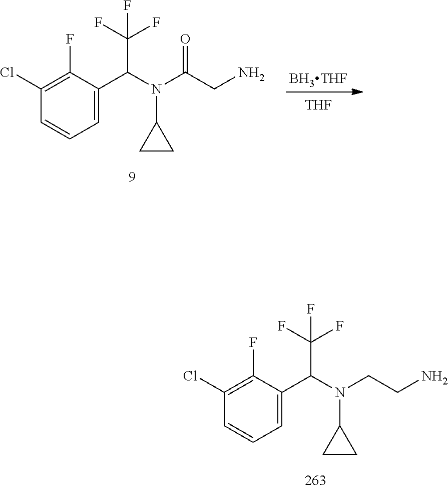 [SMILES: NCC(=O)N(C1CC1)C(c1cccc(Cl)c1F)C(F)(F)F ; NCCN(C1CC1)C(c1cccc(Cl)c1F)C(F)(F)F ; "BH3·THF" ; "THF"]

A mixture of 2-amino-N-(1-(3-chloro-2-fluorophenyl)-2,2,2-trifluoroethyl)-N-cyclopropylacetamide (0.02 g, 61.6 μmol, 1 eq) in THF (5 mL) was degassed and purged with N₂ for 3 times, and then BH₃·THF (1 M, 616.0 μL, 10 eq) was added to the reaction at 0° C. and stirred at 0° C. for 30 min, the mixture was stirred at 60° C. for 12 hr under N₂ atmosphere. LC-MS showed Reactant 9 was consumed completely and ˜41% of desired mass was detected. The reaction mixture was quenched by addition MeOH 2 mL at 0° C., and then diluted with water 5 mL and extracted with Ethyl acetate 15 mL (5 mL×3). The combined organic layers were washed with sat. NaCl 10 mL, dried over Na₂SO₄, filtered and concentrated under reduced pressure to give a residue. The crude product was purified by reversed-phase HPLC (0.04% HCl condition). Column: Phenomenex luna C18 8*30 mm*3 um; mobile phase: [H₂O (0.04% HCl)-ACN]; gradient: 20%-50% B over 8.0 min. Compound 263 (N¹-(1-(3-chloro-2-fluorophenyl)-2,2,2-trifluoroethyl)-N¹-cyclopropylethane-1,2-diamine hydrochloride) (0.02 g, 47.3 μmol, 76.7% yield, 97.93% purity, HCl) as a white solid. MS (ESI): m/z=311.2 [M+H]⁺; retention time: 1.706 min, method:M ¹H NMR (400 MHz, DMSO-d6) δ=7.80-7.64 (m, 4H), 7.53-7.46 (m, 1H), 7.39-7.33 (m, 1H), 5.18-5.07 (m, 1H), 3.06-2.67 (m, 4H), 2.00-1.92 (m, 1H), 0.68-0.44 (m, 4H)

### Example 40: Synthesis of N′-[1-(3-chloro-2-fluoro-phenyl)but-3-enyl]-N′-cyclopropyl-ethane-1,2-diamine TFA salt (Compound 271A)

### 1) (Z)-1-(3-chloro-2-fluorophenyl)-N-cyclopropylmethanimine

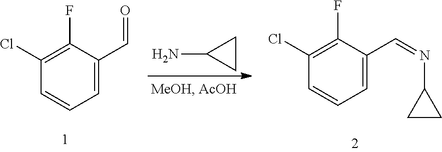

To a solution of 3-chloro-2-fluoro-benzaldehyde (5 g, 31.5 mmol, 1eq) in MeOH (70 mL) was added cyclopropanamine (2.70 g, 47.30 mmol, 3.28 mL, 1.5eq) and AcOH (5.68 g, 94.60 mmol, 5.42 mL, 3eq). The mixture was stirred at 60° C. for 12 hr. LCMS showed desired compound formed. The reaction mixture was concentrated under reduced pressure to give compound (Z)-1-(3-chloro-2-fluoro-phenyl)-N-cyclopropyl-methanimine (6 g, crude) as yellow oil.

### 2)N-(1-(3-chloro-2-fluorophenyl) but-3-en-1-yl) cyclopropanamine

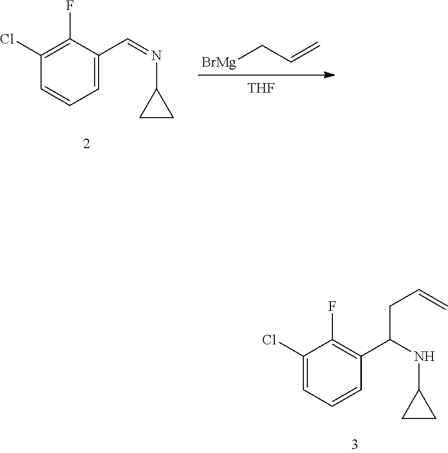

A mixture of (Z)-1-(3-chloro-2-fluoro-phenyl)-N-cyclopropyl-methanimine (6 g, 30.36 mmol, 1eq) in THF (150 mL) was added allyl(bromo)magnesium (1 M, 45.54 mL, 1.5eq). The mixture was degassed and purged with N₂ for 3 times, and then the mixture was stirred at −78° C. for 3 hr under N₂ atmosphere. LCMS showed desired compound formed. The reaction mixture was quenched by addition NH₄Cl (100 ml), and then diluted with H₂O (100 mL) and extracted with EtOAc 150 mL (50 mL*3). The combined organic layers were dried over Na2SO4, filtered and concentrated under reduced pressure to give a residue. The residue was purified by flash silica gel chromatography (40 g Silica Flash Column, Eluent of 0-3% Ethyl acetate/Petroleum ether gradient@80 mL/min) to afford compound N-[1-(3-chloro-2-fluoro-phenyl) but-3-enyl]cyclopropanamine (2.5 g, 10.43 mmol, 34.35% yield) as yellow oil.

### 3) tert-butyl (2-((1-(3-chloro-2-fluorophenyl)but-3-en-1-yl)(cyclopropyl)amino)ethyl)carbamate

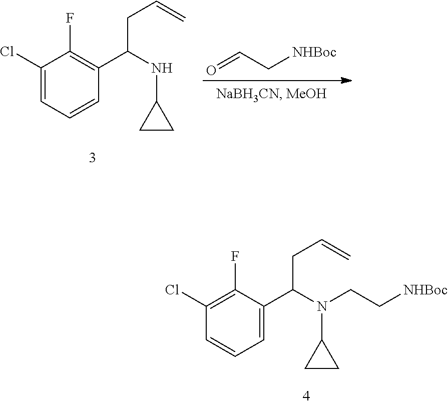 [SMILES: C=CCC(NC1CC1)c1cccc(Cl)c1F ; CC(C)(C)OC(=O)NCC=O ; C=CCC(c1cccc(Cl)c1F)N(CCNC(=O)OC(C)(C)C)C1CC1]

To a solution of N-[1-(3-chloro-2-fluoro-phenyl)but-3-enyl]cyclopropanamine (1 g, 4.17 mmol, 1eq) in MeOH (20 mL) was added tert-butyl N-(2-oxoethyl)carbamate (3.98 g, 25.03 mmol, 6eq). The mixture was stirred at 60° C. for 12 hr. Then NaBH₃CN (1.05 g, 16.6 mmol, 4eq) was added at 25° C. The mixture was stirred at 60° C. for 2 hr. LCMS showed desired compound formed. The reaction mixture was concentrated under reduced pressure to remove solvent. The residue was diluted with H₂O 10 mL and extracted with EtOAC 15 mL (5 mL*3). The combined organic layers were dried over [Na₂SO₄], filtered and concentrated under reduced pressure to give a residue. The residue was purified by flash silica gel chromatography (20 g, Silica Flash Column, Eluent of 0˜15% Ethyl acetate/Petroleum ether gradient@120 mL/min) to give compound tert-butyl N-[2-[1-(3-chloro-2-fluoro-phenyl) but-3-enyl-cyclopropyl-amino]ethyl]carbamate (314 mg, 820.06 μmol, 19.66% yield) as yellow solid.

### 4) N1-(1-(3-chloro-2-fluorophenyl) but-3-en-1-yl)-N1-cyclopropylethane-1, 2-diamine TFA salt

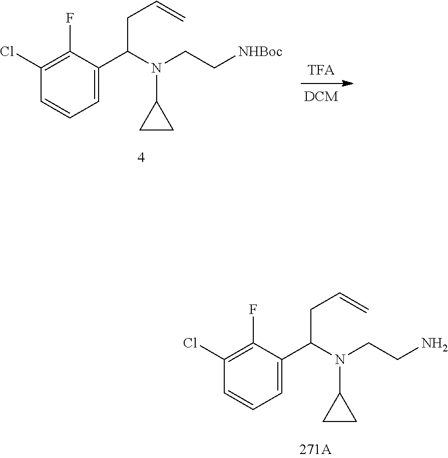

A solution of tert-butyl N-[2-[1-(3-chloro-2-fluoro-phenyl)but-3-enyl-cyclopropyl-amino]ethyl]carbamate (20 mg, 52.23 μmol, 1eq) in TFA (1 mL) and DCM (1 mL) was stirred at 25° C. for 0.5 hr. LCMS showed desired compound formed. The reaction mixture was concentrated under reduced pressure to remove solvent. The residue was purified by prep-HPLC (TFA condition, column: Phenomenex luna C18 100*40 mm*3 um; mobile phase: [H₂O (0.1% TFA)-ACN]; gradient: 15%-45% B over 8.0 min) to give compound N′-[1-(3-chloro-2-fluoro-phenyl)but-3-enyl]-N′-cyclopropyl-ethane-1,2-diamine TFA salt (21.4 mg, 53.43 μmol, 51.15% yield, 99.08% purity, TFA) as yellow oil. MS (ESI): m/z=282.13[M+H]⁺; retention time: 1.773 min, Method M ¹H NMR (400 MHz, DMSO-d₆) δ=7.60 (br s, 3H), 7.55-7.49 (m, 1H), 7.41 (t, J=6.5 Hz, 1H), 7.27-7.20 (m, 1H), 5.64 (tdd, J=6.8, 10.2, 17.0 Hz, 1H), 5.10-4.88 (m, 2H), 4.26 (dd, J=6.7, 9.1 Hz, 1H), 2.98-2.82 (m, 3H), 2.77-2.59 (m, 2H), 2.49-2.42 (m, 1H), 1.66 (br d, J=2.9 Hz, 1H), 0.60-0.27 (m, 4H)

### Example 41: Synthesis of N′-[1-(4-chloro-2-thienyl)ethyl]-N′-cyclopropyl-ethane-1,2-diamine hydrochloride (Compound 279)

### 1)N-(1-(4-chlorothiophen-2-yl)ethyl)cyclopropanamine

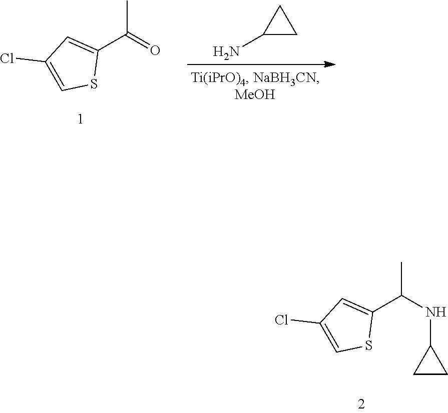

To a solution of cyclopropanamine (266.6 mg, 4.7 mmol, 323.5 μL, 1.5 eq), 1-(4-chloro-2-thienyl)ethanone (500 mg, 3.1 mmol, 1 eq) in MeOH (20 mL) was added Ti(i-PrO)₄ (2.6 g, 9.3 mmol, 2.8 mL, 3 eq) and AcOH (373.9 mg, 6.2 mmol, 356.4 μL, 2 eq). The mixture was stirred at 60° C. for 12 hr. NaBH₃CN (782.5 mg, 12.5 mmol, 4 eq) was added. The mixture was stirred at 60° C. for 2 hr. LC-MS showed desired compound was detected. The reaction mixture was concentrated under reduced pressure to remove solvent. The residue was diluted with H₂O 15 mL and extracted with EtOAc (15 mL*3). The combined organic layers were washed with brine (15 mL*2), dried over Na₂SO₄, filtered and concentrated under reduced pressure to give compound N-(1-(4-chlorothiophen-2-yl)ethyl)cyclopropanamine (300 mg, crude) as yellow oil. ¹H NMR (400 MHz, CHLOROFORM-d) δ=7.06 (d, J=1.3 Hz, 1H), 6.96 (s, 1H), 4.25 (q, J=6.7 Hz, 1H), 2.26-2.18 (m, 1H), 1.61 (d, J=6.8 Hz, 3H), 0.70-0.53 (m, 5H).

### 2) tert-butyl (2-((1-(4-chlorothiophen-2-yl)ethyl)(cyclopropyl)amino)ethyl)carbamate

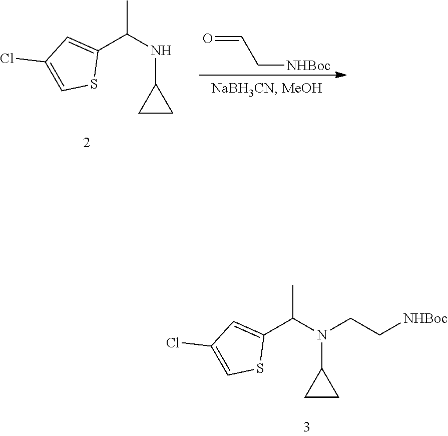 [SMILES: CC(NC1CC1)c1cc(Cl)cs1 ; CC(C)(C)OC(=O)NCC=O ; CC(c1cc(Cl)cs1)N(CCNC(=O)OC(C)(C)C)C1CC1]

To a solution of tert-butyl N-(2-oxoethyl)carbamate (1.2 g, 7.4 mmol, 6 eq) and N-[1-(4-chloro-2-thienyl)ethyl]cyclopropanamine (250 mg, 1.2 mmol, 1 eq) in MeOH (10 mL), then AcOH (89.31 mg, 1.5 mmol, 85.1 μL, 1.2 eq) was added to added pH=5. The mixture was stirred at 60° C. for 12 hr. Then NaBH₃CN (311.5 mg, 5.0 mmol, 4 eq) was added. The mixture was stirred at 60° C. for 2 hr. LC-MS showed desired compound was detected. The reaction mixture was concentrated under reduced pressure to remove solvent. The residue was diluted with H₂O 10 mL and extracted with EtOAc (10 mL*3). The combined organic layers were washed with brine (10 mL*2), dried over Na₂SO₄, filtered and concentrated under reduced pressure to give a residue. The residue was purified by prep-TLC (SiO₂, Petroleum ether: Ethyl acetate=5:1) to give compound tert-butyl (2-((1-(4-chlorothiophen-2-yl)ethyl)(cyclopropyl) amino)ethyl)carbamate (66.23% purity, 500 mg) as a white solid.

### 3) N1-(1-(4-chlorothiophen-2-yl)ethyl)-N1-cyclopropylethane-1,2-diamine hydrochloride

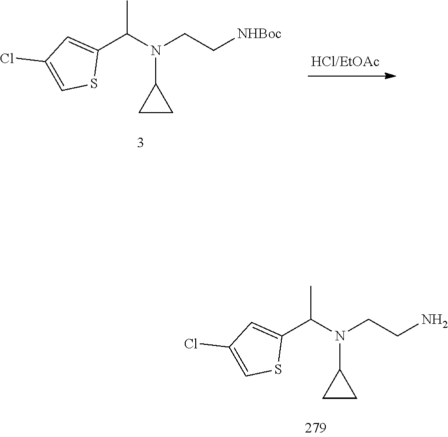 [SMILES: CC(c1cc(Cl)cs1)N(CCNC(=O)OC(C)(C)C)C1CC1 ; CC(c1cc(Cl)cs1)N(CCN)C1CC1]

A solution of tert-butyl N-[2-[1-(4-chloro-2-thienyl)ethyl-cyclopropyl-amino]ethyl]carbamate (200 mg, 579.9 μmol, 1 eq) in HCl/EtOAc (4 mL, 4M) was stirred at 20° C. for 2 hr. LC-MS showed desired compound was detected. The reaction mixture was concentrated under reduced pressure to remove solvent. The residue was purified by prep-HPLC (HCl condition; column: Phenomenex Luna C18 75*30 mm*3 um; mobile phase: [H₂O (0.04% HCl)-ACN]; gradient: 5%-35% B over 8.0 min) to give compound N′-[1-(4-chloro-2-thienyl)ethyl]-N′-cyclopropyl-ethane-1,2-diamine hydrochloride (20.0 mg, 71.2 mol, 12.3 yield, 100% purity, HCl) as a white solid. MS (ESI): m/z=245.1 [M+H]+ retention time: 1.441 min, method:M. ¹H NMR (400 MHz, DMSO-d6) δ=8.03-7.70 (m, 3H), 7.47 (s, 1H), 7.06 (br s, 1H), 4.48-4.31 (m, 1H), 3.08-2.73 (m, 4H), 2.08 (br s, 1H), 1.53 (br d, J=6.4 Hz, 3H), 0.62 (br d, J=5.5 Hz, 4H).

### Example 42: Synthesis of 3-[1-[2-aminoethyl(cyclopropyl)amino]-2-fluoro-2-methyl-propyl]-2-fluoro-benzonitrile TFA salt (Compound 287)

### 1) 2-fluoro-3-(2-fluoro-2-methyl-propanoyl)benzonitrile

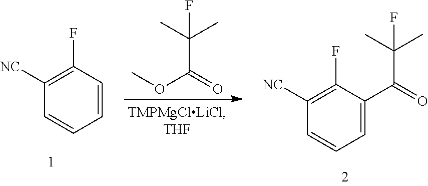

To a solution of 2-fluorobenzonitrile (5.50 g, 45.41 mmol, 4.83 mL, 1 eq) in THF (50 mL) was added lithium; chloro-(2,2,6,6-tetramethyl-1-piperidyl)magnesium; chloride (1 M, 59.03 mL, 1.3 eq). The mixture was stirred at −78° C. for 1 hr, then methyl 2-fluoro-2-methyl-propanoate (12 g, 99.90 mmol, 2.2 eq) was added. The mixture was stirred at 20° C. for 11 hrs. TLC indicated 1 was consumed completely and many new spots formed. The reaction mixture was concentrated under reduced pressure to remove solvent. The residue was diluted with H₂O 100 mL and extracted with EtOAc (80 mL*3). The combined organic layers were dried over Na₂SO₄, filtered and concentrated under reduced pressure to give a residue. The residue was purified by flash silica gel chromatography (Silica Flash Column, Eluent of 0-3% Ethyl acetate/Petroleum ether gradient @120 m/min) to give 2-fluoro-3-(2-fluoro-2-methyl-propanoyl)benzonitrile (1.7 g, 8.13 mmol, 17.90% yield) as a yellow oil. ¹H NMR (400 MHz, CHLOROFORM-d) δ=7.83-7.70 (m, 2H), 7.42-7.34 (m, 1H), 1.74-1.69 (m, 3H), 1.68-1.63 (m, 3H)

### 2) 3-[(E)-N-cyclopropyl-C-(1-fluoro-1-methyl-ethyl)carbonimidoyl]-2-fluoro-benzonitrile

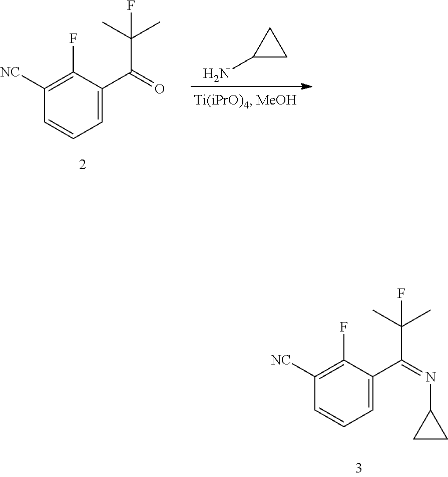

To a solution of 2-fluoro-3-(2-fluoro-2-methyl-propanoyl)benzonitrile (500 mg, 2.39 mmol, 1 eq) in MeOH (4 mL) was added cyclopropanamine (204.70 mg, 3.59 mmol, 248.42 L, 1.5 eq) and Ti(i-PrO)₄ (2.04 g, 7.17 mmol, 2.12 mL, 3 eq). The mixture was stirred at 60° C. for 12 hrs. LC-MS showed 2 was consumed and ˜66% of desired compound was detected. The reaction mixture was concentrated under reduced pressure to remove solvent. The residue was diluted with H₂O 5 mL and extracted with EtOAc (4 mL*3). The combined organic layers were dried over Na₂SO₄, filtered and concentrated under reduced pressure to give a residue. The residue was purified by flash silica gel chromatography (Silica Flash Column, Eluent of 0-3% Ethyl acetate/Petroleum ether gradient @60 m/min) to give 3-[(E)-N-cyclopropyl-C-(1-fluoro-1-methyl-ethyl)carbonimidoyl]-2-fluoro-benzonitrile (380 mg, 1.30 mmol, 54.44% yield, 85.01% purity) as a yellow oil.

### 3) 3-[1-(cyclopropylamino)-2-fluoro-2-methyl-propyl]-2-fluoro-benzonitrile

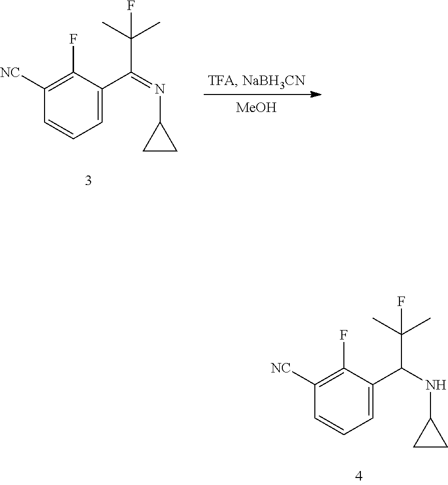

To a solution of 3-[(E)-N-cyclopropyl-C-(1-fluoro-1-methyl-ethyl)carbonimidoyl]-2-fluoro-benzonitrile (360 mg, 1.45 mmol, 1 eq) in MeOH (3 mL) was added TFA (181.87 mg, 1.60 mmol, 118.48 μL, 1.1 eq) and NaBH₃CN (364.49 mg, 5.80 mmol, 4 eq). The mixture was stirred at 20° C. for 12 hr. LC-MS showed ˜36% of 3 remained and ˜46% of desired compound was detected. The reaction mixture was concentrated under reduced pressure to remove solvent. The residue was diluted with H₂O 5 mL and extracted with EtOAc (4 mL*3). The combined organic layers were dried over Na₂SO₄, filtered and concentrated under reduced pressure to give a residue. The residue was purified by flash silica gel chromatography (Silica Flash Column, Eluent of 0-3% Ethyl acetate/Petroleum ether gradient @40 mL/min). The residue was purified by prep-HPLC (TFA condition; column: Phenomenex luna C18 100*40 mm*5 um; mobile phase: [H₂O (0.1% TFA)-ACN]; gradient: 1%-35% B over 7.0 min) to give 3-[1-(cyclopropylamino)-2-fluoro-2-methyl-propyl]-2-fluoro-benzonitrile (230 mg, 748.48 μmol, 51.62% yield, 81.45% purity) as a white solid. ¹H NMR (400 MHz, CHLOROFORM-d) δ=8.22-8.08 (m, 1H), 7.78-7.67 (m, 1H), 7.44 (t, J=7.9 Hz, 1H), 4.67 (d, J=17.4 Hz, 1H), 2.27-2.15 (m, 1H), 1.62-1.47 (m, 3H), 1.41-1.28 (m, 3H), 0.99-0.84 (m, 2H), 0.68-0.51 (m, 2H)

### 4) tert-butyl N-[2-[[1-(3-cyano-2-fluoro-phenyl)-2-fluoro-2-methyl-propyl]-cyclopropyl-amino]ethyl]carbamate

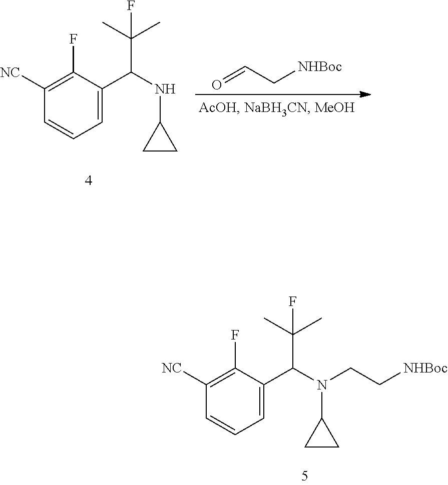

To a solution of 3-[1-(cyclopropylamino)-2-fluoro-2-methyl-propyl]-2-fluoro-benzonitrile (200 mg, 799.08 μmol, 1 eq) and tert-butyl N-(2-oxoethyl)carbamate (763.20 mg, 4.79 mmol, 6 eq) in MeOH (3 mL) was added AcOH to pH=6, the mixture was stirred at 20° C. for 1 hr, then NaBH₃CN (200.86 mg, 3.20 mmol, 4 eq) was added, the mixture was stirred at 20° C. for 1 hr. LC-MS showed ˜74% of 4 remained and ˜18% of desired compound was detected. The reaction mixture was concentrated under reduced pressure to remove solvent. The residue was diluted with H₂O 5 mL and extracted with EtOAc (4 mL*3). The combined organic layers were dried over Na₂SO₄, filtered and concentrated under reduced pressure to give a residue. The residue was purified by flash silica gel chromatography (SepaFlash® Silica Flash Column, Eluent of 0-10% Ethyl acetate/Petroleum ether gradient @40 m/min) to give tert-butyl N-[2-[[1-(3-cyano-2-fluoro-phenyl)-2-fluoro-2-methyl-propyl]-cyclopropyl-amino]ethyl]carbamate (30 mg, 72.26 μmol, 9.04% yield, 94.77% purity) as a white solid.

### 5) 3-[1-[2-aminoethyl(cyclopropyl)amino]-2-fluoro-2-methyl-propyl]-2-fluoro-benzonitrile TFA salt

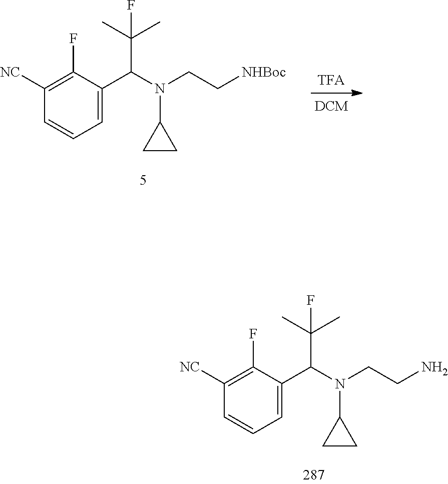

To a solution of tert-butyl N-[2-[[1-(3-cyano-2-fluoro-phenyl)-2-fluoro-2-methyl-propyl]-cyclopropyl-amino]ethyl]carbamate (30 mg, 76.24 μmol, 1 eq) in DCM (1 mL) was added TFA (335.01 mg, 2.94 mmol, 218.25 μL, 38.54 eq). The mixture was stirred at 20° C. for 0.5 hr. LC-MS showed 4% of 5 remained and ˜82% of desired compound was detected. The reaction mixture was concentrated under reduced pressure to give a residue. The residue was purified by prep-HPLC (TFA condition; column: Phenomenex luna C18 100*40 mm*5 um; mobile phase: [H₂O (0.1% TFA)-ACN]; gradient: 25%-55% B over 8.0 min) to give 3-[1-[2-aminoethyl(cyclopropyl)amino]-2-fluoro-2-methyl-propyl]-2-fluoro-benzonitrile TFA salt (10.13 mg, 24.87 μmol, 32.61% yield, 100% purity, TFA) as a white solid. MS (ESI): m/z=293.17[M+H]+, retention time: 1.603 min, method:Bhalo. ¹H NMR (400 MHz, CHLOROFORM-d) δ=7.98-7.92 (m, 1H), 7.92-7.72 (m, 2H), 7.68-7.58 (m, 1H), 7.36-7.29 (m, 1H), 4.34-4.20 (m, 1H), 3.46-3.06 (m, 3H), 2.50-2.39 (m, 1H), 1.76-1.66 (m, 1H), 1.62-1.48 (m, 3H), 1.27-1.12 (m, 3H), 0.53 (s, 4H)

### Example 43: Synthesis of N′-[1-(3-chloro-2-fluoro-phenyl)-2-methyl-propyl]-N′-(2,2-difluoroethyl)ethane-1,2-diamine TFA salt (Compound 282)

### 1) 1-(3-chloro-2-fluoro-phenyl)-2-methyl-propan-1-ol

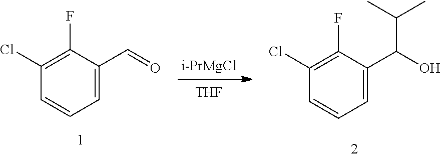

To a solution 3-chloro-2-fluoro-benzaldehyde (10 g, 63.07 mmol, 1 eq) in THF (100 mL), was added chloro(isopropyl)magnesium (2 M, 47.30 mL, 1.5 eq) at −78° C. under N₂. Then the mixture was stirred at 25° C. for 1 h (under N₂). LC-MS showed no 1 remained and ˜33% of desired compound was detected. The reaction was quenched by addition of 30 mL of NH₄Cl very slowly over 10 min under N₂. The aqueous phase was extracted with ethyl acetate (200 mL*3). The combined organic layers were washed with brine (100 mL), dried over anhydrous Na₂SO₄, filtered and concentrated under reduced pressure to give a crude product. The residue was purified by flash silica gel chromatography (20 g Silica Flash Column, Eluent of 0˜2% Ethyl acetate/Petroleum ether gradient @100 m/min) to give 1-(3-chloro-2-fluoro-phenyl)-2-methyl-propan-1-ol (3.5 g, 17.27 mmol, 27.38% yield) as yellow oil.

### 2) 1-(3-chloro-2-fluoro-phenyl)-2-methyl-propan-1-one

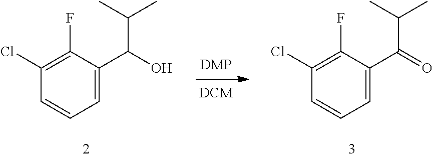

To a solution of 1-(3-chloro-2-fluoro-phenyl)-2-methyl-propan-1-ol (2 g, 9.87 mmol, 1 eq) in DCM (20 mL) was added DMP (12.56 g, 29.61 mmol, 9.17 mL, 3 eq) at 0° C. (under N₂). The mixture was stirred at 25° C. for 1 h. LC-MS showed no 2 remained and ˜30% of desired compound was detected. The reaction mixture was diluted with H₂O 100 mL and extracted with EtOAc (100 mL*3). The combined organic layers were washed with brine (100 mL*1), dried over anhydrous Na₂SO₄, filtered and concentrated under reduced pressure to give a crude product. The crude product was washed by petroleum ether (20 mL), the liquid was concentrated by vacuum under reduced pressure to give 1-(3-chloro-2-fluoro-phenyl)-2-methyl-propan-1-one (1.5 g, 7.48 mmol, 75.75% yield) as a yellow oil.

### 3) 1-(3-chloro-2-fluoro-phenyl)-N-(2,2-difluoroethyl)-2-methyl-propan-1-amine

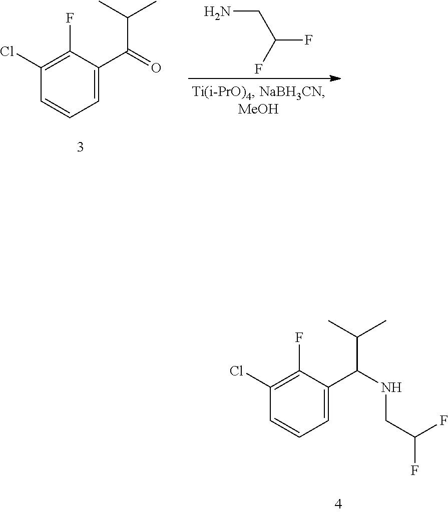 [SMILES: CC(C)C(=O)c1cccc(Cl)c1F ; NCC(F)F ; CC(C)C(NCC(F)F)c1cccc(Cl)c1F]

To a solution of 2,2-difluoroethanamine (4.85 g, 59.81 mmol, 8 eq) and 1-(3-chloro-2-fluoro-phenyl)-2-methylpropan-1-one (1.50 g, 7.48 mmol, 1 eq) in MeOH (10 mL) was added tetraisopropoxytitanium (17.00 g, 59.81 mmol, 17.65 mL, 8 eq). The mixture was stirred at 60° C. for 12 hr. sodium; cyanoboranuide (2.82 g, 44.86 mmol, 6 eq) was added to the reaction and stirred at 60° C. for 2 hr. LC-MS showed ˜10% of 3 remained and ˜55% of desired compound was detected. The reaction mixture was diluted with H₂O 50 mL and extracted with EtOAc (50 mL*3). The combined organic layers were washed with brine (50 mL), dried over anhydrous Na₂SO₄, filtered and concentrated under reduced pressure to give a crude product. The crude product was purified by pre-HPLC (column: Phenomenex luna C18 250*150 mm*15 um; mobile phase: [H₂O (0.1% TFA)-ACN]; gradient: 30%-60% B over 20.0 min) to give 1-(3-chloro-2-fluoro-phenyl)-N-(2,2-difluoroethyl)-2-methyl-propan-1-amine (1.4 g, 5.27 mmol, 70.48% yield) as a yellow oil.

### 4) tert-butyl N-[2-[[1-(3-chloro-2-fluoro-phenyl)-2-methyl-propyl]-(2,2-difluoroethyl)amino]ethyl]carbamate

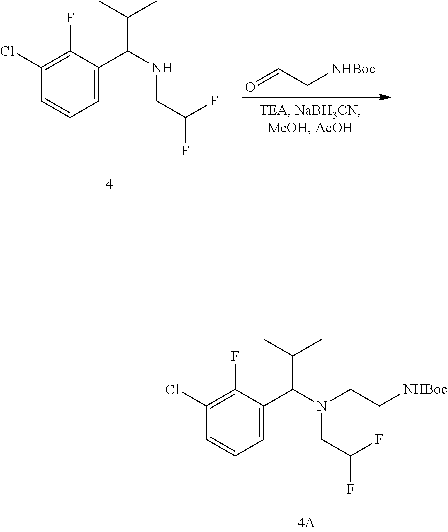

To a solution of 1-(3-chloro-2-fluoro-phenyl)-N-(2,2-difluoroethyl)-2-methyl-propan-1-amine (500 mg, 1.65 mmol, 1 eq, HCl), TEA (167.44 mg, 1.65 mmol, 230.32 μL, 1 eq) and tert-butyl N-(2-oxoethyl)carbamate (1.05 g, 6.62 mmol, 4 eq) in MeOH (4 mL) was added AcOH to pH=6. The mixture was stirred at 20° C. for 1 hr, then NaBH₃CN (415.93 mg, 6.62 mmol, 4 eq) was added, the mixture was stirred at 20° C. for 11 hr. LC-MS showed ˜86% of 4 remained and ˜10% of desired compound was detected. The reaction mixture was concentrated under reduced pressure to give a residue. The residue was purified by prep-HPLC (TFA condition; column: Phenomenex luna C18 100*40 mm*5 um; mobile phase: [H₂O (0.1% TFA)-ACN]; gradient: 45%-100% B over 8.0 min) to give tert-butyl N-[2-[[1-(3-chloro-2-fluoro-phenyl)-2-methyl-propyl]-(2,2-difluoroethyl)amino]ethyl]carbamate (40 mg, 84.09 μmol, 5.08% yield, 85.96% purity) as a white solid.

### 5) N′-[1-(3-chloro-2-fluoro-phenyl)-2-methyl-propyl]-N′-(2,2-difluoroethyl)ethane-1,2-diamine

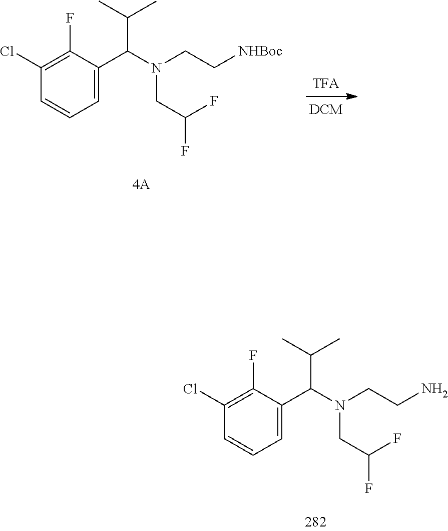

To a solution of tert-butyl N-[2-[[1-(3-chloro-2-fluoro-phenyl)-2-methyl-propyl]-(2,2-difluoroethyl)amino]ethyl]carbamate (40 mg, 97.83 μmol, 1 eq) in DCM (2 mL) was added TFA (429.84 mg, 3.77 mmol, 280.02 μL, 38.54 eq). The mixture was stirred at 20° C. for 1 hr. LC-MS showed no 4A remained and ˜82% of desired compound was detected. The reaction mixture was concentrated under reduced pressure to give a residue. The residue was purified by prep-HPLC (TFA condition; column: Phenomenex luna C18 100*40 mm*5 um; mobile phase: [H₂O (0.1% TFA)-ACN]; gradient: 15%-65% B over 7.0 min) to give N′-[1-(3-chloro-2-fluoro-phenyl)-2-methyl-propyl]-N′-(2,2-difluoroethyl)ethane-1,2-diamine TFA salt (41 mg, 96.97 μmol, 99.13% yield, 100% purity, TFA) as a colorless oil. MS (ESI): m/z=308.13 [M+H]+, retention time: 1.726 min, method:Bhalo. ¹H NMR (400 MHz, CHLOROFORM-d) δ=8.12-7.71 (m, 3H), 7.45-7.31 (m, 1H), 7.20-7.04 (m, 2H), 5.90 (br t, J=55.7 Hz, 1H), 3.67 (br d, J=11.0 Hz, 1H), 3.12 (br s, 2H), 3.05-2.84 (m, 2H), 2.77-2.55 (m, 2H), 2.26 (tt, J=5.9, 11.6 Hz, 1H), 1.15 (br d, J=6.4 Hz, 3H), 0.89-0.61 (m, 3H)

### Example 44: Synthesis of N′-cyclopropyl-N′-[1-(3-ethynyl-2-fluoro-phenyl)ethyl]ethane-1,2-diamine TFA salt (Compound 295)

### 1)N-[1-(3-bromo-2-fluoro-phenyl)ethyl]cyclopropanamine

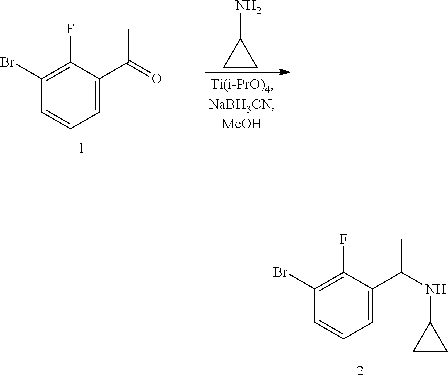

To a solution of 1-(3-bromo-2-fluoro-phenyl)ethanone (5 g, 23.04 mmol, 1 eq) in MeOH (50 mL) was added cyclopropanamine (1.97 g, 34.56 mmol, 2.39 mL, 1.5 eq) and Ti(i-PrO)₄ (19.64 g, 69.11 mmol, 20.40 mL, 3 eq). The mixture was stirred at 60° C. for 12 hrs, then NaBH₃CN (5.79 g, 92.15 mmol, 4 eq) was added, the mixture was stirred at 60° C. for 2 hrs. LC-MS showed no 1 remained and ˜86% of desired compound was detected. The reaction mixture was concentrated under reduced pressure to remove solvent. The residue was diluted with H₂O 50 mL and extracted with ethyl acetate (40 mL*3). The combined organic layers were dried over Na₂SO₄, filtered and concentrated under reduced pressure to give a residue. The residue was purified by flash silica gel chromatography (20 g Silica Flash Column, Eluent of 0-2% Ethyl acetate/Petroleum ether gradient @100 m/min) to give compound N-[1-(3-bromo-2-fluoro-phenyl)ethyl]cyclopropanamine (3.8 g, 14.54 mmol, 63.10% yield, 98.75% purity) as a pale yellow oil.

### 2) tert-butyl N-[2-[1-(3-bromo-2-fluoro-phenyl)ethyl-cyclopropyl-amino]ethyl]carbamate

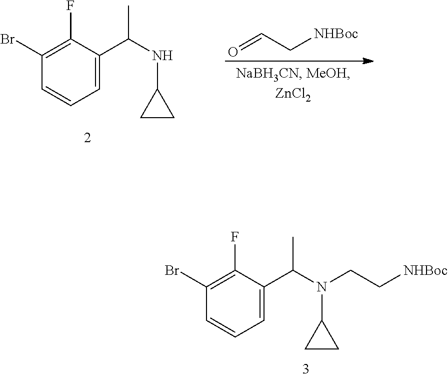 [SMILES: CC(NC1CC1)c1cccc(Br)c1F ; CC(C)(C)OC(=O)NCC=O ; CC(c1cccc(Br)c1F)N(CCNC(=O)OC(C)(C)C)C1CC1]

To a solution of N-[1-(3-bromo-2-fluoro-phenyl)ethyl]cyclopropanamine (1 g, 3.87 mmol, 1 eq) and tert-butyl N-(2-oxoethyl)carbamate (1.54 g, 9.69 mmol, 2.5 eq) in MeOH (10 mL) was added NaBH₃CN (486.89 mg, 7.75 mmol, 2 eq) and ZnCl₂ (1.06 g, 7.75 mmol, 363.27 μL, 2 eq). The mixture was stirred at 20° C. for 12 hrs. LC-MS showed no 2 remained and ˜95% of desired compound was detected. The reaction mixture was concentrated under reduced pressure to remove solvent. The residue was diluted with H₂O 10 mL and extracted with ethyl acetate (10 mL*3). The combined organic layers were dried over Na₂SO₄, filtered and concentrated under reduced pressure to give tert-butyl N-[2-[1-(3-bromo-2-fluoro-phenyl)ethyl-cyclopropyl-amino]ethyl]carbamate (2 g, crude) as a white solid.

### 3) tert-butyl N-[2-[cyclopropyl-[1-[2-fluoro-3-(2-trimethylsilylethynyl)phenyl]ethyl]amino]ethyl]carbamate

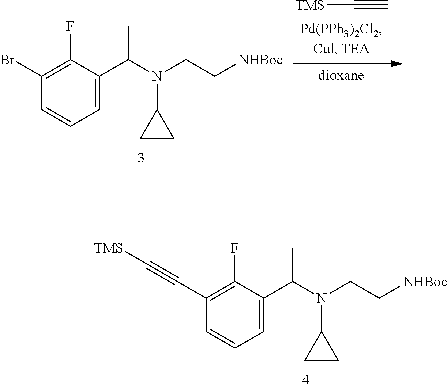

To a solution of tert-butyl N-[2-[1-(3-bromo-2-fluoro-phenyl)ethyl-cyclopropyl-amino]ethyl]carbamate (1 g, 2.49 mmol, 1 eq) and ethynyl(trimethyl)silane (1.22 g, 12.46 mmol, 1.73 mL, 5 eq) in dioxane (10 mL) was added CuI (47.46 mg, 249.18 μmol, 0.1 eq), Pd(PPh₃)₂Cl₂ (174.90 mg, 249.18 μmol, 0.1 eq) and TEA (1.26 g, 12.46 mmol, 1.73 mL, 5 eq). The mixture was stirred at 80° C. for 6 hr under N₂ atmosphere. LC-MS showed ˜13% of 3 remained and ˜11% of desired compound was detected. The reaction mixture was concentrated under reduced pressure to remove solvent. The residue was diluted with H₂O 15 mL and extracted with ethyl acetate (10 mL*3). The combined organic layers were dried over Na₂SO₄, filtered and concentrated under reduced pressure to give a residue. The residue was purified by prep-HPLC (TFA condition; column: Phenomenex luna C18 100*40 mm*3 um; mobile phase: [H₂O (0.1% TFA)-ACN]; gradient: 40%-70% B over 8.0 min) to give tert-butyl N-[2-[cyclopropyl-[1-[2-fluoro-3-(2-trimethylsilylethynyl)phenyl]ethyl]amino]ethyl]carbamate (300 mg, 701.31 μmol, 28.14% yield, 97.86% purity) as a pale yellow oil.

### 4) tert-butyl N-[2-[cyclopropyl-[1-(3-ethynyl-2-fluoro-phenyl)ethyl]amino]ethyl]carbamate

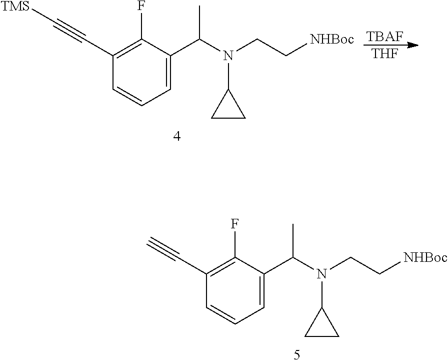

To a solution of tert-butyl N-[2-[cyclopropyl-[1-[2-fluoro-3-(2-trimethylsilylethynyl)phenyl]ethyl]amino]ethyl]carbamate (185 mg, 441.93 μmol, 1 eq) in THF (2 mL) was added TBAF (1 M, 662.89 μL, 1.5 eq). The mixture was stirred at 20° C. for 1 hr. LC-MS showed no 4 remained and ˜93% of desired compound was detected. The reaction mixture was concentrated under reduced pressure to remove solvent. The residue was diluted with H₂O 4 mL and extracted with ethyl acetate (3 mL*3). The combined organic layers were dried over Na₂SO₄, filtered and concentrated under reduced pressure to give tert-butyl N-[2-[cyclopropyl-[1-(3-ethynyl-2-fluoro-phenyl)ethyl]amino]ethyl]carbamate (200 mg, crude) as a yellow oil.

### 5) N′-cyclopropyl-N′-[1-(3-ethynyl-2-fluoro-phenyl)ethyl]ethane-1,2-diamine TFA salt

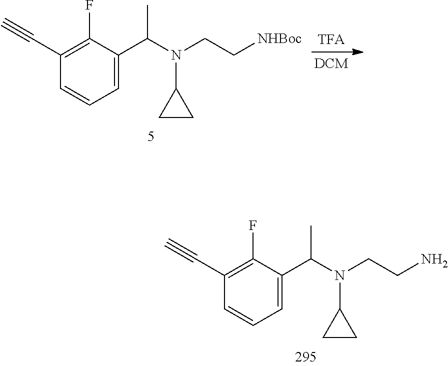 [SMILES: C#Cc1cccc(C(C)N(CCNC(=O)OC(C)(C)C)C2CC2)c1F ; C#Cc1cccc(C(C)N(CCN)C2CC2)c1F]

To a solution of tert-butyl N-[2-[cyclopropyl-[1-(3-ethynyl-2-fluoro-phenyl)ethyl]amino]ethyl]carbamate (180 mg, 519.57 μmol, 1 eq) in DCM (2 mL) was added TFA (767.50 mg, 6.73 mmol, 0.5 mL, 12.96 eq). The mixture was stirred at 20° C. for 1 hr. LC-MS showed no 5 remained and ˜86% of desired compound was detected. The reaction mixture was concentrated under reduced pressure to give a residue. The residue was purified by prep-HPLC (TFA condition; column: Phenomenex luna C18 100*40 mm*5 um; mobile phase: [H₂O (0.1% TFA)-ACN]; gradient: 1%-40% B over 8.0 min) to give N′-cyclopropyl-N′-[1-(3-ethynyl-2-fluoro-phenyl)ethyl]ethane-1,2-diamine TFA salt (70.09 mg, 193.98 μmol, 37.33% yield, 99.73% purity, 1TFA) as a pale yellow oil. MS (ESI): m/z=246.15[M+H]⁺, retention time: 1.360 min, method:Bhalo. ¹H NMR (400 MHz, CHLOROFORM-d) δ=9.18-8.08 (m, 3H), 7.62-7.42 (m, 2H), 7.25-7.18 (m, 1H), 4.94 (q, J=6.7 Hz, 1H), 3.79-3.64 (m, 1H), 3.62-3.41 (m, 2H), 3.39-3.25 (m, 2H), 2.26 (br s, 1H), 1.73 (br d, J=7.0 Hz, 3H), 1.07 (br s, 2H), 0.94-0.74 (m, 2H)

Example 45: Synthesis of 5-[1-[2-aminoethyl(cyclopropyl)amino]ethyl]-4-fluoro-thiophene-3-carbonitrile TFA salt and N′-[1-(4-bromo-3-fluoro-2-thienyl)ethyl]-N′-cyclopropyl-ethane-1,2-diamine TFA salt (Compounds 297 and 297A)

### 1) 4-bromo-3-fluoro-thiophene-2-carboxylic acid

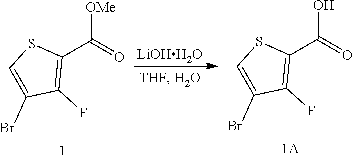

To a solution of methyl 4-bromo-3-fluoro-thiophene-2-carboxylate (0.7 g, 2.93 mmol, 1 eq) in THF (10 mL) and H₂O (10 mL) was added LiOH·H₂O (245.75 mg, 5.86 mmol, 2 eq) and the mixture was stirred at 20° C. for 1 hr. LC-MS showed 1 was consumed completely and one main peak was detected. The reaction mixture was concentrated under reduced pressure to remove THF. The residue was acidified by HCl (1M) to adjust pH around 3, then extracted with EtOAc (5 mL*2). The combined organic layers were washed with brine (5 mL), dried over Na₂SO₄, filtered and concentrated under reduced pressure to give 4-bromo-3-fluoro-thiophene-2-carboxylic acid (0.69 g, crude) as a white solid.

### 2) 4-bromo-3-fluoro-N-methoxy-N-methyl-thiophene-2-carboxamide

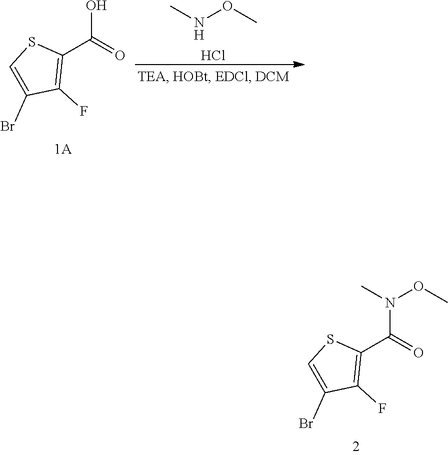

To a solution of 4-bromo-3-fluoro-thiophene-2-carboxylic acid (0.69 g, 3.07 mmol, 1 eq) and N-methoxymethanamine (448.63 mg, 4.60 mmol, 1.5 eq, HCl) in DCM (20 mL) was added HOBt (497.18 mg, 3.68 mmol, 1.2 eq) and EDCI (705.35 mg, 3.68 mmol, 1.2 eq) and TEA (775.66 mg, 7.67 mmol, 1.07 mL, 2.5 eq). The mixture was stirred at 20° C. for 2 hr. LC-MS showed 1A was consumed completely and one main peak with desired mass was detected. The reaction mixture was quenched by addition H₂O 10 mL at 20° C., and then extracted with EtOAc (10 mL*2). The combined organic layers were washed with brine (10 mL), dried over Na₂SO₄, filtered and concentrated under reduced pressure to give a residue. The residue was purified by flash silica gel chromatography (12 g Silica Flash Column, Eluent of 0-20% Ethyl acetate/Petroleum ether gradient @60 mL/min) to give compound 4-bromo-3-fluoro-N-methoxy-N-methyl-thiophene-2-carboxamide (0.7 g, 2.61 mmol, 85.15% yield) as a colorless oil. ¹H NMR (400 MHz, CHLOROFORM-d) δ=7.39 (d, J=4.0 Hz, 1H), 3.75 (s, 3H), 3.35 (s, 3H)

### 3) 1-(4-bromo-3-fluoro-2-thienyl)ethanone

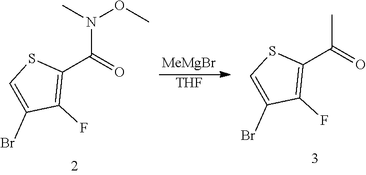

To a solution of 4-bromo-3-fluoro-N-methoxy-N-methyl-thiophene-2-carboxamide (0.6 g, 2.24 mmol, 1 eq) in THF (10 mL) was added MeMgBr (3 M, 895.18 μL, 1.2 eq) at −78° C., then the mixture was stirred at 20° C. for 1 hr. TLC indicated 2 was consumed completely and one new spot formed. The reaction mixture was quenched by addition NH₄Cl 10 mL at 20° C., and then extracted with EtOAc (10 mL*2). The combined organic layers were washed with brine (10 mL), dried over Na₂SO₄, filtered and concentrated under reduced pressure to give compound 1-(4-bromo-3-fluoro-2-thienyl)ethanone (0.55 g, crude) as a colorless oil. ¹H NMR (400 MHz, CHLOROFORM-d) δ ppm 7.55-7.48 (m, 1H) 2.61 (d, J=2.88 Hz, 3H)

### 4) 1-(3-bromo-2-fluoro-phenyl)-2-methyl-propan-1-one

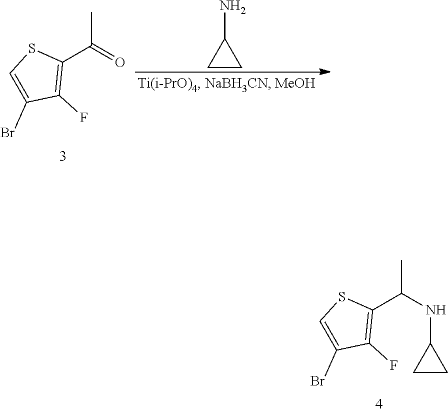

To a solution of 1-(4-bromo-3-fluoro-2-thienyl)ethanone (500 mg, 2.24 mmol, 1 eq) in MeOH (5 mL) was added cyclopropanamine (191.97 mg, 3.36 mmol, 232.97 μL, 1.5 eq) and Ti(i-PrO)₄ (1.91 g, 6.72 mmol, 1.98 mL, 3 eq). The mixture was stirred at 60° C. for 12 hrs, then NaBH₃CN (563.43 mg, 8.97 mmol, 4 eq) was added. The mixture was stirred at 60° C. for 2 hrs. LC-MS showed no 3 remained and ˜66% of desired compound was detected. The reaction mixture was concentrated under reduced pressure to remove solvent. The residue was diluted with H₂O 5 mL and extracted with EtOAc (4 mL*3). The combined organic layers were dried over Na₂SO₄, filtered and concentrated under reduced pressure to give a residue. The residue was purified by flash silica gel chromatography (4 g Silica Flash Column, Eluent of 0-2% Ethyl acetate/Petroleum ether gradient @40 mL/min) to give compound N-[1-(4-bromo-3-fluoro-2-thienyl)ethyl]cyclopropanamine (350 mg, 1.29 mmol, 57.50% yield, 97.27% purity) as a yellow oil.

### 5) tert-butyl N-[2-[1-(4-bromo-3-fluoro-2-thienyl)ethyl-cyclopropyl-amino]ethyl]carbamate

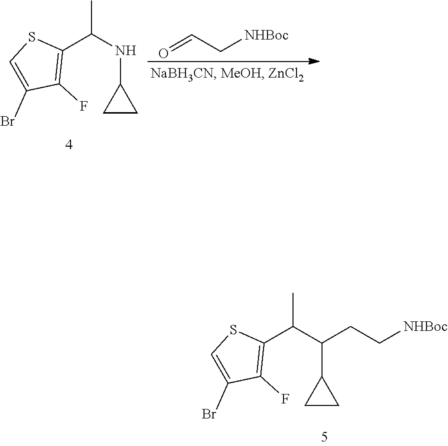

To a solution of N-[1-(4-bromo-3-fluoro-2-thienyl)ethyl]cyclopropanamine (300 mg, 1.14 mmol, 1 eq) and tert-butyl N-(2-oxoethyl)carbamate (542.34 mg, 3.41 mmol, 3 eq) in MeOH (3 mL) was added NaBH₃CN (142.73 mg, 2.27 mmol, 2 eq) and ZnCl₂ (309.59 mg, 2.27 mmol, 106.50 μL, 2 eq). The mixture was stirred at 20° C. for 3 hrs. LC-MS showed no 4 remained and ˜94% of desired compound was detected. The reaction mixture was concentrated under reduced pressure to remove solvent. The residue was diluted with H₂O 5 mL and extracted with EtOAc (3 mL*3). The combined organic layers were dried over Na₂SO₄, filtered and concentrated under reduced pressure to give a residue. The residue was purified by flash silica gel chromatography (4 g Silica Flash Column, Eluent of 0-2% Ethyl acetate/Petroleum ether gradient @40 mL/min) to give compound tert-butyl N-[2-[1-(4-bromo-3-fluoro-2-thienyl)ethyl-cyclopropyl-amino]ethyl]carbamate (350 mg, 776.40 μmol, 68.36% yield, 90.36% purity) as a yellow oil.

### 6) tert-butyl N-[2-[1-(4-cyano-3-fluoro-2-thienyl)ethyl-cyclopropyl-amino]ethyl]carbamate

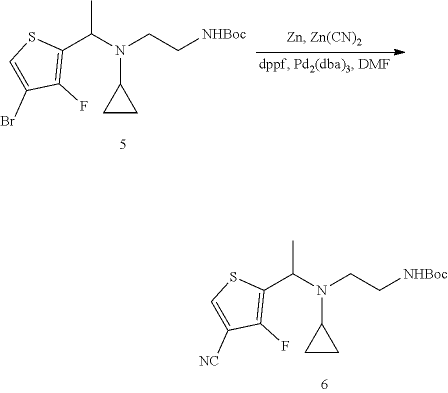 [SMILES: CC(c1scc(Br)c1F)N(CCNC(=O)OC(C)(C)C)C1CC1 ; CC(c1scc(C#N)c1F)N(CCNC(=O)OC(C)(C)C)C1CC1]

To a solution of tert-butyl N-[2-[1-(4-bromo-3-fluoro-2-thienyl)ethyl-cyclopropyl-amino]ethyl]carbamate (200 mg, 490.99 μmol, 1 eq) and dppf (108.88 mg, 196.40 μmol, 0.4 eq) in DMF (2 mL) was added Zn (16.05 mg, 245.49 μmol, 0.5 eq), Zn(CN)₂ (130 mg, 1.11 mmol, 70.27 μL, 2.25 eq) and Pd₂(dba)₃ (89.92 mg, 98.20 μmol, 0.2 eq). The mixture was stirred at 80° C. for 12 hr under N₂ atmosphere. LC-MS showed no 5 remained and ˜17% of desired compound was detected. The reaction mixture was concentrated under reduced pressure to remove solvent. The residue was diluted with H₂O 5 mL and extracted with EtOAc (4 mL*3). The combined organic layers were dried over Na₂SO₄, filtered and concentrated under reduced pressure to give a residue. The residue was purified by flash silica gel chromatography (4 g Silica Flash Column, Eluent of 0˜3% Ethyl acetate/Petroleum ether gradient @40 mL/min) to give compound tert-butyl N-[2-[1-(4-cyano-3-fluoro-2-thienyl)ethyl-cyclopropyl-amino]ethyl]carbamate (160 mg, 307.14 μmol, 62.56% yield, 67.85% purity) as a yellow solid.

### 7) 5-[1-[2-aminoethyl(cyclopropyl)amino]ethyl]-4-fluoro-thiophene-3-carbonitrile TFA salt

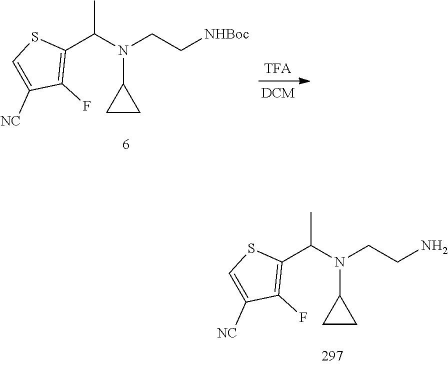 [SMILES: CC(c1scc(C#N)c1F)N(CCNC(=O)OC(C)(C)C)C1CC1 ; CC(c1scc(C#N)c1F)N(CCN)C1CC1]

To a solution of tert-butyl N-[2-[1-(4-cyano-3-fluoro-2-thienyl)ethyl-cyclopropyl-amino]ethyl]carbamate (160 mg, 452.68 μmol, 1 eq) in DCM (2 mL) was added TFA (767.50 mg, 6.73 mmol, 0.5 mL, 14.87 eq). The mixture was stirred at 20° C. for 1 hr. LC-MS showed no 6 remained and ˜47% of desired compound was detected. The reaction mixture was concentrated under reduced pressure to give a residue. The residue was purified by prep-HPLC (TFA condition; column: Phenomenex luna C18 100*40 mm*5 um; mobile phase: [H₂O (0.1% TFA)-ACN]; gradient: 1%-40% B over 8.0 min) to give compound 5-[1-[2-aminoethyl(cyclopropyl)amino]ethyl]-4-fluoro-thiophene-3-carbonitrile TFA salt (49.6 mg, 134.44 μmol, 29.70% yield, 99.57% purity, TFA) as a colorless oil. MS (ESI): m/z=253.10[M+H]⁺, retention time: 1.362 min, method:M. ¹H NMR (400 MHz, CHLOROFORM-d) δ ppm 8.63-7.95 (m, 2H) 7.88 (d, J=3.38 Hz, 1H) 4.74-4.61 (m, 1H) 3.48-3.20 (m, 3H) 3.12-2.94 (m, 1H) 2.12-1.99 (m, 1H) 1.76-1.52 (m, 3H) 0.96-0.61 (m, 4H)

### 8) N′-[1-(4-bromo-3-fluoro-2-thienyl)ethyl]-N′-cyclopropyl-ethane-1,2-diamine TFA salt

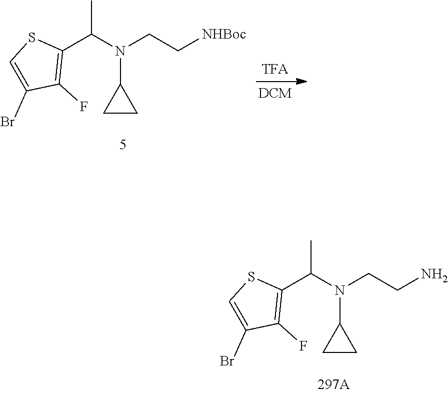 [SMILES: CC(c1scc(Br)c1F)N(CCNC(=O)OC(C)(C)C)C1CC1 ; CC(c1scc(Br)c1F)N(CCN)C1CC1]

To a solution of tert-butyl N-[2-[1-(4-bromo-3-fluoro-2-thienyl)ethyl-cyclopropyl-amino]ethyl]carbamate (110 mg, 270.04 μmol, 1 eq) in DCM (1 mL) was added TFA (1.19 g, 10.41 mmol, 772.99 μL, 38.54 eq). The mixture was stirred at 20° C. for 1 hr. LC-MS showed ˜5% 5 remained and ˜60% of desired compound was detected. The reaction mixture was concentrated under reduced pressure to give a residue. The residue was purified by prep-HPLC (TFA condition; column: Phenomenex luna C18 100*40 mm*5 um; mobile phase: [H₂O (0.1% TFA)-ACN]; gradient: 10%-45% B over 8.0 min) to give compound N′-[1-(4-bromo-3-fluoro-2-thienyl)ethyl]-N′-cyclopropyl-ethane-1,2-diamine TFA salt (16.85 mg, 39.79 μmol, 14.74% yield, 99.48% purity, TFA) as a colorless oil. MS (ESI): m/z=306.02 [M+H]⁺, retention time: 1.535 min, method:B. ¹H NMR (400 MHz, CHLOROFORM-d) δ ppm 8.94-7.74 (m, 3H) 7.32-7.23 (m, 1H) 4.85-4.70 (m, 1H) 3.48 (br d, J=7.25 Hz, 2H) 3.41-3.28 (m, 1H) 3.15 (br dd, J=9.94, 4.57 Hz, 1H) 2.19-2.05 (m, 1H) 1.65 (br d, J=7.00 Hz, 3H) 1.07-0.72 (m, 4H)

### Example 46: Synthesis of 3-[1-[2-aminoethyl(cyclopropyl)amino]-2,2,2-trifluoro-ethyl]-2-fluoro-benzonitrile hydrochloride (Compound 261)

### 1) tert-butyl (2-((1-(3-bromo-2-fluorophenyl)-2,2,2-trifluoroethyl)(cyclopropyl)amino) ethyl)carbamate

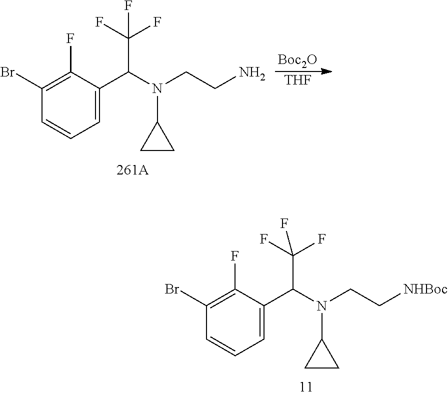 [SMILES: "Boc2O" ; NCCN(C1CC1)C(c1cccc(Br)c1F)C(F)(F)F ; CC(C)(C)OC(=O)NCCN(C1CC1)C(c1cccc(Br)c1F)C(F)(F)F ; "THF"]

To a solution of N′-[1-(3-bromo-2-fluoro-phenyl)-2,2,2-trifluoro-ethyl]-N′-cyclopropyl-ethane-1,2-diamine (2 g, 5.63 mmol, 1eq) in THF (40 mL) was added Boc₂O (6.14 g, 28.16 mmol, 6.47 mL, 5eq). The mixture was stirred at 25° C. for 12 hr. LCMS showed desired compound formed. The reaction mixture was concentrated under reduced pressure to remove solvent. The residue was diluted with H₂O 50 mL and extracted with DCM 150 mL (50 mL*3). The combined organic layers were dried over Na₂SO₄, filtered and concentrated under reduced pressure to give a residue. The residue was purified by flash silica gel chromatography (20 g Silica Flash Column, Eluent of 0-5% Ethyl acetate/Petroleum ether gradient@80 mL/min) to give compound tert-butyl N-[2-[[1-(3-bromo-2-fluoro-phenyl)-2,2,2-trifluoro-ethyl]-cyclopropyl-amino]ethyl]carbamate (750 mg, 1.65 mmol, 29.25% yield) as a white solid.

### 2) tert-butyl (2-((1-(3-cyano-2-fluorophenyl)-2,2,2-trifluoroethyl)(cyclopropyl)amino) ethyl)carbamate

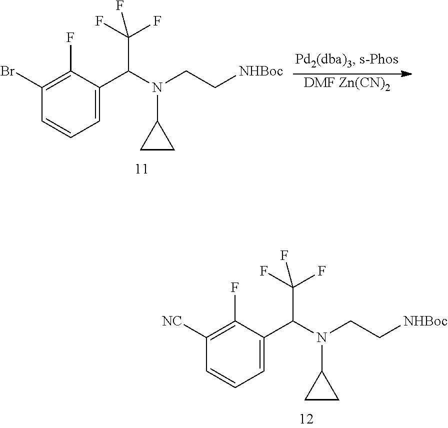

A mixture of tert-butyl N-[2-[[1-(3-bromo-2-fluoro-phenyl)-2,2,2-trifluoro-ethyl]-cyclopropyl-amino]ethyl]carbamate (200 mg, 439.29 μmol, 1eq), Pd₂(dba)₃ (40.23 mg, 43.93 μmol, 0.1eq), Zn(CN)₂ (154.75 mg, 1.32 mmol, 83.65 μL, 3eq) and s-Phos (18.03 mg, 43.93 μmol, 0.1eq) in DMF (4 mL) was degassed and purged with N₂ for 3 times, and then the mixture was stirred at 120° C. for 12 hr under N₂ atmosphere. LCMS showed desired compound formed. The mixture was filtered and concentrated under reduced pressure to give a residue. The residue was diluted with H₂O 10 mL and extracted with EtOAC 30 mL (10 mL*3). The combined organic layers were washed with brine 30 mL (10 mL*3), dried over Na₂SO₄, filtered and concentrated under reduced pressure to give a residue. The residue was purified by prep-TLC (SiO₂, Petroleum ether/Ethyl acetate=3:1) to give compound tert-butyl N-[2-[[1-(3-cyano-2-fluoro-phenyl)-2,2,2-trifluoro-ethyl]-cyclopropyl-amino]ethyl]carbamate (110 mg, 274.04 μmol, 62.38% yield) as yellow oil.

### 3) 3-(1-((2-aminoethyl)(cyclopropyl)amino)-2,2,2-trifluoroethyl)-2-fluorobenzonitrile hydrochloride

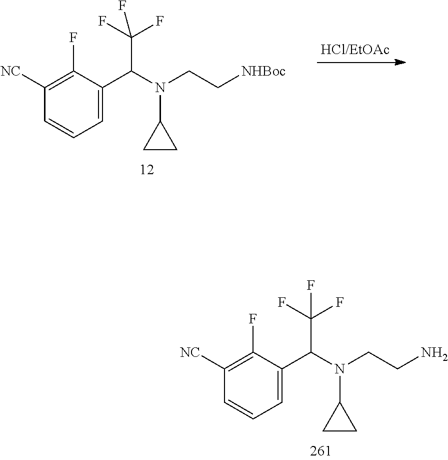 [SMILES: CC(C)(C)OC(=O)NCCN(C1CC1)C(c1cccc(C#N)c1F)C(F)(F)F ; N#Cc1cccc(C(N(CCN)C2CC2)C(F)(F)F)c1F]

A solution of tert-butyl N-[2-[[1-(3-cyano-2-fluoro-phenyl)-2,2,2-trifluoro-ethyl]-cyclopropyl-amino]ethyl]carbamate (110 mg, 274.04 μmol, 1eq) in HCl/EtOAc (2 mL) was stirred at 25° C. for 0.5 hr. LCMS showed desired compound formed. The reaction mixture was concentrated under reduced pressure to remove solvent. The residue was purified by prep-HPLC (HCL condition, column: Phenomenex Luna C18 75*30 mm*3 um; mobile phase: [H₂O (0.04% HCl)-ACN]; gradient: 10%-40% B over 8.0 min) to give compound 3-[1-[2-aminoethyl(cyclopropyl)amino]-2,2,2-trifluoro-ethyl]-2-fluoro-benzonitrile hydrochloride (90.84 mg, 268.69 μmol, 98.05% yield, 99.9% purity, HCl) as a white solid. MS (ESI): m/z=301.12 [M+H]⁺; retention time: 1.584 min, method: M ¹H NMR (400 MHz, DMSO-d₆) δ=8.14-8.01 (m, 1H), 7.92-7.81 (m, 4H), 7.54 (t, J=7.9 Hz, 1H), 5.18 (q, J=9.3 Hz, 1H), 3.10-2.68 (m, 4H), 1.99 (br d, J=2.1 Hz, 1H), 0.69-0.41 (m, 4H)

### Example 47: Synthesis of N′-[1-(3-bromo-2-fluoro-phenyl)-2,2,2-trifluoro-ethyl]-N′-cyclopropyl-ethane-1,2-diamine hydrochloride (Compound 261A)

### 1) 1-(3-bromo-2-fluorophenyl)-2,2,2-trifluoroethan-1-ol

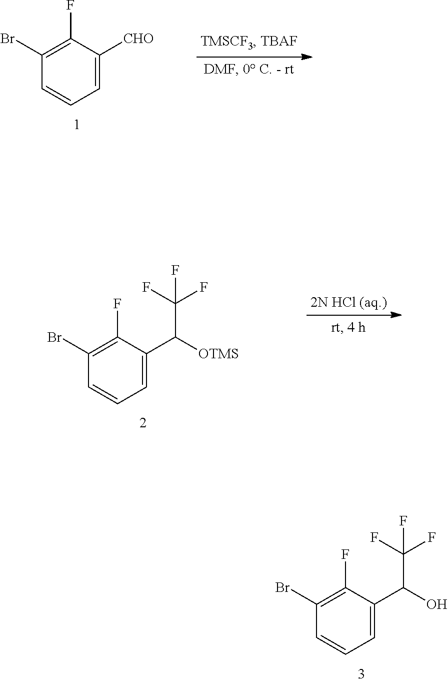

A mixture of 3-bromo-2-fluoro-benzaldehyde (10 g, 49.26 mmol, 1eq) in DMF (100 mL), then TMSCF₃ (9.11 g, 64.04 mmol, 1.3eq) was added and the mixture was stirred at 0° C. After 15 min, TBAF (1 M, 492.59 μL, 0.01eq) was added dropwise via a syringe. The mixture was degassed and purged with N₂ for 3 times, and then the mixture was stirred at 20° C. for 12 hr under N₂ atmosphere. When the reaction was complete, the HCl (2M, 49.26 mL, 2eq) was added to the solution and stirred at 20° C. for 4 hr. TLC indicated Reactant 1 was consumed completely and one new spot formed. The reaction mixture was concentrated under reduced pressure to remove solvent. The residue was diluted with H₂O 20 mL and extracted with EtOAC 90 mL (30 mL*3). The combined organic layers were washed with brine 90 mL (30 mL*3), dried over Na₂SO₄, filtered and concentrated under reduced pressure to give a residue. The residue was purified by flash silica gel chromatography (40 g Silica Flash Column, Eluent of 0-3% Ethyl acetate/Petroleum ethergradient@80 mL/min) to give compound 1-(3-bromo-2-fluoro-phenyl)-2,2,2-trifluoro-ethanol (9.6 g, 35.16 mmol, 71.38% yield) as yellow oil.

### 2) 1-(3-bromo-2-fluorophenyl)-2,2,2-trifluoroethan-1-one

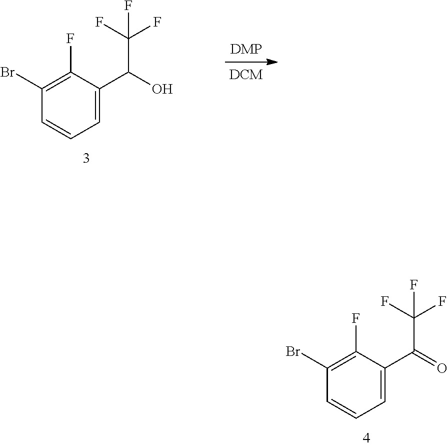 [SMILES: OC(c1cccc(Br)c1F)C(F)(F)F ; O=C(c1cccc(Br)c1F)C(F)(F)F]

To a solution of 1-(3-bromo-2-fluoro-phenyl)-2,2,2-trifluoro-ethanol (9.6 g, 35.16 mmol, 1eq) in DCM (200 mL) was added DMP (16.41 g, 38.6 mmol, 11.98 mL, 1.1eq). The mixture was stirred at 25° C. for 12 hr. TLC indicated 3 was consumed completely and one new spot formed. The reaction mixture was concentrated under reduced pressure to remove solvent. The residue was purified by flash silica gel chromatography (40 g Silica Flash Column, Eluent of 0-2% Ethyl acetate/Petroleum ether gradient@80 mL/min) to give compound 1-(3-bromo-2-fluoro-phenyl)-2,2,2-trifluoro-ethanone (6.62 g, 24.43 mmol, 69.47% yield) as yellow oil.

### 3) (E)-1-(3-bromo-2-fluorophenyl)-N-cyclopropyl-2,2,2-trifluoroethan-1-imine

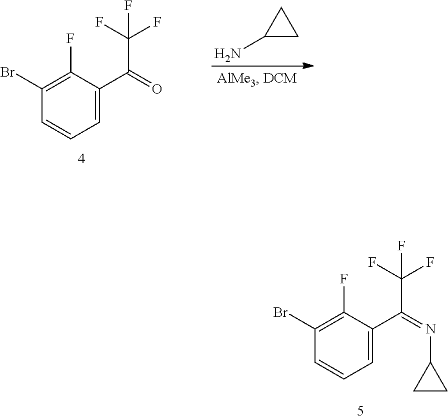

A mixture of 1-(3-bromo-2-fluoro-phenyl)-2,2,2-trifluoro-ethanone (6.6 g, 24.35 mmol, 1eq), cyclopropanamine (2.09 g, 36.53 mmol, 2.53 mL, 1.5eq) in DCM (70 mL), then AlMe₃ (2 M, 12.18 mL, 1 eq) was added at N₂, the mixture was stirred at 25° C. for 12 hr under N₂ atmosphere. LCMS showed desired compound formed. The residue was diluted with NaOH (20%) 50 mL and extracted with DCM 90 mL (30 mL*3). The combined organic layers were dried over Na₂SO₄, filtered and concentrated under reduced pressure to give a residue to give compound (E)-1-(3-bromo-2-fluoro-phenyl)-N-cyclopropyl-2,2,2-trifluoro-ethanimine (8 g, crude) as yellow oil.

### 4)N-(1-(3-bromo-2-fluorophenyl)-2,2,2-trifluoroethyl)cyclopropanamine

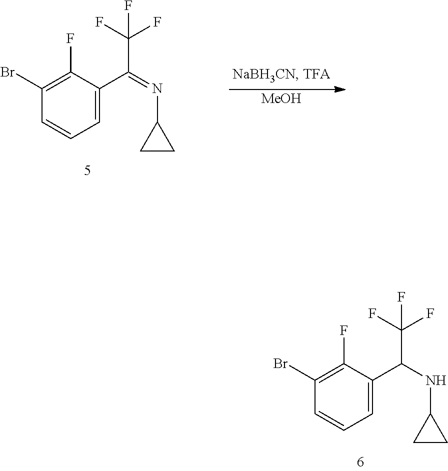 [SMILES: "MeOH" ; Fc1c(Br)cccc1/C(=N\C1CC1)C(F)(F)F ; Fc1c(Br)cccc1C(NC1CC1)C(F)(F)F]

To a solution of (E)-1-(3-bromo-2-fluoro-phenyl)-N-cyclopropyl-2,2,2-trifluoro-ethanimine (8 g, 25.80 mmol, 1eq) in MeOH (90 mL) was added TFA (2.94 g, 25.80 mmol, 1.92 mL, 1eq) and NaBH₃CN (6.49 g, 103.20 mmol, 4eq). The mixture was stirred at 60° C. for 1 hr. LCMS showed desired compound formed. The reaction mixture was concentrated under reduced pressure to remove solvent. The residue was purified by flash silica gel chromatography (40 gSepaFlash@Silica Flash Column, Eluent of 0˜4% Ethyl acetate/Petroleum ether gradient@120 mL/min) to give compound N-[1-(3-bromo-2-fluoro-phenyl)-2,2,2-trifluoro-ethyl]cyclopropanamine (3.14 g, 9.68 mmol, 37.51% yield, 96.2% purity) as yellow oil.

### 5) 2-bromo-N-(1-(3-bromo-2-fluorophenyl)-2,2,2-trifluoroethyl)-N-cyclopropylacetamide

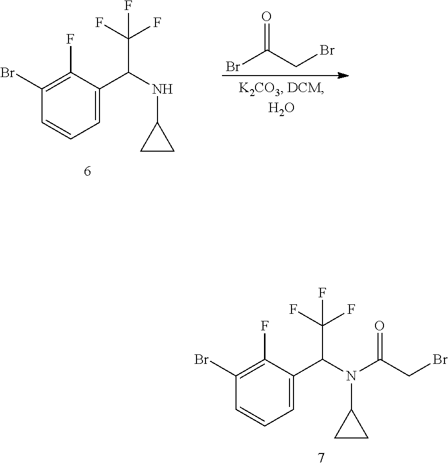 [SMILES: Fc1c(Br)cccc1C(NC1CC1)C(F)(F)F ; O=C(Br)CBr ; O=C(CBr)N(C1CC1)C(c1cccc(Br)c1F)C(F)(F)F]

To a solution of N-[1-(3-bromo-2-fluoro-phenyl)-2,2,2-trifluoro-ethyl]cyclopropanamine (2.4 g, 7.69 mmol, 1eq) in DCM (30 mL) and H₂O (30 mL) was added K₂CO₃ (2.13 g, 15.38 mmol, 2eq) and 2-bromoacetyl bromide (2.79 g, 13.84 mmol, 1.21 mL, 1.8eq) at 0° C. The mixture was stirred at 25° C. for 0.5 hr. LCMS showed desired compound formed. The residue was diluted with H₂O 20 mL and extracted with DCM 90 mL (30 mL*3). The combined organic layers were dried over Na₂SO₄, filtered and concentrated under reduced pressure to give a residue. The residue was purified by flash silica gel chromatography (20 g Silica Flash Column, Eluent of 0-7% Ethyl acetate/Petroleum ether gradient@80 mL/min) to give compound 2-bromo-N-[1-(3-bromo-2-fluoro-phenyl)-2,2,2-trifluoro-ethyl]-N-cyclopropyl-acetamide (3 g, 6.93 mmol, 90.09% yield) as a white solid.

### 6)N-(1-(3-bromo-2-fluorophenyl)-2,2,2-trifluoroethyl)-N-cyclopropyl-2-(1,3-dioxoisoindolin-2-yl)acetamide

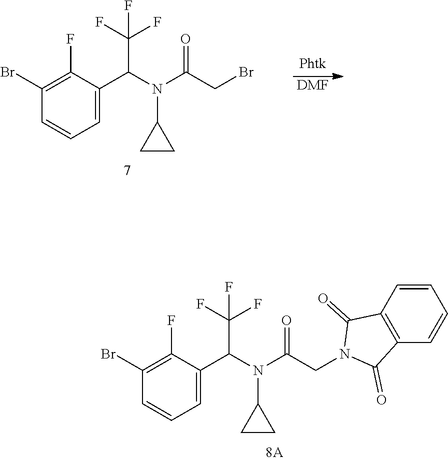

To a solution of 2-bromo-N-[1-(3-bromo-2-fluoro-phenyl)-2,2,2-trifluoro-ethyl]-N-cyclopropyl-acetamide (3.18 g, 7.34 mmol, 1eq) in DMF (40 mL) was added (1,3-dioxoisoindolin-2-yl)potassium (1.50 g, 8.08 mmol, 1.1eq). The mixture was stirred at 20° C. for 4 hr. LCMS showed desired compound formed. The residue was diluted with H₂O 20 mL and extracted with EtOAc 60 mL (20 mL*3). The combined organic layers were washed with brine 60 mL (20 mL*3), dried over Na₂SO₄, filtered and concentrated under reduced pressure to give a residue. The residue was purified by flash silica gel chromatography (20 g Silica Flash Column, Eluent of 0-20% Ethyl acetate/Petroleum ether gradient@100 m/min) to give compound N-[1-(3-bromo-2-fluoro-phenyl)-2,2,2-trifluoro-ethyl]-N-cyclopropyl-2-(1,3-dioxoisoindolin-2-yl)acetamide (4.11 g, 5.66 mmol, 77.01% yield, 68.7% purity) as a white solid.

### 7) 2-amino-N-(1-(3-bromo-2-fluorophenyl)-2,2,2-trifluoroethyl)-N-cyclopropylacetamide

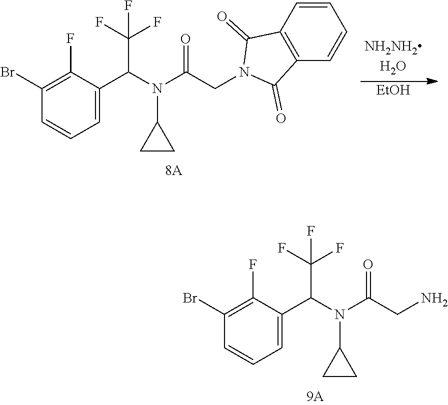 [SMILES: "EtOH" ; O=C1c2ccccc2C(=O)N1CC(=O)N(C1CC1)C(c1cccc(Br)c1F)C(F)(F)F ; NCC(=O)N(C1CC1)C(c1cccc(Br)c1F)C(F)(F)F]

To a solution of N-[1-(3-bromo-2-fluoro-phenyl)-2,2,2-trifluoro-ethyl]-N-cyclopropyl-2-(1,3-dioxoisoindolin-2-yl)acetamide (4.11 g, 8.23 mmol, 1eq) in EtOH (40 mL) was added NH₂NH₂·H₂O (841.04 mg, 16.46 mmol, 814.96 μL, 98% purity, 2eq). The mixture was stirred at 80° C. for 3 hr. LCMS showed desired compound formed. The reaction mixture was concentrated under reduced pressure to remove solvent. The mixture was filtered and concentrated under reduced pressure to give a residue to give compound 2-amino-N-[1-(3-bromo-2-fluoro-phenyl)-2,2,2-trifluoro-ethyl]-N-cyclopropyl-acetamide (2.4 g, crude) as a white solid.

### 8) N1-(1-(3-bromo-2-fluorophenyl)-2,2,2-trifluoroethyl)-N1-cyclopropylethane-1,2-diamine hydrochloride

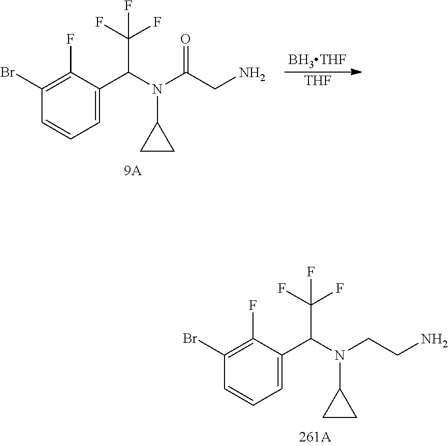 [SMILES: NCC(=O)N(C1CC1)C(c1cccc(Br)c1F)C(F)(F)F ; NCCN(C1CC1)C(c1cccc(Br)c1F)C(F)(F)F ; "BH3·THF" ; "THF"]

To a solution of 2-amino-N-[1-(3-bromo-2-fluoro-phenyl)-2,2,2-trifluoro-ethyl]-N-cyclopropyl-acetamide (47 mg, 127.32 μmol, 1eq) in THF (2 mL) was added BH₃·THF (1M, 1.27 mL, 10eq). The mixture was stirred at 70° C. for 12 hr. LCMS showed desired compound formed. The reaction mixture was quenched by addition MeOH (5 ml) at 70° C. for 2 hr. The reaction mixture was concentrated under reduced pressure to remove solvent. The residue was purified by prep-HPLC (HCL condition).column: Phenomenex luna C18 80*30 mm*3 um; mobile phase: [H₂O (0.04% HCl)-ACN]; gradient: 22%-52% B over 8.0 min to give compound N′-[1-(3-bromo-2-fluoro-phenyl)-2,2,2-trifluoro-ethyl]-N′-cyclopropyl-ethane-1,2-diamine hydrochloride (16 mg, 40.32 μmol, 31.67% yield, 98.68% purity, HCl) as a white solid. MS (ESI): m/z=354.04 [M+H]⁺; retention time: 1.765 min, method: M ¹H NMR (400 MHz, DMSO-d₆) δ=7.92 (br s, 3H), 7.86-7.79 (m, 1H), 7.55 (br t, J=7.1 Hz, 1H), 7.29 (t, J=7.9 Hz, 1H), 5.11 (q, J=9.5 Hz, 1H), 3.13-2.71 (m, 4H), 2.03-1.90 (m, 1H), 0.71-0.40 (m, 4H)

### Example 48: Synthesis of N1-(1-(3-chloro-2-fluorophenyl)-2,2,3,3,3-pentafluoropropyl)-N1-cyclopropylethane-1,2-diamine TFA salt (Compound 270)

### 1) 1-(3-chloro-2-fluorophenyl)-2,2,3,3,3-pentafluoropropan-1-ol

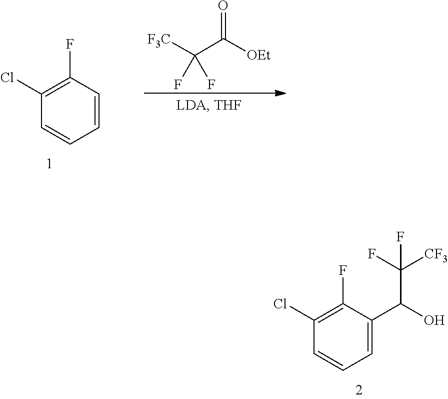 [SMILES: Fc1ccccc1Cl ; CCOC(=O)C(F)(F)C(F)(F)F ; OC(c1cccc(Cl)c1F)C(F)(F)C(F)(F)F]

A dry round-bottom flask flushed with N₂ was charged with the desired 1-chloro-2-fluoro-benzene (2 g, 15.3 mmol, 1 eq) and THF (45 mL). The base, LDA (2 M, 8.4 mL, 1.1 eq), was added dropwise, and the reaction was kept at −70° C. for 0.25 hr. Then ethyl 2,2,3,3,3-pentafluoropropanoate (3.5 g, 18.4 mmol, 1.2 eq) was added, and the mixture was allowed to react at 20° C. for 12 hr. TLC indicated Reactant 1 was consumed completely and one major new spot formed. The reaction was quenched with saturated aqueous NH₄Cl and extracted with ethyl acetate (10 mL×3). The combined organic phases were dried over Na₂SO₄, filtered and concentrated under reduced pressure. The residue was purified by flash silica gel chromatography (12 g SepaFlash® Silica Flash Column, Eluent of 0-0% Ethyl acetate/Petroleum ether gradient @60 m/min) to give 1-(3-chloro-2-fluorophenyl)-2,2,3,3,3-pentafluoropropan-1-ol (1.6 g, 5.7 mmol, 37.5% yield) as a yellow oil.

### 2) 1-(3-chloro-2-fluorophenyl)-2,2,3,3,3-pentafluoropropan-1-one

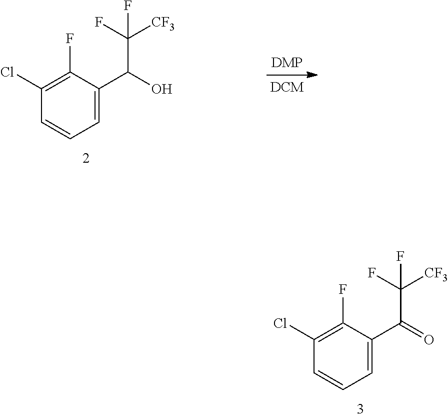 [SMILES: OC(c1cccc(Cl)c1F)C(F)(F)C(F)(F)F ; O=C(c1cccc(Cl)c1F)C(F)(F)C(F)(F)F]

To a solution of 1-(3-chloro-2-fluoro-phenyl)-2,2,3,3,3-pentafluoro-propan-1-ol (1.7 g, 6.1 mmol, 1 eq) in DCM (30 mL) was added DMP (3.1 g, 7.3 mmol, 1.2 eq). The mixture was stirred at 20° C. for 12 hr. TLC indicated 2 was consumed completely and one major new spot formed. The reaction mixture was diluted with H₂O 5 mL and extracted with DCM 15 mL (5 mL*3), dried over Na₂SO₄, filtered and concentrated under reduced pressure to give a residue. The residue was purified by flash silica gel chromatography (12 g SepaFlash® Silica Flash Column, Eluent of 0-0% Ethyl acetate/Petroleum ether gradient @60 mL/min) to give 1-(3-chloro-2-fluorophenyl)-2,2,3,3,3-pentafluoropropan-1-one (952 mg, 3.4 mmol, 56.4% yield) as a yellow oil.

### 3) (E)-1-(3-chloro-2-fluorophenyl)-N-cyclopropyl-2,2,3,3,3-pentafluoropropan-1-imine

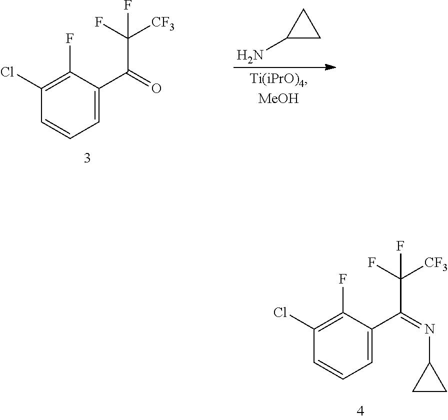

To a solution of 1-(3-chloro-2-fluoro-phenyl)-2,2,3,3,3-pentafluoro-propan-1-one (1 g, 3.6 mmol, 1 eq) in MeOH (15 mL) was added cyclopropanamine (309.7 mg, 5.4 mmol, 375.8 L, 1.5 eq) and Ti(i-PrO)₄ (3.1 g, 10.9 mmol, 3.2 mL, 3 eq). The mixture was stirred at 60° C. for 12 hr. LC-MS showed one main peak with desired m/z was detected. The reaction mixture was concentrated under reduced pressure to remove solvent. The residue was diluted with H₂O 100 mL and extracted with EtOAc 300 mL (100 mL*3), dried over Na₂SO₄, filtered and concentrated under reduced pressure to give a residue to give (E)-1-(3-chloro-2-fluorophenyl)-N-cyclopropyl-2,2,3,3,3-pentafluoropropan-1-imine (860 mg, crude) as yellow oil and it was used into the next step without further purification.

### 4)N-(1-(3-chloro-2-fluorophenyl)-2,2,3,3,3-pentafluoropropyl)cyclopropanamine

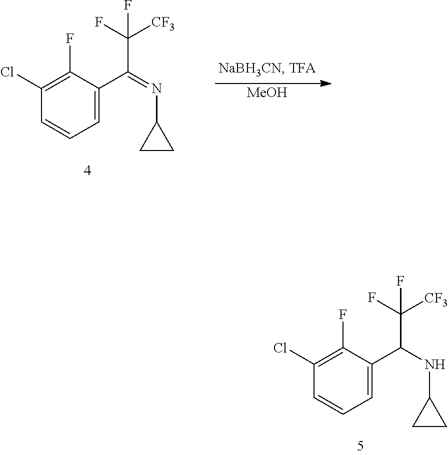 [SMILES: "MeOH" ; Fc1c(Cl)cccc1/C(=N\C1CC1)C(F)(F)C(F)(F)F ; Fc1c(Cl)cccc1C(NC1CC1)C(F)(F)C(F)(F)F]

To a solution of (E)-1-(3-chloro-2-fluoro-phenyl)-N-cyclopropyl-2,2,3,3,3-pentafluoro-propan-1-imine (500 mg, 1.6 mmol, 1 eq) in MeOH (15 mL) was added NaBH₃CN (398.2 mg, 6.3 mmol, 4 eq) and TFA (198.7 mg, 1.7 mmol, 129.4 μL, 1.1 eq). The mixture was stirred at 20° C. for 12 hr. LC-MS showed 4 was consumed completely and one main peak with desired m/z was detected. The reaction mixture was concentrated under reduced pressure to remove solvent. The residue was diluted with H₂O 8 mL and extracted with EtOAc 30 mL (10 mL*3), dried over Na₂SO₄, filtered and concentrated under reduced pressure to give a residue. The residue was purified by prep-TLC (SiO₂, Petroleum ether/Ethyl acetate=10/1) to give N-(1-(3-chloro-2-fluorophenyl)-2,2,3,3,3-pentafluoropropyl)cyclopropanamine (360 mg, 1.1 mmol, 71.5% yield) as a white solid.

### 5) 2-bromo-N-(1-(3-chloro-2-fluorophenyl)-2,2,3,3,3-pentafluoropropyl)-N-cyclopropylacetamide

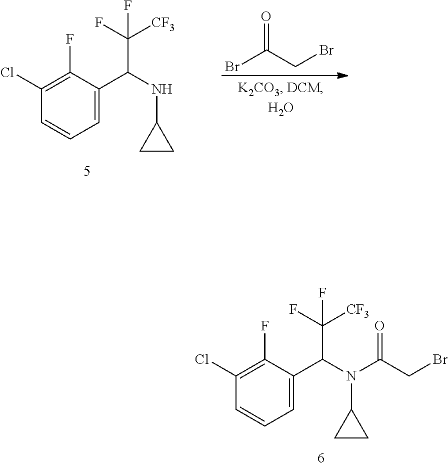 [SMILES: Fc1c(Cl)cccc1C(NC1CC1)C(F)(F)C(F)(F)F ; O=C(Br)CBr ; O=C(CBr)N(C1CC1)C(c1cccc(Cl)c1F)C(F)(F)C(F)(F)F]

To a stirred cooled solution of K₂CO₃ (261.1 mg, 1.9 mmol, 2 eq) in H₂O (10 mL) was added a solution of N-[1-(3-chloro-2-fluoro-phenyl)-2,2,3,3,3-pentafluoro-propyl]cyclopropanamine (300.0 mg, 944.4 μmol, 1 eq) in DCM (10 mL), followed by addition of 2-bromoacetyl bromide (228.8 mg, 1.1 mmol, 98.7 μL, 1.2 eq). The reaction mixture was stirred at 20° C. for 1 h. LC-MS showed 5 was remained partly. The reaction mixture was added K₂CO₃ (261.1 mg, 1.9 mmol, 2 eq) and 2-bromoacetyl bromide (228.8 mg, 1.1 mmol, 98.7 μL, 1.2 eq) and then the mixture was stirred at 20° C. for 1 hr. LC-MS showed Reactant 5 was consumed completely and one main peak with desired m/z was detected. The reaction mixture was diluted with H₂O 10 mL and extracted with DCM 30 mL (10 mL*3), dried over Na₂SO₄, filtered and concentrated under reduced pressure to give 2-bromo-N-(1-(3-chloro-2-fluorophenyl)-2,2,3,3,3-pentafluoropropyl)-N-cyclopropylacetamide (460 mg, crude) as yellow oil and it was used into the next step without further purification.

### 6)N-(1-(3-chloro-2-fluorophenyl)-2,2,3,3,3-pentafluoropropyl)-N-cyclopropyl-2-(1,3-dioxoisoindolin-2-yl)acetamide

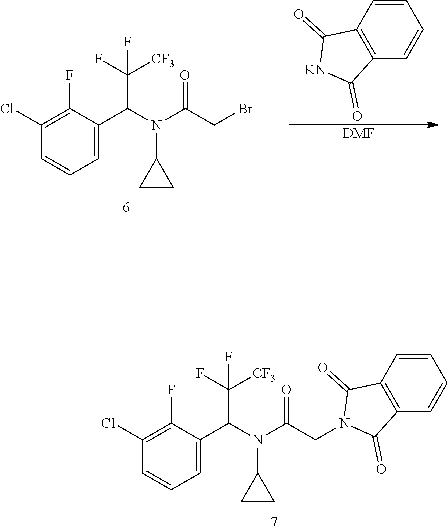

To a solution of 2-bromo-N-[1-(3-chloro-2-fluoro-phenyl)-2,2,3,3,3-pentafluoro-propyl]-N-cyclopropyl-acetamide (400 mg, 912.0 μmol, 1 eq) in DMF (20 mL) was added (1,3-dioxoisoindolin-2-yl)potassium (202.7 mg, 1.1 mmol, 1.2 eq). The mixture was stirred at 20° C. for 12 hr. LC-MS showed 6 was consumed completely and one main peak with desired m/z was detected. The reaction mixture was diluted with H₂O 25 mL and extracted with EtOAc 75 mL (25 mL*3). The combined organic layers were washed with aqueous NaCl 20 mL (10 mL*2), dried over Na₂SO₄, filtered and concentrated under reduced pressure to give N-(1-(3-chloro-2-fluorophenyl)-2,2,3,3,3-pentafluoropropyl)-N-cyclopropyl-2-(1,3-dioxoisoindolin-2-yl)acetamide (750 mg, crude) as yellow oil.

### 7) 2-amino-N-(1-(3-chloro-2-fluorophenyl)-2,2,3,3,3-pentafluoropropyl)-N-cyclopropylacetamide

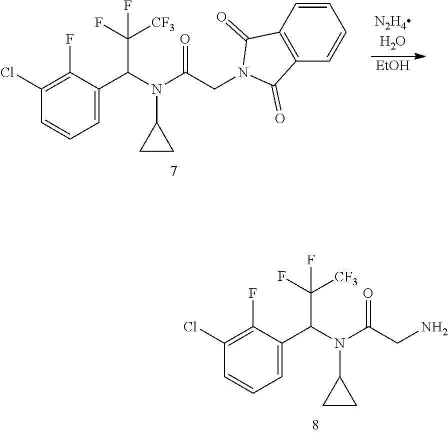 [SMILES: "EtOH" ; O=C1c2ccccc2C(=O)N1CC(=O)N(C1CC1)C(c1cccc(Cl)c1F)C(F)(F)C(F)(F)F ; NCC(=O)N(C1CC1)C(c1cccc(Cl)c1F)C(F)(F)C(F)(F)F]

To a solution of N-[1-(3-chloro-2-fluoro-phenyl)-2,2,3,3,3-pentafluoro-propyl]-N-cyclopropyl-2-(1,3-dioxoisoindolin-2-yl)acetamide (300 mg, 594.3 μmol, 1 eq) in EtOH (15 mL) was added N₂H₄·H₂O (74.4 mg, 1.2 mmol, 72.1 μL, 80% purity, 2 eq). The mixture was stirred at 80° C. for 2.5 hr. LC-MS showed 7 was consumed completely and one main peak with desired m/z was detected. The mixture was cooled to 4° C. and the phthalyl hydrazide removed by filtration. The ethanol was removed in vacuo. The solution was diluted with H₂O 20 mL, then extracted with EtOAc 30 mL (10 mL*3) and the organic layer was dried over Na₂SO₄, filtered and concentrated under reduced pressure to give 2-amino-N-[1-(3-chloro-2-fluoro-phenyl)-2,2,3,3,3-pentafluoro-propyl]-N-cyclopropylacetamide (176 mg, crude) as yellow oil and it was used into the next step without further purification. 8) N1-(1-(3-chloro-2-fluorophenyl)-2,2,3,3,3-pentafluoropropyl)-N1-cyclopropylethane-1,2-diamine

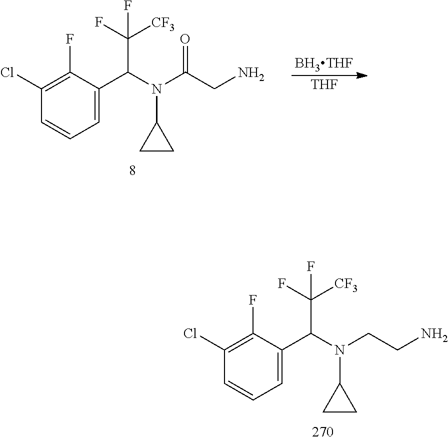 [SMILES: NCC(=O)N(C1CC1)C(c1cccc(Cl)c1F)C(F)(F)C(F)(F)F ; NCCN(C1CC1)C(c1cccc(Cl)c1F)C(F)(F)C(F)(F)F ; "BH3·THF" ; "THF"]

To a solution of 2-amino-N-[1-(3-chloro-2-fluoro-phenyl)-2,2,3,3,3-pentafluoro-propyl]-N-cyclopropyl-acetamide (176 mg, 469.7 μmol, 1 eq) in THF (15 mL) was added BH₃·THF (1 M, 2.8 mL, 6 eq). The mixture was stirred at 60° C. for 12 hr under N₂ atmosphere. LC-MS showed 8 was consumed completely and one main peak with desired m/z was detected. The reaction mixture was quenched by addition MeOH 2 mL at 0° C. and stirred at 60° C. for 2 hr, and then diluted with H₂O 10 mL and extracted with EtOAc 30 mL (10 mL*3), dried over Na₂SO₄, filtered and concentrated under reduced pressure to give a residue. The residue was purified by prep-HPLC (TFA conditions). Column: Phenomenex Luna C18 75*30 mm*3 um; mobile phase: [H2O (0.1% TFA)-ACN]; gradient: 15%-45% B over 8.0 min to give N1-(1-(3-chloro-2-fluorophenyl)-2,2,3,3,3-pentafluoropropyl)-N1-cyclopropylethane-1,2-diamine TFA salt (17.5 mg, 48.1 μmol, 10.2% yield, 99.3% purity) as a yellow oil. MS (ESI): m/z=361.1 [M+H]+ retention time: 1.914 min, method:M. ¹H NMR (400 MHz, DMSO-d6) δ=7.96-7.66 (m, 4H), 7.62 (br t, J=7.1 Hz, 1H), 7.36 (t, J=8.1 Hz, 1H), 5.12-4.97 (m, 1H), 3.19-3.13 (m, 1H), 3.03-2.86 (m, 2H), 2.81-2.70 (m, 1H), 1.91 (br d, J=4.4 Hz, 1H), 0.73-0.47 (m, 4H).

### Example 49: Synthesis of 3-(1-((2-aminoethyl)(cyclopropyl)amino)-3,3,3-trifluoropropyl)-2-fluorobenzonitrile hydrochloride (Compound 280)

### 1) ((1-(3-bromo-2-fluorophenyl)vinyl)oxy)triethylsilane

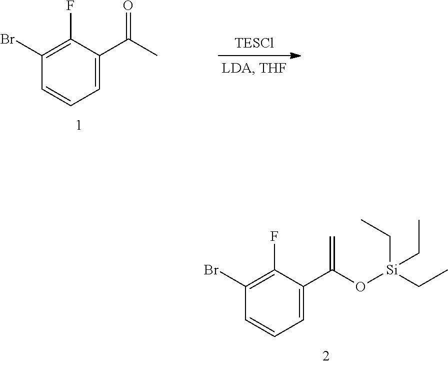 [SMILES: CC(=O)c1cccc(Br)c1F ; C=C(O[Si](CC)(CC)CC)c1cccc(Br)c1F]

A dry round-bottom flask flushed with N₂ was charged with the desired 1-(3-bromo-2-fluoro-phenyl)ethanone (2.5 g, 11.5 mmol, 1 eq) and THF (15 mL). The base, LDA (2 M, 6.9 mL, 1.2 eq), was added dropwise, and the reaction was kept at −70° C. for 1 hr. Chloro(triethyl)silane (2.1 g, 13.8 mmol, 2.4 mL, 1.2 eq) was added to the reaction at −70° C. and stirred at 20° C. for 12 hr. TLC indicated Reactant 1 was consumed completely and one new spot formed. The reaction was quenched with saturated aqueous NH₄Cl 50 mL and extracted with ethyl acetate (3×80 mL). The combined organic phases were dried over Na₂SO₄, filtered and concentrated under reduced pressure. The residue was purified by flash silica gel chromatography (12 g Silica Flash Column, Eluent of 0˜0% Ethyl acetate/Petroleum ether gradient @80 m/min) to give ((1-(3-bromo-2-fluorophenyl)vinyl)oxy)triethylsilane (1.0 g, 3.1 mmol, 27.0% yield) as a colourless oil.

### 2) 1-(3-bromo-2-fluorophenyl)-3,3,3-trifluoropropan-1-one

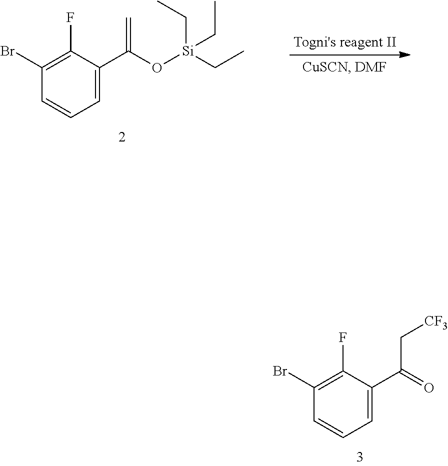 [SMILES: C=C(O[Si](CC)(CC)CC)c1cccc(Br)c1F ; O=C(CC(F)(F)F)c1cccc(Br)c1F]

1-(trifluoromethyl)-1,2-benziodoxol-3-one (1.4 g, 4.5 mmol, 1.5 eq) and CuSCN (37.9 mg, 301.9 μmol, 0.1 eq) under N₂ atmosphere. To the mixture were added 1-(3-bromo-2-fluoro-phenyl)vinyloxy-triethyl-silane (1.0 g, 3.0 mmol, 1 eq) and DMF (20 mL). The mixture was stirred at 20° C. for 12 h. TLC indicated 2 was consumed completely and many new spots formed. The reaction mixture was diluted with H₂O 25 mL and extracted with EtOAc 75 mL (25 mL*3). The combined organic layers were washed with brine 20 mL (10 mL*2), dried over Na₂SO₄, filtered and concentrated under reduced pressure to give a residue. The residue was purified by flash silica gel chromatography (12 g Silica Flash Column, Eluent of 0˜3% Ethyl acetate/Petroleum ether gradient @80 mL/min) to give 1-(3-bromo-2-fluorophenyl)-3,3,3-trifluoropropan-1-one (530 mg, 1.9 mmol, 61.6% yield) as a yellow oil.

### 3)N-(1-(3-bromo-2-fluorophenyl)-3,3,3-trifluoropropyl)cyclopropanamine

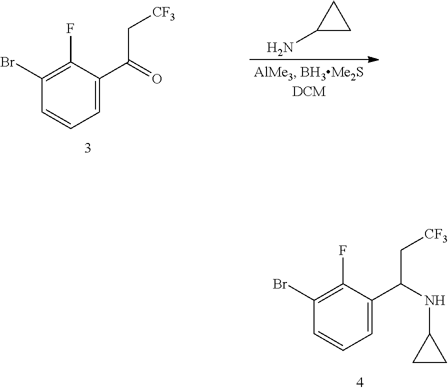

Dissolve the 1-(3-bromo-2-fluoro-phenyl)-3,3,3-trifluoro-propan-1-one (300 mg, 1.1 mmol, 1 eq) and the cyclopropanamine (90.1 mg, 1.6 mmol, 109.4 μL, 1.5 eq) under N₂ in dry DCM (15 mL). Add AlMe₃ (2 M, 789.4 μL, 1.5 eq) dropwise through syringe to the reaction mixture. Stir the solution at 20° C. for 15 hr. Add BH₃-Me₂S (10 M, 210.5 μL, 2 eq) dropwise to the reaction mixture. Stir the mixture at 20° C. for 2 hr. LC-MS showed one main peak with desired m/z was detected. Quench the reaction mixture by dropwise addition of 20% aqueous NaOH. Extract the aqueous layer with CH₂Cl₂ (3×30 mL). Dry the organic layer over Na₂SO₄. Remove the solvent under reduced pressure. The residue was purified by flash silica gel chromatography (12 g Silica Flash Column, Eluent of 0-3% Ethyl acetate/Petroleum ether gradient @60 mL/min) to give N-(1-(3-bromo-2-fluorophenyl)-3,3,3-trifluoropropyl)cyclopropanamine (120 mg, 368.0 μmol, 35.0% yield) as a yellow oil.

### 4) tert-butyl (2-((1-(3-bromo-2-fluorophenyl)-3,3,3-trifluoropropyl)(cyclopropyl)amino) ethyl)carbamate

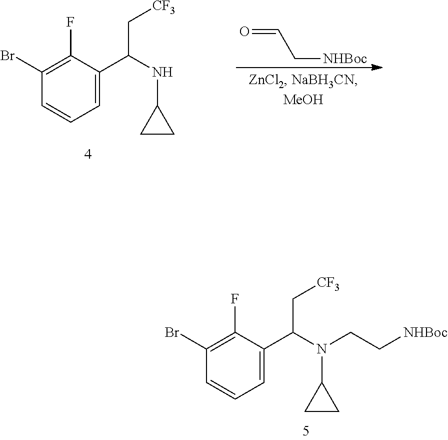 [SMILES: Fc1c(Br)cccc1C(CC(F)(F)F)NC1CC1 ; CC(C)(C)OC(=O)NCC=O ; CC(C)(C)OC(=O)NCCN(C1CC1)C(CC(F)(F)F)c1cccc(Br)c1F]

To a solution of N-[1-(3-bromo-2-fluoro-phenyl)-3,3,3-trifluoro-propyl]cyclopropanamine (120 mg, 368.0 μmol, 1 eq) and tert-butyl N-(2-oxoethyl)carbamate (175.7 mg, 1.1 mmol, 3 eq) in MeOH (2 mL) was added ZnCl₂ (100.3 mg, 735.9 μmol, 34.5 μL, 2 eq) and NaBH₃CN (92.5 mg, 1.5 mmol, 4 eq). The mixture was stirred at 20° C. for 12 hrs. LC-MS showed 4 was remained and one main peak with desired m/z was detected. The reaction mixture was concentrated under reduced pressure to remove solvent. The residue was diluted with H₂O 5 mL and extracted with EtOAc 15 mL (5 mL*3), dried over Na₂SO₄, filtered and concentrated under reduced pressure to give a residue. The residue was purified by prep-TLC (SiO₂, Petroleum ether/Ethyl acetate=4/1) to give tert-butyl (2-((1-(3-bromo-2-fluorophenyl)-3,3,3-trifluoropropyl)(cyclopropyl)amino) ethyl) carbamate (103 mg, 219.5 μmol, 59.7% yield) as a yellow oil.

### 5) tert-butyl (2-((1-(3-cyano-2-fluorophenyl)-3,3,3-trifluoropropyl)(cyclopropyl)amino)ethyl)carbamate

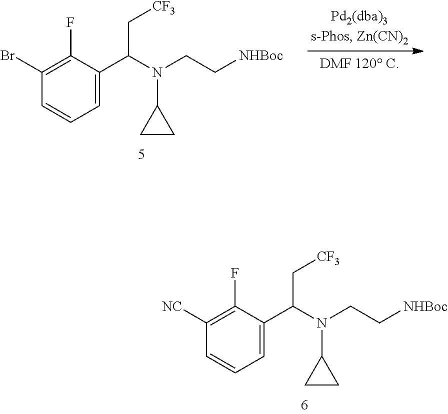 [SMILES: CC(C)(C)OC(=O)NCCN(C1CC1)C(CC(F)(F)F)c1cccc(Br)c1F ; CC(C)(C)OC(=O)NCCN(C1CC1)C(CC(F)(F)F)c1cccc(C#N)c1F]

A mixture of tert-butyl N-[2-[[1-(3-bromo-2-fluoro-phenyl)-3,3,3-trifluoro-propyl]-cyclopropyl-amino]ethyl]carbamate (100 mg, 213.1 μmol, 1 eq), Zn(CN)₂ (75.1 mg, 639.2 mol, 40.6 μL, 3 eq), Pd₂(dba)₃ (19.5 mg, 21.3 μmol, 0.1 eq) and s-Phos (8.8 mg, 21.1 μmol, 0.1 eq) in DMF (3 mL) was degassed and purged with N₂ for 3 times, and then the mixture was stirred at 120° C. for 12 hr under N₂ atmosphere. LC-MS showed 5 was consumed partly and one main peak with desired m/z was detected. The reaction mixture was quenched by addition water 10 mL at 0° C., and then extracted with Ethyl acetate 15 mL (5 mL*3). The combined organic layers were dried over Na₂SO₄, filtered and concentrated under reduced pressure to give a residue. The residue was purified by prep-TLC (SiO₂, Petroleum ether/Ethyl acetate=3/1) to give tert-butyl (2-((1-(3-cyano-2-fluorophenyl)-3,3,3 trifluoropropyl)(cyclopropyl)amino)ethyl)carbamat (35 mg, 84.6 μmol, 39.5% yield) as a yellow oil.

### 6) 3-(1-((2-aminoethyl)(cyclopropyl)amino)-3,3,3-trifluoropropyl)-2-fluorobenzonitrile hydrochloride

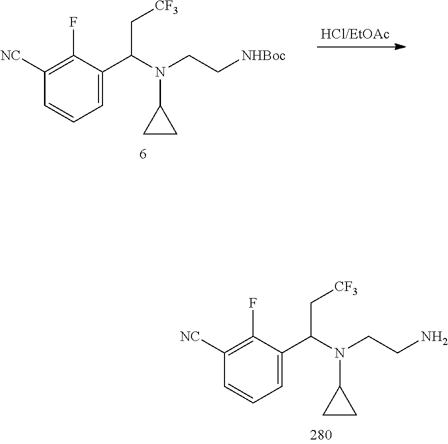 [SMILES: CC(C)(C)OC(=O)NCCN(C1CC1)C(CC(F)(F)F)c1cccc(C#N)c1F ; N#Cc1cccc(C(CC(F)(F)F)N(CCN)C2CC2)c1F]

A solution of tert-butyl N-[2-[[1-(3-cyano-2-fluoro-phenyl)-3,3,3-trifluoro-propyl]-cyclopropyl-amino]ethyl]carbamate (35 mg, 84.6 μmol, 1 eq) in HCl/EtOAc (5 mL) was stirred at 20° C. for 1 hr. LC-MS showed 6 was consumed completely and one main peak with desired mass was detected. The reaction mixture was concentrated under reduced pressure to remove solvent. The residue was purified by prep-HPLC (HCl condition, column: Phenomenex Luna C18 75*30 mm*3 um; mobile phase: [H₂O (0.04% HCl)-ACN]; gradient: 10%-40% B over 8.0 min) to give 3-(1-((2-aminoethyl)(cyclopropyl)amino)-3,3,3-trifluoropropyl)-2-fluorobenzonitrile hydrochloride (27.7 mg, 78.4 μmol, 93.0% yield, 99.7% purity, HCl) as a yellow oil. MS (ESI): m/z=316.2 [M+H]⁺, retention time: 1.638 min, method:M. ¹H NMR (400 MHz, DMSO-d6) δ=8.19-8.05 (m, 3H), 8.00 (br t, J=7.1 Hz, 1H), 7.97-7.89 (m, 1H), 7.51-7.42 (m, 1H), 4.59 (br dd, J=5.6, 8.6 Hz, 1H), 3.30-3.02 (m, 3H), 2.90 (br d, J=5.5 Hz, 2H), 2.61-2.51 (m, 1H), 1.78-1.66 (m, 1H), 0.72-0.40 (m, 4H).

### Example 50: Synthesis of N′-[1-(3-bromo-2-fluoro-phenyl)-2-methyl-propyl]-N′-cyclopropyl-ethane-1,2-diamine TFA salt (Compound 294)

### 1) 1-(3-bromo-2-fluoro-phenyl)-2-methyl-propan-1-ol

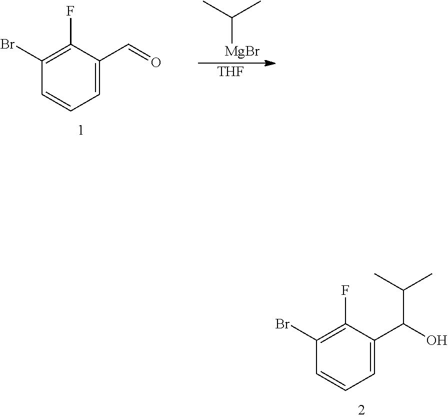

To a solution of 3-bromo-2-fluoro-benzaldehyde (6.8 g, 33.50 mmol, 1 eq) in THF (50 mL) was dropwise added i-PrMgBr (1 M, 40.20 mL, 1.2 eq). The mixture was stirred at 0° C. for 2 hr. TLC indicated 1 was consumed completely and one major new spot formed. The reaction was quenched with saturated aqueous NH₄Cl and extracted with ethyl acetate (3*30 mL). The combined organic phases were dried over Na₂SO₄, filtered and concentrated under reduced pressure. The residue was purified by flash silica gel chromatography (Silica Flash Column, Eluent of 0˜3% Ethyl acetate/Petroleum ether gradient @100 m/min) to give 1-(3-bromo-2-fluoro-phenyl)-2-methyl-propan-1-ol (1.9 g, 6.91 mmol, 20.62% yield, 89.84% purity) as a pale yellow oil. ¹H NMR (400 MHz, DMSO-d₆) δ=7.61-7.54 (m, 1H), 7.49-7.43 (m, 1H), 7.16 (t, J=7.8 Hz, 1H), 5.38 (d, J=4.6 Hz, 1H), 4.57 (t, J=5.4 Hz, 1H), 1.91-1.72 (m, 1H), 0.87 (d, J=6.8 Hz, 3H), 0.79 (d, J=6.9 Hz, 3H)

### 2) 1-(3-bromo-2-fluoro-phenyl)-2-methyl-propan-1-one

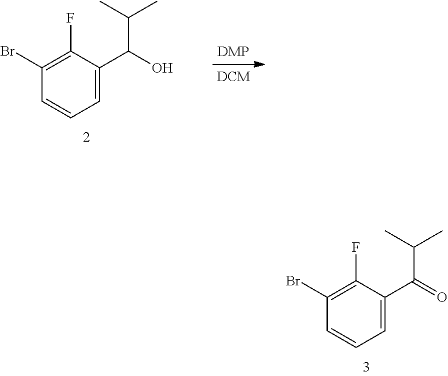

To a solution of 1-(3-bromo-2-fluoro-phenyl)-2-methyl-propan-1-ol (1.9 g, 7.69 mmol, 1 eq) in DCM (20 mL) was added Dess-Martin (6.52 g, 15.38 mmol, 4.76 mL, 2 eq). The mixture was stirred at 20° C. for 2 hr. LC-MS showed no 2 remained and ˜20% of desired compound was detected. The reaction mixture filtered to remove the insoluble and concentrated under reduced pressure to give a residue. The residue was purified by flash silica gel chromatography (12 g Silica Flash Column, Eluent of 0-0% Ethyl acetate/Petroleum ether gradient @80 m/min) to give 1-(3-bromo-2-fluoro-phenyl)-2-methyl-propan-1-one (1.3 g, 5.09 mmol, 66.16% yield, 95.91% purity) as a yellow solid.

### 3)N-[1-(3-bromo-2-fluoro-phenyl)-2-methyl-propyl]cyclopropanamine

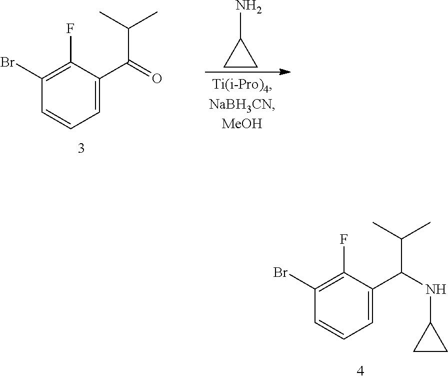

To a solution of 1-(3-bromo-2-fluoro-phenyl)-2-methyl-propan-1-one (1.2 g, 4.90 mmol, 1 eq) in MeOH (10 mL) was added cyclopropanamine (419.32 mg, 7.34 mmol, 508.88 μL, 1.5 eq) and Ti(i-PrO)₄ (4.17 g, 14.69 mmol, 4.34 mL, 3 eq). The mixture was stirred at 60° C. for 12 hrs, then NaBH₃CN (1.23 g, 19.58 mmol, 4 eq) was added, the mixture was stirred at 60° C. for 2 hrs. LC-MS showed no 3 remained and ˜47% of desired compound was detected. The reaction mixture was concentrated under reduced pressure to remove solvent. The residue was diluted with H₂O 20 mL and extracted with ethyl acetate (15 mL*3). The combined organic layers were dried over Na₂SO₄, filtered and concentrated under reduced pressure to give a residue. The residue was purified by flash silica gel chromatography (12 g Silica Flash Column, Eluent of 0-3% Ethyl acetate/Petroleum ether gradient @80 mL/min) to give N-[1-(3-bromo-2-fluoro-phenyl)-2-methyl-propyl]cyclopropanamine (1.2 g, 3.69 mmol, 75.45% yield, 88.10% purity) as a colorless oil.

### 4) tert-butyl N-[2-[[1-(3-bromo-2-fluoro-phenyl)-2-methyl-propyl]-cyclopropyl-amino]ethyl]carbamate

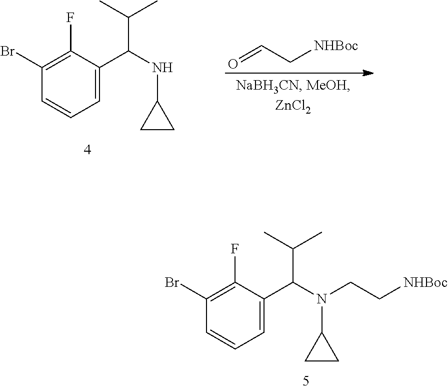 [SMILES: CC(C)C(NC1CC1)c1cccc(Br)c1F ; CC(C)(C)OC(=O)NCC=O ; CC(C)C(c1cccc(Br)c1F)N(CCNC(=O)OC(C)(C)C)C1CC1]

To a solution of N-[1-(3-bromo-2-fluoro-phenyl)-2-methyl-propyl]cyclopropanamine (600 mg, 2.10 mmol, 1 eq) and tert-butyl N-(2-oxoethyl)carbamate (1.33 g, 8.39 mmol, 4 eq) in MeOH (8 mL) was added NaBH₃CN (263.50 mg, 4.19 mmol, 2 eq) and ZnCl₂ (571.52 mg, 4.19 mmol, 196.60 μL, 2 eq). The mixture was stirred at 20° C. for 12 hrs. LC-MS showed no 4 remained and ˜84% of desired compound was detected. The reaction mixture was concentrated under reduced pressure to remove solvent. The residue was diluted with H₂O 10 mL and extracted with ethyl acetate (5 mL*3). The combined organic layers were dried over Na₂SO₄, filtered and concentrated under reduced pressure to give tert-butyl N-[2-[[1-(3-bromo-2-fluoro-phenyl)-2-methyl-propyl]-cyclopropyl-amino]ethyl]carbamate (300 mg, crude) as a colorless oil.

### 5) N′-[1-(3-bromo-2-fluoro-phenyl)-2-methyl-propyl]-N′-cyclopropyl-ethane-1,2-diamine TFA salt

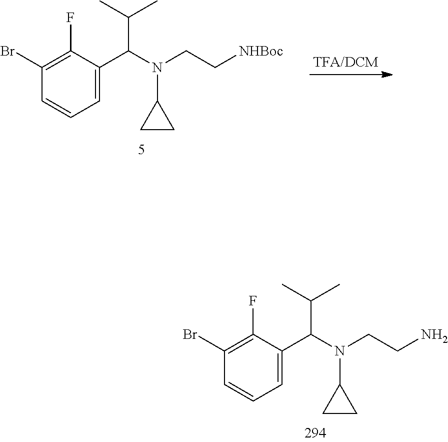 [SMILES: CC(C)C(c1cccc(Br)c1F)N(CCNC(=O)OC(C)(C)C)C1CC1 ; CC(C)C(c1cccc(Br)c1F)N(CCN)C1CC1]

To a solution of tert-butyl N-[2-[[1-(3-bromo-2-fluoro-phenyl)-2-methyl-propyl]-cyclopropyl-amino]ethyl]carbamate (150 mg, 349.35 μmol, 1 eq) in DCM (2 mL) was added TFA (1.54 g, 13.46 mmol, 1 mL, 38.54 eq). The mixture was stirred at 20° C. for 1 hr. LC-MS showed no 5 remained and ˜60% of desired compound was detected. The reaction mixture was concentrated under reduced pressure to give a residue. The residue was purified by prep-HPLC (TFA condition; column: Phenomenex luna C18 100*40 mm*5 um; mobile phase: [H₂O (0.1% TFA)-ACN]; gradient: 25%-55% B over 8.0 min) to give N′-[1-(3-bromo-2-fluoro-phenyl)-2-methyl-propyl]-N′-cyclopropyl-ethane-1,2-diamine TFA salt (32.96 mg, 74.27 μmol, 21.26% yield, 99.88% purity, 1TFA) as a colorless oil. MS (ESI): m/z=328.1[M+H]⁺, retention time: 1.878 min, method:Bhalo. ¹H NMR (400 MHz, CHLOROFORM-d) δ=8.44-7.77 (m, 3H), 7.55 (t, J=7.0 Hz, 1H), 7.30 (br t, J=6.6 Hz, 1H), 7.09 (t, J=7.8 Hz, 1H), 3.97 (br d, J=10.5 Hz, 1H), 3.40 (br s, 1H), 3.26 (br s, 2H), 2.89-2.75 (m, 1H), 2.48-2.32 (m, 1H), 1.70-1.60 (m, 1H), 1.13-0.97 (m, 3H), 0.88 (br s, 1H), 0.79-0.59 (m, 6H).

### Example 51: Description of LCMS Methods

The above specified LCMS methods were as follows:

| A | Instrument: Agilent 1260 HPLC MSD: | | |
|---|---|---|---|
| | 6120 single quadrupole MSD | | |
| | Column: Luna C18, 2*50 mm, 5 μm | | |
| | Column Temp: 40° C. | | |
| | Mobile Phase: A: H₂O + TFA(0.04%) | | |
| | Mobile Phase: B: ACN + TFA(0.02%) | | |
| | Flow Rate: 1.0 ml/min | | |
| | Time | B % | Flow(ml/min) |
| | 0.00 | 0 | 1.0 |
| | 0.40 | 0 | 1.0 |
| | 3.00 | 60 | 1.0 |
| | 4.00 | 100 | 1.0 |
| | 4.01 | 0 | 1.0 |
| | 4.50 | 0 | 1.0 |
| B | Instrument: Agilent 1200 HPLC MSD: | | |
| | 6120 single quadrupole MSD | | |
| | Column: Luna C18, 2.0*50 mm, 5 μm | | |
| | Column Temp: 40° C. | | |
| | Mobile Phase: A: 0.04% TFA in H₂O | | |
| | Mobile Phase: B: 0.02% TFA in ACN | | |
| | Flow Rate: 1 ml/min | | |
| | Time | B % | Flow(ml/min) |
| | 0.01 | 5 | 1 |
| | 0.40 | 5 | 1 |
| | 3.00 | 95 | 1 |
| | 4.00 | 95 | 1 |
| | 4.01 | 5 | 1 |
| | 4.50 | 5 | 1 |
| C | Instrument: Agilent 1200 HPLC MSD: | | |
| | 6120 single quadrupole MSD | | |
| | Column: XBridge C18, 2.1*50 mm, 5 μm | | |
| | Column Temp: 40° C. | | |
| | Mobile Phase: A: H₂O + 10 mM NH₄HCO₃ | | |
| | Mobile Phase: B: ACN | | |
| | Flow Rate: 0.8 ml/min | | |
| | Time | B % | Flow(ml/min) |
| | 0.00 | 5 | 0.8 |
| | 3.40 | 95 | 0.8 |
| | 3.85 | 95 | 0.8 |
| | 3.86 | 5 | 0.8 |
| | 4.50 | 5 | 0.8 |
| D | Instrument: Agilent 1260 HPLC MSD: | | |
| | 6120 single quadrupole MSD | | |
| | Column: Luna C18, 2.0*50 mm, 5 μm | | |
| | Column Temp: 40° C. | | |
| | Mobile Phase: A: 0.04% TFA in H₂O | | |
| | Mobile Phase: B: 0.02% TFA in ACN | | |
| | Flow Rate: 1 ml/min | | |
| | Time | B % | Flow(ml/min) |
| | 0.01 | 5 | 1 |
| | 0.40 | 5 | 1 |
| | 3.00 | 95 | 1 |
| | 4.00 | 95 | 1 |
| | 4.01 | 5 | 1 |
| | 4.50 | 5 | 1 |
| E | Instrument: Agilent 1200 HPLC MSD: | | |
| | 6130 single quadrupole MSD | | |
| | Column: XBridge C18, 2.1*50 mm, 5 μm | | |
| | Column Temp: 40° C. | | |
| | Mobile Phase: A: H₂O + 10 mM NH₄HCO₃ | | |
| | Mobile Phase: B: ACN | | |
| | Flow Rate: 0.8 ml/min | | |
| | Time | B % | Flow(ml/min) |
| | 0.00 | 5 | 0.8 |
| | 0.40 | 5 | 0.8 |
| | 3.40 | 95 | 0.8 |
| | 3.85 | 95 | 0.8 |
| | 3.86 | 5 | 0.8 |
| | 4.50 | 5 | 0.8 |
| F | Instrument: Agilent 1260 HPLC MSD: | | |
| | 6120 single quadrupole MSD | | |
| | Column: Luna C18, 2.0*50 mm, 5 μm | | |
| | Column Temp: 40° C. | | |
| | Mobile Phase: A: 0.04% TFA in H₂O | | |
| | Mobile Phase: B: 0.02% TFA in ACN | | |
| | Flow Rate: 1 ml/min | | |
| | Time | B % | Flow(ml/min) |
| | 0.00 | 5 | 1 |
| | 0.40 | 5 | 1 |
| | 3.00 | 95 | 1 |
| | 4.00 | 95 | 1 |
| | 4.01 | 5 | 1 |
| | 4.50 | 5 | 1 |
| G | Instrument: Agilent 1200 HPLC MSD: | | |
| | 6110 single quadrupole MSD | | |
| | Column: Luna C18, 2.0*50 mm, 5 μm | | |
| | Column Temp: 40° C. | | |
| | Mobile Phase: A: 0.04% TFA in H₂O | | |
| | Mobile Phase: B: 0.02% TFA in ACN | | |
| | Flow Rate: 1 ml/min | | |
| | Time | B % | Flow(ml/min) |
| | 0.01 | 5 | 1 |
| | 0.40 | 5 | 1 |
| | 3.00 | 95 | 1 |
| | 4.00 | 95 | 1 |
| | 4.01 | 5 | 1 |
| | 4.50 | 5 | 1 |
| H | Instrument: Agilent 1200 HPLC MSD: | | |
| | 6130 single quadrupole MSD | | |
| | Column: XBridge C18, 2.1*50 mm, 5 μm | | |
| | Column Temp: 40° C. | | |
| | Mobile Phase: A: H₂O + 10 mM NH₄HCO₃ | | |
| | Mobile Phase: B: ACN | | |
| | Flow Rate: 1 ml/min | | |
| | Time | B % | Flow(ml/min) |
| | 0.00 | 0 | 1.0 |
| | 0.40 | 0 | 1.0 |
| | 3.00 | 30 | 1.0 |
| | 4.00 | 30 | 1.0 |
| | 4.01 | 0 | 1.0 |
| | 4.50 | 0 | 1.0 |
| I | Instrument: Agilent 1200 HPLC MSD: | | |
| | 1956A single quadrupole MSD | | |
| | Column: Luna C18, 2.0*50 mm, 5 μm | | |
| | Column Temp: 40° C. | | |
| | Mobile Phase: A: 0.04% TFA in H₂O | | |
| | Mobile Phase: B: 0.02% TFA in ACN | | |
| | Flow Rate: 1 ml/min | | |
| | Time | B % | Flow(ml/min) |
| | 0.01 | 5 | 1 |
| | 0.40 | 5 | 1 |
| | 3.00 | 95 | 1 |
| | 4.00 | 95 | 1 |
| | 4.01 | 5 | 1 |
| | 4.50 | 5 | 1 |
| J | Instrument: Agilent 1260 HPLC MSD: | | |
| | 6125B single quadrupole MSD | | |
| | Column: Luna C18, 2.0*50 mm, 5 μm | | |
| | Column Temp: 40° C. | | |
| | Mobile Phase: A: 0.04% TFA in H₂O | | |
| | Mobile Phase: B: 0.02% TFA in ACN | | |
| | Flow Rate: 1 ml/min | | |
| | Time | B % | Flow(ml/min) |
| | 0.01 | 5 | 1 |
| | 0.40 | 5 | 1 |
| | 3.00 | 95 | 1 |
| | 4.00 | 95 | 1 |
| | 4.01 | 5 | 1 |
| | 4.50 | 5 | 1 |
| K | Instrument: Agilent 1260 HPLC MSD: | | |
| | 6120 single quadrupole MSD | | |
| | Column: Kinetex C18, 2.1*50 mm, 5 μm | | |
| | Column Temp: 40° C. | | |
| | Mobile Phase: A: H₂O + TFA(0.04%) | | |
| | Mobile Phase: B: ACN + TFA(0.02%) | | |
| | Flow rate: 1 ml/min | | |
| | Time | B % | Flow(ml/min) |
| | 0.00 | 5 | 1 |
| | 0.40 | 5 | 1 |
| | 3.00 | 95 | 1 |
| | 4.00 | 95 | 1 |
| | 4.01 | 5 | 1 |
| | 4.50 | 5 | 1 |
| L | Analytical SFC Waters UPCC with PDA | | |
| | Column: Chiralpak IH-3, 100 × 4.6 mm I.D., 3 μm | | |
| | Mobile phase: A: CO2 B: MeOH(0.1% IPAm, v/v) | | |
| | Gradient: | | |
| | Time | A % | B % |
| | 0.0 | 90 | 10 |
| | 0.2 | 90 | 10 |
| | 2.4 | 50 | 50 |
| | 3.4 | 50 | 50 |
| | 4.0 | 90 | 10 |
| | Flow rate: 3.4 mL/min | | |
| | Column temp.: 35° C. | | |
| | ABPR: 2000 psi″ | | |
| M | Instrument: Shimadzu LC-20AD XR MSD: LCMS-2020 | | |
| | Column: Luna C18, 2.0*50 mm, 5 μm | | |
| | Column Temp: 40° C. | | |
| | Mobile Phase: A: 0.04% TFA in H2O | | |
| | Mobile Phase: B: 0.02% TFA in ACN | | |
| | Flow Rate: 1 ml/min | | |
| | Time | B % | Flow(ml/min) |
| | 0.01 | 5 | 1 |
| | 0.40 | 5 | 1 |
| | 3.00 | 95 | 1 |
| | 4.00 | 95 | 1 |
| | 4.01 | 5 | 1 |
| | 4.50 | stop | |
| Bhalo | Instrument: Shimadzu LC-20AD XR&MS 2020 | | |
| | Column: Halo C18, 3.0*30 mm,5 μm | | |
| | Column Temp: 40° C. | | |
| | Mobile Phase: A: H₂O + 0.04%(v/v) TFA | | |
| | Mobile Phase: B: ACN + 0.02%(v/v) TFA | | |
| | Flow Rate: 1 ml/min | | |
| | Time | MPA(%) | MPB(%) |
| | 0.00 | 95 | 5 |
| | 0.40 | 95 | 5 |
| | 3.00 | 5 | 95 |
| | 4.00 | 5 | 95 |
| | 4.01 | 95 | 5 |
| | 4.50 | 95 | 5 |

### Example 52: Primary Neuronal Cultures and Excitotoxic Cell Death

Primary mouse hippocampal neurons were prepared as previously described (Bading & Greenberg, Science, 1991; 253: 912-914; Zhang et al, Neuron, 2007, 53: 549-562) and maintained in Neurobasal-A medium supplemented with 2% B27, 5 mM L-Glutamax, and 0.5% Penicillin/Streptomycin until challenged with NMDA on days in vitro (DIV) 10. Glutamate-induced cell death was analyzed in a real-time manner by monitoring nuclear localized mCherry with IncuCyte® S3 Live-Cell Analysis System (Sartorius AG, Germany). Primary neurons plated in 24 well plates were infected with rAAV-hSyn-mCherry on DIV3 and challenged with 1 μM or 20 μM glutamate on DIV10. All compounds were added to the culture 30 min prior to glutamate/NMDA insult. Images were acquired following the glutamate application for 24 h with a 2 h interval, where 9-16 images were obtained via a 20×objective for each condition at each time point. Cell deaths were quantified by analyzing the existence of nuclear localized mCheery via the Basic Analyzer with Incucyte® 2021 software, size and average intensity were used as exclusion criteria.

### Rating of Compounds

In order to evaluate the protective effects of various compounds, a quantification method was developed to determine each compound's protection index. The protection index (from 0.0 to 10.0) was calculated by the area above the curve (AAC) during a 24 h excitotoxic stimuli, where the cell survival was normalized to the 0 h (FIG. 2), following the equation:

$$Protection index = 10 * ( 1 - \frac{AAC - {AAC}_{basal}}{{AAC}_{Veh} - {AAC}_{basal}} )$$

Where AAC represents for each compound, AAC_{basal} represents for the basal condition without excitotoxic insult, AAC_{Veh} represents for the vehicle control with excitotoxic insult. Based on the equation, vehicle (DMSO) has a protection index at 0.0 and compound P401 of WO 2020/079244 has a protection index at 6.0 (at 10 μM). Therefore, the inventors grouped the compounds into A, B, C and D, where:
A. With a protection index over 7.0, provides a significantly better protection over compound P401 (10 μM) at 10 μM.
B. With a protection index between 5.0 to 7.0, provides a comparable protection to compound P401 (10 μM) at 10 μM.
C. With a protection index between 3.0 to 5.0, provides a significantly lower protection compared to compound P401 (10 μM) at 10 μM.
D. With a protection index below 3.0, where no significant protection at 10 μM.

The inventors also performed experiments with Group A compounds at lower concentration in a 3-fold dilution matter (in M: 3.0, 1.0, 0.3, 0.1, 0.03) and were able to achieve a similar protection effect as 10 μM compound P401, but at a lower concentration. These compounds were grouped into A+, A++, A+++, and A++++ compounds:
A+ With a protection index between 5.0 to 7.0, provides a comparable protection to compound P401 (10 μM) at 3.0 μM.
A++ With a protection index between 5.0 to 7.0, provides a comparable protection to compound P401 (10 μM) at 1.0 μM.
A+++ With a protection index between 5.0 to 7.0, provides a comparable protection to compound P401 (10 μM) at 0.3 μM.
A++++ With a protection index between 5.0 to 7.0, provides a comparable protection to compound P401 (10 μM) at 0.1 μM.

### Example 53: Biological Activity of Various Compounds

Using the above-mentioned analysis method for excitotoxic cell death in primary neuronal cultures, a first subset of compounds achieved the following rating results:

| | | | Biological |
|---|---|---|---|
| | Compound | Salt | Activity Rating |
| 1. | 91 | 2HCl | A |
| 2. | 92 | 2HCl | A |
| 3. | 94 | 2HCl | B |
| 4. | 95 | 2HCl | B |
| 5. | 118 | 1HCL | C |
| 6. | 119 | 1HCl | A |
| 7. | 120 | 1HCl | A+++ |
| 8. | 122 | 1HCl | B |
| 9. | 123 | 1HCl | B |
| 10. | 124 | 1HCl | B |
| 11. | 126 | 2HCl | B |
| 12. | 130 | 1HCl | B |
| 13. | 134 | 1HCl | B |
| 14. | 135 | 1HCl | B |
| 15. | 136 | 2HCl | A |
| 16. | 137 | 1HCl | A |
| 17. | 153 | 2HCl | C |
| 18. | 159 | 2HCl | A++ |
| 19. | 160 | 2HCl | A |
| 20. | 175 | 1TFA | A+ |
| 21. | 175A | 1HCl | A |

In a further round of experiments, a second subset of compounds achieved the following rating results (using the above-mentioned analysis method for excitotoxic cell death in primary neuronal cultures):

| | | | Biological |
|---|---|---|---|
| | Compound | Salt | Activity Rating |
| 1. | 169 | 1HCl | B |
| 2. | 172 | 1HCl | A++ |
| 3. | 109 | 2HCl | A |
| 4. | 176 | 2HCl | A |
| 5. | 177 | 2HCl | A |
| 6. | 180 | 2HCl | A++ |
| 7. | 182 | 1HCl | A+ |
| 8. | 184 | 2HCl | A |
| 9. | 185 | 1HCl | A++ |
| 10. | 188 | 1HCl | A++ |
| 11. | 220 | 1HCl | A++++ |
| 12. | 220A | 1HCl | A++ |
| 13. | 220B | 1HCl | A++++ |

In a further round of experiments, a third subset of compounds achieved the following rating results (using the above-mentioned analysis method for excitotoxic cell death in primary neuronal cultures):

| | | Biological | |
|---|---|---|---|
| | Compound | Activity Rating | IC 50 [μM] |
| 1. | 186 | A+++ | 0.800 |
| 2. | 187 | A++ | 2.000 |
| 3. | 190 | A++++ | 0.110 |
| 4. | 191 | A++++ | 0.230 |
| 5. | 218 | A++++ | 0.250 |
| 6. | 219 | A+++ | 0.480 |
| 7. | 219B | A++ | 1.450 |
| 8. | 224 | A++++++ | 0.026 |
| 9. | 225 | A+++++++ | 0.005 |
| 10. | 227 | A+++ | 0.370 |
| 11. | 227B | A+++ | 0.300 |
| 12. | 242 | A+++ | 0.300 |
| 13. | 243 | A+++ | 0.300 |
| 14. | 244 | A+++ | 0.630 |
| 15. | 245 | A+++ | 0.330 |
| 16. | 246 | A+++ | 0.500 |
| 17. | 247 | A++++++ | 0.010 |
| 18. | 248 | A++++ | 0.120 |
| 19. | 257 | A++++++ | 0.011 |
| 20. | 258 | A++++++ | 0.013 |
| 21. | 259 | A+++++ | 0.032 |
| 22. | 261 | A++++ | 0.109 |
| 23. | 261A | A+++++ | 0.033 |
| 24. | 262 | A++++++ | 0.010 |
| 25. | 263 | A++++ | 0.255 |
| 26. | 264 | A+++++++ | 0.003 |
| 27. | 267 | A++++ | 0.130 |
| 28. | 268 | A++++ | 0.280 |
| 29. | 270 | A++++++ | 0.013 |
| 30. | 271A | A+++++++ | 0.007 |
| 31. | 273 | A+++ | 0.940 |
| 32. | 274 | A+++++++ | 0.006 |
| 33. | 275 | A+++ | 0.468 |
| 34. | 279 | A++++ | 0.230 |
| 35. | 280 | A+++++ | 0.036 |
| 36. | 282 | A+++++ | 0.051 |
| 37. | 287 | A+++++++ | 0.003 |
| 38. | 294 | A++++++ | 0.010 |
| 39. | 295 | A+++++ | 0.032 |
| 40. | 297 | A+++ | 0.974 |
| 41. | 297A | A+++++ | 0.081 |

In this third set of experiments, the biological activity rating for compounds rated as A++++ or above correlates with IC50 (μM) as follows:

| | Biological | |
|---|---|---|
| | Activity Rating | IC 50 [μM] |
| 1. | A++++ | 0.1-0.3 |
| 2. | A+++++ | 0.03-0.1 |
| 3. | A++++++ | 0.01-0.03 |
| 4. | A+++++++ | 0.003-0.01 |
| 5. | A++++++++ | 0.001-0.003 |

### Example 54: Reactive Oxygen Species (ROS) Induced Neuronal Death

ROS is a common cause for a damaging effect on neurons; ROS accumulates in the brain and can cause neuronal death and neurodegenerative diseases (Barnham et al, Nat Rev Drug Discov, 2004, 3: 205-214; Singh et al, Molecules, 2019, 24(8), 1583). Previous studies have shown that NMDARs mediate ROS-induced neuronal damage (Avshalumov & Rice, J Neurophysiol, 2002, 87: 2896-2903) and therefore the inventors tested whether the NMDA receptor/TRPM4 interaction inhibitors provide neuroprotection against ROS toxicity.

Primary neurons plated in 24 well plates were infected with rAAV-hSyn-mCherry on DIV3 and challenged with 300 μM H₂O₂ on DIV10. All compounds were added to the culture 30 min prior to H₂O₂ insult. Images were acquired following the glutamate application for 24 h with a 2 h interval, where 9-16 images were obtained via a 20×objective for each condition at each time point. Cell deaths were quantified by analyzing the existence of nuclear localized mCherry via the Basic Analyzer with Incucyte® 2021 software, size and average intensity were used as exclusion criteria.

Using this assay, the inventors demonstrated that prior art compound P401 (WO 2020/079244) is provides better protection against H₂O₂ insult than the FDA-approved ALS drug Riluzole and Edaravone. Furthermore, a compound according to the present invention (compound 220) provides even better protection than the prior art compound P401.

### Example 55: Co-Immunoprecipitation and Disruption of NMDAR/TRPM4 Complex Formation

### Co-Immunoprecipitation

All procedures for co-immunoprecipitation were carried out at 4° C. Human iPSC derived brain organoids were cultured in Falcon@6 well TC-treated cell culture plate to week 25 as described (Bauersachs H G et al, Neuroscience, 2022; 484:83-97), which allows an abundant expression of both GluN2A/GluN2B/TRPM4 expression. 6 brain organoids were incubated with 10 μM compound 120 for 30 min, washed twice with ice-cold PBS and then lysed in 500 μL immunoprecipitation buffer (10 mM Tris, pH 8.0, 150 mM NaCl, 1 mM EDTA, 1% NP-40, 10% glycerol with EDTA-free Protease Inhibitor Cocktail (Roche). The lysate was incubated while rotating for 60 min, centrifuged for 12 min at 1,200×g to remove cell debris and nuclei. 10% of the supernatant was boiled with Laemmli buffer for input samples analysis. The rest of the supernatant was mixed with anti-TRPM4 antibody (1:200) overnight. The mixture was then incubated with Pierce™ Protein A Magnetic Beads for another 12 h to pull down antibody complex, and washed with immunoprecipitation buffer to remove any unspecific bindings. After the last washing, the supernatant was carefully removed, and the precipitate pellets were boiled in Laemmli buffer. Both input and precipitate samples were stored at −20° C. until analysed with western blots.

### Western Blots

Input samples and/or immunoprecipitates were separated in 7.5% sodium dodecyl sulphate and polyacrylamide gel (SDS-PAGE), transferred onto a 0.45 μm nitrocellulose membrane, and finally blotted with indicated antibodies. Running buffer (in mM: 190 glycine, 25 Tris, 0.1% SDS) and transfer buffer (in mM: 150 glycine, 20 Tris, 0.1% SDS and 20% Methanol) were diluted from 10× stocks.

Using this co-immunoprecipitation procedure, the inventors demonstrated that Compound 120 disrupts the NMDAR/TRPM4 complex.

### Example 56: Protection of Human iPSC-Derived Organoids

To explore the efficacy of the inventive compounds in a wide range of neurodegenerative diseases, several human induced pluripotent stem cells (iPSCs)-based in vitro models have been or will be used.

Human iPSCs-Derived Prefrontal Cortex Organoids

Prefrontal cortex organoids were generated based on published literature (Bauersachs H G et al, Neuroscience, 2022; 484:83-97). Around week 20, organoids were pre-incubated with compound 120 for 30 min before challenged with 200 μM NMDA for 24 h, where 80% of neuron will undergo necrosis. The cell death was monitored and analyzed with a RealTime-Glo™ MT Cell Viability Assay (Promega, G9711) following the manufacture's instruction using a plate reader (luciferase). The cell survival (%) can be calculated by the following equation:

$$Cell survival ( \% ) = \frac{{F}_{t}}{{F}_{basal}} / \frac{{F}_{Control}}{{F}_{ControlBasal}} * 100 \%$$

Where Fₜ is the luciferase intensity at each time point and F_{basal} is the basal luciferase intensity before glutamate/NMDA insult to diminish the difference between different organoids. The Cₒₙₜᵣₒₗ and F_{ControlBasal} stands for the same in untreated organoids, therefore the cell death can be calculated by normalization to the healthy organoids.

Using this test, the inventors demonstrated that Compound 120 improves cell survival after glutamate/NMDA treatment in human iPSC-derived prefrontal cortex organoids as compared to compound P401 known from the prior art (WO 2020/079244).

Human iPSCs-Derived Motor Neurons

Human iPSCs-derived motor neurons cultures will be generated from healthy, sporadic ALS, SOD1&TDP43&C9orf72 mutation related ALS based on publications (Horner S J et al, Cells, 2021; 10 (12); Du Z W et al, Nat Commun., 2015; 6:6626; Shi Y et al, JCI Insight. 2019; 5). The iPSCs will be cultured and differentiated to motor neurons, and they will be treated with 10 μM Glutamate to induce glutamate neurotoxicity and cell death on day 17 of differentiation with or without compounds of the invention. Tracking of neuronal survival will be performed by Incucyte with a mCherry-NLS expressed in the nuclear. The survival of neurons will be calculated as dead neurons are no longer detectable by the mCherry fluorescence in the nuclear.

### Example 57: Mice Models of ALS

Heterozygous SOD1G93A, C9orf72 and TDP43 transgenic mice on a C57BL/6 background (Jackson Laboratory, 004435) will be used in this study (Gurney et al., Science. 1994; 264(5166):1772-5; Pitzer C et al., Brain, 2008; 131 (Pt 12): 3335-47). The heterozygous was maintained by mating heterozygous transgenic males with C57BL/6 wild-type females. They will be housed in groups (maximally three mice/cage) and kept in standard cages (15×21×13.5 cm) on a 12:12 h light:dark cycle with ad libitum access to food, water, and nesting material. Animals will be randomly allocated to treatment groups. Compounds of the invention will be given to animals before and after the disease onset at different doses (in mg per kilo body weight per day: 0.1, 0.3, 1, 3, 10, 30). The humane endpoint is defined as the mouse's inability to rectify itself in 30 s and examined daily after paralysis was developed, without knowing the treatment group.

### Example 58: Mouse Model of Retinal Ganglion Cell (RGC) Degeneration

C57BL/6J mice will receive vehicle (40% Propylene Glycol) or compounds according to the present invention as set out above (40 mg/kg body weight, dissolved in 40% Propylene Glycol) through intraperitoneal injection at −16 h, −3 h, 0 h (intravitreal NMDA/saline injection), +3 h and +24 h in a volume of 50 μL per injection. At 0 h, mice received 20 nmol of NMDA/Glutamate (total volume 2.0 μL) by intravitreal injection in the left eye and saline (total volume 2.0 μL) in the right eye. Both eyes will be removed from euthanized mice 7 days after intravitreal injections and fixed in formalin for 15 min before retinas were dissected and processed for whole-mount immunohistochemistry. Retinas will be incubated in blocking solution (10% normal donkey serum and 1% Triton-X 100 in PBS) for 6 h, followed by 24 h incubation with anti-Brn3a antibody in blocking solution at 4° C. Retinas will then be washed 3 times with PBS and incubated with donkey anti-rabbit Alexa Fluor-594 for 24 h at room temperature. Retinas will be washed again, cut, and mounted onto slides. For each retina, images will be obtained from eight fields (554 μm×554 μm) around the peripheral retina (two from each quadrant located ˜600 μm and ˜1400 μm from the macular hole) to minimize the influence of location-associated variability in RGC density on cell counts. All images will be obtained using Las X software via an HC PL APO 20× objective on a Leica TCS SP8LIA in a DM6 CFS upright confocal microscope. Brn3a-positive cells will be identified and counted with a macro in CellProfiler. Data analysis will be performed on a single-blind basis without knowledge.

### Example 59: Further Compounds

In a further round of synthesis, the inventors will synthesize the following inventive compounds and assess their activity:

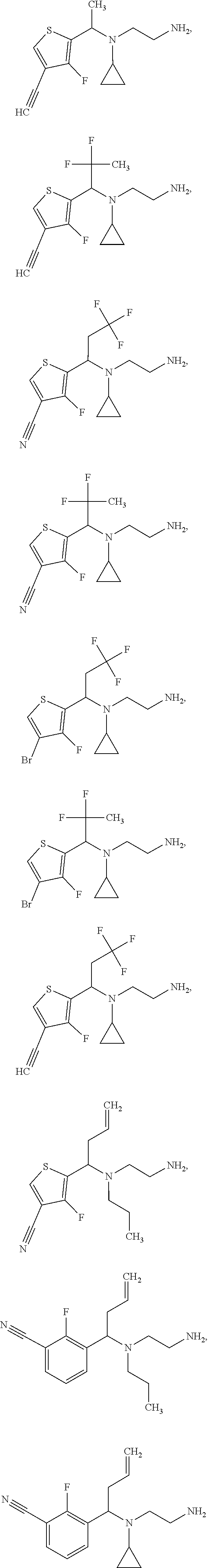

1. A compound according to the following general formula I: 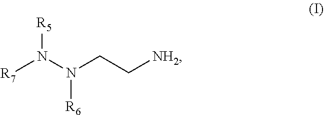
wherein:
R₇ is selected from 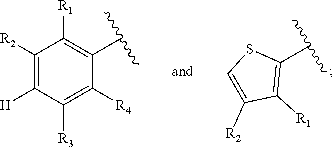
R₁, R₂, R₃ and R₄ are each independently selected from H, F, Cl, Br, I, —CN and ethynyl, and wherein at least one of R₁, R₂, R₃ and R₄ is selected from: F, Cl, Br, I, —CN and ethynyl;
R₅ is selected from unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl;
R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the proviso that if R₅ is methyl, R₇ is 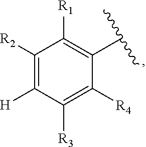
one of R₂ and R₃ is H and the other is F, Cl or —CN and R₁ and R₄ are H, then R₆ is selected from unsubstituted linear C₃-C₆ alkyl, unsubstituted branched C₄-C₆ alkyl, substituted branched or linear C₂-C₆ alkyl, unsubstituted C₄-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if R₅ is methyl, R₇ is 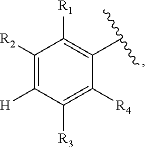
two of R₁, R₂, R₃ and R₄ are Cl, while the other two are H, wherein either R₁ and R₂, R₃ and R₄, R₁ and R₃ or R₂ and R₄ are Cl, then R₆ is selected from unsubstituted branched or linear C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the proviso that if Rₛ is methyl, R₇ is 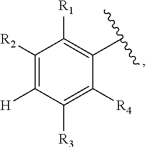
one of R₁ and R₄ is H and the other is F or Br and R₂ and R₃ are H, then R₆ is selected from unsubstituted linear C₃-C₆ alkyl, unsubstituted branched C₄-C₆ alkyl, substituted branched or linear C₂-C₆ alkyl, unsubstituted C₄-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if R₅ is ethyl, R₇ is 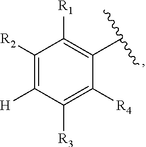
one of R₂ and R₃ is H and the other is Cl and R₁ and R₄ are H, then R₆ is selected from unsubstituted linear C₃-C₆ alkyl, unsubstituted branched C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
wherein substituted alkyl refers to alkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted cycloalkyl refers to cycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted bicycloalkyl refers to bicycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted alkylcycloalkyl refers to alkylcycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted alkenyl refers alkenyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH3, —C(O)N(CH₃)₂, —OC(O)CH₃, —NHC(O)CH₃, —S(O)₂CH₃, or —S(O)₂NH₂;
and a pharmaceutically acceptable salt, racemate, (R)- or (S)-enantiomer of any of these compounds.

2-3. (canceled)

4. The compound according to claim 1, wherein R₁, R₂, R₃ and R₄ are each independently selected from H, F, Cl, Br, I and —CN.

5. The compound according to claim 1, wherein at least one of R₁, R₂, R₃ and R₄ is ethynyl.

6. The compound according to claim 1, wherein two of R₁,_R₂, R₃ and R₄ are each independently selected from H, F, Cl, Br, I, —CN and ethynyl.

7. The compound according to claim 1, wherein R₇ is 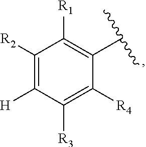
and wherein one of R₂ and R₃ is selected from H, F, Cl, Br, I, —CN and ethynyl, while the other is H.

8. The compound according to claim 1, wherein R₇ is 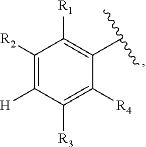
wherein at least two of R₁, R₂, R₃ and R₄ are H and one of R₂ and R₃ is Cl.

9. The compound according to claim 1, wherein R₁ is H or F and wherein R₂ is selected from F, Cl, Br, I, CN and ethynyl.

10. (canceled)

11. The compound according to claim 1, wherein R₇ is 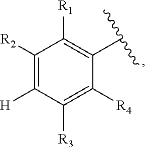
and wherein R₄ is H or F and wherein R₃ is selected from F Cl, Br, I, CN and ethynyl.

12. (canceled)

13. The compound according to claim 1, wherein i) R₁ is F, R₂ is Cl and R₃ and R₄ are H, or wherein ii) R₁ and R₂ are H, R₃ is Cl and R₄ is F.

14. (canceled)

15. The compound according to claim 1, wherein R₅ is selected from unsubstituted branched or linear C₁-C₄ alkyl, fluoro-substituted branched or linear C₁-C₄ alkyl, unsubstituted C₃-C₆ cycloalkyl, and fluoro-substituted C₃-C₆ cycloalkyl.

16-17. (canceled)

18. The compound according to claim 1, wherein R₅ is selected from methyl, ethyl, isopropyl, —CH₂CF₃, —CF₂CF₃, —CF₂CH₃, —CHF₂, —CF₃, cyclopropyl, fluoro-substituted isopropyl, propenyl, cyclopropyl, cyclobutyl, fluoro-substituted cyclobutyl, and cyclopentyl.

19. The compound according to claim 1, wherein R₆ is selected from unsubstituted branched or linear C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl; and wherein the substituents of substituted branched or linear C₂-C₆ alkyl, substituted C₃-C₆ cycloalkyl, substituted C₄-C₈ bicycloalkyl, substituted C₄-C₇ alkylcycloalkyl, and substituted C₃-C₆ alkenyl are preferably each independently selected from F, Cl, CN, OH, alkylthio, and alkoxy.

20-22. (canceled)

23. The compound according to claim 1, wherein the compound has one of the following formulas 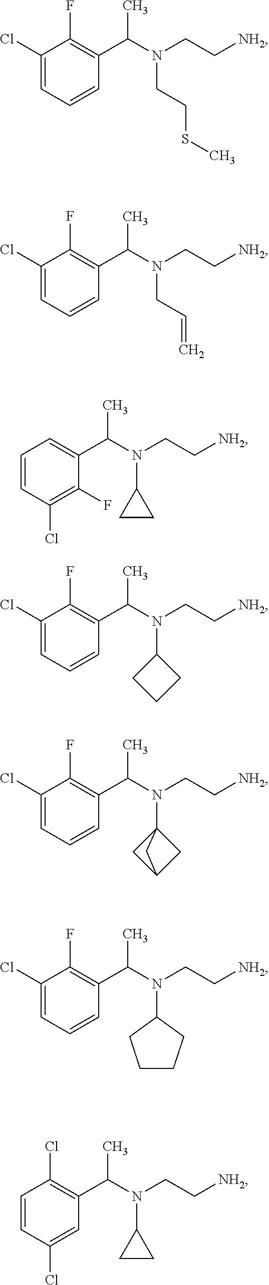 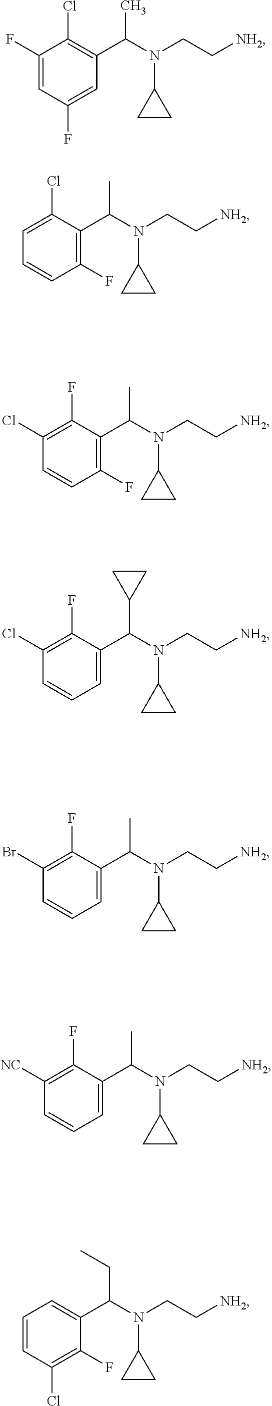 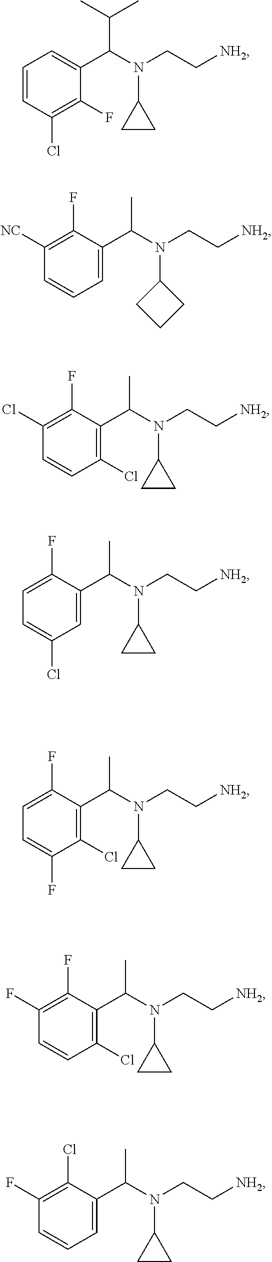 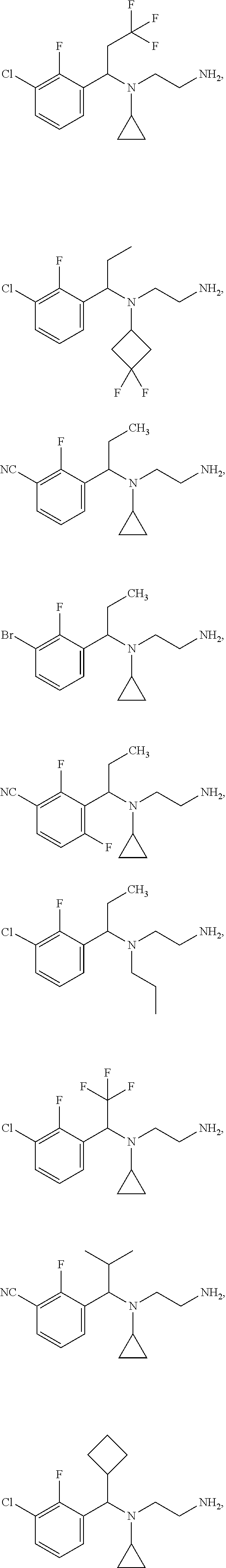 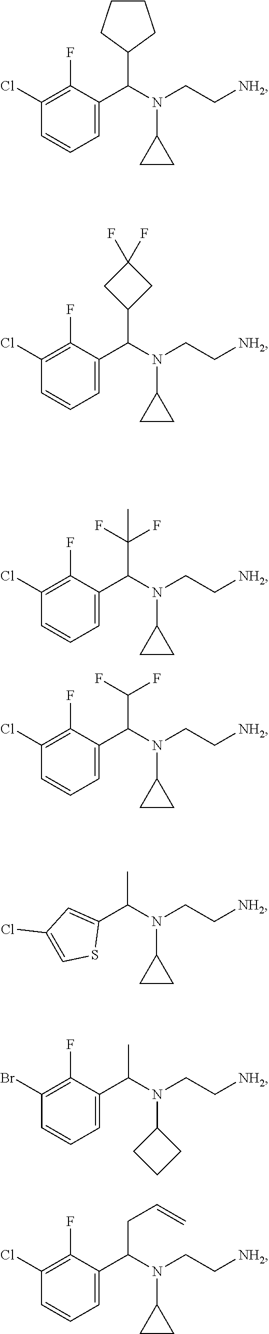 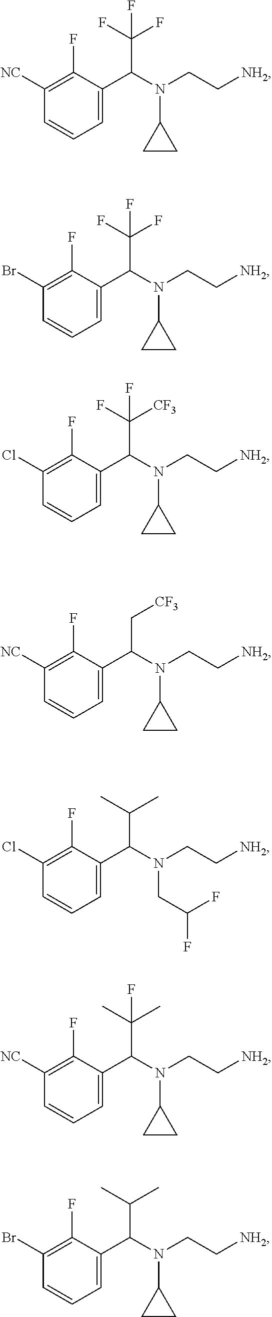 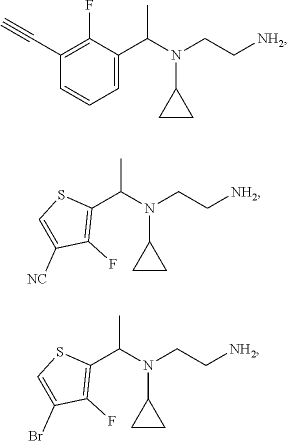 [SMILES: C#Cc1cccc(C(C)N(CCN)C2CC2)c1F ; CC(c1scc(C#N)c1F)N(CCN)C1CC1 ; CC(c1scc(Br)c1F)N(CCN)C1CC1] 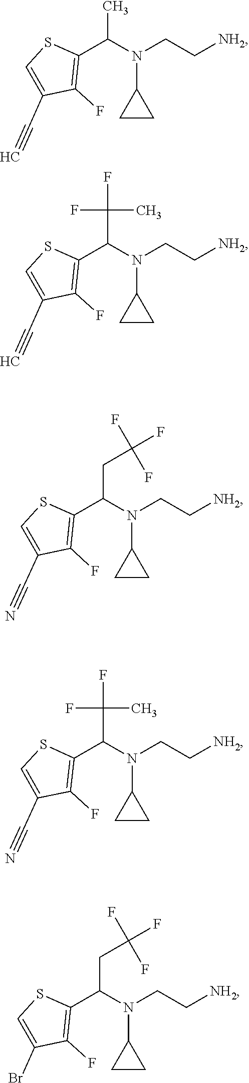 [SMILES: C#Cc1csc(C(C)N(CCN)C2CC2)c1F ; C#Cc1csc(C(N(CCN)C2CC2)C(C)(F)F)c1F ; N#Cc1csc(C(CC(F)(F)F)N(CCN)C2CC2)c1F ; CC(F)(F)C(c1scc(C#N)c1F)N(CCN)C1CC1 ; NCCN(C1CC1)C(CC(F)(F)F)c1scc(Br)c1F] 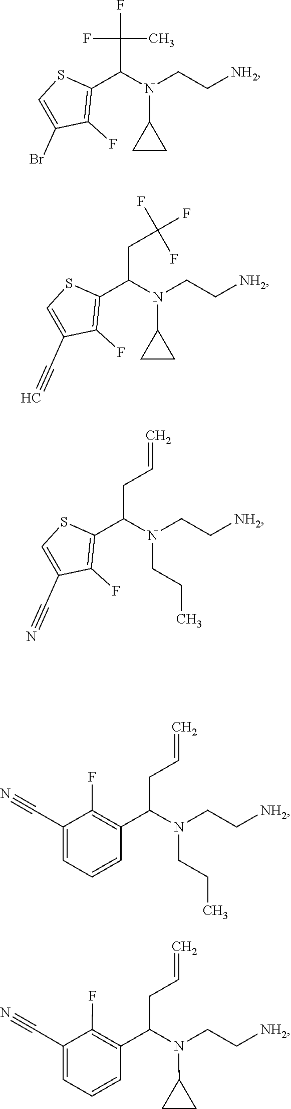
and a pharmaceutically acceptable salt, racemate, (R)- or (S)-enantiomer of any of these compounds.

24. (canceled)

25. The compound according to claim 1, wherein the pharmaceutically acceptable salt is selected from halides, formiates and trifluoroacetates.

26. A method for treating or preventing a disease of the human or animal body, comprising administering to a subject a compound according to the following general formula I: 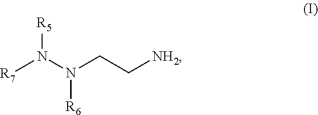
wherein:
R₇ is selected from 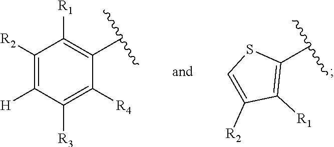
and wherein
R₁, R₂, R₃ and R₄ are each independently selected from H, F, Cl, Br, I, —CN and ethynyl;
R₅ is selected from unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl;
R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the proviso that if one of R₁ and R₄ is H and the other is Cl, and R₂, R₃, and R₅ are H, then R₆ is selected from unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if one of R₂ and R₃ is H and the other is Br, Cl or I, and R₁, R₄, and R₅ are H, then R₆ is selected from unsubstituted C₃-C₆ cycloalkyl, substituted C₃-C₆cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
wherein substituted alkyl refers to alkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted cycloalkyl refers to cycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted bicycloalkyl refers to bicycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted alkylcycloalkyl refers to alkylcycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted alkenyl refers alkenyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
or wherein the compound is a pharmaceutically acceptable salt, racemate, (R)- or (S)-enantiomer of a compound according to formula I.

27-47. (canceled)

48. The method according to claim 26, wherein the compound is a compound according to any one of claims 1, 4 to 9, 11, 13, 15, 18, 19, 23 and 25.

49-51. (canceled)

52. The method according to claim 26, wherein the disease is selected from the group consisting of a neurological disease and a neurodegenerative disease.

53-54. (canceled)

55. The method according to claim 26, wherein the disease is selected from the group consisting of stroke, Alzheimer's disease (AD), amyotrophic lateral sclerosis (ALS), Huntington's disease (HD), traumatic brain injury, post traumatic brain injury, absent-mindedness, age-related loss of memory, aging-related memory decline, progressive nuclear palsy, multiple sclerosis, thalamic degeneration, glutamate induced excitotoxicity, dystonia, epilepsy, optic nerve disease, diabetic retinopathy, glaucoma, pain, anti-NMDA receptor encephalitis, viral encephalopathy, dementia, such as post stroke dementia, HIV dementia, Creutzfeldt-Jakob dementia, dementia with Lewy bodies (DLB), dementia with degeneration of the frontal lobes including Pick's disease, dementia with corticobasal degeneration, vascular dementia, microangiopathy, Binswanger's disease, cerebral ischemia, hypoxia, Parkinson's disease, Batten disease, schizophrenia, Korsakoffs psychosis, depression, cerebral malaria, toxoplasmosis (due to the risk of toxoplasmosis-associated brain damage), HIV infection/AIDS (due to the risk of HIV)-associated brain damage, Zika virus infection (due to the possibility of Zika virus-associated brain damage), other viral infection potentially leading to neurodegenerative events and corresponding neuronal or brain damage, respectively, such as viral meningitis or SARS-COV2 virus induced encephalitis; brain tumour, diseases of the central nervous system such as states of anxiety, tension and depression, sexual dysfunction disorders, sleep disorders, pathological disturbances of the intake of food, stimulants and addictive substances.

56. A compound according to the following general formula II: 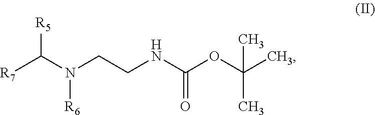
wherein:
R₇ is selected from 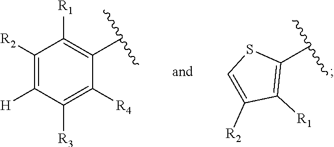
R₁, R₂, R₃ and R₄ are each independently selected from H, F, Cl, Br, I, —CN and ethynyl, and wherein at least one of R₁, R₂, R₃ and R₄ is selected from: F, Cl, Br, I, —CN and ethynyl;
R₅ is selected from unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl;
R₆ is selected from H, unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
with the proviso that if R₅ is methyl, R₇ is 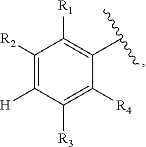
one of R₂ and R₃ is H and the other is Cl and R₁ and R₄ are H, then R₆ is selected from unsubstituted linear C₃-C₆ alkyl, unsubstituted branched C₄-C₆ alkyl, substituted branched or linear C₂-C₆ alkyl, unsubstituted C₄-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if R₅ is methyl, R₇ is 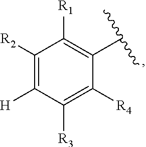
two of R₁, R₂, R₃ and R₄ are Cl, while the other two are H, wherein either R₁ and R₂, R₃ and R₄, R₁ and R₃ or R₂ and R₄ are Cl, then R₆ is selected from unsubstituted branched or linear C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if R₅ is methyl, R₇ is 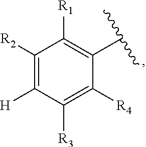
R₁ and R₄ are Cl, one of R₂ and R₃ is H and the other is F, then R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if Rₛ is methyl, R₇ is 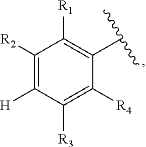
one of R₁ and R₄ is H and the other is F, and R₂ and R₃ are Cl, then R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if R₅ is methyl, one of R₁ and R₄ is H and the other is Cl, and R₂ and R₃ are H, then R₆ is selected from unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl;
with the further proviso that if R₅ is ethyl, one of R₂ and R₃ is H and the other is Cl and R₁ and R₄ are H, then R₆ is selected from unsubstituted linear C₃-C₆ alkyl, unsubstituted branched C₃-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
wherein substituted alkyl refers to alkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted cycloalkyl refers to cycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted bicycloalkyl refers to bicycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted alkylcycloalkyl refers to alkylcycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted alkenyl refers alkenyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂; 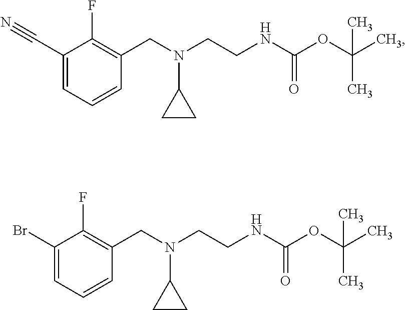 [SMILES: CC(C)(C)OC(=O)NCCN(Cc1cccc(C#N)c1F)C1CC1 ; CC(C)(C)OC(=O)NCCN(Cc1cccc(Br)c1F)C1CC1]
and a salt, racemate, (R)- or (S)-enantiomer, hydrate or isotope of any of these compounds.

57. A compound according to the following general formula III or formula IV: 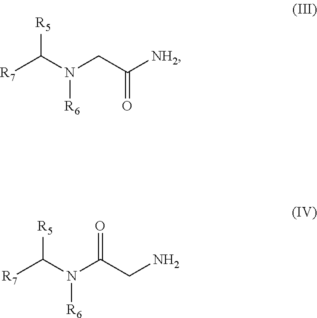
wherein:
R₇ is selected from 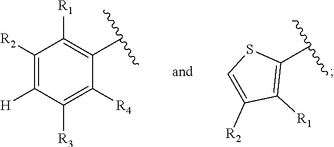
R₁, R₂, R₃ and R₄ are each independently selected from H, F, Cl, Br, I, —CN and ethynyl, and wherein at least one of R₁, R₂, R₃ and R₄ is selected from: F, Cl, Br, I, —CN and ethynyl;
R₅ is selected from unsubstituted branched or linear C₁-C₄ alkyl, fluoro-substituted branched or linear C₁-C₄ alkyl, unsubstituted propenyl, unsubstituted C₃-C₆ cycloalkyl, and fluoro-substituted C₃-C₆ cycloalkyl;
R₆ is selected from H, unsubstituted branched or linear C₂-C₆ alkyl, substituted branched or linear C₂-C₆ alkyl, unsubstituted C₃-C₆ cycloalkyl, substituted C₃-C₆ cycloalkyl, unsubstituted C₄-C₈ bicycloalkyl, substituted C₄-C₈ bicycloalkyl, unsubstituted C₄-C₇ alkylcycloalkyl, substituted C₄-C₇ alkylcycloalkyl, unsubstituted C₃-C₆ alkenyl and substituted C₃-C₆ alkenyl,
wherein substituted alkyl refers to alkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted cycloalkyl refers to cycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted bicycloalkyl refers to bicycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted alkylcycloalkyl refers to alkylcycloalkyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
wherein substituted alkenyl refers alkenyl where one or more hydrogen atoms have been independently replaced by —OH, —F, —Cl, —Br, —I, —NH₂, —NO₂, —CO₂H, —CO₂CH₃, —CN, —OCH₃, —SCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, or —S(O)₂NH₂;
and a salt, racemate, (R)- or (S)-enantiomer, hydrate or isotope thereof.

58. (canceled)

59. The method according to claim 55, wherein i) stroke is selected from ischemic stroke and hemorrhagic stroke, ii) pain is neuropathic pain, iii) schizophrenia is schizophrenia with dementia, and iv) the brain tumour is glioblastoma.
